(19) United States
(12) Patent Application Publication
(10) Pub. No.: US 2020/0069764 A1
(43) Pub. Date: Mar. 5, 2020

(54) ANTIBODIES AND ANTIBODY FRAGMENTS FOR SITE-SPECIFIC CONJUGATION

(71) Applicant: Pfizer Inc., New York, NY (US)

(72) Inventors: Dangshe MA, Millwood, NY (US); Kimberly Ann MARQUETTE, Somerville, MA (US); Edmund Idris GRAZIANI, Chestnut Ridge, NY (US); Puja SAPRA, River Edge, NJ (US); Lawrence Nathan TUMEY, Pawcatuk, CT (US); Nadira Anarkali PRASHAD, New City, NY (US); Kiran Manohar KHANDKE, Nanuet, NY (US); Eric M. BENNETT, Arlington, MA (US); Lioudmila TCHISTIAKOVA, Stoneham, MA (US)

(73) Assignee: Pfizer Inc., New York, NY (US)

(21) Appl. No.: 16/688,173

(22) Filed: Nov. 19, 2019

Related U.S. Application Data
(63) Continuation of application No. 15/356,953, filed on Nov. 21, 2016, now abandoned.
(60) Provisional application No. 62/409,323, filed on Oct. 17, 2016.
(60) Provisional application No. 62/289,744, filed on Feb. 1, 2016.
(60) Provisional application No. 62/260,854, filed on Nov. 30, 2015.

(51) Int. Cl. A61K 38/05; C07K 16/28; C07K 16/32; A61K 47/68

(52) U.S. Cl. CPC A61K 38/05; C07K 16/283; C07K 16/32; A61K 47/6851; A61K 47/6803; C07K 2317/52; C07K 2317/92; C07K 2317/732; C07K 2317/24; C07K 2317/94; C07K 2317/73; A61K 47/6855

(57) ABSTRACT

The invention relates to polypeptides, antibodies, and antigen-binding fragments thereof, that comprise a substituted cysteine for site-specific conjugation.

### CROSS-REFERENCE TO RELATED APPLICATIONS

This application claims the benefit of U.S. provisional application 62/260,854, filed Nov. 30, 2015; U.S. provisional application 62/289,744, filed Feb. 1, 2016; and U.S. provisional application 62/409,323, filed Oct. 17, 2016. The complete content of all of the above-referenced patent applications are hereby incorporated by reference for all purposes.

### REFERENCE TO SEQUENCE LISTING

This application contains a Sequence Listing which has been submitted electronically in ASCII format and is hereby incorporated by reference in its entirety. Said ASCII copy, created on Nov. 11, 2016, is named PC72296A_Seq_Listing_ST25.txt and is 146,747 bytes in size.

### FIELD OF THE INVENTION

This invention relates to antibodies, and antigen-binding fragments thereof, engineered to introduce amino acids for site-specific conjugation.

### BACKGROUND OF THE INVENTION

Antibodies have been conjugated to a variety of cytotoxic drugs, including small molecules that alkylate DNA (e.g., duocarmycin and calicheamicin), disrupt microtubules (e.g., maytansinoids and auristatins) or bind DNA (e.g., anthracyclins). One such antibody-drug conjugate (ADC) comprising a humanized anti-CD33 antibody conjugated to calicheamicin—Mylotarg™ (gemtuzumab ozogamicin)—has been approved for treating acute myeloid leukemia. Adcetris™ (brentuximab vedotin), an ADC comprising a chimeric antibody to CD30 conjugated to the auristatin monomethyl auristatin E (MMAE) has been approved for treatment of Hodgkin's lymphoma and anaplastic large cell lymphoma.

Although ADCs hold promise for cancer therapy, cytotoxic drugs are generally conjugated to the antibodies via lysine side chains or by reducing interchain disulfide bonds present in the antibodies to provide activated cysteine sulfhydryl groups. This non-specific conjugation approach, however, has numerous drawbacks. For example, drug conjugation to antibody lysine residues is complicated by the fact that there are many lysine residues (˜30) in an antibody available for conjugation. Since the optimal number of drug to antibody ratio (DAR) is much lower (e.g., around 4:1), lysine conjugation often generates a very heterogeneous profile. Furthermore, many lysines are located in critical antigen binding sites of CDR region and drug conjugation may lead to a reduction in antibody affinity. On the other hand, while thiol mediated conjugation mainly targets the eight cysteines involved in hinge disulfide bonds, it is still difficult to predict and identify which four of eight cysteines are consistently conjugated among the different preparations.

Recently, genetic engineering of free cysteine residues has enabled site-specific conjugation with thiol-based chemistries. The site-specific ADCs have homogeneous profiles and well-defined conjugation sites, and showed potent in vitro cytotoxicity and strong in vivo antitumor activity.

WO 2013/093809 discloses engineered antibody constant regions (Fc, Cγ, Cκ, Cλ), or a fragment thereof, that comprise amino acid substitutions at specific sites to introduce a cysteine residue for conjugation. A number of Cys-mutation sites in IgG heavy chain and lambda/kappa light chain constant regions are disclosed.

The success of using introduced Cys residues for site-specific conjugation relies on the ability to select proper sites in which Cys-substitution does not alter protein structure or function. Further, using different conjugation sites result in different characteristics, such as biological stability of the ADC, or conjugatability. Therefore, a site-specific conjugation strategy which generates an ADC with a defined conjugation site and desired ADC characteristics would be highly useful.

### SUMMARY OF THE INVENTION

The invention relates to polypeptides, antibodies, and antigen-binding fragments thereof, that comprise a substituted cysteine for site-specific conjugation.

Those skilled in the art will recognize, or be able to ascertain using no more than routine experimentation, many equivalents to the specific embodiments of the invention described herein. Such equivalents are intended to be encompassed by the following embodiments (E).
E1. A polypeptide comprising an antibody heavy chain constant domain comprising an engineered cysteine residue at position 290, according to the numbering of the EU index of Kabat.
E2. The polypeptide of E1, wherein said constant domain comprises an IgG heavy chain CH₂ domain.
E3. The polypeptide of E2, wherein said IgG is IgG₁, IgG₂, IgG₃, or IgG₄.
E4. A polypeptide comprising an antibody heavy chain constant domain comprising an engineered cysteine residue at a position corresponding to residue 60 of SEQ ID NO:61, when said constant domain is aligned with SEQ ID NO:61.
E5. The polypeptide of E4, wherein the engineered cysteine residue is located at position 290 of an IgG CH₂ domain, according to the numbering of the EU index of Kabat.
E6. The polypeptide of E5, wherein said IgG is IgG₁, IgG₂, IgG₃, or IgG₄.
E7. The polypeptide of E1 or E4, wherein said constant domain comprises an IgA (e.g., IgA₁ or IgA₂) heavy chain CH₂ domain.
E8. The polypeptide of E1 or E4, wherein said constant domain comprises an IgD heavy chain CH₂ domain.
E9. The polypeptide of E1 or E4, wherein said constant domain comprises an IgE heavy chain CH₂ domain.
E10. The polypeptide of E1 or E4, wherein said constant domain comprises an IgM heavy chain CH₂ domain.
E11. The polypeptide of any one of E1-E10, wherein said constant domain is a human antibody constant domain.
E12. The polypeptide of any one of E1-E11, wherein said constant domain further comprises an engineered cysteine residue at a position selected from the group consisting of: 118, 246, 249, 265, 267, 270, 276, 278, 283, 292, 293, 294, 300, 302, 303, 314, 315, 318, 320, 332, 333, 334, 336, 345, 347, 354, 355, 358, 360, 362, 370, 373, 375, 376, 378, 380, 382, 386, 388, 390, 392, 393, 401, 404, 411, 413, 414, 416, 418, 419, 421, 428, 431, 432, 437, 438, 439, 443, 444, and any combination thereof, according to the numbering of the EU index of Kabat.
E13. The polypeptide of any one of E1-E12, wherein said constant domain further comprises an engineered cysteine residue at a position selected from the group consisting of: 118, 334, 347, 373, 375, 380, 388, 392, 421, 443, and any combination thereof, according to the numbering of the EU index of Kabat.
E14. The polypeptide of any one of E1-E13, wherein said constant domain further comprises an engineered cysteine residue at position 334, according to the numbering of the EU index of Kabat.
E15. An antibody or antigen binding fragment thereof comprising a polypeptide of any one of E1-E14.
E16. An antibody or antigen binding fragment thereof comprising:
   (a) a polypeptide of one of E1-E14, and
   (b) an antibody light chain constant region comprising (i) an engineered cysteine residue at position 183, according to the numbering of Kabat; or (ii) an engineered cysteine residue at a position corresponding to residue 76 of SEQ ID NO:63, when said constant domain is aligned with SEQ ID NO:63.
E17. An antibody or antigen binding fragment thereof comprising:
   (a) a polypeptide of one of E1-E14, and
   (b) an antibody light chain constant region comprising (i) an engineered cysteine residue at position 110, 111, 125, 149, 155, 158, 161, 185, 188, 189, 191, 197, 205, 206, 207, 208, 210, or any combination thereof, according to the numbering of Kabat; (ii) an engineered cysteine residue at a position corresponding to residue 4, 42, 81, 100, 103, or any combination thereof, of SEQ ID NO:63, when said constant domain is aligned with SEQ ID NO:63 (kappa light chain); or (iii) an engineered cysteine residue at a position corresponding to residue 4, 5, 19, 43, 49, 52, 55, 78, 81, 82, 84, 90, 96, 97, 98, 99, 101, or any combination thereof, of SEQ ID NO:64, when said constant domain is aligned with SEQ ID NO:64 (lambda light chain).
E18. The antibody or antigen binding fragment thereof of E16 or E17, wherein said light chain constant region comprises a kappa light chain constant domain (CLκ).
E19. The antibody or antigen binding fragment thereof of E16 or E17, wherein said light chain constant region comprises a lambda light chain constant domain (CLλ).
E20. A compound comprising the polypeptide of any of E1-E14,or the antibody or antigen binding fragment thereof of any of E15-E19, wherein the polypeptide or antibody is conjugated to one or more therapeutic agents via said engineered cysteine site.
E21. The compound of E20, wherein the therapeutic agent is conjugated to the polypeptide or the antibody or antigen binding fragment thereof via a linker.
E22. A polypeptide comprising an antibody heavy chain constant domain that comprises an engineered cysteine residue at a position selected from the group consisting of: 334, 375, 392, and any combination thereof, according to the numbering of the EU index of Kabat.
E23. The polypeptide of E22, wherein constant domain comprises an IgG heavy chain CH₂ domain, CH₃ domain, or both.
E24. A polypeptide comprising an antibody heavy chain constant domain comprising an engineered cysteine residue at a position corresponding to residue 104, 145, 162, or any combination thereof, of SEQ ID NO:62, when said constant domain is aligned with SEQ ID NO:62.
E25. The polypeptide of E24, wherein the engineered cysteine residue is located at position 334, 375, 392, or any combination thereof, of an IgG CH₂ domain, CH₃ domain, or both, according to the numbering of the EU index of Kabat.
E26. The polypeptide of E22 or E24, wherein said constant domain comprises an IgA (e.g., IgA₁ or IgA₂), IgD, IgE, or IgM heavy chain CH₂ domain, CH₃ domain, or both.
E27. A polypeptide comprising an antibody heavy chain constant domain that comprises an engineered cysteine residue at a position selected from the group consisting of: 347, 388, 421, 443, and any combination thereof, according to the numbering of the EU index of Kabat.
E28. The polypeptide of E27, wherein constant domain comprises an IgG CH₃ domain.
E29. A polypeptide comprising an antibody heavy chain constant domain comprising an engineered cysteine residue ata position corresponding to 117, 158, 191, 213, or any combination thereof, of SEQ ID NO:62, when said constant domain is aligned with SEQ ID NO:62.
E30. The polypeptide of E29, wherein the engineered cysteine residue is located at position 347, 388, 421, 443, or any combination thereof, of an IgG CH₃ domain, according to the numbering of the EU index of Kabat.
E31. The polypeptide of E27 or E29, wherein said constant domain comprises an IgA (e.g., IgA₁ or IgA₂), IgD, IgE, or IgM heavy chain CH₃ domain.
E32. A polypeptide comprising an antibody heavy chain constant domain that comprises an engineered cysteine residue at a position selected from the group consisting of: 347, 388, 421, and any combination thereof, according to the numbering of the EU index of Kabat.
E33. The polypeptide of E32, wherein constant domain comprises an IgG heavy chain CH₃ domain.
E34. A polypeptide comprising an antibody heavy chain constant domain comprising an engineered cysteine residue at a position corresponding to residue 117, 158, 191, or any combination thereof, of SEQ ID NO:62, when said constant domain is aligned with SEQ ID NO:62.
E35. The polypeptide of E34, wherein the engineered cysteine residue is located at position 347, 388, 421, or any combination thereof, of an IgG CH₃ domain, according to the numbering of the EU index of Kabat.
E36. The polypeptide of E32 or E34, wherein said constant domain comprises an IgA (e.g., IgA₁ or IgA₂), IgD, IgE, or IgM heavy chain CH₃ domain.
E37. A polypeptide comprising an antibody heavy chain constant domain that comprises an engineered cysteine residue at a position selected from the group consisting of: 290, 334, 392, 443, and any combination thereof, according to the numbering of the EU index of Kabat.
E38. The polypeptide of E37, wherein constant domain comprises an IgG heavy chain CH₂ domain, CH₃ domain, or both.
E39. A polypeptide comprising an antibody heavy chain constant domain comprising an engineered cysteine residue at a position corresponding to residue 60, 104, 162, 213, or any combination thereof, of SEQ ID NO:62, when said constant domain is aligned with SEQ ID NO:62.
E40. The polypeptide of E39, wherein the engineered cysteine residue is located at position 290, 334, 392, 443, or any combination thereof, of an IgG CH₂ domain, CH₃ domain, or both, according to the numbering of the EU index of Kabat.
E41. The polypeptide of E37 or E39, wherein said constant domain comprises an IgA (e.g., IgA₁ or IgA₂), IgD, IgE, or IgM heavy chain CH₂ domain, CH₃ domain, or both.
E42. A polypeptide comprising an antibody heavy chain constant domain that comprises an engineered cysteine residue at a position selected from the group consisting of: 334, 388, 421, 443, and any combination thereof, according to the numbering of the EU index of Kabat.
E43. The polypeptide of E42, wherein constant domain comprises an IgG heavy chain CH₂ domain, CH₃ domain, or both.
E44. A polypeptide comprising an antibody heavy chain constant domain comprising an engineered cysteine residue ata position corresponding to 104, 158, 191, 213, or any combination thereof, of SEQ ID NO:62, when said constant domain is aligned with SEQ ID NO:62.
E45. The polypeptide of E44, wherein the engineered cysteine residue is located at position 334, 388, 421, 443, or any combination thereof, of an IgG CH₂ domain, CH₃ domain, or both, according to the numbering of the EU index of Kabat.
E46. The polypeptide of E42 or E44, wherein said constant domain comprises an IgA (e.g., IgA₁ or IgA₂), IgD, IgE, or IgM heavy chain CH₂ domain, CH₃ domain, or both.
E47. A polypeptide comprising an antibody heavy chain constant domain that comprises an engineered cysteine residue at a position selected from the group consisting of: 334, 392, 421, and any combination thereof, according to the numbering of the EU index of Kabat.
E48. The polypeptide of E47, wherein constant domain comprises an IgG heavy chain CH₂ domain, CH₃ domain, or both.
E49. A polypeptide comprising an antibody heavy chain constant domain comprising an engineered cysteine residue at a position corresponding to 104, 162, 191, or any combination thereof, of SEQ ID NO:62, when said constant domain is aligned with SEQ ID NO:62.
E50. The polypeptide of E49, wherein the engineered cysteine residue is located at position 334, 392, 421, or any combination thereof, of an IgG CH₂ domain, CH₃ domain, or both, according to the numbering of the EU index of Kabat.
E51. The polypeptide of E47 or E49, wherein said constant domain comprises an IgA (e.g., IgA₁ or IgA₂), IgD, IgE, or IgM heavy chain CH₂ domain, CH₃ domain, or both.
E52. A polypeptide comprising an antibody heavy chain constant domain that comprises an engineered cysteine residue at position 392, according to the numbering of the EU index of Kabat.
E53. A polypeptide comprising an antibody heavy chain constant domain that comprises an engineered cysteine residue at position 290, 443, or both, according to the numbering of the EU index of Kabat.
E54. The polypeptide of E52 or E53, wherein the constant domain comprises an IgG heavy chain CH₂ domain, CH₃ domain, or both.
E55. A polypeptide comprising an antibody heavy chain constant domain comprising an engineered cysteine residue at a position corresponding to residue 162 of SEQ ID NO:62, when said constant domain is aligned with SEQ ID NO:62.
E56. The polypeptide E55, wherein the engineered cysteine residue is located at position 392 of an IgG CH₃ domain, according to the numbering of the EU index of Kabat.
E57. A polypeptide comprising an antibody heavy chain constant domain comprising an engineered residue at a position corresponding to residue 60, 213, or both, of SEQ ID NO:62, when said constant domain is aligned with SEQ ID NO:62.
E58. The polypeptide E57, wherein the engineered cysteine residue is located at position 290, 443, or both, of an IgG CH₂ domain, CH₃ domain, or both, according to the numbering of the EU index of Kabat.
E59. The polypeptide of any one of E52, E53, E55, and E57, wherein said constant domain comprises an IgA (e.g., IgA₁ or IgA₂), IgD, IgE, or IgM heavy chain CH₂ domain, CH₃ domain, or both.
E60. A polypeptide comprising an antibody heavy chain constant domain that comprises an engineered cysteine residue at a position selected from the group consisting of: 290, 388, 443, and any combination thereof, according to the numbering of the EU index of Kabat.
E61. The polypeptide of E60, wherein constant domain comprises an IgG heavy chain CH₂ domain, CH₃ domain, or both.
E62. A polypeptide comprising an antibody heavy chain constant domain comprising an engineered cysteine residue at a position corresponding to residue 60, 158, 213, or any combination thereof, of SEQ ID NO:62, when said constant domain is aligned with SEQ ID NO:62.
E63. The polypeptide of E62, wherein the engineered cysteine residue is located at residue 290, 388, 443, or any combination thereof, of an IgG CH₂ domain, CH₃ domain, or both, according to the numbering of the EU index of Kabat.
E64. The polypeptide of E60 or E62, wherein said constant domain comprises an IgA (e.g., IgA₁ or IgA₂), IgD, IgE, or IgM heavy chain CH₂ domain, CH₃ domain, or both.
E65. A polypeptide comprising an antibody heavy chain constant domain that comprises an engineered cysteine residue at a position selected from the group consisting of: 334, 375, 392, and any combination thereof, according to the numbering of the EU index of Kabat.
E66. The polypeptide of E65, wherein constant domain comprises an IgG heavy chain CH₂ domain, CH₃ domain, or both.
E67. A polypeptide comprising an antibody heavy chain constant domain comprising an engineered cysteine residue at a position corresponding to residue 104, 145, 162, or any combination thereof, of SEQ ID NO:62, when said constant domain is aligned with SEQ ID NO:62.
E68. The polypeptide of E67, wherein the engineered cysteine residue is located at position 334, 375, 392, or any combination thereof, of an IgG CH₂ domain, CH₃ domain, or both, according to the numbering of the EU index of Kabat.
E69. The polypeptide E65 or E67, wherein said constant domain comprises an IgA (e.g., IgA₁ or IgA₂), IgD, IgE, or IgM heavy chain CH₂ domain, CH₃ domain, or both.
E70. A polypeptide comprising an antibody heavy chain constant domain that comprises an engineered cysteine residue at a position selected from the group consisting of: 334, 347, 375, 380, 388, 392, and any combination thereof, according to the numbering of the EU index of Kabat.
E71. The polypeptide of E70, wherein the constant domain comprises an IgG heavy chain CH₂ domain, CH₃ domain, or both.
E72. A polypeptide comprising an antibody heavy chain constant domain comprising an engineered cysteine residue ata position corresponding to residue 104, 117, 145, 150, 158, 162, or any combination thereof, of SEQ ID NO:62, when said constant domain is aligned with SEQ ID NO:62.
E73. The polypeptide of E72, wherein the engineered cysteine residue is located at position 334, 347, 375, 380, 388, 392, or any combination thereof, of an IgG CH₂ domain, CH₃ domain, or both, according to the numbering of the EU index of Kabat.
E74. The polypeptide of E70 or E72, wherein the constant domain comprises an IgA (e.g., IgA₁ or IgA₂), IgD, IgE, or IgM heavy chain CH₂ domain, CH₃ domain, or both.
E75. The polypeptide of any one of E23, E25, E28, E30, E33, E35, E38, E40, E43, E45, E48, E50, E54, E56, E58, E61, E63, E66, D68, E71, and E73 wherein said IgG is IgG₁, IgG₂, IgG₃, or IgG₄.
E76. An antibody or antigen binding fragment thereof comprising a polypeptide selected from of any one of E21-E75.
E77. An antibody or antigen binding fragment thereof comprising:

(a) a polypeptide of any one of E21-E75, and

(b) an antibody light chain constant region comprising (i) an engineered cysteine residue at position 183, according to the numbering of Kabat; or (ii) an engineered cysteine residue at a position corresponding to residue 76 of SEQ ID NO:63, when said constant domain is aligned with SEQ ID NO:63.
E78. An antibody or antigen binding fragment thereof comprising:
   (a) a polypeptide of any one of E21-E75, and
   (b) an antibody light chain constant region comprising (i) an engineered cysteine residue at position 110, 111, 125, 149, 155, 158, 161, 185, 188, 189, 191, 197, 205, 206, 207, 208, 210, or any combination thereof, according to the numbering of Kabat; (ii) an engineered cysteine residue at a position corresponding to residue 4, 42, 81, 100, 103, or any combination thereof, of SEQ ID NO:63, when said constant domain is aligned with SEQ ID NO:63 (kappa light chain); or (iii) an engineered cysteine residue at a position corresponding to residue 4, 5, 19, 43, 49, 52, 55, 78, 81, 82, 84, 90, 96, 97, 98, 99, 101, or any combination thereof, of SEQ ID NO:64, when said constant domain is aligned with SEQ ID NO:64 (lambda light chain).
E79. A compound comprising the polypeptide of any one of E21-E75, or the antibody or antigen binding fragment thereof of E76-E78, wherein the polypeptide or antibody is conjugated to a therapeutic agent via the engineered cysteine site.
E80 The compound of E79, wherein the therapeutic agent is conjugated to the polypeptide or the antibody or antigen binding fragment thereof via a linker.
E81. The compound of E79 or E80, wherein:
   (a) the heavy chain constant domain comprises an engineered cysteine residue at a position selected from the group consisting of: 334, 375, 392, and any combination thereof, according to the numbering of the EU index of Kabat; or an engineered cysteine residue at a position corresponding to residue 104, 145,162, or any combination thereof, of SEQ ID NO:62, when the constant domain is aligned with SEQ ID NO:62; and
   (b) the hydrophobicity change of the compound, relative to the polypeptide or unconjugated antibody, as measured by HIC relative retention time, is between about 1.0 to about 1.5, between about 1.0 to about 1.4, between about 1.0 to about 1.3, or between about 1.0 to about 1.2.
E82. The compound of E79 or E80, wherein:
   (a) the heavy chain constant domain that comprises an engineered cysteine residue at a position selected from the group consisting of: 347, 388, 421, 443, and any combination thereof, according to the numbering of the EU index of Kabat; or an engineered cysteine residue at a position corresponding to residue 117, 158, 191, 213, or any combination thereof, of SEQ ID NO:62, when the constant domain is aligned with SEQ ID NO:62; and
   (b) the hydrophobicity change of the compound, relative to the polypeptide or antibody unconjugated, as measured by HIC relative retention time, is about 1.5 or more, about 1.6 or more, about 1.7 or more, about 1.8 or more, about 1.9 or more, or about 2.0 or more.
E83. The compound of E80, wherein:
   (a) the heavy chain constant domain that comprises an engineered cysteine residue at a position selected from the group consisting of: 347, 388, 421, and any combination thereof, according to the numbering of the EU index of Kabat; or an engineered cysteine residue at a position corresponding to residue 117, 158, 191, or any combination thereof, of SEQ ID NO:62, when the constant domain is aligned with SEQ ID NO:62;
   (b) the linker comprises a succinimide group; and
   (c) the percent of succinamide hydrolysis in plasma, at 37° C. under 5% CO₂ at 72 hours, is at least about 45%, at least about 50%, at least about 55%, at least about 60%, at least about 65%, at least about 70%, at least about 75%, at least about 80%, at least about 85%, or at least about 90%.
E84. The compound of E80, wherein:
   (a) the heavy chain constant domain that comprises an engineered cysteine residue at a position selected from the group consisting of: 347, 388, 421, and any combination thereof, according to the numbering of the EU index of Kabat; or an engineered cysteine residue at a position corresponding to residue 117, 158, 191, or any combination thereof, of SEQ ID NO:62, when said constant domain is aligned with SEQ ID NO:62;
   (b) the linker comprises a succinimide group; and
   (c) the percent of succinamide hydrolysis in 0.5 mM glutathione (pH 7.4), at 37° C. at 72 hours, is at least about 45%, at least about 50%, at least about 55%, at least about 60%, at least about 65%, at least about 70%, at least about 75%, at least about 80%, at least about 85%, or at least about 90%.
E85. The compound of E80, wherein:
   (a) the heavy chain constant domain that comprises an engineered cysteine residue at a position selected from the group consisting of: 290, 334, 392, 443, and any combination thereof, according to the numbering of the EU index of Kabat; or an engineered cysteine residue at a position corresponding to residue 60, 104, 162, 213, or any combination thereof, of SEQ ID NO:62, when said constant domain is aligned with SEQ ID NO:62;
   (b) the linker comprises a succinimide group; and
   c) the percent of succinamide hydrolysis in plasma, at 37° C. under 5% CO₂ at 72 hours, is about 50% or less, about 45% or less, about 40% or less, about 35% or less, or about 30% or less.
E86. The compound of E80, wherein:
   (a) the heavy chain constant domain that comprises an engineered cysteine residue at a position selected from the group consisting of: 290, 334, 392, 443, and any combination thereof, according to the numbering of the EU index of Kabat; or an engineered cysteine residue at a position corresponding to residue 60, 104, 162, 213, or any combination thereof, of SEQ ID NO:62, when said constant domain is aligned with SEQ ID NO:62;
   (b) the linker comprises a succinimide group; and
   (c) the percent of succinamide hydrolysis in 0.5 mM glutathione (pH 7.4), at 37° C. at 72 hours, is about 50% or less, about 45% or less, about 40% or less, about 35% or less, or about 30% or less.
E87. The compound of E79 or E80, wherein:
   (a) the heavy chain constant domain that comprises an engineered cysteine residue at a position selected from the group consisting of: 334, 388, 421, 443, and any combination thereof, according to the numbering of the EU index of Kabat; or an engineered cysteine residue at a position corresponding to residue 104, 158, 191, 213, or any combination thereof, of SEQ ID NO:62, when said constant domain is aligned with SEQ ID NO:62; and
   (b) the percent of drug-to-antibody ratio (DAR) loss in plasma, at 37° C. under 5% CO₂ at 72 hours, is no more than about 10%, no more than about 9%, no more than about 8%, no more than about 7%, no more than about 6%, no more than about 5%, no more than about 4%, no more than about 3%, no more than about 2%, or no more than about 1%.
E88. The compound of E79 or E80, wherein:
   (a) the heavy chain constant domain that comprises an engineered cysteine residue at a position selected from the group consisting of: 334, 392, 421, and any combination thereof, according to the numbering of the EU index of Kabat; or an engineered cysteine residue at a position corresponding to residue 104, 162, 191, or any combination thereof, of SEQ ID NO:62, when said constant domain is aligned with SEQ ID NO:62; and
   (b) the percent of DAR loss in 0.5 mM glutathione (pH 7.4), at 37° C. at 72 hours, is no more than about 10%, no more than about 9%, no more than about 8%, no more than about 7%, no more than about 6%, no more than about 5%, no more than about 4%, no more than about 3%, no more than about 2%, or no more than about 1%.
E89. The compound of E82, wherein:
   (a) the heavy chain constant domain that comprises an engineered cysteine residue at a position selected from the group consisting of: 290, 388, 443, and any combination thereof, according to the numbering of the EU index of Kabat; or an engineered cysteine residue at a position corresponding to residue 60, 158, 213, or any combination thereof, of SEQ ID NO:62, when said constant domain is aligned with SEQ ID NO:62; and
   (b) the percent of Cathepsin-mediated linker cleavage (200 to 20000 ng/mL Cathepsin), at 37° C. at 20 minutes, is at least about 50%, at least about 55%, at least about 60%, at least about 65%, at least about 70%, at least about 75%, at least about 80%, at least about 85%, or at least about 90%.
E90. The compound of E80, wherein:
   (a) the heavy chain constant domain that comprises an engineered cysteine residue at a position selected from the group consisting of: 334, 375, 392, and any combination thereof, according to the numbering of the EU index of Kabat; or an engineered cysteine residue at a position corresponding to residue 104, 145, 162, or any combination thereof, of SEQ ID NO:62, when said constant domain is aligned with SEQ ID NO:62; and
   (b) the percent of Cathepsin-mediated (200 to 20000 ng/mL Cathepsin) linker cleavage, at 37° C. at 4 hours, is about 50% or less, about 45% or less, about 40% or less, about 35% or less, about 30% or less, about 25% or less, about 20% or less, or about 15% or less.
E91. The compound of E79 or E80, wherein:
   (a) the heavy chain constant domain that comprises an engineered cysteine residue at a position selected from the group consisting of: 334, 347, 375, 380, 388, 392, and any combination thereof, according to the numbering of the EU index of Kabat; or an engineered cysteine residue at a position corresponding to residue 104, 117, 145, 150, 158, 162, or any combination thereof, of SEQ ID NO:62, when said constant domain is aligned with SEQ ID NO:62; and
   (b) the percent of compound in monomeric form, at 5 mg/mL concentration at 45° C. on day 21, is about 96.0% or more, about 96.5% or more, about 97.0% or more, about 97.5% or more, about 98.0% or more.
E92. The compound of any one of E21 and E80-E91, wherein the linker is cleavable.
E93. The compound of any one of E21 and E80-E92, where the linker comprises vc, mc, MalPeg6, m(H20)c, m(H20)cvc, or a combination thereof.
E94. The compound of any one of E21 and E80-E93, wherein the linker comprises vc.
E95. The compound of any one of E20-E21 and E79-E94, wherein the therapeutic agent is selected from the group consisting of: a cytotoxic agent, a cytostatic agent, a chemotherapeutic agent, a toxin, a radionuclide, a DNA, an RNA, an siRNA, a microRNA, a peptide nucleic acid, a non-natural amino acid, a peptide, an enzyme, a fluorescent tag, biotin, a tubulysin, and any combination thereof.
E96. The compound of any one of E20-E21 and E79-E95, wherein the therapeutic agent is selected from the group consisting of: an auristatin, a maytansinoid, a calicheamicin, a tubulysin, and any combination thereof.
E97. The compound of any one of E20-E21 and E79-E96, wherein the therapeutic agent is an auristatin.
E98. The compound of E97, wherein the auristatin is selected from the group consisting of: 0101, 8261, 6121, 8254, 6780, 0131, MMAD, MMAE, MMAF, and any combination thereof.
E99. The compound of any one of E20-E21 and E79-E96, wherein the therapeutic agent is a tubulysin.
E100. An antibody drug conjugate of formula Ab-(L-D), wherein (a) Ab is an antibody of any one of E76-E78; and (b) L-D is a linker-drug moiety, wherein L is a linker, and D is a drug.
E101. The antibody drug conjugate of E100, wherein L-D comprises a succinimide group, a maleimide group, a hydrolyzed succinimide group, or a hydrolyzed maleimide group.
E102. The antibody drug conjugate of E100 or E101, wherein L-D comprises a maleimide group or a hydrolyzed maleimide group.
E103. The antibody drug conjugate of any one of E100-E102, wherein L-D comprises 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio) pentanoate (SPP), N-succinimidyl 4-(N-maleimidomethyl) cyclohexane-1carboxylate (SMCC), N-Succinimidyl (4-iodo-acetyl) aminobenzoate (SIAB), or 6-maleimidocaproyl-valine-citrulline-p-aminobenzyloxycarbonyl (MC-vc-PAB).
E104. The antibody drug conjugate of any one of E100-E102, comprising the compound of formula I:

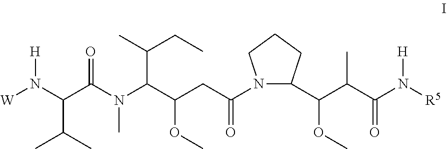

or a pharmaceutically acceptable salt or solvate thereof, wherein, independently for each occurrence,
W is

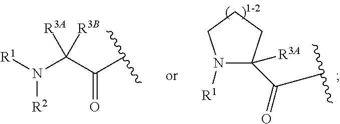

R¹ is hydrogen or C₁-C₈ alkyl;
R² is hydrogen or C₁-C₈ alkyl;
R^{3A} and R^{3B} are either of the following:
(i) R^{3A} is hydrogen or C₁-C₈ alkyl;
R^{3B} is C₁-C₈ alkyl;
(ii) R^{3A} and R^{3B} taken together are C₂-C₈ alkylene or C₁-C₈ heteroalkylene;
R⁵ is

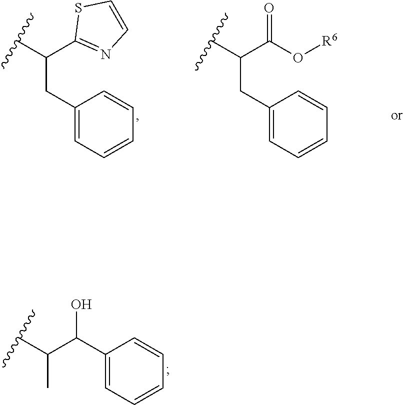

and
R⁶ is hydrogen or —C₁-C₈ alkyl.
E105. The antibody drug conjugate of any one of E100-E102, comprising the compound of formula IIa:

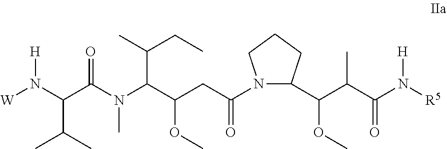

or a pharmaceutically acceptable salt or solvate thereof, wherein, independently for each occurrence,
W is

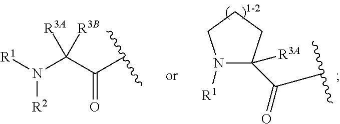

R¹ is

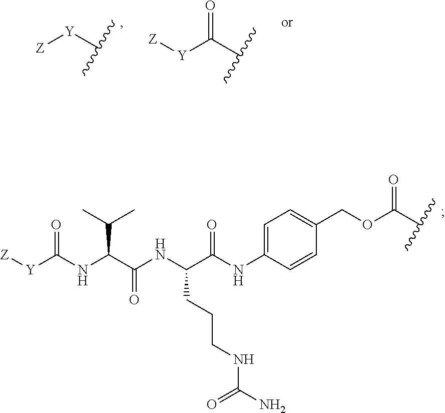

Y is one or more of the group selected from —C₂-C₂₀ alkylene-, —C₂-C₂₀ heteroalkylene-, —C₃-C₈ carbocyclo-, -arylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈heterocyclo)- or —(C₃-C₈ heterocyclo)-C₁-C₁₀alkylene-, —C₁₋₆alkyl(OCH₂OH₂)₁₋₁₀—, —(OCH₂CH₂)₁₋₁₀—, —(OCH₂CH₂)₁₋₁₀—C₁₋₆alkyl, —C(O)—C₁₋₆alkyl(OCH₂OH₂)₁₋₆—, —C₁₋₆alkyl(OCH₂OH₂)₁₋₆—C(O)—, —C₁₋₆alkyl-(OCH₂CH₂)₁₋₆—NRC(O)CH₂—, —C(O)—C₁₋₆alkyl(OCH₂OH₂)₁₋₆—NRC(O)—, and —C(O)—C₁₋₆alkyl-(OCH₂CH₂)₁₋₆—NRC(O)C₁₋₆alkyl-;
Z is

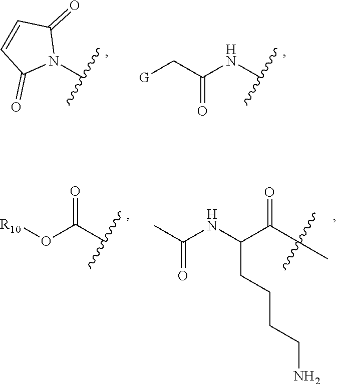

### or —NH₂;

G is halogen, —OH, —SH, or —S—C₁-C₆ alkyl;
R² is hydrogen or C₁-C₈ alkyl;
R^{3A} and R^{3B} are either of the following:
(i) R^{3A} is hydrogen or C₁-C₈ alkyl; and
   R^{3B} is C₁-C₈ alkyl; or
(ii) R^{3A} and R^{3B} taken together are C₂-C₈ alkylene or C₁-C₈ heteroalkylene;
R⁵ is

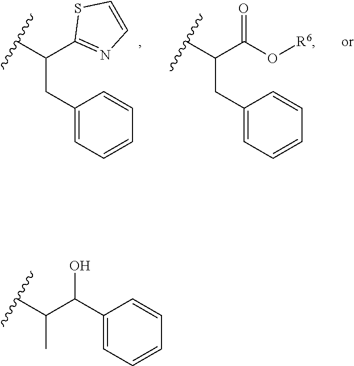

R⁶ is hydrogen or —C₁-C₈ alkyl;
R¹⁰ is hydrogen, —C₁-C₁₀alkyl, —C₃-C₈carbocyclyl, -aryl, —C₁-C₁₀heteroalkyl, —C₃-C₈heterocyclo, —C₁-C₁₀alkylene-aryl, -arylene-C₁-C₁₀alkyl, —C₁-C₁₀alkylene-(C₃-C₈carbocyclo), —(C₃-0₈ carbocyclo)-C₁-C₁₀alkyl, —C₁-C₁₀alkylene-(C₃-C₈heterocyclo), or —(C₃-C₈ heterocyclo)-C₁-C₁₀alkyl, where aryl on R₁₀ comprising aryl is optionally substituted with [R₇]ₕ;
R⁷ is independently selected for each occurrence from the group consisting of F, Cl, I, Br, NO₂, CN and CF₃; and
h is 1, 2, 3, 4 or 5.
E106. The antibody drug conjugate of any one of E100-E102, comprising the compound of formula IIb:

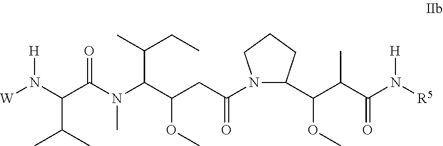

or a pharmaceutically acceptable salt or solvate thereof, wherein, independently for each occurrence,
W is

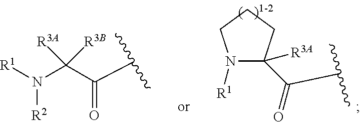

R¹ is

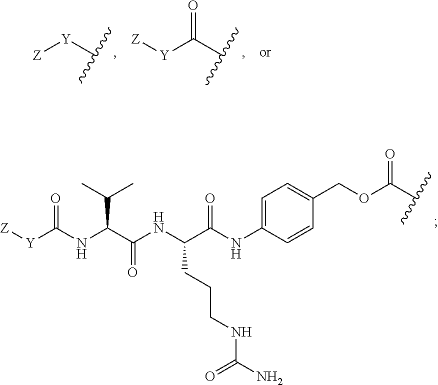

Y is —C₂-C₂₀ alkylene-, —C₂-C₂₀ heteroalkylene-, —C₃-C₈ carbocyclo-, -arylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈heterocyclo)-, or —(C₃-C₈ heterocyclo)-C₁-C₁₀alkylene-;
Z is

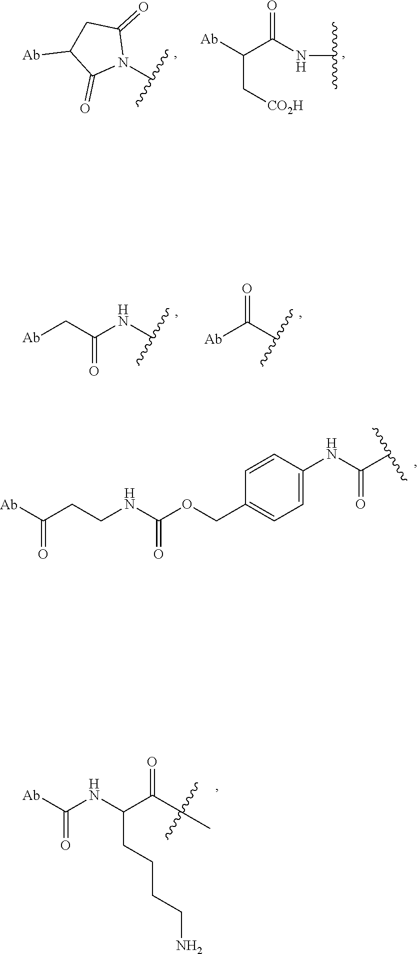

### or —NH-Ab;

Ab is an antibody;
R² is hydrogen, or C₁-C₈ alkyl;
R^{3A} and R^{3B} are either of the following:
(i) R^{3A} is hydrogen or C₁-C₈ alkyl;
   R^{3B} is C₁-C₈ alkyl;
(ii) R^{3A} and R^{3B} taken together are C₂-C₈ alkylene or C₁-C₈ heteroalkylene;
R⁵ is and

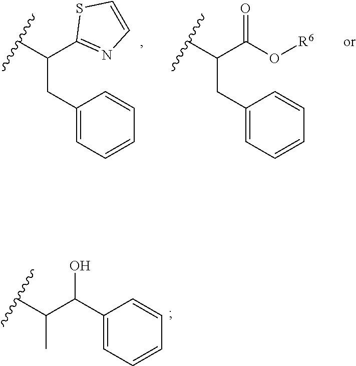

R⁶ is hydrogen or —C₁-C₈ alkyl.
E107. A pharmaceutical composition comprising: the compound of any one of E20-E21 and E79-E99, or the antibody drug conjugate of any one of E100-E107; and a pharmaceutically acceptable carrier.
E108. A method of treating cancer, an autoimmune disease, an inflammatory disease, or an infectious disease, comprising administering to a subject in need thereof a therapeutically effective amount of the compound of any one of E20-E21 and E79-E99, the antibody drug conjugate of any one of E100-E107, or the composition of E108.
E109. The compound of any one of E20-E21 and E79-E99, the antibody drug conjugate of any one of E100-E107, or the composition of E108, for use in treating cancer, an autoimmune disease, an inflammatory disease, or an infectious disease.
E110. Use of the compound of any one of E20-E21 and E79-E99, the antibody drug conjugate of any one of E100-E107, or the composition of E108, for treating cancer, an autoimmune disease, an inflammatory disease, or an infectious disease.
E111. Use of the compound of any one of E20-E21 and E79-E99, the antibody drug conjugate of any one of E100-E107, or the composition of E108, in the manufacture of a medicament for treating cancer, an autoimmune disease, an inflammatory disease, or an infectious disease.
E112. An antibody drug conjugate of the formula Ab-(L-D), wherein:
   (a) Ab is an antibody that binds to HER2 and comprises
      (1) a heavy chain variable region comprising three CDRs comprising SEQ ID NOs:2, 3 and 4;
      (2) a heavy chain constant region of any of SEQ ID NOs:17, 5, 13, 21, 23, 25, 27, 29, 31, 33, 35, 37 or 39;
      (3) a light chain variable region comprising three CDRs comprising SEQ ID NOs:8, 9 and 10;
      (4) a light chain constant region of any of SEQ ID NOs:41, 11 or 43; and
   (b) L-D is a linker-drug moiety, wherein L is a linker, and D is a drug,
   with the proviso that when the heavy chain constant region is SEQ ID NO:5 the light chain constant region is not SEQ ID NO:11.
E113. The antibody drug conjugate of E112, wherein
   (a) the heavy chain constant region is SEQ ID NO:17 and the light chain constant region is SEQ ID NO:41;
   (b) the heavy chain constant region is SEQ ID NO:5 and the light chain constant region is SEQ ID NO:41;
   (c) the heavy chain constant region is SEQ ID NO:17 and the light chain constant region is SEQ ID NO:11;
   (d) the heavy chain constant region is SEQ ID NO:21 and the light chain constant region is SEQ ID NO:11;
   (e) the heavy chain constant region is SEQ ID NO:23 and the light chain constant region is SEQ ID NO:11;
   (f) the heavy chain constant region is SEQ ID NO:25 and the light chain constant region is SEQ ID NO:11;
   (g) the heavy chain constant region is SEQ ID NO:27 and the light chain constant region is SEQ ID NO:11;
   (h) the heavy chain constant region is SEQ ID NO:23 and the light chain constant region is SEQ ID NO:41;
   (i) the heavy chain constant region is SEQ ID NO:25 and the light chain constant region is SEQ ID NO:41;
   (j) the heavy chain constant region is SEQ ID NO:27 and the light chain constant region is SEQ ID NO:41;
   (k) the heavy chain constant region is SEQ ID NO:29 and the light chain constant region is SEQ ID NO:11;
   (l) the heavy chain constant region is SEQ ID NO:31 and the light chain constant region is SEQ ID NO:11;
   (m) the heavy chain constant region is SEQ ID NO:33 and the light chain constant region is SEQ ID NO:43;
   (n) the heavy chain constant region is SEQ ID NO:35 and the light chain constant region is SEQ ID NO:11;
   (o) the heavy chain constant region is SEQ ID NO:37 and the light chain constant region is SEQ ID NO:11;
   (p) the heavy chain constant region is SEQ ID NO:39 and the light chain constant region is SEQ ID NO:11;or
   (q) the heavy chain constant region is SEQ ID NO:13 and the light chain constant region is SEQ ID NO:43.
E114. The antibody drug conjugate of E112, wherein
   (a) the heavy chain comprises any of SEQ ID NOs:18, 6, 14, 22, 24, 26, 28, 30, 32, 34, 36, 38 or 40; and
   (b) the light chain comprises any of SEQ ID NOs: 42, 12 or 44,
   with the proviso that when the heavy chain is SEQ ID NO:6 the light chain is not SEQ ID NO:12.
E115. The antibody drug conjugate of E114, wherein
   (a) the heavy chain is SEQ ID NO:18 and the light chain is SEQ ID NO:42;
   (b) the heavy chain is SEQ ID NO:6 and the light chain is SEQ ID NO:42;
   (c) the heavy chain is SEQ ID NO:18 and the light chain is SEQ ID NO:12;
   (d) the heavy chain is SEQ ID NO:22 and the light chain is SEQ ID NO:12;
   (e) the heavy chain is SEQ ID NO:24 and the light chain is SEQ ID NO:12;
   (f) the heavy chain is SEQ ID NO:26 and the light chain is SEQ ID NO:12;
   (g) the heavy chain is SEQ ID NO:28 and the light chain is SEQ ID NO:12;
   (h) the heavy chain is SEQ ID NO:24 and the light chain is SEQ ID NO:42;
   (i) the heavy chain is SEQ ID NO:26 and the light chain is SEQ ID NO:42;
   (j) the heavy chain is SEQ ID NO:28 and the light chain is SEQ ID NO:42;
   (k) the heavy chain is SEQ ID NO:30 and the light chain is SEQ ID NO:12;
   (l) the heavy chain is SEQ ID NO:32 and the light chain is SEQ ID NO:12;
   (m) the heavy chain is SEQ ID NO:34 and the light chain is SEQ ID NO:44;
   (n) the heavy chain is SEQ ID NO:36 and the light chain is SEQ ID NO:12;
   (o) the heavy chain is SEQ ID NO:38 and the light chain is SEQ ID NO:12;
   (p) the heavy chain is SEQ ID NO:40 and the light chain is SEQ ID NO:12; or
   (q) the heavy chain is SEQ ID NO:14 and the light chain is SEQ ID NO:44.
E116. The antibody drug conjugate of any of E112-E115, wherein the linker is selected from the group consisting of vc, mc, MalPeg6, m(H20)c, and m(H20)cvc.
E117. The antibody drug conjugate of E116, wherein the linker is cleavable.
E118. The antibody drug conjugate of E116 or E117, wherein the linker is vc.
E119. The antibody drug conjugate of any of E112-E118, wherein the drug is membrane permeable.
E120. The antibody drug conjugate of any of E112-E119, wherein the drug is an auristatin.
E121. The antibody drug conjugate of E120, wherein the auristatin is selected from the group consisting of:
   2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide;
   2-methylalanyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide;
   2-methyl-L-prolyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl-1-oxoheptan-4-yl]-N-methyl-L-valinamide, trifluoroacetic acid salt;
   2-methylalanyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl-1-oxoheptan-4-yl]-N-methyl-L-valinamide;
   2-methylalanyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide;
   2-methyl-L-prolyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide, trifluoroacetic acid salt;
   N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide;
   N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide; and
   N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide,
   or a pharmaceutically acceptable salt or solvate thereof.
E122. The antibody drug conjugate of E120, wherein the auristatin is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide or a pharmaceutically acceptable salt or solvate thereof.
E123. An antibody drug conjugate of the formula Ab-(L-D), wherein:
   (a) Ab is an antibody that binds to HER2 and comprises a heavy chain comprising SEQ ID NO:18 and a light chain comprising SEQ ID NO:42; and
   (b) L-D is a linker-drug moiety, wherein L is a linker of vc and D is an auristatin of 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide or a pharmaceutically acceptable salt or solvate thereof.
E124. A pharmaceutical composition comprising the antibody drug conjugate of any of E112-E123 and a pharmaceutically acceptable carrier.
E125. An antibody drug conjugate of the formula Ab-(L-D), wherein Ab is an antibody that binds to extra-domain B (EDB) of fibronectin (FN), and L-D is a linker-drug moiety, wherein L is a linker, and D is a drug.
E126. The antibody drug conjugate of E125, wherein said antibody comprises:
   (i) a heavy chain variable region (VH) that comprises:
      (a) a VH complementarity determining region one (CDR-H1) comprising the amino acid sequence of SEQ ID NO: 66 or 67,
      (b) a VH CDR-H2 comprising the amino acid sequence of SEQ ID NO: 68 or 69; and
      (c) a VH CDR-H3 comprising the amino acid sequence of SEQ ID NO: 70; and
   (ii) a light chain variable region (VL) that comprises:
      (a) a VL complementarity determining region one (CDR-L1) comprising the amino acid sequence of SEQ ID NO: 73,
      (b) a VL CDR-L2 comprising the amino acid sequence of SEQ ID NO: 74; and
      (c) a VL CDR-L3 comprising the amino acid sequence of SEQ ID NO: 75.
E127. The antibody drug conjugate of E125 or E126, wherein the linker is selected from the group consisting of vc, mc, MalPeg6, m(H20)c, and m(H20)cvc.
E128. The antibody drug conjugate of E127, wherein the linker is cleavable.
E129. The antibody drug conjugate of E127 or E128, wherein the linker is vc.
E130. The antibody drug conjugate of any of E125-E129, wherein the drug is membrane permeable.
E131. The antibody drug conjugate of any of E125-E130, wherein the drug is an auristatin.
E132. The antibody drug conjugate of E131, wherein the auristatin is selected from the group consisting of:
   2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide;
   2-methylalanyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide;
   2-methyl-L-prolyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl-1-oxoheptan-4-yl]-N-methyl-L-valinamide, trifluoroacetic acid salt;
   2-methylalanyl-N-[(3R,4S,5S)-3-methoxy-1-{(2S)-2-[(1R,2R)-1-methoxy-3-{[(2S)-1-methoxy-1-oxo-3-phenylpropan-2-yl]amino}-2-methyl-3-oxopropyl]pyrrolidin-1-yl}-5-methyl-1-oxoheptan-4-yl]-N-methyl-L-valinamide;
   2-methylalanyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide;
   2-methyl-L-prolyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide, trifluoroacetic acid salt;
   N-methyl-L-valyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide;
   N-methyl-L-valyl-N-[(3R,4S,5S)-1-{(2S)-2-[(1R,2R)-3-{[(1S,2R)-1-hydroxy-1-phenylpropan-2-yl]amino}-1-methoxy-2-methyl-3-oxopropyl]pyrrolidin-1-yl}-3-methoxy-5-methyl-1-oxoheptan-4-yl]-N-methyl-L-valinamide; and
   N-methyl-L-valyl-N-[(3R,4S,5S)-1-{(2S)-2-[(1R,2R)-3-{[(1S)-1-carboxy-2-phenylethyl]amino}-1-methoxy-2-methyl-3-oxopropyl]pyrrolidin-1-yl}-3-methoxy-5-methyl-1-oxoheptan-4-yl]-N-methyl-L-valinamide, or a pharmaceutically acceptable salt or solvate thereof.
E133. The antibody drug conjugate of E132, wherein the auristatin is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide or a pharmaceutically acceptable salt or solvate thereof.
E134. A pharmaceutical composition comprising the antibody drug conjugate of any of E125-E133 and a pharmaceutically acceptable carrier.
E135. A nucleic acid encoding the polypeptide of any one of E1-E14 and E22-E75,
E136. A nucleic acid encoding the antibody of any one of E15-E19 and E76-E78.
E137. A nucleic acid encoding the antibody moiety of the compound of any one of E20, E21, and E79-E99.
E138. A nucleic acid encoding the antibody moiety of the antibody drug conjugate of any one of E100-E106, E112-E123, and E125-E133.
E139. A nucleic acid encoding a polypeptide that comprises an antibody heavy chain constant domain, wherein said heavy chain constant domain comprises an engineered cysteine residue at position 290, according to the numbering of the EU index of Kabat.
E140. An isolated nucleic acid encoding an antibody, or antigen-binding fragment thereof, wherein said antibody, or antigen-binding fragment thereof, comprises:
   (i) a heavy chain variable region (VH) that comprises:
      (a) a VH complementarity determining region one (CDR-H1) comprising the amino acid sequence of SEQ ID NO: 66 or 67,
      (b) a VH CDR-H2 comprising the amino acid sequence of SEQ ID NO: 68 or 69; and
      (c) a VH CDR-H3 comprising the amino acid sequence of SEQ ID NO: 70; and
   (ii) a light chain variable region (VL) that comprises:
      (a) a VL complementarity determining region one (CDR-L1) comprising the amino acid sequence of SEQ ID NO: 73,
      (b) a VL CDR-L2 comprising the amino acid sequence of SEQ ID NO: 74; and
      (c) a VL CDR-L3 comprising the amino acid sequence of SEQ ID NO: 75.
E141. A host cell comprising the nucleic acid of any one of E135-E140.
E142. A method of producing a polypeptide or an antibody, comprising culturing the host cell of E141 under suitable conditions for expressing said polypeptide or antibody, and isolating said polypeptide or antibody.

### BRIEF DESCRIPTION OF THE DRAWINGS

FIGS. 1A-1B depict (A) T(kK183C+K290C)-vc0101 and (B) T(LCQ05+K222R)-AcLysvc0101 ADCs. Each black circle represents a linker/payload that is conjugated to the monoclonal antibody. The structure of one such linker/payload is shown for each ADC. The underlined entity is supplied by the amino acid residue on the antibody through which conjugation occurs.

FIGS. 2A-2E depict spectra of selected ADCs from hydrophobic interaction chromatography (HIC) showing changes in retention times upon conjugation of trastuzumab derived antibodies to different linker payloads.

FIGS. 3A-3B depict graphs of ADCs binding to HER2. (A) direct binding to HER2 positive BT474 cells and (B) competitive binding with PE labelled trastuzumab to BT474 cells. These results indicate that the binding properties of antibody in these ADCs were unaltered by the conjugation process.

FIG. 4 depicts ADCC activities of trastuzumab derived ADCs.

FIG. 5 depicts in vitro cytotoxicity data (IC₅₀) reported in nM payload concentration for a number of trastuzumab derived ADCs on a number of cell lines with different levels of HER2 expression.

FIG. 6 depicts in vitro cytotoxicity data (IC₅₀) reported in ng/ml antibody concentration for a number of trastuzumab derived ADCs on a number of cell lines with different levels of HER2 expression.

FIGS. 7A-7I depict anti-tumor activity of nine trastuzumab derived ADCs on N87 xenografts with tumor volume was plotted over time. (A) T(kK183C+K290C)-vc0101; (B) T(kK183C)-vc0101; (C) T(K290C)-vc0101; (D) T(LCQ05+K222 R)-AcLysvc0101; (E) T(K290C+K334C)-vc0101; (F) T(K334C+K392C)-vc0101; (G) T(N297Q+K222R)-AcLysvc0101; (H) T-vc0101; (I) T-DM1. N87 gastric cancer cells express high levels of HER2.

FIGS. 8A-8E depict anti-tumor activity of six trastuzumab derived ADCs on HCC1954 xenografts with tumor volume plotted over time. (A) T(LCQ05+K222R)-AcLysvc0101; (B) T(K290C+K334C)-vc0101; (C) T(K334C+K392C)-vc0101; (D) T(N297Q+K222 R)-AcLysvc0101; (E) T-DM1. HCC1954 breast cancer cells express high levels of HER2.

FIGS. 9A-9G depict anti-tumor activity of seven trastuzumab derived ADCs on JIMT-1 xenografts with tumor volume plotted over time. (A) T(kK183C+K290C)-vc0101; (B) T(LCQ05+K222 R)-AcLysvc0101; (C) T(K290C+K334C)-vc0101; (D) T(K334C+K392C)-vc0101; (E) T(N297Q+K222R)-AcLysvc0101; (F) T-vc0101; (G) T-DM1. JIMT-1 breast cancer cells express moderate/low levels of HER2.

FIGS. 10A-10D depict anti-tumor activity of five trastuzumab derived ADCs on MDA-MB-361(DYT2) xenografts with tumor volume plotted over time. (A) T(LCQ05+K222R)-AcLysvc0101; (B) T(N297Q+K222R)-AcLysvc0101; (C) T-vc0101; (D) T-DM1. MDA-MB-361(DYT2) breast cancer cells express moderate/low levels of HER2.

FIGS. 11A-11E depict anti-tumor activity of five trastuzumab derived ADCs on PDX-144580 patient derived xenografts with tumor volume plotted over time. (A) T(kK183C+K290C)-vc0101; (B) T(LCQ05+K222R)-AcLysvc0101; (C) T(N297Q+K222R)-AcLysvc0101; (D) T-vc0101; (E) T-DM1. PDX-144580 patient derived cells are a TNBC PDX model.

FIGS. 12A-12D depict anti-tumor activity of four trastuzumab derived ADCs on PDX-37622 patient derived xenografts with tumor volume plotted over time. (A) T(kK183C+K290C)-vc0101; (B) T(N297Q+K222R)-AcLysvc0101; (C) T(K297C+K334C)-vc0101; (D) T-DM1. PDX-37622 patient derived cells are a NSCLC PDX model expressing moderate levels of HER2.

FIGS. 13A-13B depict immunohistocytochemistry of N87 tumor xenografts treated with either (A) T-DM1 or (B) T-vc0101 and stained for phosphohistone H3 and IgG antibody. Bystander activity is observed with T-vc0101.

FIG. 14 depicts in vitro cytotoxicity data (IC₅₀) reported in nM payload concentration and ng/ml antibody concentration for a number of trastuzumab derived ADCs and free payloads on cells made resistant to T-DM1 in vitro (N87-TM1 and N87-TM2) or parental cells sensitive to T-DM1 (N87cells). N87 gastric cancer cells express high levels of HER2.

FIGS. 15A-15G depict anti-tumor activity of seven trastuzumab derived ADCs on T-DM1 sensitive (N87 cells) and resistant (N87-TM1 and N87-TM2) gastric cancer cells. (A) T-DM1; (B) T-mc8261; (C) T(297Q+K222R)-AcLysvc0101; (D) T(LCQ05+K222R)-AcLysvc0101; (E) T(K290C+K334C)-vc0101; (F) T(K334C+K392C)-vc0101; (G) T(kK183C+K2900)-vc0101.

FIGS. 16A-16B depict western blots showing (A) MRP1 drug efflux pump and (B) MDR1 drug efflux pump protein expression on T-DM1 sensitive (N87 cells) and resistant (N87-TM1 and N87-TM2) gastric cancer cells.

FIGS. 17A-17B depict HER2 expression and binding to trastuzumab of T-DM1 sensitive (N87 cells) and resistant (N87-TM1 and N87-TM2) gastric cancer cells. (A) a western blot showing HER2 protein expression and (B) trastuzumab binding to cell surface HER2.

FIGS. 18A-18D depict characterization of protein expression levels in T-DM1 sensitive (N87 cells) and resistant (N87-TM1 and N87-TM2) gastric cancer cells. (A) protein expression level changes in 523 proteins; (B) western blots showing protein expression of IGF2R, LAMP1 and CTSB; (C) western blot showing protein expression of CAV1; (D) IHC of CAV1 protein expression in tumors generated in vivo from implantation of N87 and N87-TM2 cells.

FIGS. 19A-19C depict sensitivity to trastuzumab and various trastuzumab derived ADCs of tumors generated in vivo from implantation of (A) T-DM1 sensitive N87 parental cells; (B) T-DM1 resistant N87-TM1 cells; (C) T-DM1 resistant N87-TM2 cells.

FIGS. 20A-20F depict sensitivity to trastuzumab and various trastuzumab derived ADCs of tumors generated in vivo from implantation of T-DM1 sensitive N87 parental cells and T-DM1 resistant N87-TM2 or N87-TM1 cells. (A) N87 tumor size was plotted over time in the presence of trastuzumab or two trastuzumab derived ADCs; (B) N87-TM2 tumor size was plotted over time in the presence of trastuzumab or two trastuzumab derived ADCs; (C) time for N87 cell tumor to double in size in the presence of in the presence of trastuzumab or two trastuzumab derived ADCs; (D) time for N87-TM2 cell tumor to double in size in the presence of trastuzumab or two trastuzumab derived ADCs; (E) N87-TM2 tumor size was plotted over time in the presence of seven different trastuzumab derived ADCs; (F) N87-TM1 tumor size was plotted over time with a trastuzumab derived ADC added at day 14.

FIGS. 21A-21E depict generation and characterization of T-DM1 resistant cells generated in vivo. (A) N87 gastric cancer cells were initially sensitive to T-DM1 when implanted in vivo. (B) Over time, the implanted N87 cells became resistant to T-DM1 but remained sensitive to (C) T-vc0101, (D) T(N297Q+K222R)-AcLysvc0101 and (E) T(kK183+K290C)-vc0101.

FIGS. 22A-22D depict in vitro cytotoxicity of four trastuzumab derived ADCs on T-DM1 resistant cells (N87-TDM) generated in vivo compared to T-DM1 sensitive parental N87 cells with tumor volume plotted over time. (A) T-DM1; (B) T(kK183+K290C)-vc0101; (C) T(LCQ05+K222 R)-AcLysvc0101; (D) T(N297Q+K222 R)-AcLysvc0101.

FIGS. 23A-23B depict HER2 protein expression levels on T-DM1 resistant cells (N87-TDM1, from mice 2, 17 and 18) generated in vivo compared to T-DM1 sensitive parental N87 cells. (A) FACS analysis and (B) western blot analysis. No significant difference in HER2 protein expression was observed.

FIGS. 24A-24D depict that T-DM1 resistance in N87-TDM1 (mice 2, 7 and 17) is not due to drug efflux pumps. (A) a western blot showing MDR1 protein expression. In vitro cytotoxicity of T-DM1 resistant cells (N87-TDM1) and T-DM1 sensitive N87 parental cells in the presence of free drug (B) 0101; (C) doxorubicin; (D) T-DM1.

FIGS. 25A-25B depict Concentration vs time profiles and pharmacokinetics/toxicokinetics of (A) both total Ab and trastuzumab ADC (T-vc0101) or T(kK183C+K290C) site specific ADC after dose administration to cynomolgus monkeys and (B) the ADC analyte of trastuzumab (T-vc0101) or various site specific ADCs after dose administration to cynomolgus monkeys.

FIG. 26 depicts relative retention values by hydrophobic interaction chromatography (HIC) vs exposure (AUC) in rats. The X-axis represents Relative Retention Time by HIV; while the Y-axis represents pharmacokinetic dose-normalized exposure in rats (“area under curve”, AUC for antibody, from 0 to 336 hours, divided by drug dose of 10 mg/kg). Symbol shape denotes approximate drug loading (DAR): diamond=DAR 2; circle=DAR 4. Arrow indicates T(kK183C+K290C)-vc0101.

FIG. 27 depicts a toxicity study using T-vc0101 conventional conjugate ADC and T(kK183C+K290C)-vc0101 site specific ADC. T-vc0101 induced severe neutropenia at 5 mg/kg while the T(kK183C+K290C)-vc0101 caused a minimal drop in neutrophil counts at 9 mg/kg.

FIGS. 28A-28C depict the crystal structure of (A) T(K290C+K334C)-vc0101; (B) T(K290C+K392C)-vc0101; and (C) T(K334C+K392C)-vc0101. As shown in FIG. 28C, considering payload geometry, conjugation at any of K290, K334, K392 sites could potentially perturb the overall trajectory of the glycan away from the CH2 surface destabilizing the glycan, and the CH2 structure itself, and as a result of the Ch2-Ch3 interface.

FIG. 29 is a tumor growth plots (N87) for the 3mpk dosing of various vc0101 site mutant ADCs.

FIG. 30 shows raw SEC traces illustrating the behavior of various site mutants when conjugated to LP#2.

FIG. 31 shows the Plasma stability of ADCs of Example 22. Heavy chain or light chain containing the acetylated product (mass shift=993) was counted as “loaded” while those containing the deacetylated product (mass shift=951) was counted as “un-loaded” for DAR calculations.

FIG. 32 shows the in vivo Stability of ADCs of Example 22, as measured by DAR.

FIG. 33 shows EDB+ FN expression by western blot in WI38-VA13 and HT-29 cells.

FIGS. 34A-34F show anti-tumor efficacy in PDX-NSX-11122, a high EDB+ FN expressing NSCLC patient derived xenograft (PDX) model of human cancer, of (A) EDB-L19-vc-0101 at 0.3, 0.75, 1.5 and 3 mg/kg; (B) EDB-L19-vc-0101 at 3 mg/kg and 10 mg/kg of disulfide linked EDB-L19-diS-DM1; (C) EDB-L19-vc-0101 at 1 and 3 mg/kg and 5 mg/kg of disulfide linked EDB-L19-diS-C₂OCO-1569; (D) site-specific conjugated EDB-(κK183C+K2900)-vc-0101 and conventionally conjugated EDB-L19-vc-0101 (ADC1) at the doses of 0.3, 1 and 3 mg/kg and 1.5 mg/kg, respectively; (E) site-specific conjugated EDB-mut1(κK183C-K2900)-vc-0101 at the doses of 0.3, 1 and 3 mg/kg; and (F) EDB-mut1(κK183C-K2900)-vc-0101 group dosed at 3 mg/kg as tumor growth inhibition curves for each individual tumor bearing mouse.

FIGS. 35A-35F show anti-tumor efficacy in H-1975, a moderate to high EDB+FN expressing NSCLC cell line xenograft (CLX) model of human cancer, of (A) EDB-L19-vc-0101 at 0.3, 0.75, 1.5 and 3 mg/mg; (B) EDB-L19-vc-0101 and EDB-L19-vc-1569 at 0.3, 1 and 3 mg/kg; (C) EDB-L19-vc-0101 and EDB-(H16-K222R)-AcLys-vc-CPI at 0.5, 1.5 and 3 mg/kg and 0.1, 0.3 and 1 mg/kg, respectively; (D) site-specific conjugated EDB-(κK183C+K290C)-vc-0101 and conventionally conjugated EDB-L19-vc-0101 at 0.5, 1.5 and 3 mg/kg; (E) EDB-L19-vc-0101 and EDB-(K94R)-vc-0101 at 1 and 3 mg/kg; and (F) EDB-(KK183C+K290C)-vc-0101 and EDB-mut1(κK183C-K290C)-vc-0101 at 1 and 3 mg/kg.

FIG. 36 shows anti-tumor efficacy in HT29, a moderate EDB+ FN expressing colon CLX model of human cancer, of EDB-L19-vc-0101 and EDB-L19-vc-9411 at 3 mg/kg.

FIGS. 37A-37B show anti-tumor efficacy of EDB-L19-vc-0101 at 0.3, 1 and 3 mg/kg in (A) PDX-PAX-13565, a moderate to high EDB+FN expressing pancreatic PDX; and (B) PDX-PAX-12534, a low to moderate EDB+FN expressing pancreatic PDX.

FIG. 38 shows anti-tumor efficacy of EDB-L19-vc-0101 at 1 and 3 mg/kg in Ramos, a moderate EDB+ FN expressing lymphoma CLX model of human cancer.

FIGS. 39A-39B show the anti-tumor efficacy in EMT-6, a mouse syngeneic breast carcinoma model, of (A) EDB-mut1κK183C-K290C)-vc-0101 at 4.5 mg/kg; and (B) EDB-(κK183C-K94R-K290C)-vc-0101 group dosed at 4.5 mg/kg as tumor growth inhibition curves for each individual tumor bearing mouse.

FIG. 40 shows absolute neutrophil counts for conventionally conjugated EDB-L19-vc-0101 at 5 mg/kg compared to site-specific conjugated EDB-mut1(κK183C-K290C)-vc-0101 (ADC4) at 6 mg/kg.

FIG. 41 shows the competitive binding of antibody X and cys mutant X(kK183C+K290C) to target antigen. X and X(kK183C+K290C) were tested in a competition ELISA in which target antigen was immobilized on the plate, and both antibody X and cys mutant X(kK183C+K290C) were applied in serial dilutions in the presence of biotinylated parental antibody at a constant concentration. The amount of biotinylated parental antibody that remained bound on the target antigen on the ELISA plate was determined by applying streptavidin conjugated with horse radish peroxidase (see methods).

FIG. 42 shows the growth curves of Calu-6 human NSCLC xenograft tumors in female athymic mice treated with ADCs or vehicle. Average tumor volumes (mm³, mean±SEM) of individual mice in each treatment group were plotted against days after initial dosing.

### DETAILED DESCRIPTION OF THE INVENTION

The invention relates to polypeptides, antibodies, and antigen-binding fragments thereof, that comprise a substituted cysteine for site-specific conjugation. In particular, it was discovered position 290 in the antibody heavy chain constant region (according to the numbering of the EU index of Kabat) can be used for site specific conjugation to make antibody drug conjugates (ADCs) with antibodies to various targets (including but not limited to HER2). Data exemplified herein demonstrate that ADC constructs conjugated at position 290 show superior in vivo properties, as compared to other conjugation sites.

For example, as shown in the Examples, conjugation at various conjugation sites can result in different ADC characteristics, such as biophysical property (e.g., hydrophobicity), biological stability, conjugatability, and ADC efficacy (e.g., payload release kinetics and ADC metabolism).

Hydrophobic linker-payloads, such as vc-101 used in the Examples, create particular challenges for ADCs. It has been reported that plasma clearance rate increases as the hydrophobicity of the linker-payload increases, resulting in reduced in vivo efficacy. Thus, it has been proposed that reducing overall hydrophobicity can improve in vivo PK (Lyon et al, Nature Biotechnology 33, 733-735 (2015)). However, the inventors observed through a series of experiments that reduced hydrophobicity does not always correlates with improved PK. In fact, in many circumstances, hydrophobicity is not a reliable predictor for favorable PK profile. Furthermore, PK profiles for the Cys-based site-specific conjugates do not behave like transglutaminase conjugates. Thus, new design schemes and criteria were needed for evaluating desirable conjugation sites.

Structural studies by the inventors provided some initial insight on the selection of desirable conjugation sites. For example, ADC conjugation at certain site might alter the structure of the Fc domain, or may interfere with glycosylation of the antibody because of the geometry of the payload at this site. In addition, certain sites may provide a proper balance of surface exposure: it is exposed enough to allow a drug to be conjugated, but not too exposed such that the drug is metabolized in vivo and cleared from plasma too quickly. Based on structural studies, a number of candidate sites were identified as potential conjugation site (e.g., heavy chain 290, 392, light chain 183).

Following the structural studies, additional assays were designed and conducted. Notably, among several conjugation sites evaluate by the inventors, position 290 initially did not shown superior properties as compared to other conjugation sites. For example, in vivo pharmacokinetic (PK) data based on mouse model fails to suggest that position 290 is particularly desirable. However, in vivo PK data from cynomolgus monkey surprisingly showed that ADC molecules conjugated at position 290 have superior PK profile, which makes this conjugation site more advantageous for clinical uses. The advantages of site 290 could not have been predicted based on hydrophobicity of linker-payload.

Other conjugation sites that also provided superior in vivo PK profile include 392 (heavy chain) and 183 (light chain).

In addition to favorable in vivo PK, Cys-290 conjugates have also shown very low levels of high molecular weight (HMVV) aggregation, and favorable antibody-dependent cell-mediated cytotoxicity (ADCC). In particular, it has been reported that conjugation event often results in loss of ADCC function. For example, Anti-CD70, the antibody component for SGN-70A ADC, has shown ADCC, antibody-dependent cellular phagocytosis (ADCP), and complement-dependent cytotoxicity (CDC) functions. Nonetheless, anti-CD70-MMAF conjugates lack FcγR binding (Kim et al, Biomol Ther (Seoul). 2015 Nov; 23(6): 493-509). In contrast, the ADCC function has not been compromised in the Cys-290 ADC conjugates disclosed herein.

Further, hematologic and microscopic data exemplified herein show that the site-specific conjugation using Cys-290 also improved the ADC (e.g., Ab-vc0101) induced toxicity (such as neutropenia and bone marrow toxicity), as compared to conventional conjugates.

Finally, examples provided herein also showed that depending on the specific applications of the ADC molecules, a number of candidate conjugation sites can be used to solve specific problems. For example, certain sites provide better payload metabolism, some sites reduce the overall hydrophobicity of the molecule, and some sites allow for faster or slower linker cleavage. These preferred conjugation sites can be used for the optimization of ADC molecules. See Examples 21 and 22.

### 1. Antibody-Drug Conjugates (ADCs)

ADCs comprise an antibody component conjugated to a drug payload, typically through the use of a linker. Conventional conjugation strategies for ADCs rely on randomly conjugating the drug payload to the antibody through lysines or cysteines that are endogenously found on the antibody heavy and/or light chain. Accordingly, such ADCs are a heterogeneous mixture of species showing different drug:antibody ratios (DAR). In contrast, the ADCs disclosed herein are site specific ADCs that conjugate the drug payload to the antibody at particular engineered residues on the antibody heavy and/or light chain. As such, the site specific ADCs are a homogeneous population of ADCs comprised of a species with a defined drug:antibody ratio (DAR). Thus, the site specific ADCs demonstrate uniform stoichiometry resulting in improved pharmacokinetics, biodistribution and safety profile of the conjugate. ADCs of the invention include antibodies and polypeptides of the invention conjugated to linkers and/or payloads.

The present invention provides antibody drug conjugates of the formula Ab-(L-D), wherein (a) Ab is an antibody, or antigen-binding fragment thereof, that binds to an antigen, and (b) L-D is a linker-drug moiety, wherein L is a linker, and D is a drug.

Also encompassed by the present invention are antibody drug conjugates of the formula Ab-(L-D)ₚ, wherein (a) Ab is an antibody, or antigen-binding fragment thereof, that binds to an antigen, (b) L-D is a linker-drug moiety, wherein L is a linker, and D is a drug and (c) p is the number of linker/drug moieties are attached to the antibody. For site specific ADCs, p is a whole number due to the homogeneous nature of the ADC. In some embodiments, p is 4. In other embodiments, p is 3. In other embodiments, p is 2. In other embodiments, p is 1. In other embodiments, p is greater than 4.

A. Antibodies and Conjugation Sites

The polypeptides and antibodies of the invention are conjugated to the payload in a site specific manner. To accommodate this type of conjugation, the constant domain is modified to provide for either a reactive cysteine residue engineered at one or more specific sites (sometimes referred to as “Cys” mutants). Also disclosed are antibodies that can be used for transglutaminase-based conjugation, in which an acyl donor glutamine-containing tag or an endogenous glutamine is made reactive by polypeptide engineering in the presence of transglutaminase and an amine.

In general, the regions of an antibody heavy or light chain are defined as “constant” (C) region or “variable” (V) regions, based on the relative lack of sequence variation within the regions of various class members. A constant region of an antibody may refer to the constant region of the antibody light chain or the constant region of the antibody heavy chain, either alone or in combination. The constant domains are not involved directly in binding an antibody to an antigen, but exhibit various effector functions, such as Fc receptor (FcR) binding, participation of the antibody in antibody-dependent cellular toxicity (ADCC), opsonization, initiation of complement dependent cytotoxicity, and mast cell degranulation.

The constant and variable regions of an antibody heavy and light chains are folded into domains. Constant region on the light chain of an immunoglobulin is generally referred to as “CL domain.” Constant domains on the heavy chain (e.g. hinge, CH1, CH2 or CH3 domains) are referred to as “CH domains.” The constant regions of the polypeptide or antibody (or fragment thereof) of the invention may be derived from constant regions of any one of IgA, IgD, IgE, IgG, IgM, or any isotypes thereof as well as subclasses and mutated versions thereof.

CH1 domain includes the first (most amino terminal) constant region domain of an immunoglobulin heavy chain that extends, e.g., from about positions 118-215 according to the numbering of the EU index of Kabat. The CH1 domain is adjacent to the VH domain and amino terminal to the hinge region of an immunoglobulin heavy chain molecule, and does not form a part of the Fc region of an immunoglobulin heavy chain.

The hinge region includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains.

CH2 domain includes the portion of a heavy chain immunoglobulin molecule that extends, e.g., from about positions 231-340 according to the numbering of the EU index of Kabat. The CH2 domain is unique in that it is not closely paired with another domain. Rather, two N-linked branched carbohydrate chains are interposed between the two CH2 domains of an intact native IgG molecule. In certain embodiments, the polypeptide or antibody (or fragment thereof) of the invention comprises a CH2 domain derived from an IgG molecule, such as IgG1, IgG2, IgG3, or IgG4. In certain embodiments, the IgG is a human IgG.

CH3 domain includes the portion of a heavy chain immunoglobulin molecule that extends approximately 110 residues from N-terminus of the CH2 domain, e.g., from about positions 341-447 according to the numbering of the EU index of Kabat. The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE). In certain embodiments, the polypeptide or antibody (or fragment thereof) of the invention comprises a CH3 domain derived from an IgG molecule, such as IgG1, IgG2, IgG3, or IgG4. In certain embodiments, the IgG is a human IgG.

CL domain includes the constant region domain of an immunoglobulin light chain that extends, e.g. from about positions 108-214 according to the numbering of the EU index of Kabat. The CL domain is adjacent to the VL domain. In certain embodiments, the polypeptide or antibody (or fragment thereof) of the invention comprises a kappa light chain constant domain (CLK). In certain embodiments, the polypeptide or antibody (or fragment thereof) of the invention comprises a lambda light chain constant domain (CLX). CLK has known polymorphic loci CLK-V/A45and CLK-L/V83 (using Kabat numbering) thus allowing for polymorphisms Km(1): CLK-V45/L83; Km(1,2): CLK-A45/L83; and Km(3): CLK-A45/V83. Polypeptides, antibodies and ADCs of the invention can have antibody components with any of these light chain constant regions.

The Fc region generally comprises a CH2 domain and a CH3 domain. Although the boundaries of the Fc region of an immunoglobulin heavy chain might vary, the human IgG heavy chain Fc region is usually defined to stretch from an amino acid residue at position Cys226, or from Pro230 (according to the numbering of the EU index of Kabat), to the carboxyl-terminus thereof. A Fc region of the invention may be a native sequence Fc region or a variant Fc region.

In one aspect, the invention provides a polypeptide comprising an antibody heavy chain constant domain that comprises a substituted cysteine residue at position 290, according to the numbering of the EU index of Kabat. As disclosed and exemplified herein, conjugation at position 290 provided surprisingly desirable in vivo PK profiles.

Additional cysteine substitution may be introduced, such as positions 118, 246, 249, 265, 267, 270, 276, 278, 283, 292, 293, 294, 300, 302, 303, 314, 315, 318, 320, 332, 333, 334, 336, 345, 347, 354, 355, 358, 360, 362, 370, 373, 375, 376, 378, 380, 382, 386, 388, 390, 392, 393, 401, 404, 411, 413, 414, 416, 418, 419, 421, 428, 431, 432, 437, 438, 439, 443, 444, or any combination thereof, according to the numbering of the EU index of Kabat. In particular, positions 118, 334, 347, 373, 375, 380, 388, 392, 421, 443, or any combination thereof may be used. Residue 118 is also referred to as A114, A114C, C114, or 114C in the examples because the initial publication of this site used Kabat numbering (114) instead of EU index (118), and has since been generally referred in the art as the 114 site.

In another aspect, the invention provides an antibody or antigen binding fragment thereof comprising (a) a polypeptide disclosed herein and (b) an antibody light chain constant region comprising (i) an engineered cysteine residue at position 183, according to the numbering of the EU index of Kabat; or (ii) an engineered cysteine residue at a position corresponding to residue 76 of SEQ ID NO:63, when said constant domain is aligned with SEQ ID NO:63.

In another aspect, the invention provides an antibody or antigen binding fragment thereof comprising (a) a polypeptide disclosed herein and (b) an antibody light chain constant region comprising (i) an engineered cysteine residue at position 110, 111, 125, 149, 155, 158, 161, 185, 188, 189, 191, 197, 205, 206, 207, 208, 210, or any combination thereof, according to the numbering of Kabat; (ii) an engineered cysteine residue at a position corresponding to residue 4, 42, 81, 100, 103, or any combination thereof, of SEQ ID NO:63, when said constant domain is aligned with SEQ ID NO:63 (kappa light chain); or (iii) an engineered cysteine residue at a position corresponding to residue 4, 5, 19, 43, 49, 52, 55, 78, 81, 82, 84, 90, 96, 97, 98, 99, 101, or any combination thereof, of SEQ ID NO:64, when said constant domain is aligned with SEQ ID NO:64 (lambda light chain).

In another aspect, the invention provides an antibody or antigen binding fragment thereof comprising (a) a polypeptide disclosed herein and (b) an antibody kappa light chain constant region comprising (i) an engineered cysteine residue at position 111, 149, 188, 207, 210, or any combination thereof (preferably 111 or 210), according to the numbering of Kabat; or (ii) an engineered cysteine residue at a position corresponding to residue 4, 42, 81, 100, 103, or any combination thereof, of SEQ ID NO:63 (preferably residue 4 or 103), when said constant domain is aligned with SEQ ID NO:63.

In another aspect, the invention provides an antibody or antigen binding fragment thereof comprising (a) a polypeptide disclosed herein and (b) an antibody lambda light chain constant region comprising (i) an engineered cysteine residue at position 110, 111, 125, 149, 155, 158, 161, 185, 188, 189, 191, 197, 205, 206, 207, 208, 210, or any combination thereof (preferably 110, 111, 125, 149, or 155), according to the numbering of Kabat; or (ii) an engineered cysteine residue at a position corresponding to residue 4, 5, 19, 43, 49, 52, 55, 78, 81, 82, 84, 90, 96, 97, 98, 99, 101, or any combination thereof of SEQ ID NO:64 (preferably residue 4, 5, 19, 43, or 49), when said constant domain is aligned with SEQ ID NO:64.

Amino acid modifications can be made by any method known in the art and many such methods are well known and routine for the skilled artisan. For example, but not by way of limitation, amino acid substitutions, deletions and insertions may be accomplished using any well-known PCR-based technique. Amino acid substitutions may be made by site-directed mutagenesis (see, for example, Zoller and Smith, 1982, Nucl. Acids Res. 10:6487-6500; and Kunkel, 1985, PNAS 82:488).

In applications where retention of antigen binding is required, such modifications should be at sites that do not disrupt the antigen binding capability of the antibody. In preferred embodiments, the one or more modifications are made in the constant region of the heavy and/or light chains.

Typically, the K_{D} for the antibody with respect to the target will be 2-fold, preferably 5-fold, more preferably 10-fold less than the K_{D} with respect to another, non-target molecule such as, but not limited to, unrelated material or accompanying material in the environment. More preferably, the K_{D} will be 50-fold less, such as 100-fold less or 200-fold less; even more preferably 500-fold less, such as 1,000-fold less, or 10,000-fold less than the K_{D} with respect the non-target molecule.

The value of this dissociation constant can be determined directly by well-known methods, and can be computed even for complex mixtures by methods such as those, for example, set forth in Caceci et al., 1984, Byte 9: 340-362. For example, the K_{D} may be established using a double-filter nitrocellulose filter binding assay such as that disclosed by Wong and Lohman, 1993, Proc. Natl. Acad. Sci. USA 90: 5428-5432. Other standard assays to evaluate the binding ability of ligands such as antibodies towards targets are known in the art, including for example, ELISAs, Western blots, RIAs, and flow cytometry analysis. The binding kinetics and binding affinity of the antibody also can be assessed by standard assays known in the art, such as Surface Plasmon Resonance (SPR), e.g. by using a Biacore™ system.

A competitive binding assay can be conducted in which the binding of the antibody to the target is compared to the binding of the target by another ligand of that target, such as another antibody. The concentration at which 50 percent binding inhibition occurs is known as the Kᵢ. Under ideal conditions, the Kᵢ is equivalent to K_{D}. The Kᵢ value will never be less than the K_{D}, so measurement of Kᵢ can conveniently be substituted to provide an upper limit for K_{D}.

An antibody of the invention may have a K_{D} for its target of no more than about 1×10⁻³ M, such as no more than about 1×10⁻³ M, no more than about 9×10⁻⁴ M, no more than about 8×10⁻⁴ M, no more than about 7×10⁻⁴ M, no more than about 6×10⁻⁴ M, no more than about 5×10⁻⁴ M, no more than about 4×10⁻⁴ M, no more than about 3×10⁻⁴ M, no more than about 2×10⁻⁴ M, no more than about 1×10⁻⁴ M, no more than about 9×10⁻⁵ M, no more than about 8×10⁻⁵ M, no more than about 7×10⁻⁵ M, no more than about 6×10⁻⁵ M, no more than about 5×10⁻⁵ M, no more than about 4×10⁻⁵ M, no more than about 3×10⁻⁵ M, no more than about 2×10⁻⁵ M, no more than about 1×10⁻⁵ M, no more than about 9×10⁻⁶ M, no more than about 8×10⁻⁶ M, no more than about 7×10⁻⁶ M, no more than about 6×10⁻⁶ M, no more than about 5×10⁻⁶ M, no more than about 4×10⁻⁶ M, no more than about 3×10⁻⁶ M, no more than about 2×10⁻⁶ M, no more than about 1×10⁻⁶ M, no more than about 9×10⁻⁷ M, no more than about 8×10⁻⁷ M, no more than about 7×10⁻⁷ M, no more than about 6×10⁻⁷ M, no more than about 5×10⁻⁷ M, no more than about 4×10⁻⁷ M, no more than about 3×10⁻⁷ M, no more than about 2×10⁻⁷ M, no more than about 1×10⁻⁷ M, no more than about 9×10⁻⁸ M, no more than about 8×10⁻⁸ M, no more than about 7×10⁻⁸ M, no more than about 6×10⁻⁸ M, no more than about 5×10⁻⁸ M, no more than about 4×10⁻⁸ M, no more than about 3×10⁻⁸ M, no more than about 2×10⁻⁸ M, no more than about 1×10⁻⁸ M, no more than about 9×10⁻⁹ M, no more than about 8×10⁻⁹ M, no more than about 7×10⁻⁹ M, no more than about 6×10⁻⁹ M, no more than about 5×10⁻⁹ M, no more than about 4×10⁻⁹ M, no more than about 3×10⁻⁹ M, no more than about 2×10⁻⁹ M, no more than about 1×10⁻⁹ M, from about 1×10⁻³ M to about 1×10⁻¹³ M, 1×10⁻⁴ M to about 1×10⁻¹³ M, 1×10⁻⁵ M to about 1×10⁻¹³ M, from about 1×10⁻⁶ M to about 1×10⁻¹³ M, from about 1×10⁻⁷ M to about 1×10⁻¹³ M, from about 1×10⁻⁸ M to about 1×10⁻¹³ M, from about 1×10⁻⁹ M to about 1×10⁻¹³ M, 1×10⁻³ M to about 1×10⁻¹² M, 1×10⁻⁴ M to about 1×10⁻¹² M, from about 1×10⁻⁵ M to about 1×10⁻¹² M, from about 1×10⁻⁶ M to about 1×10⁻¹² M, from about 1×10⁻⁷ M to about 1×10⁻¹² M, from about 1×10⁻⁸ M to about 1×10⁻¹² M, from about 1×10⁻⁹ M to about 1×10⁻¹² M, 1×10⁻³ M to about 1×10⁻¹¹ M, 1×10⁻⁴ M to about 1×10⁻¹¹ M, from about 1×10⁻⁵ M to about 1×10⁻¹¹ M, from about 1×10⁻⁶ M to about 1×10⁻¹¹ M, from about 1×10⁻⁷ M to about 1×10⁻¹¹ M, from about 1×10⁻⁸ M to about 1×10⁻¹¹ M, from about 1×10⁻⁹ M to about 1×10⁻¹¹ M, 1×10⁻³ M to about 1×10⁻¹⁰ M, 1×10⁻⁴ M to about 1×10⁻¹⁰ M, from about 1×10⁻⁵ M to about 1×10⁻¹⁰ M, from about 1×10⁻⁶ M to about 1×10⁻¹⁰ M, from about 1×10⁻⁷ M to about 1×10⁻¹⁰ M, from about 1×10⁻⁸ M to about 1×10⁻¹⁰ M, or from about 1×10⁻⁹ M to about 1×10⁻¹⁰ M.

Although in general, K_{D} at nanomolar range is desired, in certain embodiments, low affinity antibodies may be preferred, for example, for targeting highly expressed receptors in compartments and avoiding off-target binding. Further, some therapeutic applications may benefit from an antibody with lower binding affinity to facilitate antibody recycling.

Antibodies of the disclosure should retain the antigen binding capability of their native counterparts. In one embodiment, the antibodies of the disclosure exhibit essentially the same affinity as compared to an antibody prior to Cys substitution. In another embodiment, antibodies of the disclosure exhibit a reduced affinity as compared to an antibody prior to Cys substitution. In another embodiment, antibodies of the disclosure exhibit an enhanced affinity as compared to an antibody prior to Cys substitution.

In one embodiment, an antibody of the disclosure may have a dissociation constant (K_{D}) about equal to the K_{D} of the antibody prior to Cys substitution. In one embodiment, an antibody of the disclosure may have a dissociation constant (K_{D}) about 1-fold, about 2-fold, about 3-fold, about 4-fold, about 5-fold, about 10-fold, about 20-fold, about 50-fold, about 100-foldabout 150-fold, about 200-fold, about 250-fold, about 300-fold, about 400-fold, about 500-fold, about 600-fold, about 700-fold, about 800-fold, about 900-fold, or about 1000-fold greater for its cognate antigen compared with the K_{D} of the antibody prior to Cys substitution.

In yet another embodiment, an antibody of the disclosure may have a K_{D} about 1-fold, about 2-fold, about 3-fold, about 4-fold, about 5-fold, about 10-fold, about 20-fold, about 50-fold, about 100-fold, about 150-fold, about 200-fold, about 250-fold, about 300-fold, about 400-fold, about 500-fold, about 600-fold, about 700-fold, about 800-fold, about 900-fold, or about 1000-fold lower for its cognate antigen compared with the K_{D} of the antibody prior to Cys substitution.

Nucleic acids encoding the heavy and light chains of the antibodies used to make the ADCs of the invention can be cloned into a vector for expression or propagation. The sequence encoding the antibody of interest may be maintained in vector in a host cell and the host cell can then be expanded and frozen for future use.

Table 1 provides the amino acid (protein) sequences of humanized HER2 antibodies used in constructing the site specific ADCs of the invention. The CDRs shown are defined by Kabat numbering.

The antibody heavy chains and light chains shown in Table 1 have the trastuzumab heavy chain variable region (VH) and light chain variable region (VL). The heavy chain constant region and light chain constant region are derivatized from trastuzumab and contain on or more modification to allow for site specific conjugation when making the ADCs of the invention.

Modifications to the amino acid sequences in the antibody constant region to allow for site specific conjugation are underlined and bolded. The nomenclature for the antibodies derivatized from trastuzumab is T (for trastuzumab) and then the position of the amino acid of modification flanked by the single letter amino acid code for the wild type residue and the single letter amino acid code for the residue that is now in that position in the deriviatized antibody. Two exceptions to this nomenclature are “kK183C” which denotes that position 183 on the light (kappa) chain has been modified from a lysine to a cysteine and “LCQ05” which denotes an eight amino acid glutamine-containing tag that has been attached to the C terminus of the light chain constant region.

One of the modifications shown in Table 1 is not used for conjugation. The residue at position 222 on the heavy chain (using the EU index of Kabat) can be altered to result in a more homogenous antibody and payload conjugate, better intermolecular crosslinking between the antibody and the payload and/or significant decrease in interchain crosslinking.

TABLE 1
Sequences of humanized HER2 antibodies

| SEQ | | |
|---|---|---|
| ID | | |
| NO. | Description | Sequence |
| 1 | Trastuzumab | EVQLVESGGGLVQPGGSLRLSCAASGFNIKDTYIHWVRQAPGKGLEWVARTYPTNG |
| | VH protein | YTRYADSVKGRFTISADTSKNTAYLQMNSLRAEDTAVYYCSRWGGDGFYAMDYWGQ |
| | | GTLVTVSS |
| 2 | VH CDR1 | DTYIH |
| | protein | |
| 3 | VH CDR2 | RIYPTNGYTRYADSVKG |
| | protein | |
| 4 | VH CDR3 | WGGDGFYAMDY |
| | protein | |
| 5 | Trastuzumab | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLFPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPG |
| 6 | Trastuzumab | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | G |
| 7 | Trastuzumab | DIQMTQSPSSLSASVGDRVTITCRASQDVNTAVAWYQQKPGKAPKLLIYSASFLYS |
| | VL protein | GVPSRFSGSRSGTDFTLTISSLQPEDFATYYCQQHYTTPPTFGQGTKVEIK |
| 8 | VL CDR1 | RASQDVNTAVA |
| | protein | |
| 9 | VL CRD2 | SASFLYS |
| | protein | |
| 10 | VL CDR3 | QQHYTTPPT |
| | protein | |
| 11 | Trastuzumab | RTVAAPSVFIFPPSDEQLKSGTASVVCLLNNFYPREAKVQWKVDNALQSGNSQESV |
| | light chain | TEQDSKDSTYSLSSTLTLSKADYEKHKVYACEVTHQGLSSPVTKSENRGEC |
| | constant | |
| | region | |
| | protein | |
| 12 | Trastuzumab | DIQMTQSPSSLSASVGDRVTITCRASQDVNTAVAWYQQKPGKAPKLLIYSASFLYS |
| | light chain | GVPSRFSGSRSGTDFTLTISSLQPEDFATYYCQQHYTTPPTEGQGTKVEIKRTVAA |
| | protein | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 13 | T(K222R) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDRTHTCPPC |
| | constant | RAPELLGGPSVFLFPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 14 | T(K222R) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | RTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | GK |
| 15 | T(K246C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLFPPCPKDTLMISRTPEVTCVVVDVSHEDPEVKFNWYVDGVEVH |
| | region | NAKTKPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPG |
| 16 | T(K246C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPCPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | G |
| 17 | T(K290C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLFPPKPKDTLMISRTPEVTCVVVDVSHEDPEVKFNWYVDGVEVH |
| | region | NAKTCPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPG |
| 18 | T(K290C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTCPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | G |
| 19 | T(N297A) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTKPREEQYASTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 20 | T(N297A) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | GK |
| 21 | T(N297Q) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTKPREEQYQSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 22 | T(N297Q) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYQSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | GK |
| 23 | T(K334C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIECTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPG |
| 24 | T(K334C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | CTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | G |
| 25 | T(K392C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYCTTPPV |
| | | LDSDGSFFLYSKLTVDKSRWQQGNVFSCSVMHEALHNHYTQKSLSLSPG |
| 26 | T(K392C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYCTTPPVLDSDGSFFLYSKLTVDKSRWQQGNVFSCSVMHEALHNHYTQKSLSLSP |
| | | G |
| 27 | T(L443C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSCSPG |
| 28 | T(L443C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSCSP |
| | | G |
| 29 | T(K290C + K334C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTCPREEQYNSTYRVVSVLTVLHQDWLNGKEYKCKVSNKALPAPIECTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPG |
| 30 | T(K290C + K334C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTCPREEQYNSTYRVVSVLTVLHQDWLNGKEYKCKVSNKALPAPIE |
| | | CTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | G |
| 31 | T(K290C + K392C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLEPPKPKDTLMISRTPEVTCVVVDVSHEDPEVKFNWYVDGVEVH |
| | region | NAKTCPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYCTTPPV |
| | | LDSDGSFFLYSKLTVDKSRWQQGNVFSCSVMHEALHNHYTQKSLSLSPG |
| 32 | T(K290C + K392C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTCPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYCTTPPVLDSDGSFFLYSKLTVDKSRWQQGNVFSCSVMHEALHNHYTQKSLSLSP |
| | | G |
| 33 | T(N297A + K222R) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDRTHTCPPC |
| | constant | RAPELLGGPSVFLEPPKPKDTLMISRTPEVTCVVVDVSHEDPEVKFNWYVDGVEVH |
| | region | NAKTKPREEQYASTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 34 | T(N297A + K222R) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | RTHTCPPCPAPELLGGPSVFLFPPKPKDTLMISRTPEVTCVVVDVSHEDPEVKFNW |
| | | YVDGVEVHNAKTKPREEQYASTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | GK |
| 35 | T(N297Q + K222R) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDRTHTCPPC |
| | constant | RAPELLGGPSVFLFPPKPKDTLMISRTPEVTCVVVDVSHEDPEVKFNWYVDGVEVH |
| | region | NAKTKPREEQYQSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 36 | T(N297Q + K222R) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | RTHTCPPCPAPELLGGPSVFLFPPKPKDTLMISRTPEVTCVVVDVSHEDPEVKFNW |
| | | YVDGVEVHNAKTKPREEQYQSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYKTTPPVLDSDGSFFLYSKLTVDKSRWQQGNVFSCSVMHEALHNHYTQKSLSLSP |
| | | GK |
| 37 | T(K334C + K392C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLFPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIECTISKAKG |
| | protein | QPREPQVYTLPPSREEMTKNQVSLTCLVKGFYPSDIAVEWESNGQPENNYCTTPPV |
| | | LDSDGSFFLYSKLTVDKSRWQQGNVFSCSVMHEALHNHYTQKSLSLSPG |
| 38 | T(K334C + K392C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | CTISKAKGQPREPQVYTLPPSREEMTKNQVSLTCLVKGFYPSDIAVEWESNGQPEN |
| | | NYCTTPPVLDSDGSFFLYSKLTVDKSRWQQGNVFSCSVMHEALHNHYTQKSLSLSP |
| | | G |
| 39 | T(K392C + L443C) | ASTKGPSVFPLAPSSKSTSGGTAALGCLVKDYFPEPVTVSWNSGALTSGVHTFPAV |
| | heavy chain | LQSSGLYSLSSVVTVPSSSLGTQTYICNVNHKPSNTKVDKKVEPKSCDKTHTCPPC |
| | constant | RAPELLGGPSVFLFPPKPKDTLMISRTPEVTCVVVDVSHEDPEVKFNWYVDGVEVH |
| | region | NAKTKPREEQYNSTYRVVSVLTVLHQDWLNGKEYKCKVSNKALPAPIEKTISKAKG |
| | protein | QPREPQVYTLPPSREEMTKNQVSLTCLVKGFYPSDIAVEWESNGQPENNYCTTPPV |
| | | LDSDGSFFLYSKLTVDKSRWQQGNVFSCSVMHEALHNHYTQKSLSCSPG |
| 40 | T(K392C + L443C) | EVQLVESGGGLVQPGGSLRLSCAASGFNIKDTYIHWVRQAPGKGLEWVARTYPTNG |
| | heavy chain | YTRYADSVKGRFTISADTSKNTAYLQMNSLRAEDTAVYYCSRWGGDGFYAMDYWGQ |
| | protein | GTLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKKVEPKSCD |
| | | KTHTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEWESNGQPEN |
| | | NYCTTPPVLDSDGSFFLYSKLTVDKSRWQQGNVFSCSVMHEALHNHYTQKSLSCSP |
| | | G |
| 41 | T(kK183C) | RTVAAPSVFIFPPSDEQLKSGTASVVCLLNNFYPREAKVQWKVDNALQSGNSQESV |
| | light chain | TEQDSKDSTYSLSSTLTLSCADYEKHKVYACEVTHQGLSSPVTKSFNRGEC |
| | constant | |
| | region | |
| | protein | |
| 42 | T(kK183C) | DIQMTQSPSSLSASVGDRVTITCRASQDVNTAVAWYQQKPGKAPKLLIYSASFLYS |
| | light chain | GVPSRFSGSRSGTFTLTISSLQPEDFATYYCQQHYTTPPTFGQGTKVEIKRTVAAP |
| | protein | SVFIFPPSDEQLKSGTASVVCLLNNFYPREAKVQWKVDNALQSGNSQESVTEQDSK |
| | | DSTYSLSSTLTLSCADYEKHKVYACEVTHQGLSSPVTKSFNRGEC |
| 43 | T(LCQ05) | RTVAAPSVFIFPPSDEQLKSGTASVVCLLNNFYPREAKVQWKVDNALQSGNSQESV |
| | light chain | TEQDSKDSTYSLSSTLTLSKADYEKHKVYACEVTHQGLSSPVTKSENRGECGGLLQ |
| | constant | GPP |
| | region | |
| | protein | |
| 44 | T(LCQ05) | DIQMTQSPSSLSASVGDRVTITCRASQDVNTAVAWYQQKPGKAPKLLIYSASFLYS |
| | light chain | GVPSRFSGSRSGTDFTLTISSLQPEDFATYYCQQHYTTPPTEGQGTKVEIKRTVAA |
| | protein | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSENRGECGGLLQGPP |

ADCs can be made with an antibody component directed to any antigen using site specific conjugation through an engineered cysteine at position 290 (according to EU index of Kabat) either alone or in combination with other positions.

In some embodiments, the antigen binding domain (i.e., variable region having all 6 CDRs, or an equivalent region that is at least 90 percent identical to an antibody variable region), selected from the group consisting of: abagovomab, abatacept (ORENCIA®), abciximab (REOPRO®, c7E3 Fab), adalimumab (HUMIRA®), adecatumumab, alemtuzumab (CAMPATH®, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (SIMULECT®), bavituximab, bectumomab (LYMPHOSCAN®), belimumab (LYMPHO-STAT-B®), bertilimumab, besilesomab, βcept (ENBREL®), bevacizumab (AVASTIN®), biciromab brallobarbital, bivatuzumab mertansine, brentuximab vedotin (ADCETRIS®), canakinumab (ACZ885), cantuzumab mertansine, capromab (PROSTASCINT®), catumaxomab (REMOV ABC), cedelizumab (CIMZIA®), certolizumab pegol, cetuximab (ERBITUX®), clenoliximab, dacetuzumab, dacliximab, daclizumab (ZENAP AX(®), denosumab (AMG 162), detumomab, dorlimomab aritox, dorlixizumab, duntumumab, durimulumab, durmulumab, ecromeximab, eculizumab (SOLI RIS®), edobacomab, edrecolomab (Mabl7-1A, PANOREX®), efalizumab (RAPTIVA®), efungumab (MYCOGRAB®), elsilimomab, enlimomab pegol, epitumomab cituxetan, efalizumab, epitumomab, epratuzumab, erlizumab, ertumaxomab (REXOMUN®), etaracizumab (etaratuzumab, VITAXIN®, ABEGRIN™), exbivirumab, fanolesomab (NEUTROSPEC®), faralimomab, felvizumab, fontolizumab (HUZAF®), galiximab, gantenerumab, gavilimomab (ABX-CBL®), gemtuzumab ozogamicin (MYLOTARG®), golimumab (ONTO 148), gomiliximab, ibalizumab (TNX-355), ibritumomab tiuxetan (ZEVALIN®), igovomab, imciromab, infliximab (REMICAD E®), inolimomab, inotuzumab ozogamicin, ipilimumab (YERVOY®, MDX-010), iratumumab, keliximab, labetuzumab, lemalesomab, lebrilizumab, lerdelimumab, lexatumumab (HGS-ETR2, ETR2-ST01), lexitumumab, libivirumab, lintuzumab, lucatumumab, lumiliximab, mapatumumab (HGS-ETRI, TRM-I), maslimomab, matuzumab (EMD72000), mepolizumab (BOSATRIA®), metelimumab, milatuzumab, minretumomab, mitumomab, morolimumab, motavizumab (NUMAX™), muromonab (OKT3), nacolomab tafenatox, naptumomab estafenatox, natalizumab (TYSABRI®, ANTEGREN®), nebacumab, nerelimomab, nimotuzumab (THERACIM hR3®, THERA-CIM-hR3®, THERALOC®), nofetumomab merpentan (VERLUMA®), ocrelizumab, odulimomab, ofatumumab, omalizumab (XOLAIR®), oregovomab (OVAREX®), otelixizumab, pagibaximab, palivizumab (SYNAGIS®), panitumumab (ABX-EGF, VECTIBIX®), pascolizumab, pemtumomab (THERAGYN®), pertuzumab (2C4, OMNITARG®), pexelizumab, pintumomab, ponezumab, priliximab, pritumumab, ranibizumab (LUCENTIS®), raxibacumab, regavirumab, reslizumab, rituximab (RITUXAN®, MabTHERA®), rovelizumab, ruplizumab, satumomab, sevirumab, sibrotuzumab, siplizumab (MEDI-507), sontuzumab, stamulumab (Myo-029), sulesomab (LEUKOSCAN®), tacatuzumab tetraxetan, tadocizumab, talizumab, taplitumomab paptox, tefibazumab (AUREXIS®), telimomab aritox, teneliximab, teplizumab, ticilimumab, tocilizumab (ACTEMRA®), toralizumab, tositumomab, trastuzumab, tremelimumab (CP-675,206), tucotuzumab celmoleukin, tuvirumab, urtoxazumab, ustekinumab (ONTO 1275), vapaliximab, veltuzumab, vepalimomab, visilizumab (NUVION®), volociximab (M200), votumumab (HUMASPECT®), zalutumumab, zanolimumab (HuMAX-CD4), ziralimumab, or zolimomab aritox.

In some embodiments the antigen binding domain comprises a heavy and light chain variable domain having six CDRs, and/or competes for binding with an antibody selected from the preceding list. In some embodiments the antigen binding domain binds to the same epitope as the antibodies in the preceding list. In some embodiments the antigen binding domain comprises a heavy and light chain variable domain having six total CDRs, and binds to the same antigen as the antibodies in the preceding list.

In some embodiments the antigen binding domain comprises a heavy and light chain variable domain having six (6) total CDRs, and specifically binds to an antigen selected from the group consisting of: PDGFRα, PDGFRβ, PDGF, VEGF, VEGF-A, VEGF-B, VEGF-C, VEGF-D, VEGF-E, VEGF-F, VEGFR1, VEGFR2, VEGFR3, FGF, FGF2, HGF, KDR, FLT-1, FLK-1, Ang-2, Ang-1, PLGF, CEA, CXCL13, BAFF, IL-21, CCL21, TNF-α, CXCL12, SDF-I, bFGF, MAC-I, IL23p19, FPR, IGFBP4, CXCR3, TLR4, CXCR2, EphA2, EphA4, EphrinB2, EGFR (ErbBI), HER2 (ErbB2 or pI85neu), HER3 (ErbB3), HER4 ErbB4 or tyro2), SCI, LRPS, LRP6, RAGE, s100A8, s100A9, NavI.7, GLPI, RSV, RSV F protein, Influenza HA protein, Influenza NA protein, HMGBI, CD16, CD19, CD20, CD21, CD28, CD32, CD32b, CD64, CD79, CD22, ICAM-I, FGFRI, FGFR2, HDGF, EphB4, GITR, β-amyloid, hMPV, PIV-I, PIV-2, OX40L, IGFBP3, cMet, PD-I, PLGF, Neprolysin, CTD, IL-18, IL-6, CXCL-13, IL-IRI, IL-15, IL-4R, IgE, PAI-I, NGF, EphA2, uPARt, DLL-4, αvβ5, αvβ6, α5β1, α3β1, interferon receptor type I and type II, CD 19, ICOS, IL-17, Factor II, Hsp90, IGF, IGF-I, IGF-II, CD 19, GM-CSFR, PIV-3, CMV, IL-13, IL-9, and EBV.

In some embodiments the antigen binding domain specifically binds to a member (receptor or ligand) of the TNF superfamily. The TNF superfamily member may be selected from the group including, but not limited to, Tumor Necrosis Factor-α (“TNF-α”), Tumor Necrosis Factor-β (“TNF-β”), Lymphotoxin-α (“LT-α”), CD30 ligand, CD27 ligand, CD40 ligand, 4-1 BB ligand, Apo-1 ligand (also referred to as Fas ligand or CD95 ligand), Apo-2 ligand (also referred to as TRAIL), Apo-3 ligand (also referred to as TWEAK), osteoprotegerin (OPG), APRIL, RANK ligand (also referred to as TRANCE), TALL-1 (also referred to as BlyS, BAFF or THANK), DR4, DR5 (also known as Apo-2, TRAIL-R2, TR6, Tango-63, hAPO8, TRICK2, or KILLER), DR6, DcRI, DcR2, DcR3 (also known as TR6 or M68), CARI, HVEM (also known as ATAR or TR2), GITR, ZTNFR-5, NTR-I, TNFLI, CD30, LTBr, 4-1BB receptor and TR9.

In some embodiments the antigen binding domain is capable of binding one or more targets chosen from the group including, but not limited to, 5T4, ABL, ABCB5, ABCFI, ACVRI, ACVRIB, ACVR2, ACVR2B, ACVRLI, ADORA2A, Aggrecan, AGR2, AICDA, AIFI, AIGI, AKAPI, AKAP2, AMH, AMHR2, angiogenin (ANG), ANGPTI, ANGPT2, ANGPTL3, ANGPTL4, Annexin A2, ANPEP, APC, APOCI, AR, aromatase, ATX, AXI, AZGPI (zinc-a-glycoprotein), B7.1, B7.2, B7-H1, BAD, BAFF, BAG1, BAII, BCR, BCL2, BCL6, BDNF, BLNK, BLRI (MDR15), BlyS, BMP1, BMP2, BMP3B (GDFIO), BMP4, BMP6, BMP7, BMP8, BMP9, BMP11, BMP12, BMPR1A, BMPR1B, BMPR2, BPAGI (plectin), BRCAI, C19orfIO (IL27w), C3, C4A, C5, C5R1, CANTI, CASPI, CASP4, CAVI, CCBP2 (D6/JAB61), CCLI (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-I), CCL23 (MPIF-1), CCL24 (MPIF-2/eotaxin-2), CCL25 (TECK), CCL26(eotaxin-3), CCL27 (CTACK/ILC), CCL28, CCL3 (MIP-Ia), CCL4 (MIP-Ib), CCL5(RANTES), CCL7 (MCP-3), CCL8 (mcp-2), CCNAI, CCNA2, CCNDI, CCNEI, CCNE2, CCRI (CKRI/HM145), CCR2 (mcp-IRB/RA), CCR3 (CKR3/CMKBR3), CCR4, CCR5(CMKBR5/ChemR13), CCR6 (CMKBR6/CKR-L3/STRL22/DRY6), CCR7 (CKR7/EBI1),CCR8 (CMKBR8/TERI/CKR-LI), CCR9 (GPR-9-6), CCRLI (VSHKI), CCRL2 (L-CCR),CD164, CD19, CDIC, CD20, CD200, CD-22, CD24, CD28, CD3, CD33, CD35, CD37, CD38, CD3E, CD3G,CD3Z, CD4, CD40, CD4OL, CD44, CD45RB, CD46, CD52, CD69, CD72, CD74, CD79A, CD79B, CD8, CD80, CD81, CD83, CD86, CD105, CD137, CDHI (E-cadherin), CDCP1CDH10, CDH12, CDH13, CDH18,CDH19, CDH20, CDH5, CDH7, CDH8, CDH9, CDK2, CDK3, CDK4, CDK5, CDK6, CDK7, CDK9, CDKNIA (p21WapI/CipI), CDKNIB (p27KipI), CDKNIC, CDKN2A (p16INK4a),CDKN2B, CDKN2C, CDKN3, CEBPB, CERI, CHGA, CHGB, Chitinase, CHSTIO, CKLFSF2,CKLFSF3, CKLFSF4, CKLFSF5, CKLFSF6, CKLFSF7, CKLFSF8, CLDN3, CLDN7 (claudin-7), CLN3, CLU (clusterin), CMKLRI, CMKORI (RDCI), CNRI, COLI 8A1, COL1A1.COL4A3, COL6A1, CR2, Cripto, CRP, CSF1 (M-CSF), CSF2 (GM-CSF), CSF3 (GCSF), CTLA4, CTL8, CTNNBI (b-catenin), CTSB (cathepsin B), CX3CL1 (SCYDI), CX3CR1 (V28), CXCLI(GROI), CXCLIO (IP-IO), CXCL11 (I-TAC/IP-9), CXCL12 (SDFI), CXCL13, CXCL 14,CXCL 16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo),CYB5, CYCI, Cyr61, CYSLTRI, c-Met, DAB2IP, DES, DKFZp451J0118, DNCLI, DPP4, E2F1, ECGFI5EDGI, EFNAI, EFNA3, EFNB2, EGF, ELAC2, ENG, endoglin, ENOI, EN02, EN03, EPHAI, EPHA2, EPHA3, EPHA4, EPHA5, EPHA6, EPHA7, EPHA8, EPHA9, EPHAIO, EPHBI, EPHB2, EPHB3, EPHB4, EPHB5, EPHB6, EPHRIN-AI, EPHRIN-A2, EPHRIN-A3, EPHRIN-A4, EPHRIN-A5, EPHRIN-A6, EPHRIN-BI, EPHRIN-B2, EPHRTN-B3, EPHB4, EPG, ERBB2 (Her-2), EREG, ERK8, Estrogen receptor, ESRI, ESR2, F3 (TF), FADD, farnesyltransferase, FasL, FASNf, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF), FGF20, FGF21 (such as mimAb1), FGF22, FGF23, FGF3 (int-2),FGF4 (HST), FGF5, FGF6 (HST-2), FGF7 (KGF), FGF8, FGF9, FGFR3, FIGF (VEGFD), FILI (EPSILON), FBLI (ZETA), FLJ12584, FLJ25530, FLRTI (fibronectin), FLTI, FLT-3, FOS, FOSLI (FRA-I), FY (DARC), GABRP (GABAa), GAGEBI, GAGECI, GALNAC4S-6ST, GATA3, GD2, GD3, GDF5, GDF8, GFII, GGTI, GM-CSF, GNASI, GNRHI, GPR2 (CCRIO), GPR31, GPR44, GPR81 (FKSG80), GRCCIO (C10), gremlin, GRP, GSN (Gelsolin), GSTPI, HAVCR2, HDAC, HDAC4, HDAC5, HDAC7A, HDAC9, Hedgehog, HGF, HIFIA, HIPI, histamine and histamine receptors, HLA-A, HLA-DRA, HM74, HMOXI, HSP90, HUMCYT2A, ICEBERG, ICOSL, ID2, IFN-a, IFNAI, IFNA2, IFNA4,IFNA5, EFNA6, BFNA7, IFNB1, IFNgamma, IFNWI, IGBPI, IGF1, IGF1R, IGF2, IGFBP2,IGFBP3, IGFBP6, DL-I, ILIO, ILIORA, ILIORB, IL-1, ILIRI (CD121a), ILIR2(CD121b), IL-IRA, IL-2, IL2RA (CD25), IL2RB (CD122), IL2RG (CD132), IL-4, IL-4R(CD123), IL-5, IL5RA (CD125), IL3RB (CD131), IL-6, IL6RA (CD126), IR6RB (CD130), IL-7, IL7RA(CD127), IL-8, CXCRI (IL8RA), CXCR2 (IL8RB/CD128), IL-9, IL9R (CD129), IL-10, IL10RA (CD210), IL10RB (CDW210B), IL-11, ILI IRA, IL-12, IL-12A, IL-12B, IL-12RB1, IL-12RB2, IL-13, IL13RA1, IL13RA2, IL14, IL15, IL15RA, 1L16, IL17, IL17A, IL17B, IL17C, IL17R, IL18, IL18BP, IL18R1, IL18RAP, IL19, IL1A, IL1B, IL1F10, IL1F5, IL1F6, IL1F7, IL1F8, DL1F9, IL1HYI, IL1R1, IL1R2, IL1RAP, IL1RAPL1, IL1RAPL2, IL1RL1, IL1RL2, IL1RN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4R, IL6ST (glycoprotein 130), ILK, INHA, INHBA, INSL3, INSL4, IRAKI, IRAK2, ITGA1, ITGA2, ITGA3, ITGA6 (α 6 integrin), ITGAV, ITGB3, ITGB4 (β 4 integrin), JAK1, JAK3, JTB, JUN, K6HF, KAII, KDR, KIM-1, KITLG, KLFS (GC Box BP), KLF6, KLK10, KLK12, KLK13, KLK14, KLK15, KLK3, KLK4, KLK5, KLK6, KLK9, KRT1, KRT19 (Keratin 19), KRT2A, KRTHB6 (hair-specific type II keratin), LAMAS, LEP (leptin), Lingo-p75, Lingo-Troy, LPS, LRP5, LRP6, LTA (TNF-b), LTB, LTB4R (GPR16), LTB4R2, LTBR, MACMARCKS, MAG or Omgp, MAP2K7 (c-Jun), MCP-1, MDK, MIBI, midkine, MIF, MISRII, MJP-2, MK, MKI67 (Ki-67), MMP2, MMP9, MS4A1, MSMB, MT3 (metallothionectin-Ui), mTOR, MTSSI, MUCI (mucin), MYC, MYD88, NCK2, neurocan, neuregulin-1, neuropilin-1, NFKBI, NFKB2, NGFB (NGF), NGFR, NgR-Lingo, NgR-Nogo66 (Nogo), NgR-p75, NgR-Troy, NMEI (NM23A), NOTCH, NOTCH1, NOX5, NPPB, NROBI, NROB2, NRIDI, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRPI, NRP2, NT5E, NTN4, OCT-1, ODZ1, OPN1, OPN2, OPRDI, P2RX7, PAP, PARTI, PATE, PAWR, PCA3, PCDGF, PCNA, PDGFA, PDGFB, PDGFRA, PDGFRB, PECAMI, peg-asparaginase, PF4 (CXCL4), Plexin B2 (PLXNB2), PGF, PGR, phosphacan, PIAS2, P13 Kinase, PIK3CG, PLAU (uPA), PLG5PLXDCI, PKC, PKC-β, PPBP (CXCL7), PPID, PRI, PRKCQ, PRKDI, PRL, PROC, PROK2, pro-NGF, prosaposin, PSAP, PSCA, PTAFR, PTEN, PTGS2 (COX-2), PTN, RAC2 (P21Rac2), RANK, RANK ligand, RARB, RGSI, RGS13, RGS3,RNFI10 (ZNF144), Ron, ROB02, RXR, selectin, S100A2, S100A8, S100A9, SCGB 1D2 (lipophilin B), SCGB2A1 (mammaglobin 2), SCGB2A2 (mammaglobin 1), SCYEI (endothelial Monocyte-activating cytokine), SDF2, SERPENA1, SERPINA3, SERPINB5 (maspin), SERPINEI (PAI-I), SERPINFI, SHIP-1, SHIP-2, SHBI, SHB2, SHBG, SfcAZ, SLC2A2, SLC33A1, SLC43A1, SLIT2, SPPI, SPRRIB (SprI), ST6GAL1, STABI, STATE, STEAP, STEAP2, SULF-1, Sulf-2, TB4R2, TBX21, TCPIO, TDGFI, TEK, TGFA, TGFB1, TGFBIII, TGFB2,TGFB3, TGFBI, TGFBRI, TGFBR2, TGFBR3, THIL, THBSI (thrombospondin-1), THBS2/THBS4, THPO, TIE (Tie-1), TIMP3, tissue factor, TIKI2, TLR10, TLR2, TLR3, TLR4, TLR5, TLR6JLR7, TLR8, TLR9, TM4SF1, TNF, TNF-a, TNFAIP2 (B94), TNFAIP3, TNFRSFIIA, TNFRSFIA, TNFRSFIB, TNFRSF21, TNFRSF5, TNFRSF6 (Fas), TNFRSF7, TNFRSF8, TNFRSF9, TNFSFIO (TRAIL), TNFSFI1 (TRANCE), TNFSF12 (APO3L), TNFSF13 (April), TNFSF13B, TNFSF14 (HVEM-L), TNFSF15 (VEGI), TNFSF 18, TNFSF4 (OX40 ligand), TNFSFS (CD40 ligand), TNFSF6 (FasL), TNFSF7 (CD27 ligand), TNFSF8 (CD30 ligand), TNFSF9 (4-1BB ligand), TOLLIP, Toll-like receptors, TLR2, TLR4, TLR9, TOP2A (topoisomerase Iia), TP53, TPMI, TPM2,TRADD, TRAFI, TRAF2, TRAF3, TRAF4, TRAF5, TRAF6, TRKA, TREMI, TREM2, TRPC6, TROY, TSLP, TWEAK, Tyrosinase, uPAR, VEGF, VEGFB, VEGFC, versican, VHL C5, VLA-4, Wnt-1, XCLI (Iymphotactin), XCL2 (SCM-Ib), XCRI (GPRS/CCXCRI), YYI, and ZFPM2.

In some embodiments, the antibody, or antigen-binding fragment thereof, binds to extra-domain B (EDB) of fibronectin (FN). FN-EDB is a small domain of 91 amino acids, which can be inserted into fibronectin molecules by a mechanism of alternative splicing. The amino acid sequence of FN-EDB is 100% conserved between human, cynomolgus monkey, rat and mouse. FN-EDB is overexpressed during embryonic development and broadly expressed in human cancers, but virtually undetectable in normal adult except female reproductive tissues.

In certain embodiments, the antibody, or antigen-binding fragment thereof, described herein comprises the following heavy chain CDR sequences: (i) a VH complementarity determining region one (CDR-H1) sharing at least 90%, at least 91%, at least 92%, at least 93%, at least 94%, or at least 95% identical to SEQ ID NO: 66 or 67, a CDR-H2 sharing at least 90%, at least 91%, at least 92%, at least 93%, at least 94%, or at least 95% identity with SEQ ID NO: 68 or 69, and a CDR-H3 sharing at least 90%, at least 91%, at least 92%, at least 93%, at least 94%, or at least 95% identity with SEQ ID NO: 70; and/or (ii) the following light chain CDR sequences: a VL complementarity determining region one (CDR-L1) sharing at least 90%, at least 91%, at least 92%, at least 93%, at least 94%, or at least 95% identity with SEQ ID NO: 73, a CDR-L2 sharing at least 90%, at least 91%, at least 92%, at least 93%, at least 94%, or at least 95% identity with SEQ ID NO: 74, and a CDR-L3 sharing at least 90%, at least 91%, at least 92%, at least 93%, at least 94%, or at least 95% identity with SEQ ID NO: 75.

In certain embodiments, the antibody or antigen-binding fragment thereof described herein comprises (i) a heavy chain variable region (VH) comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 65, and/or (ii) light chain variable region (VL) comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 72. Any combination of these VL and VH sequences is also encompassed by the invention.

In certain embodiments, the antibody or antigen-binding fragment thereof described herein comprises an Fc domain. The Fc domain can be derived from IgA (e.g., IgA₁ or IgA₂), IgG, IgE, or IgG (e.g., IgG₁, IgG₂, IgG₃, or IgG₄).

In certain embodiments, the antibody or antigen-binding fragment thereof described herein comprises (i) a heavy chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 71 or 77, and/or (ii) a light chain comprising an amino acid sequence that is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO: 76 or 78. Any combination of these heavy chain and light chain sequences is also encompassed by the invention.

Also provided by the invention is an antibody, or antigen-binding fragment thereof, that competes for binding to EDB with any of the anti-EDB antibody or antigen-binding fragment thereof described herein, such as any one of the antibodies listed in Table 33, or antigen-binding fragments thereof.

The invention provides a nucleic acid encoding an engineered polypeptide described herein. The invention also provides a nucleic acid encoding an antibody comprising an engineered polypeptide described herein.

The invention also provides a host cell comprising a nucleic acid encoding the engineered polypeptide described herein. The invention also provides a host cell comprising a nucleic acid encoding an antibody comprising the engineered polypeptide described herein.

The invention provides a nucleic acid encoding an antibody, or antigen-binding fragment thereof, of any one of the HER2 antibodies disclosed herein, and a host cell comprising such a nucleic acid.

The invention provides a nucleic acid encoding an antibody, or antigen-binding fragment thereof, of any one of the anti-EDB antibodies disclosed herein, and a host cell comprising such a nucleic acid.

The invention provides a method of producing an engineered polypeptide described herein, or antibody, or antigen-binding portion thereof, comprising such an engineered polypeptide. The method comprises culturing the host cell under suitable conditions for expressing the polypeptide, the antibody, or antigen-binding portion thereof, and isolating the polypeptide, or the antibody or antigen-binding fragment.

B. Drugs

Drugs useful in preparation of the site specific ADCs of the invention include any therapeutic agent useful in the treatment of diseases (e.g., cancer) including, but not limited to, cytotoxic agents, cytostatic agents, immunomodulating agents and chemotherapeutic agents. A cytotoxic effect refers to the depletion, elimination and/or the killing of a target cells (i.e., tumor cells). A cytotoxic agent refers to an agent that has a cytotoxic effect on a cell. A cytostatic effect refers to the inhibition of cell proliferation. A cytostatic agent refers to an agent that has a cytostatic effect on a cell, thereby inhibiting the growth and/or expansion of a specific subset of cells (i.e., tumor cells). An immunomodulating agent refers to an agent that stimulates the immune response though the production of cytokines and/or antibodies and/or modulating T cell function thereby inhibiting or reducing the growth of a subset of cells (i.e., tumor cells) either directly or indirectly by allowing another agent to be more efficacious. A chemotherapeutic agent refers to an agent that is chemical compound useful in the treatment of cancer. A drug may also be a drug derivative, wherein a drug has been functionalized to enable conjugation with an antibody of the invention.

In some embodiments the drug is a membrane permeable drug. In such embodiments, the payload can elicit a bystander effect wherein cells surrounding the cell that initially internalized the ADC are killed by the payload. This occurs when the payload is released from the antibody (i.e., by cleaving of a cleavable linker) and crosses the cellular membrane and, upon diffusion, induces the killing of surrounding cells.

In accordance with the disclosed methods, the drugs are used to prepare antibody drug conjugates of the formula Ab-(L-D), wherein (a) Ab is an antibody that binds to a specific target; and (b) L-D is a linker-drug moiety, wherein L is a linker, and D is a drug.

The drug-to-antibody ratio (DAR) or drug loading indicates the number of drug (D) molecules that are conjugated per antibody. The antibody drug conjugates of the present invention use site specific conjugation such that there is essentially a homogeneous population of ADCs having one DAR in a composition of ADCs. In some embodiments, the DAR is 1. In some embodiments, the DAR is 2. In other embodiments, the DAR is 3. In other embodiments, the DAR is 4. In other embodiments, the DAR is greater than 4.

Using conventional conjugation (rather than site specific conjugation) results in a heterogeneous population of different species of ADCs, each of which with a different individual DAR. Compositions of ADCs prepared in this way include a plurality of antibodies, each antibody conjugated to a particular number of drug molecules. As such, the compositions have an average DAR. T-DM1 (Kadcyla®) uses conventional conjugation on lysine residues and has an average DAR of around 4 with a broad distribution including ADCs loaded with 0, 1, 2, 3, 4, 5, 6, 7 or 8 drug molecules (Kim et al., 2014, Bioconj Chem 25(7):1223-32).

DAR can be determined by various conventional means such as UV spectroscopy, mass spectroscopy, ELISA assay, radiometric methods, hydrophobic interaction chromatography (HIC), electrophoresis and HPLC.

In one embodiment, the drug component of the ADCs of the invention is an anti-mitotic drug. In certain embodiments, the anti-mitotic drug may be an auristatin (e.g., 0101, 8261, 6121, 8254, 6780 and 0131; see Table 2 infra). In a more specific embodiment, the auristatin drug is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as 0101).

Auristatins inhibit cell proliferation by inhibiting the formation of microtubules during mitosis through inhibition of tubulin polymerization. PCT International Publication No. WO 2013/072813, which is incorporated by reference in its entirety, discloses auristatins that are useful in the manufacture of the ADCs of the invention and provides methods of producing those auristatins

TABLE 2
Drugs

| Name | Structure | IUPAC Name |
|---|---|---|
| 0101 | 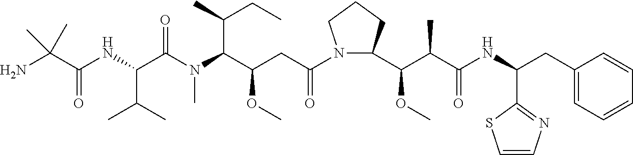 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)C(C)(C)N)C(C)C] | 2-methylalanyl-N- [(3R,4S,5S)-3-methoxy- 1-{(2S)-2-[(1R,2R)-1- methoxy-2-methyl-3- oxo-3-{[(1S)-2-phenyl- 1-(1,3-thiazol-2- yl)ethyl]amino}propyl] pyrrolidin-1-yl}-5- methyl-1-oxoheptan-4- yl]-N-methyl-L- |
| | | valinamide |
| 8261 | 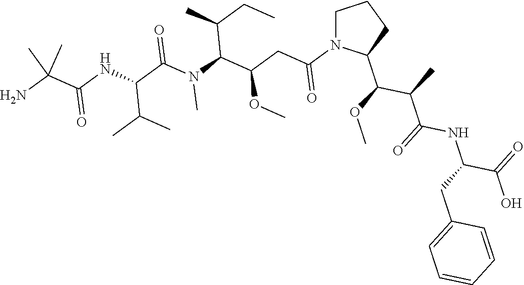 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)C(C)(C)N)C(C)C] | 2-methylalanyl-N- [(3R,4S,5S)-1-{(2S)-2- [(1R,2R)-3-{[(1S)-1- carboxy-2-phenylethyl] amino}-1-methoxy-2- methyl-3-oxopropyl] pyrrolidin-1-yl}-3- methoxy-5-methyl-1- oxoheptan-4-yl]-N- methyl-L-valinamide |
| 6121 | 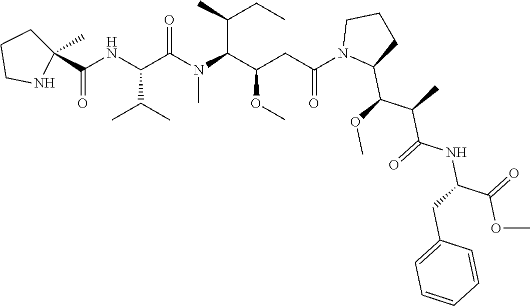 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@]1(C)CCCN1)C(C)C] | 2-methyl-L-prolyl-N- [(3R,4S,5S)-3-methoxy- 1-{(2S)-2-[(1R,2R)-1- methoxy-3-{[(2S)-1- methoxy-1-oxo-3- phenylpropan-2-yl] amino}-2-methyl-3- oxopropyl]pyrrolidin-1- yl}-5-methyl-1- oxoheptan-4-yl]-N- methyl-L-valinamide, trifluoroacetic acid salt |
| 8254 | 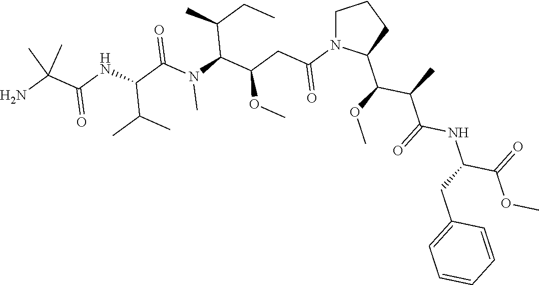 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)C(C)(C)N)C(C)C] | 2-methylalanyl-N- [(3R,4S,5S)-3-methoxy- 1-{(2S)-2-[(1R,2R)-1- methoxy-3-{[(2S)-1- methoxy-1-oxo-3- phenylpropan-2-yl] amino}-2-methyl-3- oxopropyl]pyrrolidin- 1-yl}-5-methyl-1- oxoheptan-4-yl]-N- methyl-L-valinamide |
| 6780 | 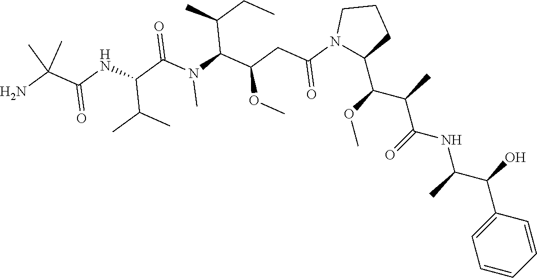 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)C(C)(C)N)C(C)C] | 2-methylalanyl-N- [(3R,4S,5S)-1-{(2S)-2- [(1R,2R)-3-{[(1S,2R)- 1-hydroxy-1- phenylpropan-2- yl]amino}-1-methoxy- 2-methyl-3- oxopropyl]pyrrolidin- 1-yl}-3-methoxy-5- methyl-1-oxoheptan-4- yl]-N-methyl-L- valinamide |
| 0131 | 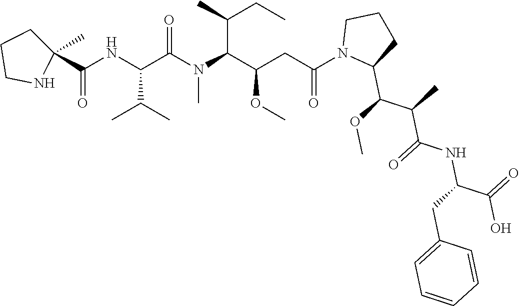 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@]1(C)CCCN1)C(C)C] | 2-methyl-L-prolyl-N- [(3R,4S,5S)-1-{(2S)-2- [(1R,2R)-3-{[(1S)-1- carboxy-2- phenylethyl]amino}- 1-methoxy-2-methyl-3- oxopropyl]pyrrolidin-1- yl}-3-methoxy-5- methyl-1-oxoheptan-4- yl]-N-methyl-L- valinamide, trifluoroacetic acid salt |
| MMAD | 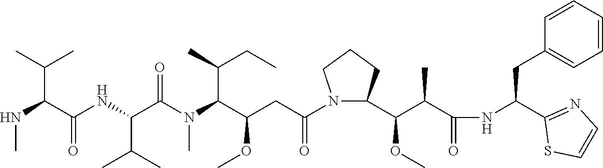 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H](NC)C(C)C)C(C)C] | N-methyl-L-valyl-N- [(3R,4S,5S)-3-methoxy- 1-{(2S)-2-[(1R,2R)-1- methoxy-2-methyl-3- oxo-3-{[(1S)-2-phenyl- 1-(1,3-thiazol-2- yl)ethyl]amino}propyl] pyrrolidin-1-yl}-5- methyl-1-oxoheptan-4- yl]-N-methyl-L- valinamide |
| MMAE | 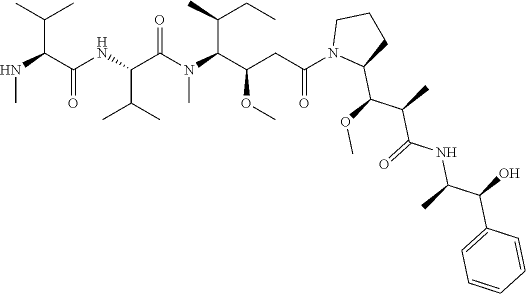 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C] | N-methyl-L-valyl-N- [(3R,4S,5S)-1-{(2S)-2- [(1R,2R)-3-{[(1S,2R)- 1-hydroxy-1- phenylpropan- 2-yl]amino}-1- methoxy-2-methyl-3- oxopropyl]pyrrolidin- 1-yl}-3-methoxy-5- methyl-1-oxoheptan- 4-yl]-N-methyl-L- valinamide |
| MMAF | 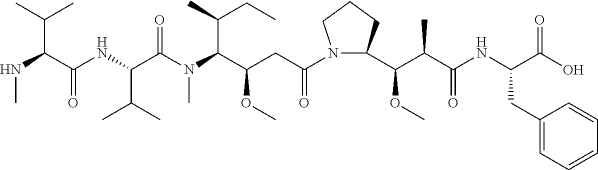 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C] | N-methyl-L-valyl-N- [(3R,4S,5S)-1-{(2S)-2- [(1R,2R)-3-{[(1S)-1- carboxy-2- phenylethyl]amino}- 1-methoxy-2-methyl-3- oxopropyl]pyrrolidin-1- yl}-3-methoxy-5- methyl-1-oxoheptan-4- yl]-N-methyl-L- valinamide |

In some aspects of the invention, the cytotoxic agent can be made using a liposome or biocompatible polymer. The antibodies as described herein can be conjugated to the biocompatible polymer to increase serum half-life and bioactivity, and/or to extend in vivo half-lives. Examples of biocompatible polymers include water-soluble polymer, such as polyethylene glycol (PEG) or its derivatives thereof and zwitterion-containing biocompatible polymers (e.g., a phosphorylcholine containing polymer).

C. Linkers

Site specific ADCs of the invention are prepared using a linker to link or conjugate a drug to an antibody. A linker is a bifunctional compound which can be used to link a drug and an antibody to form an antibody drug conjugate (ADC). Such conjugates allow the selective delivery of drugs to tumor cells. Suitable linkers include, for example, cleavable and non-cleavable linkers. A cleavable linker is typically susceptible to cleavage under intracellular conditions. Major mechanisms by which a conjugated drug is cleaved from an antibody include hydrolysis in the acidic pH of the lysosomes (hydrazones, acetals, and cis-aconitate-like amides), peptide cleavage by lysosomal enzymes (the cathepsins and other lysosomal enzymes), and reduction of disulfides. As a result of these varying mechanisms for cleavage, mechanisms of linking the drug to the antibody also vary widely and any suitable linker can be used.

Suitable cleavable linkers include, but are not limited to, a peptide linker cleavable by an intracellular protease, such as lysosomal protease or an endosomal protease such as vc, and m(H20)c-vc (Table 3 infra). In specific embodiments, the linker is a cleavable linker such that the payload can induce a bystander effect once the linker is cleaved. The bystander effect is when a membrane permeable drug is released from the antibody (i.e., by cleaving of a cleavable liner) and crosses the cellular membrane and, upon diffusion, induce killing of cells surrounding the cell that initially internalized the ADC.

Suitable non-cleavable linkers include, but are not limited to, mc, MalPeg6, Mal-PEG2C2, Mal-PEG3C2 and m(H20)c (Table 3 infra).

Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the mc linker and the like.

In particular aspects of the invention, the linker in the site specific ADCs of the invention are cleavable and may be vc.

Many of the therapeutic agents conjugated to antibodies have little, if any, solubility in water and that can limit drug loading on the conjugate due to aggregation of the conjugate. One approach to overcoming this is to add solublizing groups to the linker. Conjugates made with a linker consisting of PEG and a dipeptide can been used, including those having a PEG di-acid, thiol-acid, or maleimide-acid attached to the antibody, a dipeptide spacer, and an amide bond to the amine of an anthracycline or a duocarmycin analogue. Another example is a conjugate prepared with a PEG-containing linker disulfide bonded to a cytotoxic agent and amide bonded to an antibody. Approaches that incorporate PEG groups may be beneficial in overcoming aggregation and limits in drug loading.

TABLE 3
Linkers

| Name | Structure |
|---|---|
| vc (MC-vc-PAB) | 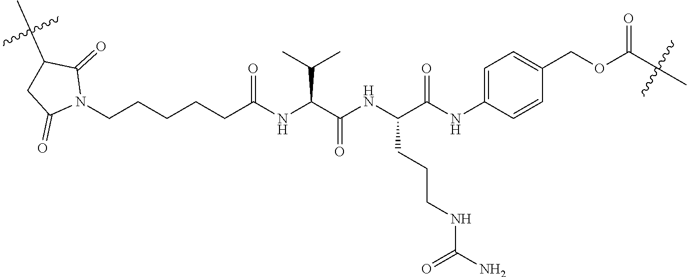 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1] |
| AcLysvc | 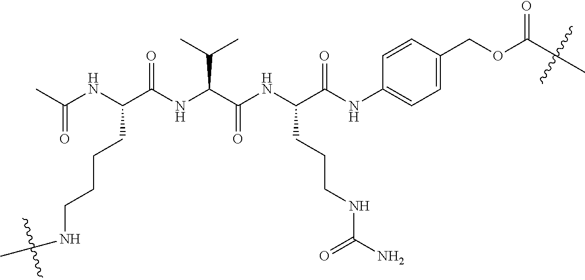 [SMILES: *NCCCC[C@H](NC(C)=O)C(=O)N[C@H](C(=O)N[C@@H](CCCNC(N)=O)C(=O)Nc1ccc(COC(*)=O)cc1)C(C)C] |
| mc | 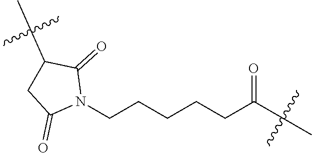 [SMILES: *C(=O)CCCCCN1C(=O)CC(*)C1=O] |
| MalPeg6 | 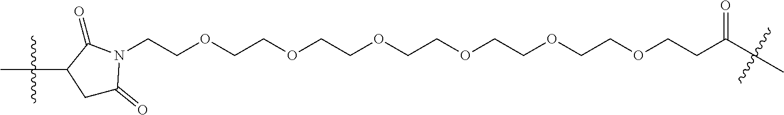 [SMILES: *C(=O)CCOCCOCCOCCOCCOCCOCCN1C(=O)CC(*)C1=O] |
| m(H20)c | 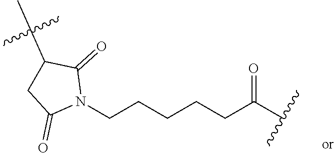 [SMILES: *C(=O)CCCCCN1C(=O)CC(*)C1=O] |
| | 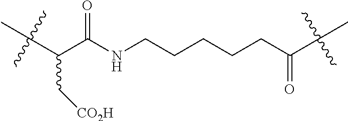 [SMILES: *C(=O)CCCCCNC(=O)C(*)CC(=O)O] |
| m(H20)c-vc (m(H2O)c-vc- PAB) | 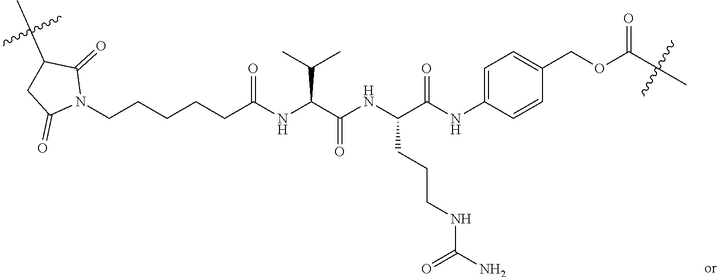 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1] |
| | 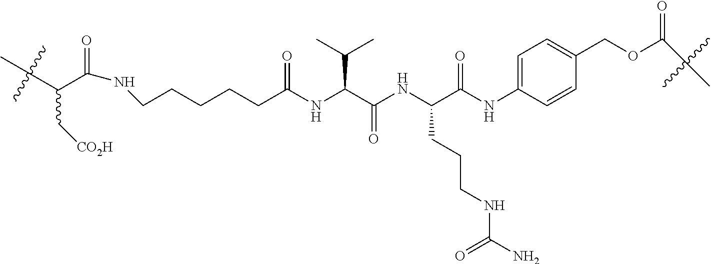 [SMILES: *C(=O)OCc1ccc(NC(=O)[C@H](CCCNC(N)=O)NC(=O)[C@@H](NC(=O)CCCCCNC(=O)C(*)CC(=O)O)C(C)C)cc1] |

Linkers are attached to the monoclonal antibody via the left side of the molecule and the drug via the right side of the molecule as depicted in Table 3.

In certain embodiment, the antibody of the invention is conjugated to a thiol-reactive agent in which the reactive group is, for example, a maleimide, an iodoacetamide, a pyridyl disulfide, or other thiol-reactive conjugation partner (Haugland, 2003, Molecular Probes Handbook of Fluorescent Probes and Research Chemicals, Molecular Probes, Inc.; Brinkley, 1992, Bioconjugate Chem. 3:2; Garman, 1997, Non-Radioactive Labelling: A Practical Approach, Academic Press, London; Means (1990) Bioconjugate Chem. 1:2; Hermanson, G. in Bioconjugate Techniques (1996) Academic Press, San Diego, pp. 40-55, 643-671).

In certain embodiments, the invention provides an antibody drug conjugate of the formula Ab-(L-D), wherein (a) Ab is an antibody that binds to a specific target; and (b) L-D is a linker-drug moiety, wherein L is a linker, and D is a drug.

In certain embodiments, the Ab-(L-D) comprises a succinimide group, a maleimide group, a hydrolyzed succinimide group, or a hydrolyzed maleimide group.

In certain embodiments, the Ab-(L-D) comprises a maleimide group or a hydrolyzed maleimide group. Maleimides such as N-ethylmaleimide are considered to be specific to sulfhydryl groups, especially at pH values below 7, where other groups are protonated.

In certain embodiments, the Ab-(L-D) comprises 6-maleimidocaproyl (MC), maleimidopropanoyl (MP), valine-citrulline (val-cit), alanine-phenylalanine (ala-phe), p-am inobenzyloxycarbonyl (PAB), N-Succinimidyl 4-(2-pyridylthio) pentanoate (SPP), N-succinimidyl 4-(N-maleimidomethyl) cyclohexane-1carboxylate (SMCC), N-Succinimidyl (4-iodo-acetyl) aminobenzoate (SIAB), or 6-maleimidocaproyl-valine-citrulline-p-aminobenzyloxycarbonyl (MC-vc-PAB).

In certain embodiment, the Ab-(L-D) comprises the compound of formula I:

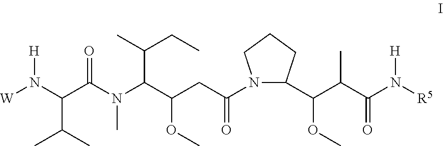

or a pharmaceutically acceptable salt or solvate thereof, wherein, independently for each occurrence,
W is

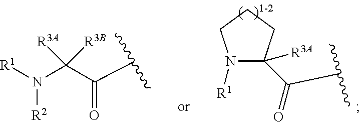

R¹ is hydrogen or C₁-C₈ alkyl;
R² is hydrogen or C₁-C₈ alkyl;
R^{3A} and R^{3B} are either of the following:
(iii) R^{3A} is hydrogen or C₁-C₈ alkyl;
   R^{3B} is C₁-C₈ alkyl;
(iv) R^{3A} and R^{3B} taken together are C₂-C₈ alkylene or C₁-C₈ heteroalkylene;
R⁵ is and

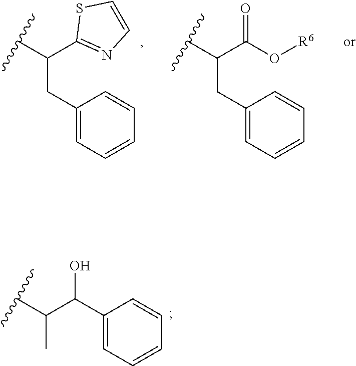

R⁶ is hydrogen or -C₁-C₈ alkyl.

In certain embodiment, the Ab-(L-D) comprises the compound of formula IIa:

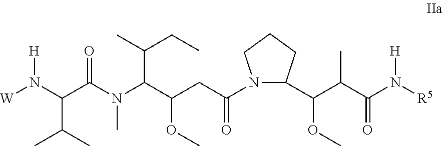

or a pharmaceutically acceptable salt or solvate thereof, wherein, independently for each occurrence,
W is

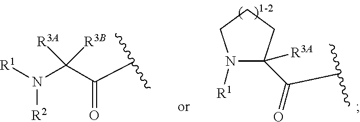

R¹ is

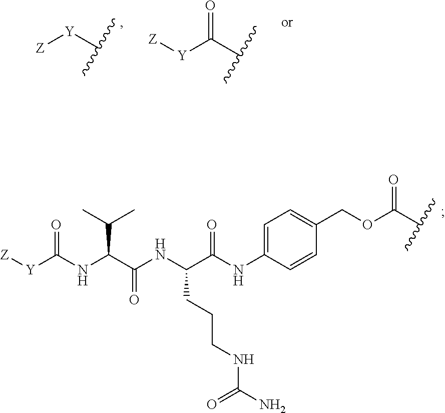

Y is one or more of the group selected from —C₂-C₂₀ alkylene-, —C₂-C₂₀ heteroalkylene-, —C₃-C₈ carbocyclo-, -arylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈heterocyclo)- or —(C₃-C₈ heterocyclo)-C₁-C₁₀alkylene-, —C₁₋₆alkyl(OCH₂CH₂)₁₋₁₀—, —(OCH₂CH₂)₁₋₁₀-, —(OCH₂CH₂)₁₋₁₀—C₁₋₆alkyl, —C(O)—C₁₋₆alkyl(OCH₂CH₂)₁₋₆—, —C₁₋₆alkyl(OCH₂CH₂)₁₋₆—C(O)—, —C₁₋₆alkyl-(OCH₂CH₂)₁₋₆—NRC(O)CH₂—, —C(O)—C₁₋₆alkyl(OCH₂CH₂)₁₋₆—NRC(O)—, and —C(O)—C₁₋₆alkyl-(OCH₂CH₂)₁₋₆≥NRC(O)C₁₋₆alkyl-;
Z is

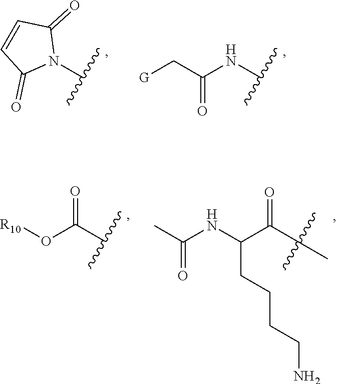

### or —NH₂;

G is halogen, —OH, —SH, or —S—C₁-C₆ alkyl;
R² is hydrogen or C₁-C₈ alkyl;
R^{3A} and R^{3B} are either of the following:
(iii) R^{3A} is hydrogen or C₁-C₈ alkyl; and
   R^{3B} is C₁-C₈ alkyl; or
(iv) R^{3A} and R^{3B} taken together are C₂-C₈ alkylene or C₁-C₈ heteroalkylene;
R⁵ is

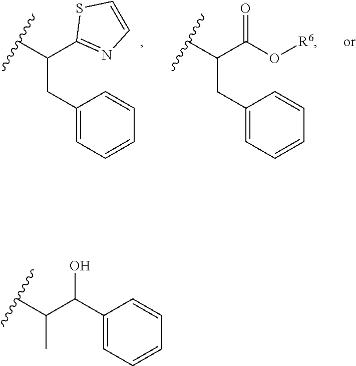

R⁶ is hydrogen or —C₁-C₈ alkyl;
R¹⁰ is hydrogen, —C₁-C₁₀alkyl, —C₃-C₈carbocyclyl, -aryl, —C₁-C₁₀heteroalkyl, —C₃-C₈heterocyclo, —C₁-C₁₀alkylene-aryl, -arylene-C₁-C₁₀alkyl, —C₁-C₁₀alkylene-(C₃-C₈carbocyclo), —(C₃-C₈ carbocyclo)-C₁-C₁₀alkyl, —C₁-C₁₀alkylene-(C₃-C₈heterocyclo), or —(C₃-C₈ heterocyclo)-C₁-C₁₀alkyl, where aryl on R₁₀ comprising aryl is optionally substituted with [R₇]ₕ;
R⁷ is independently selected for each occurrence from the group consisting of F, Cl, I, Br, NO₂, CN and CF₃; and
h is 1, 2, 3, 4 or 5.

Preferably, Y is one or more of the group selected from —C₂-C₂₀ alkylene-, —C₂-C₂₀ heteroalkylene-; —C₃-C₈ carbocyclo-, -arylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈heterocyclo)- or —(C₃-C₈ heterocyclo)-C₁-C₁₀alkylene-; —C₁₋₆alkyl(OCH₂CH₂)₁₋₁₀—, —(OCH₂CH₂)₁₋₁₀—, —(OCH₂CH₂)₁₋₁₀—C₁₋₆alkyl, —C(O)—C₁₋₆alkyl(OCH₂CH₂)₁₋₆—, and —C₁₋₆alkyl(OCH₂CH₂)₁₋₆—C(O)—;

Preferable, Z is

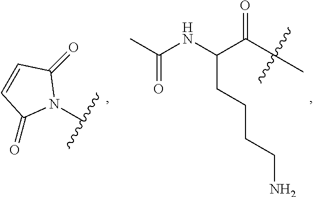 [SMILES: *N1C(=O)C=CC1=O ; *C(=O)C(CCCCN)NC(C)=O]

### or —NH₂.

In certain embodiment, the Ab-(L-D) comprises the compound of formula IIb:

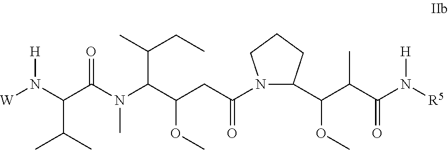

or a pharmaceutically acceptable salt or solvate thereof, wherein, independently for each occurrence,

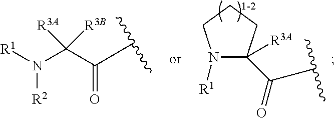

W is
R¹ is

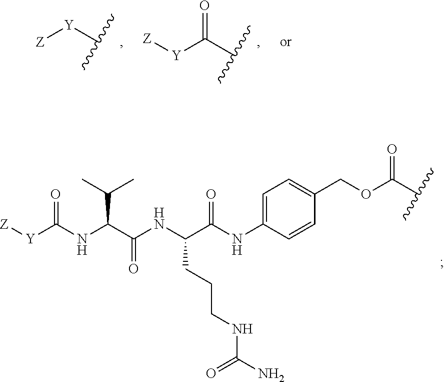

Y is —C₂-C₂₀ alkylene-, —C₂-C₂₀ heteroalkylene-, —C₃-C₈ carbocyclo-, -arylene-, —C₃-C₈heterocyclo-, —C₁-C₁₀alkylene-arylene-, -arylene-C₁-C₁₀alkylene-, —C₁-C₁₀alkylene-(C₃-C₈carbocyclo)-, —(C₃-C₈carbocyclo)-C₁-C₁₀alkylene-, —C₁-C₁₀alkylene-(C₃-C₈heterocyclo)-, or —(C₃-C₈ heterocyclo)-C₁-C₁₀alkylene-;
Z is

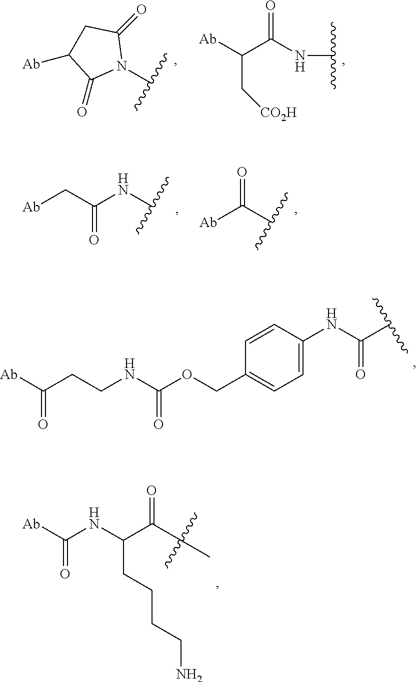

### or —NH-Ab;

Ab is an antibody;
R² is hydrogen or C₁-C₈ alkyl;
R^{3A} and R^{3B} are either of the following:
(iii) R^{3A} is hydrogen or C₁-C₈ alkyl;
   R^{3B} is C₁-C₈ alkyl;
(iv) R^{3A} and R^{3B} taken together are C₂-C₈ alkylene or C₁-C₈ heteroalkylene;
R⁵ is

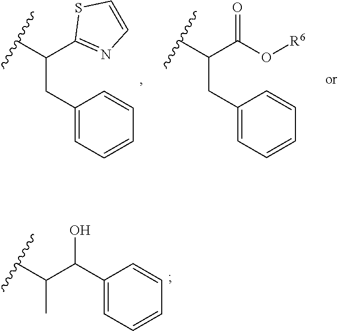

and
R⁶ is hydrogen or —C₁-C₈ alkyl.

Preferably, Z is

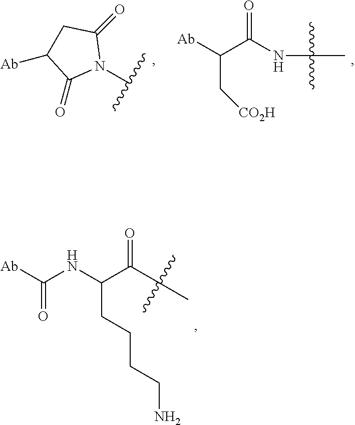

### or —NH-Ab.

In certain embodiments, the Ab-(L-D) comprises mcValCitPABC_MMAE (“vcMMAE”):

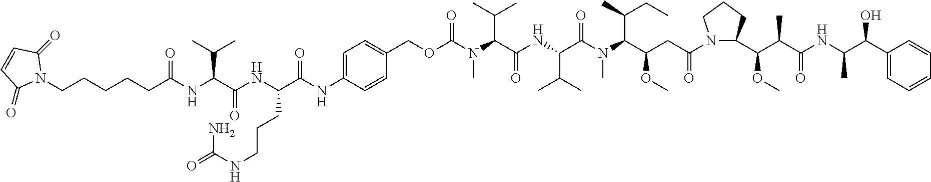 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In certain embodiments, the Ab-(L-D) comprises mcValCitPABC_MMAD (“vcMMAD”):

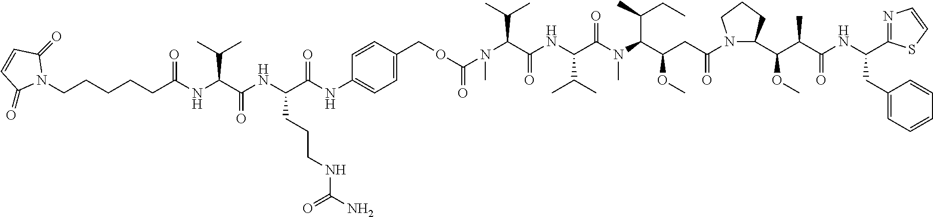 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

In certain embodiments, the Ab-(L-D) comprises mcMMAD (“maleimide caproyl MMAD):

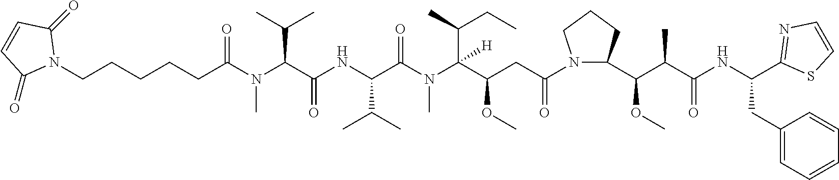 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)CCCCCN1C(=O)C=CC1=O)C(C)C]

In certain embodiments, the Ab-(L-D) comprises mcMMAF (“maleimide caproyl MMAF”):

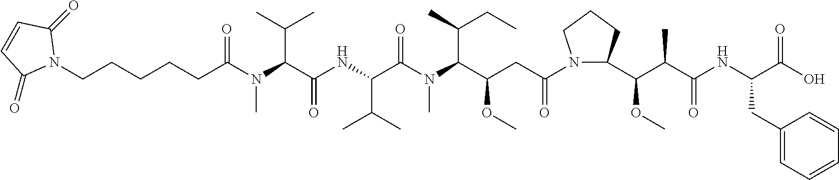 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)C=CC1=O)C(C)C]

Definitions of the generic terms used in Formulas I, IIa, and IIb, such as alkyl, alkenyl, haloalkyl; heterocyclyl, should be understood according to the ordinary and customary meaning of the terms. Specifically, these terms are defined in WO 2013/072813 (which is incorporated herein by reference in its entirety), from page 15, line 21 to page 18, line 14.

D. Methods of Preparing Site Specific ADCs

Also provided are methods for preparing antibody drug conjugates of the present invention. For example, a process for producing a site specific ADC as disclosed herein can include (a) linking the linker to the drug; (b) conjugating the linker drug moiety to the antibody; and (c) purifying the antibody drug conjugate.

The ADCs of the present invention use site specific methods to conjugate the antibody to the drug payload.

In one embodiment, the site specific conjugation occurs through one or more cysteine residues that have been engineered into an antibody constant region. Methods of preparing antibodies for site specific conjugation through cysteine residues can be performed as described in PCT Publication No. WO2013/093809, which is incorporated by reference in its entirety. One or more of the following positions can be altered to be a cysteine and thus serve as a site for conjugation: a) on the heavy chain constant region, residues 246, 249, 265, 267, 270, 276, 278, 283, 290, 292, 293, 294, 300, 302, 303, 314, 315, 318, 320, 327, 332, 333, 334, 336, 345, 347, 354, 355, 358, 360, 362, 370, 373, 376, 378, 380, 382, 386, 388, 390, 392, 393, 401, 404, 411, 413, 414, 416, 418, 419, 421, 428, 431, 432, 437, 438, 439, 443, and 444 (according to the EU index of Kabat for the heavy chain) and/or b) on the light chain constant region, residues 111, 149, 183, 188, 207, and 210 (according to the Kabat numbering for light chain).

In certain embodiments, the one or more positions that may be altered to be a cysteine a) on the heavy chain constant region are 290, 334, 392 and/or 443 (according to the EU index of Kabat for the heavy chain) and/or b) on the light chain constant region is 183 (according to the Kabat numbering for the light chain).

In a more specific embodiment, positions 290 on the heavy chain constant region according to the EU index of Kabat and position 183 on the light chain constant region are altered to cysteine for conjugation, according to Kabat numbering.

In another embodiment, the site specific conjugation occurs through one or more acyl donor glutamine residues that have been engineered into the antibody constant region. Methods of preparing antibodies for site specific conjugation through glutamine residues can be performed as described in PCT Publication No. WO2012/059882, which is incorporated by reference in its entirety. Antibodies can be engineered to express the glutamine residue used for site specific conjugation in three different ways.

The short peptide tag containing the glutamine residue can be incorporated into a number of different positions of the light and/or heavy chain (i.e., at the N-terminus, at the C-terminus, internally). In a first embodiment, short peptide tag containing the glutamine residue can be attached to the C-terminus of the heavy and/or light chain. One or more of the following glutamine containing tags can be attached to serve as the acyl donor for drug conjugation: GGLLQGPP (SEQ ID NO:45), GGLLQGG (SEQ ID NO:46), LLQGA (SEQ ID NO:47), GGLLQGA (SEQ ID NO:48), LLQ, LLQGPGK (SEQ ID NO: 49), LLQGPG (SEQ ID NO: 50), LLQGPA (SEQ ID NO: 51), LLQGP (SEQ ID NO: 52), LLQP (SEQ ID NO: 53), LLQPGK (SEQ ID NO: 54), LLQGAPGK (SEQ ID NO: 55), LLQGAPG (SEQ ID NO: 56), LLQGAP (SEQ ID NO: 57), LLQX₁X₂X₃X₄X₅, wherein X₁ is G or P, wherein X₂ is A, G, P, or absent, wherein X₃ is A, G, K, P, or absent, wherein X₄ is G, K or absent, and wherein X₅ is K or absent (SEQ ID NO: 58), or LLQX₁X₂X₃X₄X₅, wherein X₁ is any naturally occurring amino acid and wherein X₂, X₃, X₄, and X₅ are any naturally occurring amino acids or absent (SEQ ID NO: 59).

In certain embodiments, GGLLQGPP (SEQ ID NO:60) maybe attached to the C-terminus of the light chain.

In certain embodiments, a residue on the heavy and/or light chain maybe be altered to a glutamine residue by site directed mutagenesis. In certain embodiments, the residue at position 297 on the heavy chain (using EU index of Kabat) maybe be altered to be a glutamine (Q) and thus serve as a site for conjugation.

In certain embodiments, a residue on the heavy chain or light chain maybe be altered resulting in aglycosylation at that position such that one or more endogenous glutamine becomes accessible/reactive for conjugation. In certain embodiments, the residue at position 297 on the heavy chain (using EU index of Kabat) maybe altered to an alanine (A). In such cases, the glutamine (Q) at position 295 on the heavy chain is then capable for use in conjugation.

Optimal reaction conditions for formation of a conjugate may be empirically determined by variation of reaction variables such as temperature, pH, linker-payload moiety input, and additive concentration. Conditions suitable for conjugation of other drugs may be determined by those skilled in the art without undue experimentation. Site specific conjugation through engineered cysteine residues is exemplified in Example 5A infra. Site specific conjugation through glutamine residues is exemplified in Example 5B infra.

To further increase the number of drug molecules per antibody drug conjugate, the drug may be conjugated to polyethylene glycol (PEG), including straight or branched polyethylene glycol polymers and monomers. A PEG monomer is of the formula: —(CH₂CH₂O)—. Drugs and/or peptide analogs may be bound to PEG directly or indirectly, i.e. through appropriate spacer groups such as sugars. A PEG-antibody drug composition may also include additional lipophilic and/or hydrophilic moieties to facilitate drug stability and delivery to a target site in vivo. Representative methods for preparing PEG-containing compositions may be found in, e.g., U.S. Pat. Nos. 6,461,603; 6,309,633; and 5,648,095.

Following conjugation, the conjugates may be separated and purified from unconjugated reactants and/or aggregated forms of the conjugates by conventional methods. This can include processes such as size exclusion chromatography (SEC), ultrafiltration/diafiltration, ion exchange chromatography (IEC), chromatofocusing (CF) HPLC, FPLC, or Sephacryl S-200 chromatography. The separation may also be accomplished by hydrophobic interaction chromatography (HIC). Suitable HIC media includes Phenyl Sepharose 6 Fast Flow chromatographic medium, Butyl Sepharose 4 Fast Flow chromatographic medium, Octyl Sepharose 4 Fast Flow chromatographic medium, Toyopearl Ether-650M chromatographic medium, Macro-Prep methyl HIC medium or Macro-Prep t-Butyl HIC medium.

Table 4 shows HER2 ADCs used to generate data in the Examples Section. The site specific HER2 ADCs shown in Table 4 (in rows 1-17) are examples of site specific ADCs of the invention.

To make a site specific ADC of the invention any antibody disclosed herein can be conjugated using site specific techniques to any drug disclosed herein via any linker disclosed herein. In certain embodiments, the linker is cleavable (e.g., vc). In certain embodiments, the drug is an auristatin (e.g., 0101).

Polypeptides, antibodies and ADCs of the invention may be site-specific conjugated through an engineered cysteine at position 290 (according to the numbering of the EU index of Kabat). The IgG1 antibody heavy chain CH2 region is shown in SEQ ID NO:61 or SEQ ID NO: 62 (K290, using the numbering of the EU index of Kabat, is bold and underlined).

(SEQ ID NO: 61, CH2 domain)
APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK
(SEQ ID NO: 62, CH2 and CH3 domains)
APELLGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSRDELTK NQVSLTCLVK GFYPSDIAVE WESNGQPENN
YKTTPPVLDS DGSFFLYSKL TVDKSRWQQG NVFSCSVMHE
ALHNHYTQKS LSLSPGK

The engineered cysteine can be at position 290 alone or in combination with one or more engineered cysteine residues at the following positions: a) on the heavy chain constant region, residues 246, 249, 265, 267, 270, 276, 278, 283, 292, 293, 294, 300, 302, 303, 314, 315, 318, 320, 327, 332, 333, 334, 336, 345, 347, 354, 355, 358, 360, 362, 370, 373, 376, 378, 380, 382, 386, 388, 390, 392, 393, 401, 404, 411, 413, 414, 416, 418, 419, 421, 428, 431, 432, 437, 438, 439, 443, and 444 (according to the numbering of the EU index of Kabat), and/or b) on the light chain constant region, residues 111, 149, 183, 188, 207, and 210 (according to Kabat numbering).

In certain embodiments, the polypeptides, antibodies and ADCs of the invention may further comprise an antibody kappa light chain constant region comprising (i) an engineered cysteine residue at position 183, according to the numbering of Kabat; or (ii) an engineered cysteine residue at a position corresponding to residue 76 of SEQ ID NO:63, when said constant domain is aligned with SEQ ID NO:63. This engineered cysteine is also referred to as “K183C,” using the numbering of Kabat, and is shown in bold and underlined below. The peptide, antibody and ADC of the invention may comprise a lambda light chain constant region comprising an engineered cysteine residue at an amino acid position corresponding to amino acid residue 183 of a human kappa light chain constant region referred to as the “K183C” residue shown below.

(SEQ ID NO: 63, Cκ constant domain)
RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

In another aspect, the invention provides an antibody or antigen binding fragment thereof comprising (a) a polypeptide disclosed herein and (b) an antibody kappa light chain constant region comprising (i) an engineered cysteine residue at position 111, 149, 188, 207, 210, or any combination thereof (preferably 111 or 210), according to the numbering of Kabat; or (ii) an engineered cysteine residue at a position corresponding to residue 4, 42, 81, 100, 103, or any combination thereof, of SEQ ID NO:63 (preferably residue 4 or 103), when said constant domain is aligned with SEQ ID NO:63.

In another aspect, the invention provides an antibody or antigen binding fragment thereof comprising (a) a polypeptide disclosed herein and (b) an antibody lambda light chain constant region comprising (i) an engineered cysteine residue at position 110, 111, 125, 149, 155, 158, 161, 185, 188, 189, 191, 197, 205, 206, 207, 208, 210, or any combination thereof (preferably 110, 111, 125, 149, or 155), according to the numbering of Kabat; or (ii) an engineered cysteine residue at a position corresponding to residue 4, 5, 19, 43, 49, 52, 55, 78, 81, 82, 84, 90, 96, 97, 98, 99, 101, or any combination thereof of SEQ ID NO:64 (preferably residue 4, 5, 19, 43, or 49), when said constant domain is aligned with SEQ ID NO:64.

(SEQ ID NO: 64, Cλ constant domain)
GQPKANPTVT LFPPSSEELQ ANKATLVCLI SDFYPGAVTV
AWKADGSPVK AGVETTKPSK QSNNKYAASS YLSLTPEQWK
SHRSYSCQVT HEGSTVEKTV APTECS

TABLE 4
HER2 ADCs

| | Heavy Chain | Heavy Chain | | Light Chain | Light Chain | | | | |
|---|---|---|---|---|---|---|---|---|---|
| | variable | constant | Heavy | variable | constant | Light | | | Linker |
| ADC | region | region | Chain | region | region | Chain | Linker | Payload | Type¹ |
| T(kK183C)-vc0101 | 1 | 5 | 6 | 7 | 41 | 42 | vc | 0101 | C |
| T(K290C)-vc0101 | 1 | 17 | 18 | 7 | 11 | 12 | vc | 0101 | C |
| T(N297Q)-AcLysvc0101 | 1 | 21 | 22 | 7 | 11 | 12 | AcLysvc | 0101 | C |
| T(K334C)-vc0101 | 1 | 23 | 24 | 7 | 11 | 12 | vc | 0101 | C |
| T(K392C)-vc0101 | 1 | 25 | 26 | 7 | 11 | 12 | vc | 0101 | C |
| T(L443C)-vc0101 | 1 | 27 | 28 | 7 | 11 | 12 | vc | 0101 | C |
| T(kK183C + K290C)-vc0101 | 1 | 17 | 18 | 7 | 41 | 42 | vc | 0101 | C |
| T(kK183C + K334C)-vc0101 | 1 | 23 | 24 | 7 | 41 | 42 | vc | 0101 | C |
| T(kK183C + K392C)-vc0101 | 1 | 25 | 26 | 7 | 41 | 42 | vc | 0101 | C |
| T(kK183C + L443C)-vc0101 | 1 | 27 | 28 | 7 | 41 | 42 | vc | 0101 | C |
| T(K290C + K334C)-vc0101 | 1 | 29 | 30 | 7 | 11 | 12 | vc | 0101 | C |
| T(K290C + K392C)-vc0101 | 1 | 31 | 32 | 7 | 11 | 12 | vc | 0101 | C |
| T(N297A + K222R + LCQ05)- | 1 | 33 | 34 | 7 | 43 | 44 | AcLysvc | 0101 | C |
| AcLysvc0101 | | | | | | | | | |
| T(N297Q + K222R)- | 1 | 35 | 36 | 7 | 11 | 12 | AcLysvc | 0101 | C |
| AcLysvc0101 | | | | | | | | | |
| T(K334C + K392C)-vc0101 | 1 | 37 | 38 | 7 | 11 | 12 | vc | 0101 | C |
| T(K392C + L443C)-vc0101 | 1 | 39 | 40 | 7 | 11 | 12 | vc | 0101 | C |
| T(LCQ05 + K222R)- | 1 | 13 | 14 | 7 | 43 | 44 | AcLysvc | 0101 | C |
| AcLysvc0101 | | | | | | | | | |
| T-mc8261 | 1 | 5 | 6 | 7 | 11 | 12 | mc | 8261 | N |
| T-m(H20)c8261 | 1 | 5 | 6 | 7 | 11 | 12 | m(H20)c | 8261 | N |
| T-MalPeg8261 | 1 | 5 | 6 | 7 | 11 | 12 | MalPeg6 | 8261 | N |
| T-vc8261 | 1 | 5 | 6 | 7 | 11 | 12 | vc | 8261 | C |
| T-mc6121 | 1 | 5 | 6 | 7 | 11 | 12 | mc | 6121 | N |
| T-MalPeg6121 | 1 | 5 | 6 | 7 | 11 | 12 | MalPeg6 | 6121 | N |
| T-mc0101 | 1 | 5 | 6 | 7 | 11 | 12 | mc | 0101 | N |
| T-vc0101 | 1 | 5 | 6 | 7 | 11 | 12 | vc | 0101 | C |
| T-vc8254 | 1 | 5 | 6 | 7 | 11 | 12 | vc | 8254 | C |
| T-vc6780 | 1 | 5 | 6 | 7 | 11 | 12 | vc | 6780 | C |
| T-vc0131 | 1 | 5 | 6 | 7 | 11 | 12 | vc | 0131 | C |
| T-MalPegMMAD | 1 | 5 | 6 | 7 | 11 | 12 | MalPeg6 | MMAD | N |
| T-vcMMAE | 1 | 5 | 6 | 7 | 11 | 12 | vc | MMAE | C |
| T-DM1 | 1 | 5 | 6 | 7 | 11 | 12 | mcc | DM1 | N |

(¹C = cleavable; N = non-cleavable)

### 2. Formulations and Uses

Polypeptides, antibodies, and ADCs described herein can be formulated as pharmaceutical formulations. The pharmaceutical formulation may further comprise pharmaceutically acceptable carriers, excipients, or stabilizers. Further, the compositions can include more than one of the ADCs disclosed herein.

The compositions used in the present invention can further include pharmaceutically acceptable carriers, excipients, or stabilizers (Remington: The Science and practice of Pharmacy 21st Ed., 2005, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e. g. Zn-protein complexes); and/or non-ionic surfactants such as TWEEN™, PLURONICS™ or polyethylene glycol (PEG). “Pharmaceutically acceptable salt” as used herein refers to pharmaceutically acceptable organic or inorganic salts of a molecule or macromolecule. Pharmaceutically acceptable excipients are further described herein.

Various formulations of one or more ADCs of the invention may be used for administration including, but not limited to formulations comprising one or more pharmaceutically acceptable excipients. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000.

In some aspects of the invention, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

Therapeutic formulations of the ADCs of the invention are prepared for storage by mixing an ADC having the desired degree of purity with optional pharmaceutically acceptable carriers, excipients or stabilizers (Remington, The Science and Practice of Pharmacy 21st Ed. Mack Publishing, 2005), in the form of lyophilized formulations or aqueous solutions. Acceptable carriers, excipients, or stabilizers are nontoxic to recipients at the dosages and concentrations employed, and may include buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e. g. Zn-protein complexes); and/or non-ionic surfactants such as TWEEN™, PLURONICS™ or polyethylene glycol (PEG).

Liposomes containing the ADCs of the invention can be prepared by methods known in the art, such as described in Eppstein, et al., 1985, PNAS 82:3688-92; Hwang, et al., 1908, PNAS 77:4030-4; and U. S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition including phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles and nanocapsules) or in macroemulsions. Such techniques are disclosed in Remington, The Science and Practice of Pharmacy 21st Ed. Mack Publishing, 2005.

Sustained-release preparations may be prepared. Suitable examples of sustained-release preparations include semipermeable matrices of solid hydrophobic polymers containing the antibody, which matrices are in the form of shaped articles, e. g. films, or microcapsules. Examples of sustained-release matrices include polyesters, hydrogels (for example, poly(2-hydroxyethyl-methacrylate), or poly(vinylalcohol)), polylactides (U. S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic ADC compositions are generally placed into a container having a sterile access port, for example, an intravenous solution bag or vial having a stopper pierceable by a hypodermic injection needle.

Suitable surface-active agents include, in particular, non-ionic agents, such as polyoxyethylenesorbitans (e.g., TWEEN™ 20, 40, 60, 80 or 85) and other sorbitans (e. g. Span™ 20, 40, 60, 80 or 85). Compositions with a surface-active agent will conveniently include between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e. g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can include fat droplets between 0.1 and 1.0 μm, particularly 0.1 and 0.5 μm, and have a pH in the range of 5.5 to 8.0. The emulsion compositions can be those prepared by mixing an ADC with INTRALIPID™ or the components thereof (soybean oil, egg phospholipids, glycerol and water).

The invention also provides kits for use in the instant methods. Kits of the invention include one or more containers including one or more ADCs of the invention and instructions for use in accordance with any of the methods of the invention described herein. Generally, these instructions include a description of administration of the ADC for therapeutic treatments.

The instructions relating to the use of the ADCs of the invention generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an ADC of the invention. The container may further include a second pharmaceutically active agent.

Kits may optionally provide additional components such as buffers and interpretive information. Normally, the kit includes a container and a label or package insert(s) on or associated with the container.

The ADCs of the invention can be used for therapeutic, diagnostic, or non-therapeutic purposes. For example, the antibody or antigen-binding fragment thereof may be used as an affinity purification agents (e.g., for in vitro purification), as a diagnostic agent (e.g., for detecting expression of an antigen of interest in specific cells, tissues, or serum)

For therapeutic applications, the ADCs of the invention can be administered to a mammal, especially a human by conventional techniques, such as intravenously (as a bolus or by continuous infusion over a period of time), intramuscularly, intraperitoneally, intra-cerebrospinally, subcutaneously, intra-articularly, intrasynovially, intrathecally, orally, topically, or by inhalation. The antibodies or antigen-binding fragments also are suitably administered by intra-tumoral, peri-tumoral, intra-lesional, or peri-lesional routes. the ADCs of the invention can be used in prophylactic treatment or therapeutic treatment

### 3. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The term “L-D” refers to a linker-drug moiety resulting from a drug (D) linked to a linker (L). The term “Drug (D)” refers to any therapeutic agent useful in treating a disease. The drug has biological or detectable activity, for example, a cytotoxic agent, a chemotherapeutic agent, a cytostatic agent, or an immunomodulatory agent. In the context of cancer treatment, a therapeutic agent has a cytotoxic effect on tumors including the depletion, elimination and/or the killing of tumor cells. The terms drug, payload, and drug payload are used interchangeably. In certain embodiments, therapeutic agents have a cytotoxic effect on tumors including the depletion, elimination and/or the killing of tumor cells. In certain embodiments, the drug is an anti-mitotic agent. In certain embodiments, the drug is an auristatin. In certain embodiments, the drug is 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (also known as 0101). In certain embodiments, the drug is preferably membrane permeable.

The term “Linker (L)” describes the direct or indirect linkage of the antibody to the drug payload. Attachment of a linker to an antibody can be accomplished in a variety of ways, such as through surface lysines, reductive-coupling to oxidized carbohydrates, cysteine residues liberated by reducing interchain disulfide linkages, reactive cysteine residues engineered at specific sites, and acyl donor glutamine-containing tag or an endogenous glutamine made reactive by polypeptide engineering in the presence of transglutaminase and an amine. The present invention uses site specific methods to link the antibody to the drug payload. In one embodiment, conjugation occurs through cysteine residues that have been engineered into the antibody constant region. In another embodiment, conjugation occurs through acyl donor glutamine residues that have either been a) added to the antibody constant region via a peptide tag, b) engineered into the antibody constant region or c) made accessible/reactive by engineering surrounding residues). Linkers can be cleavable (i.e., susceptible to cleavage under intracellular conditions) or non-cleavable. In some embodiments, the linker is a cleavable linker.

An “antigen-binding fragment” of an antibody refers to a fragment of a full-length antibody that retains the ability to specifically bind to an antigen (preferably with substantially the same binding affinity). Examples of an antigen-binding fragment includes: an Fab fragment; an F(ab′)2 fragment; an Fd fragment; an Fv fragment; a dAb fragment (Ward et al., (1989) Nature 341:544-546); an isolated complementarity determining region (CDR); a disulfide-linked Fv (dsFv); an anti-idiotypic (anti-Id) antibodies; an intrabody; a single chain Fv (scFv, see e. g., Bird et al. Science 242:423-426 (1988) and Huston et al. Proc. Natl. Acad. Sci. USA 85:5879-5883 (1988)); and a diabody (see e. g., Holliger et al. Proc. Natl. Acad. Sci. USA 90:6444-6448 (1993); Poljak et al., 1994, Structure 2:1121-1123). The antigen-binding fragment of the invention comprises the engineered antibody constant domain described herein, but does not need to comprise the full length Fc-region of a native antibody. For example, the antigen-binding fragment of the invention can be a “minibody” (VL-VH-CH3 or (scFv-CH3)₂; see, Hu et al., Cancer Res. 1996; 56(13):3055-61, and Olafsen et al., Protein Eng Des Sel. 2004;17(4):315-23).

Residues in a variable domain of an antibody are numbered according Kabat, which is a numbering system used for heavy chain variable domains or light chain variable domains of the compilation of antibodies. See, Kabat et al., Sequences of Proteins of Immunological Interest, 5th Ed. Public Health Service, National Institutes of Health, Bethesda, Md. (1991)). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e. g. residues 82a, 82b, and 82c, according to Kabat) after heavy chain FR residue 82. The Kabat numbering of residues may be determined for a given antibody by alignment at regions of homology of the sequence of the antibody with a “standard” Kabat numbered sequence. Various algorithms for assigning Kabat numbering are available. The algorithm implemented in the 2012 release of Abysis (www.abysis.org) is used herein to assign Kabat numbering to variable regions unless otherwise noted.

Unless otherwise specified, amino acid residues in the IgG heavy constant domain of an antibody are numbered according the EU index of Edelman et al., 1969, Proc. Natl. Acad. Sci. USA 63(1):78-85 as described in Kabat et al., 1991, referred to herein as the “EU index of Kabat”. Typically, the Fc domain comprises from about amino acid residue 236 to about 447 of the human IgG1 constant domain. Correspondence between C numberings can be found, e.g., at IGMT database. Amino acid residues of the light chain constant domain are numbered according to Kabat et al., 1991. Numbering of antibody constant domain amino acid residues is also shown in International Patent Publication No. WO 2013/093809.

The only exception to the use of EU index of Kabat in IgG heavy constant domain is residue A114 described in the examples. A114 refers to Kabat numbering, and the corresponding EU index number is 118. This is because the initial publication of site specific conjugating at this site used Kabat numbering and referred this site as A114C, and has since been widely used in the art as the “114” site. See Junutula et al., Nature Biotechnology 26, 925-932 (2008). To be consistent with the common usage of this site in the art, “A114,” “A114C,” “C114” or “114C” are used in the examples.

Unless otherwise specified, amino acid residues in the light chain constant domain of an antibody are numbered according to Kabat et al., 1991.

An amino acid residue of a query sequence “corresponds to” a designated position of a reference sequence (e. g., position 60 of SEQ ID NO:61 or 62 or position 76 of SEQ ID NO:63) when, by aligning the query amino acid sequence with the reference sequence, the position of the residue matches the designated position. Such alignments can be done by hand or by using well-known sequence alignment programs such as ClustalW2, or “BLAST 2 Sequences” using default parameters.

An “Fc fusion” protein is a protein wherein one or more polypeptides are operably linked to an Fc polypeptide. An Fc fusion combines the Fc region of an immunoglobulin with a fusion partner.

The term “about”, as used here, refers to +/−10% of a value.

As used herein, the terms “engineered” (as in engineered cysteine) and “substituted” (as in substituted cysteine) are used interchangeably, and refer to mutating an amino acid to cysteine, in order to create a conjugation site for attaching another moiety to a polypeptide or antibody.

### Biological Deposit

Representative materials of the present invention were deposited in the American Type Culture Collection, 10801 University Boulevard, Manassas, Va. 20110-2209, USA, on Nov. 17, 2015. Vector T(K290C)-HC having ATCC Accession No. PTA-122672 comprises a DNA insert encoding the heavy chain sequence of SEQ ID NO:18, and vector T(kK183C)-LC having ATCC Accession No. PTA-122673 comprises a DNA insert encoding the light chain sequence of SEQ ID NO:42. The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Preparation of Trastuzumab Derived Antibodies for Conjugation

A. Conjugation via Cysteine

Methods of preparing trastuzumab derivatives for site specific conjugation through cysteine residues were generally performed as described in PCT Publication WO2013/093809 (which is incorporated herein in its entirety). One or more residues on either the light chain (183 using the Kabat numbering scheme) or the heavy chain (290, 334, 392 and/or 443 using the EU index of Kabat) were altered to a cysteine (C) residue by site directed mutagenesis.

B. Conjugation via Transglutaminase

Methods of preparing trastuzumab derivatives for site specific conjugation through glutamine residues were generally performed as described in PCT Publication WO2012/059882 (which is incorporated herein in its entirety). Trastuzumab was engineered to express the glutamine residue used for conjugation in three different ways.

For the first method, an 8 amino acid residue tag (LCQ05) containing the glutamine residue was attached to the C-terminus of the light chain.

For the second method, a residue on the heavy chain (position 297 using the EU index of Kabat) was altered from an asparagine (N) to a glutamine (Q) residue by site directed mutagenesis.

For the third method, a residue on the heavy chain (position 297 using the EU index of Kabat) was altered from an asparagine (N) to an alanine (A). This results in aglycosylation at position 297 and accessible/reactive endogenous glutamine at position 295.

Additionally, some of the trastuzumab derivatives have an alteration that is not used for conjugation. The residue at position 222 on the heavy chain (position 297 using the EU index of Kabat) was altered from a lysine (K) to an arginine (R) residue. The K222R substitution was found to result in more homogenous antibody and payload conjugate, better intermolecular crosslinking between the antibody and the payload, and/or significant decrease in interchain crosslinking with the glutamine tag on the C terminus of the antibody light chain.

### Example 2: Production of Stably Transfected Cells Expressing Trastuzumab Derived Antibodies

To determine that the single and double cysteine engineered trastuzumab derived antibody variants could be stably expressed in cells and large-scale produced, CHO cells were transfected with DNA encoding nine trastuzumab derived antibody variants (T(κK183C), T(K290C), T(K334C), T(K392C), T(κK183C+K2900), T(κK183C+K3920), T(K290C+K334C), T(K334C+K392C) and T(K290C+K392C)) and stable high production pools were isolated using standard procedures well-known in the art. To produce T(KK183C+K334C) for conjugation studies, HEK-293 cells (ATCC Accession #CRL-1573) were transiently co-transfected with heavy and light chain DNA encoding this double-cysteine engineered antibody variant using standard methods. A two-column process, i. e. Protein-A affinity capture followed by a TMAE column or a three-column process, i. e. Protein-A affinity capture followed by a TMAE column and then CHA-TI column, was used to isolate these trastuzumab variants from the concentrated CHO pool starting material. Using these purification process, all engineered cysteine trastuzumab derived antibody variant preparations contained >97% peak-of-interest (POI) as determined by analytical size-exclusion chromatography (Table 5). These results shown in Table 5 demonstrate that acceptable levels of high molecular weight (HMVV) aggregated species were detected following elution from Protein A resin for all ten trastuzumab derived cysteine variants and that this undesirable HMW species could be removed using size exclusion chromatography. Additionally, the data demonstrated that the Protein A binding site in the human IgG1 constant region was not altered by the presence of the engineered cysteine residues.

TABLE 5
Production of Trastuzumab Derived Cysteine Antibody Variants

| | Purifi- | ProA | | | |
|---|---|---|---|---|---|
| | cation | Eluate | Yield | Final | Yield |
| Variant | Process | (% POI) | (ProA) | (% POI) | (Final) |
| T(κK183C) | 2 column | ND | ND | >99% | 768 mg/L |
| T(K290C) | 2 column | >99% | ND | >99% | 100 mg/L |
| T(K334C) | 2 column | >99% | ND | >99% | 100 mg/L |
| T(K392C | 2 column | >99% | ND | >99% | 110 mg/L |
| T(κK183C + | 3 column | 93% | 567 mg/L | >99% | 248 mg/L |
| K290C) | | | | | |
| T(K290C + | 3 column | 91.2% | 470 mg/L | >99% | 240 mg/L |
| K334C) | | | | | |
| T(K334C + | 3 column | 92.4% | 410 mg/L | >99% | 220 mg/L |
| K392C) | | | | | |
| T(κK183C + | 3 column | ND | ND | >99% | 64 mg/L |
| K334C) | | | | | |
| T(K290C + | 2 column | 93.1% | 700 mg/L | 97.9% | 420 mg/L |
| K392C) | | | | | |
| T(κK183C + | 2 column | 91.4 | ND | 97.8 | 600 mg/L |
| K392C) | | | | | |

ND = Not Determined

### Example 3: Integrity of Trastuzumab Derived Antibodies

Molecular assessment of the engineered cysteine and transglutaminase variants was performed to evaluate key biophysical properties relative to the trastuzumab wild type antibody to ensure the variants would be amenable to a standard antibody manufacturing platform process.

To determine integrity of the purified engineered cysteine antibody variant preparations produced via stable CHO expression, the percent purity of peaks was calculated using non-reduced capillary gel electrophoresis (Caliper LabChip GXII: Perkin Elmer Waltham, Mass.). Results show that the engineered cysteine antibody variants T(KK183C+K290C) and T(K290C+K334C) contained low levels of both fragments and high molecular mass species (HMMS) similar to the trastuzumab wild type antibody. In contrast, T(K334C+K392C) contained high levels of fragmented antibody peaks relative to the other double engineered cysteine variants evaluated (Table 6). These results suggest that specific combinations of engineered cysteines can impact integrity of the antibody intended for site-specific conjugation.

TABLE 6
Percent Purity of Peaks Calculated
from Non-Reduced Electropherogram

| Antibody | Main Peak (%) | Fragments (%) | HMMS (%) |
|---|---|---|---|
| trastuzumab WT | 95 | 5 | 0 |
| T(κK183C + K290C) | 95.78 | 4.18 | 0.04 |
| T(K290C + K334C) | 94.6 | 5.2 | 0.2 |
| T(K334C + K392C) | 80.7 | 19.3 | 0 |

### Example 4: Generation of Payload Drug Compounds

The auristatin drug compounds 0101, 0131, 8261, 6121, 8254 and 6780 were made according to the methods described in PCT Publication W02013/072813 (which is incorporated herein in its entirety). In published application, the auristatin compounds are indicated by the numbering system shown in Table 7.

TABLE 7

| Auristatin Drug Compound | Designation in WO2013/072813 |
|---|---|
| 0101 | #54 |
| 0131 | #118 |
| 8261 | #69 |
| 6121 | #117 |
| 8254 | #70 |
| 6780 | #112 |

According to PCT Publication WO2013/072813 drug compound 0101 was made according to the following procedure.

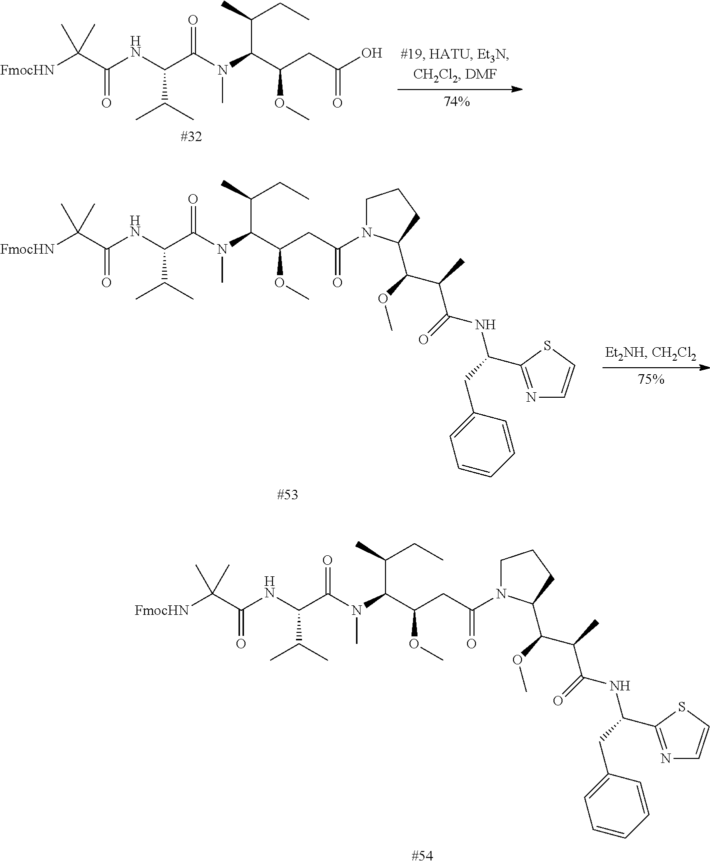

Step 1. Synthesis of N-[(9H-fluoren-9-ylmethoxy)carbonyl]-2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (#53). According to general procedure D, from #32 (2.05 g, 2.83 mmol, 1 eq.) in dichloromethane (20 mL, 0.1 M) and N,N-dimethylformamide (3 mL), the amine #19 (2.5 g, 3.4 mmol, 1.2 eq.), HATU (1.29 g, 3.38 mmol, 1.2 eq.) and triethylamine (1.57 mL, 11.3 mmol, 4 eq.) was synthesized the crude desired material, which was purified by silica gel chromatography (Gradient: 0% to 55% acetone in heptane), producing #53 (2.42 g, 74%) as a solid. LC-MS: m/z 965.7 [M+H⁺], 987.6 [M+Na⁺], retention time=1.04 minutes; HPLC (Protocol A): m/z 965. 4 [M+H⁺], retention time=11.344 minutes (purity>97%); ¹H NMR (400 MHz, DMSO-d₆), presumed to be a mixture of rotamers, characteristic signals: δ 7.86-7.91 (m, 2H), [7.77 (d, J=3.3 Hz) and 7.79 (d, J=3.2 Hz), total 1H], 7.67-7.74 (m, 2H), [7.63 (d, J=3.2 Hz) and 7.65 (d, J=3.2 Hz), total 1H], 7.38-7.44 (m, 2H), 7.30-7.36 (m, 2H), 7.11-7.30 (m, 5H), [5.39 (ddd, J=11.4, 8.4, 4.1 Hz) and 5.52 (ddd, J=11.7, 8.8, 4.2 Hz), total 1H], [4.49 (dd, J=8.6, 7.6 Hz) and 4.59 (dd, J=8.6, 6.8 Hz), total 1H], 3.13, 3.17, 3.18 and 3.24 (4 s, total 6H), 2.90 and 3.00 (2 br s, total 3H), 1.31 and 1.36 (2 br s, total 6H), [1.05 (d, J=6.7 Hz) and 1.09 (d, J=6.7 Hz), total 3H].

Step 2. Synthesis of 2-methylalanyl-N-[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl]-N-methyl-L-valinamide (#54 or 0101). According to general procedure A, from #53 (701 mg, 0.726 mmol) in dichloromethane (10 mL, 0.07 M) was synthesized the crude desired material, which was purified by silica gel chromatography (Gradient: 0% to 10% methanol in dichloromethane). The residue was diluted with diethyl ether and heptane and was concentrated in vacuo to afford #54 (or 0101) (406 mg, 75%) as a white solid. LC-MS: m/z 743.6 [M+H⁺], retention time=0.70 minutes; HPLC (Protocol A): m/z 743.4 [M+H⁺], retention time=6.903 minutes, (purity>97%); ¹H NMR (400 MHz, DMSO-d₆), presumed to be a mixture of rotamers, characteristic signals: δ [8. 64 (br d, J=8.5 Hz) and 8.86 (br d, J=8.7 Hz), total 1H], [8.04 (br d, J=9.3 Hz) and 8.08 (br d, J=9.3 Hz), total 1H], [7.77 (d, J=3.3 Hz) and 7.80 (d, J=3.2 Hz), total 1H], [7.63 (d, J=3.3 Hz) and 7.66 (d, J=3.2 Hz), total 1H], 7.13-7.31 (m, 5H), [5.39 (ddd, J=11, 8.5, 4 Hz) and 5. 53 (ddd, J=12, 9, 4 Hz), total 1H], [4.49 (dd, J=9, 8 Hz) and 4.60 (dd, J=9, 7 Hz), total 1H], 3.16, 3.20, 3.21 and 3.25 (4 s, total 6H), 2.93 and 3.02 (2 br s, total 3H), 1.21 (s, 3H), 1.13 and 1.13 (2 s, total 3H), [1.05 (d, J=6.7 Hz) and 1.10 (d, J=6.7 Hz), total 3H], 0.73-0.80 (m, 3H).

Drug compounds MMAD, MMAE and MMAF were made in-house according to methods disclosed in PCT Publication WO 2013/072813.

Drug compound DM1 was made in-house from purchased maytansinol via procedures outlined in U.S. Pat. No. 5,208,020.

### Example 5: Bioconjugation of Trastuzumab-Derived Antibodies

The trastuzumab-derived antibodies of the present invention were conjugated to payload via linkers to generate ADCs. The conjugation method used was either site specific (i.e., via particular cysteine residues or particular glutamine residues) or conventional conjugation.

A. Cysteine Site Specific

The ADCs of Table 8 were conjugated via cysteine site specific methods described below.

TABLE 8

| | |
|---|---|
| T(kK183C)-vc0101 | T(kK183C + K334C)-vc0101 |
| T(K290C)-vc0101 | T(kK183C + K392C)-vc0101 |
| T(K334C)-vc0101 | T(K290C + K334C)-vc0101 |
| T(K392C)-vc0101 | T(K290C + K392C)-vc0101 |
| T(kK183C + K290C)-vc0101 | T(K334C + K392C)-vc0101 |

A 500 mM tris(2-carboxyethyl)phosphine hydrochloride (TCEP) solution (50 to 100 molar equivalents) was added to the antibody (5 mg) such that the final antibody concentration was 5-15 mg/mL in PBS containing 20 mM EDTA. After allowing the reaction to stand at 37° C. for 2.5 hour, the antibody was buffer exchanged into PBS containing 5 mM EDTA using a gel filtration column (PD-10 desalting column, GE Healthcare). The resulting antibody (5-10 mg/mL) in PBS containing 5 mM EDTA was treated with a freshly prepared 50 mM solution of DHA in 1:1 PBS/EtOH (final DHA concentration=1 mM-4 mM) and allowed to stand at 4° C. overnight.

The antibody/DHA mixture was buffer exchanged into PBS containing 5 mM EDTA (pH of the equilibration buffer adjusted to ˜7.0 using phosphoric acid) and concentrated using a 50 KDa MW cutoff spin concentration device. The resulting antibody in PBS (antibody concentration ˜5-10 mg/ml) containing 5 mM EDTA was treated with 5-7 molar equivalents of 10 mM maleimide payload in DMA. After standing for 1.5-2.5 hours, the material was buffer exchanged (PD-10). Purification by SEC was performed (as needed) to remove any aggregated material and remaining free payload.

B. Transglutaminase Site Specific

The ADCs of Table 9 were conjugated via transglutaminase site specific methods described below.

TABLE 9

T(N297Q)-AcLysvc0101
T(LCQ05 + K222R)-AcLysvc0101
T(N297Q + K222R)-AcLysvc0101
T(N297A + K222R-LCQ05)-AcLysvc0101

In the transamidation reaction, the glutamine on the antibody acted as an acyl donor, and the amine-containing compound acted as an acyl acceptor (amine donor). Purified HER2 antibody in the concentration of 33 μM was incubated with a 10-25 M excess acyl acceptor, ranging between 33-83.3 μM AcLysvc-0101, in the presence of 2% (w/v) Streptoverticillium mobaraense transglutaminase (ACTIVA™, Ajinomoto, Japan) in 150-mM sodium chloride and Tris HCl buffer at pH range 7.5-8, with 0.31 mM reduced glutathione unless noted. The reaction conditions were adjusted for individual acyl donors, with T(LCQ05+K222R) using 10 M excess acyl acceptor at pH 8.0 without reduced glutathione, T(N297Q+K222R) and T(N297Q) using 20 M excess acyl acceptor at pH 7.5 and T(N297A+K222R+LCQ05) using 25 M excess acyl acceptor at pH 7.5. Following incubation at 37° C. for 16-20 hours, the antibody was purified on MabSelect SuReÔ resin or Butyl Sepharose High Performance (GE Healthcare, Piscataway, N.J.) using standard chromatography methods known to persons skilled in the art, such as commercial affinity chromatography and hydrophobic interaction chromatography from GE Healthcare.

C. Conventional Conjugation

The ADCs of Tables 10 and 11 were conjugated via conventional conjugation methods described below.

TABLE 10

| | |
|---|---|
| T-DM1 | T-mc0101 |
| T-mc8261 | T-vc0101 |
| T-MalPeg8261 | T-vc8261 |
| T-mc6121 | T-vc8254 |
| T-MalPeg6121 | T-vc6780 |
| T-MalPegMMAD | T-vc0131 |
| | T-vcMMAE |

TABLE 11

T-m(H20)c8261
T-m(H20)cvc0101

The antibody was dialyzed into Dulbecco's Phosphate Buffered Saline (DPBS, Lonza). The dialyzed antibody was diluted to 15 mg/mL with PBS containing 5 mM 2,2′,2″,2′″-(ethane-1,2-diyldinitrilo)tetraacetic acid (EDTA), pH 7. The resulting antibody was treated with 2-3 equivalents of tris(2-carboxyethyl)phosphine hydrochloride (TCEP, 5 mM in distilled water) and allowed to stand 37° C. for 1-2 hours. Upon cooling to room temperature, dimethylacetamide (DMA) was added to achieve 10% (v/v) total organic. The mixture was treated with 8-10 equivalents of the appropriate linker-payload as a 10 mM stock solution in DMA. The reaction was allowed to stand for 1-2 hours at room temperature and then buffer exchanged into DPBS (pH 7.4) using GE Healthcare Sephadex G-25 M buffer exchange columns per manufacturer's instructions.

Material that was to remain ring-closed (ADCs of Table 10) was purified by size exclusion chromatography (SEC) using GE AKTA Explorer system with GE Superdex200 column and PBS (pH 7.4) eluent. Final samples were concentrated to ˜5 mg/mL protein, filter sterilized, and checked for loading using the mass spectroscopy conditions outlined below.

Material used for succinimide ring hydrolysis (ADCs of Table 11) were immediately buffer exchanged into a 50 mM borate buffer (pH 9. 2) using an ultrafiltration device (50 KDa MW cutoff). The resulting solution was heated to 45° C. for 48 h. The resulting solution was cooled, buffer-exchanged into PBS, and purified by SEC (as described below) in order to remove any aggregated material. Final samples were concentrated to ˜5 mg/mL protein and filter sterilized and checked for loading using the mass spectroscopy conditions outlined below.

D. T-DM1 Conjugation

Trastuzumab-maytansinoid conjugate (T-DM1) is structurally similar to trastuzumab emtansine (Kadcyla®). T-DM1 is comprised of the trastuzumab antibody covalently bound to the DM1 maytansinoid through the bifunctional linker sulfosuccinimidyl 4-(N-maleimidomethyl) cyclohexane-1-carboxylate (sulfo-SMCC). Sulfo-SMCC is first conjugated to the free amines on the antibody for one hour at 25° C. in 50 mM potassium phosphate, 2 mM EDTA, pH 6.8, at a 10:1 reaction stoichiometry, and unbound linker is then desalted from the conjugated antibody. This antibody-MCC intermediate is then conjugated to the DM1 sulfide at the free maleimido end on the MCC linker antibody overnight at 25° C. in 50 mM potassium phosphate, 50 mM NaCl, 2 mM EDTA, pH 6.8, at a 10:1 reaction stoichiometry. Remaining unreacted maleimide is then capped with L-cysteine, and the ADC is fractionated through a Superdex200 column to remove non-monomeric species (Chari et al., 1992, Cancer Res 52:127-31).

### Example 6: Purification of ADCs

The ADCs were generally purified and characterized using size-exclusion chromatography (SEC) as described below. The loading of the drug onto the intended site of conjugation was determined using a variety of methods including mass spectrometry (MS), reverse phase HPLC, and hydrophobic interaction chromatography (HIC), as more fully described below. The combination of these three analytical methods provides a variety of ways to verify and quantitate the loading of the payload onto the antibody thereby providing an accurate determination of the DAR for each conjugate.

A. Preparative SEC

ADCs were generally purified using SEC chromatography using a Waters Superdex200 10/300GL column on an Akta Explorer FPLC system in order to remove protein aggregate and to remove traces of payload-linker left in the reaction mixture. On occasion, ADCs were free of aggregate and small molecule prior to SEC purification and were therefore not subjected to preparative SEC. The eluent used was PBS at 1 mL/min flow. Under these conditions, aggregated material (eluting at about 10 minutes at room temperature) was easily separated from non-aggregated material (eluting at about 15 minutes at room temperature). Hydrophobic payload-linker combinations frequently resulted in a “right-shift” of the SEC peaks. Without wishing to be bound by any particular theory, this SEC peak shift may be due to hydrophobic interactions of the linker-payload with the stationary phase. In some cases, this right-shift allowed for conjugated protein to be partially resolved from non-conjugated protein.

B. Analytical SEC

Analytical SEC was carried out on an Agilent 1100 HPLC using PBS as eluent to assess the purity and monomeric status of the ADCs. The eluent was monitored at 220 and 280 nM. When the column was a TSKGel G3000SW column (7.8×300 mm, catalog number R874803P), the mobile phase used was PBS with a flow rate of 0.9 mL/min for 30 minutes When the column was a BiosepSEC3000 column (7.8×300 mm), the mobile phase used was PBS with a flow rate of 1.0 mL/min for 25 minutes.

### Example 7: Characterization of ADCs

A. Mass Spectroscopy (MS)

Samples were prepped for LCMS analysis by combining approximately 20 μl of sample (approximately 1 mg/ml ADC in PBS) with 20 μl of 20 mM dithiothreitol (DTT). After allowing the mixture to stand at room temperature for 5 minutes, the samples were injected into an Agilent 110 HPLC system fitted with an Agilent Poroshell 300SB-C8 (2.1×75 mm) column. The system temperature was set to 60° C. A 5 minute gradient from 20% to 45% acetonitrile in water (with 0.1% formic acid modifier) was utilized. The eluent was monitored by UV (220 nM) and by a Waters Micromass ZQ mass spectrometer (ESI ionization; cone voltage: 20V; Source temp: 120° C.; Desolvation temp: 350° C.). The crude spectrum containing the multiple-charged species was deconvoluted using MaxEnt1 within MassLynx 4. 1 software package according to the manufacturer's instructions.

B. MS Determination of Loading Per Antibody

The total loading of the payload to the antibody to make an ADC is referred to as the Drug Antibody Ratio or DAR. The DAR was calculated for each of the ADCs made (Table 12).

The spectra for the entire elution window (usually 5 minutes) were combined into a single summed spectrum (i.e., a mass spectrum that represents the MS of the entire sample). MS results for ADC samples were compared directly to the corresponding MS of the identical non-loaded control antibody. This allowed for the identification of loaded/nonloaded heavy chain (HC) peaks and loaded/nonloaded light chain (LC) peaks. The ratio of the various peaks can be used to establish loading based on the equation below (Equation 1). Calculations are based on the assumption that loaded and non-loaded chains ionize equally which has been determined to be a generally valid assumption.

The following calculation was performed in order to establish the DAR:

Loading=2*[LC1/(LC1+LC0)]+2*[HC1/(HC0+HC1+HC2)]+4*[HC2/(HC0+HC1+HC2)] Equation 1:
Where the indicated variables are the relative abundance of: LC0=unloaded light chain, LC1=single loaded light chain, HC0=unloaded heavy chain, HC1=single loaded heavy chain, and HC2=double loaded heavy chain. One of ordinary skill in the art would appreciate that the invention encompasses expansion of this calculation to encompass higher loaded species such as LC2, LC3, HC3, HC4, HC5, and the like.

Equation 2, below, is used to estimate the amount of loading onto non-engineered cysteine residues. For engineered Fc mutants, loading onto the light chain (LC) was considered, by definition, to be nonspecific loading. Moreover, it was assumed that loading only the LC was the result of inadvertent reduction of the HC-LC disulfide bridge (i.e., the antibody was “over-reduced”). Given that a large excess of maleimide electrophile was used for the conjugation reactions (generally approximately 5 equivalents for single mutants and 10 equivalents for double mutants), it was assumed that any nonspecific loading onto the light chain was accompanied by a corresponding amount of non-specific loading onto the heavy chain (i.e., the other “half” of the broken HC-LC disulfide). With these assumptions in mind, the following equation (Equation 2) was used to estimate the amount of non-specific loading onto the protein:

Nonspecific loading=4*[LC1/(LC1+LC0)] Equation 2:
Where the indicated variables are the relative abundance of: LC0=unloaded light chain, LC1=single loaded light chain.

TABLE 12
Drug Antibody Ratio (DAR) of ADCs

| ADC | DAR |
|---|---|
| T(kK183C)-vc0101 | 2 |
| T(K290C)-vc0101 | 2 |
| T(K334C)-vc0101 | 2 |
| T(K392C)-vc0101 | 2 |
| T(kK183C + K290C)-vc0101 | 4 |
| T(kK183C + K334C)-vc0101 | 4 |
| T(kK183C + K392C)-vc0101 | 4 |
| T(K290C + K334C)-vc0101 | 4 |
| T(K290C + K392C)-vc0101 | 4 |
| T(K334C + K392C)-vc0101 | 4 |
| T(N297Q)-AcLysvc0101 | 4 |
| T(N297Q + K222R)-AcLysvc0101 | 4 |
| T(N297A + K222R + LCQ05)-AcLysvc0101 | 4 |
| T(LCQ05 + K222R)-AcLysvc0101 | 2 |
| T-mc8261 | 4.2 |
| T-m(H20)c8261 | 3.6 |
| T-MalPeg8261 | 3.1 |
| T-vc8261 | 4.3 |
| T-mc6121 | 3.5 |
| T-MalPeg6121 | 3.6 |
| T-mc0101 | 4.8 |
| T-vc0101 | 4.2 |
| T-vc8254 | 4 |
| T-vc6780 | 4.2 |
| T-vc0131 | 4.5 |
| T-MalPegMMAD | 4.4 |
| T-vcMMAE | 3.8 |
| T-DM1 | 4.2 |

C. Proteolysis with FabRICATOR® to Establish the Site of Loading

For the cysteine mutant ADCs, any nonspecific loading of the electrophillic payload onto the antibody is presumed to occur at the “interchain” also referred to as the “internal” cysteine residues (i.e., those that are typically part of the HC-HC or HC-LC disulfide bridges). In order to distinguish loading of electrophile onto the engineered cysteines in the Fc domain versus loading onto the internal cysteine residues (otherwise typically forming the S—S bonds between HC-HC or HC-LC), the conjugates were treated with a protease known to cleave between the Fab domains and the Fc domain of the antibody. One such protease is the cysteine protease IdeS, marketed as “FabRICATOR®” by Genovis, and described in von Pawel-Rammingen et al., 2002, EMBO J. 21:1607.

Briefly, following the manufacturer's suggested conditions, the ADC was treated with FabRICATOR® protease and the sample was incubated at 37° C. for 30 minutes. Samples were prepped for LCMS analysis by combining approximately 20 μl of sample (approximately 1 mg/mL in PBS) with 20 μl of 20 mM dithiothreitol (DTT) and allowing the mixture to stand at room temperature for 5 minutes. This treatment of human IgG1 resulted in three antibody fragments, all ranging from about 23 to 26 KDa in size: the LC fragment comprising an internal cysteine which typically forms an LC-HC interchain disulfide bond; the N-terminal HC fragment comprising three internal cysteines (where one typically forms an LC-HC disulfide bond and the other two cysteines found in the hinge region of the antibody and which typically form HC-HC disulfide bonds between the two heavy chains of the antibody); and the C-terminal HC fragment which contains no reactive cysteines other than those introduced by mutation in the constructs disclosed herein. The samples were analyzed by MS as described above. Loading calculations were performed in the same manner as previously described (above) in order to quantitate the loading of the LC, the N-terminal HC, and the C-terminal HC. Loading on the C-terminal HC is considered “specific” loading while loading onto the LC and the N-terminal HC is considered “nonspecific” loading.

To cross-check the loading calculations, a subset of ADCs were also assessed for loading using alternative methods (reverse phase high performance liquid chromatography [rpHPLC]-based and hydrophobic interaction chromatography [HIC]-based methods) as more fully described in the sections below.

D. Reverse phase HPLC analysis

Samples were prepped for reverse-phase HPLC analysis by combining approximately 20 μl of sample (approximately 1 mg/mL in PBS) with 20 μl of 20 mM dithiothreitol (DTT). After allowing the mixture to stand at room temperature for 5 minutes, the samples were injected into an Agilent 1100 HPLC system fitted with an Agilent Poroshell 300SB-C8 (2.1×75 mm) column. The system temperature was set to 60° C. and the eluent was monitored by UV (220 nM and 280 nM). A 20-minute gradient from 20% to 45% acetonitrile in water (with 0.1% TFA modifier) was utilized: T=0 min: 25% acetonitrile; T=2 min: 25% acetonitrile; T=19 min: 45% acetonitrile; and T=20 min: 25% acetonitrile. Using these conditions, the HC and LC of the antibody were baseline separated. The results of this analysis indicate that the LC remains largely unmodified (except for T(kK183C) and T(LCQ05) containing antibodies) while the HC is modified (data not shown).

E. Hydrophobic Interaction Chromatography (HIC)

Compounds were prepared for HIC analysis by diluting samples to approximately 1 mg/ml with PBS. The samples were analyzed by auto-injection of 15 μl onto an Agilent 1200 HPLC with a TSK-GEL Butyl NPR column (4.6×3.5 mm, 2.5 μm pore size; Tosoh Biosciences part #14947). The system includes an auto-sampler with a thermostat, a column heater and a UV detector.

The gradient method was used as follows: Mobile phase A: 1.5 M ammonium sulfate, 50 mM potassium phosphate dibasic (pH7); Mobile phase B: 20% isopropyl alcohol, 50 mM potassium phosphate dibasic (pH 7); T=0 min. 100% A; T=12 min., 0% A.

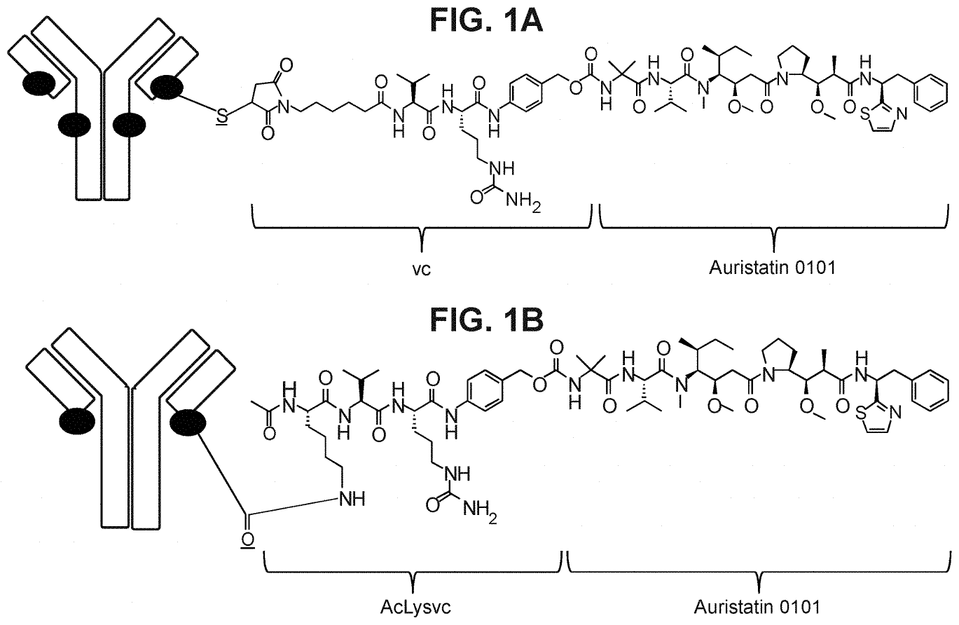

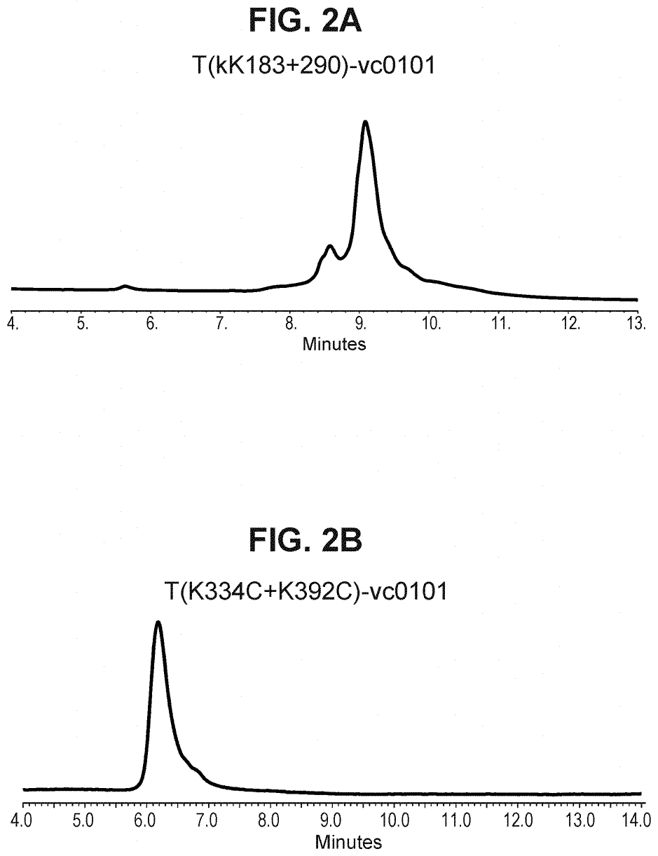

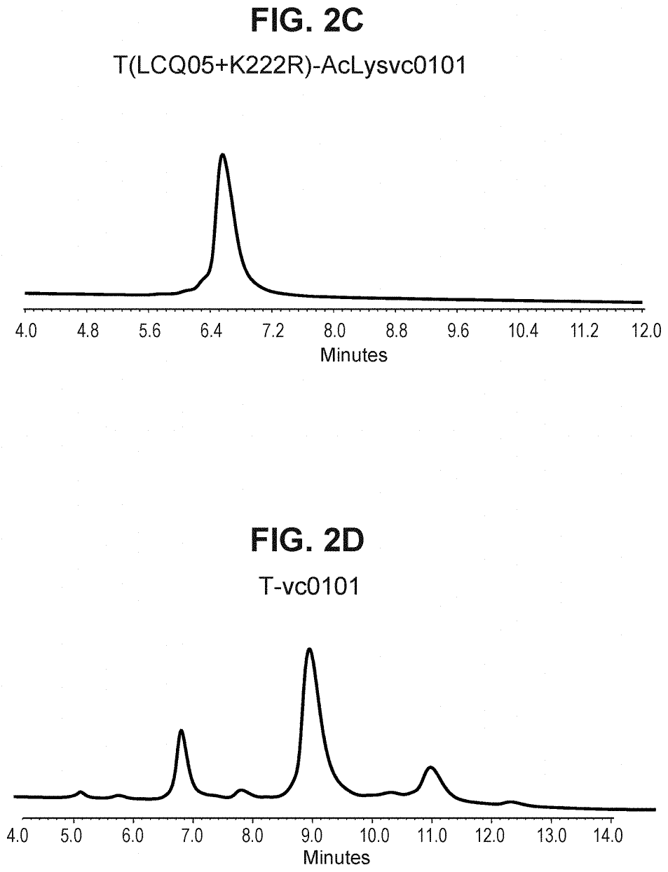

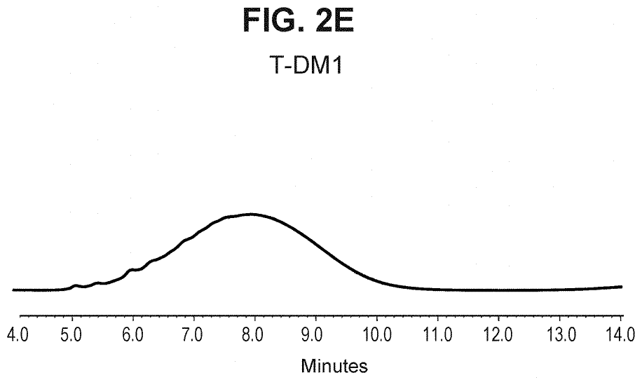

Retention times are shown in Table 13. Selected spectra are shown in FIGS. 2A-2E. ADCs using site-specific conjugation (T(kK183C+K290C)-vc0101, T(K334C+K392C)-vc0101 and T(LCQ05+K222R)-AcLysvc0101) (FIGS. 1A-1C) showed primarily one peak while ADCs using conventional conjugation (T-vc0101 and T-DM1) (FIGS. 2D-2E) showed a mixture of differentially loaded conjugates.

TABLE 13
ADC retention times by hydrophobic
interaction chromatography (HIC)

| ADC | RT (min) | RRT |
|---|---|---|
| T-vc0101 | 8.8 ± 0.1 | 1.68 |
| T(kK183C)-vc0101 | 7.2 ± 0.1 | 1.40 |
| T(K334C)-vc0101 | ND | |
| T(K392C)-vc0101 | 6.7 ± 0.1 | 1.29 |
| T(L443C)-vc0101 | 10.1 ± 0.1 | 1.98 |
| T(kK183C + K290C)-vc0101 | 9.0 ± 0.0 | 1.77 |
| T(kK183C + K334C)-vc0101 | ND | |
| T(kK183C + K392C)-vc0101 | 7.7 ± 0.1 | 1.54 |
| T(kK183C + L443C)-vc0101 | 10.6 | 2.04 |
| T(K290C + K334C)-vc0101 | 6.3 ± 0.0 | 1.21 |
| T(K290C + K392C)-vc0101 | 7.8 ± 0.0 | 1.54 |
| T(K334C + K392C)-vc0101 | 6.0 ± 0.3 | 1.18 |
| T(K392C + L443C)-vc0101 | 10.8 ± 0.0 | 2.08 |
| T(LCQ05 + K222R)- AcLys-vc0101 | 6.5 | 1.27 |
| T(N297A + K222R + LCQ05)-AcLys-vc0101 | 6.3 ± 0.1 | 1.24 |

ND = not determined
RT = retention time (min) on HIC
RRT = mean relative retention time, calculated by RT of ADC divided by RT of benchmark unconjugated wild type trastuzumab having a typical retention time of 5.0-5.2 min

F. Thermostability

Differential Scanning calorimetry (DCS) was used to determine the thermal stability of the engineered cysteine and transglutaminase antibody variants, and corresponding Aur-06380101 site-specific conjugates. For this analysis, samples formulated in PBS-CMF pH 7.2 were dispensed into the sample tray of a MicroCal VP-Capillary DSC with Autosampler (GE Healthcare Bio-Sciences, Piscataway, N.J.), equilibrated for 5 minutes at 10° C. and then scanned up to 110° C. at a rate of 100° C. per hour. A filtering period of 16 seconds was selected. Raw data was baseline corrected and the protein concentration was normalized. Origin Software 7.0 (OriginLab Corporation, Northampton, Mass.) was used to fit the data to an MN2-State Model with an appropriate number of transitions.

All single and double cysteine engineered antibody variants as well as the engineered LCQ05 acyl donor glutamine-containing tag antibody exhibited excellent thermal stability as determined by the first melting transition (Tm1) >65° C. (Table 14).

Trastuzumab derived monoclonal antibodies conjugated to 0101 using site specific conjugation methods were also evaluated and shown to have exceptional thermal stability as well (Table 15). However, the Tm1 for T(K392C+L443C)-vc0101 ADC was most impacted by conjugation of the payload since it was −4.35° C. relative to the unconjugated antibody.

Taken together these results demonstrated that both the engineered cysteine and acyl donor glutamine-containing tag antibody variants were thermally stable and that site-specific conjugation of 0101 via a vc linker yielded conjugates with excellent thermal stability. Furthermore, the lower thermal stability observed for T(K392C+L443C)-vc0101 relative to the unconjugated antibody indicated that conjugation of 0101 via a vc linker to certain combinations of engineered cysteine residues can impact stability of the ADC.

TABLE 14
Thermal Stability of Engineered Trastuzumab Derived Variants

| Antibody | Tm1 (° C.) | Tm2 (° C.) | Tm3 (° C.) |
|---|---|---|---|
| T(κK183C) | 72.17 ± 0.029 | 80.78 ± 0.37 | 82.81 ± 0.055 |
| T(L443C) | 72.02 ± 0.06 | 80.98 ± 1.10 | 82.96 ± 0.11 |
| T(LCQ05) | 72.22 ± 0.027 | 81.16 ± 0.19 | 82.88 ± 0.033 |
| T(κK183C + K290C) | 75.4 | 81.1 | 82.9 |
| T(κK183C + K392C) | 75 | 81 | 83 |
| T(κK183C + L443C) | 72.24 ± 0.05 | 80.89 ± 0.89 | 82.87 ± 0.16 |
| T(K290C + K334C) | 75.0 ± 0.14 | 83.0 ± 0.1 | 81.1 ± 0.4 |
| T(K334C + K392C) | 75.3 ± 0.25 | 82.7 ± 0.53 | 81.0 ± 2.9 |
| T(K290C + K392C) | 77 | 81 | 83 |
| T(K392C + L443C) | 73.95 ± 0.29 | 80.54 ± 0.70 | 82.81 ± 0.17 |

TABLE 15
Thermal Stability of Site-Specific Conjugates
Conjugated to Auristatin 0101

| Site-Specific | | | | Tm1_{SSC} − |
|---|---|---|---|---|
| Conjugate | Tm1 (° C.) | Tm2 (° C.) | Tm3 (° C.) | Tm1_{Ab} |
| T(κK183C)- | 70.16 ± 0.03 | 80.45 ± 0.12 | 82.04 ± 0.03 | −2.01 |
| vc0101 | | | | |
| T(L443C)- | 72.34 ± 0.10 | 80.20 ± 0.59 | 82.44 ± 0.10 | 0.32 |
| vc0101 | | | | |
| T(κK183C + | 70.11 ± 0.02 | 78.89 ± 0.59 | 81.38 ± 0.10 | −2.13 |
| L443C)-vc0101 | | | | |
| T(K392C + | 69.60 ± 0.35 | 79.21 ± 0.43 | 82.10 ± 0.05 | −4.35 |
| L443C)-vc0101 | | | | |

### Example 8: ADC Binding to HER2

A. Direct Binding

BT474 cells (HTB-20) were trypsinized, spun down and re-suspended in fresh media. The cells were then incubated with a serial of dilutions of either the ADCs or unconjugated trastuzumab with starting concentration of 1 μg/ml for one hour at 4° C. The cells were then washed twice with ice cold PBS and incubated with anti-human Alexafluor 488 secondary antibody (Cat #A-11013, Life technologies) for 30 min. The cells were then washed twice and then re-suspended in PBS. The mean fluorescence intensity was read using Accuri flow cytometer (BD Biosciences San Jose, Calif.).

TABLE 16
ADC binding to HER2

| ADC/Ab | EC₅₀ |
|---|---|
| trastuzumab | 0.37 |
| T(kK183C + K392C)-vc0101 | 0.56 |
| T(kK183C + K290C)-vc0101 | 0.47 |
| T(K290C + K392C)-vc0101 | 0.32 |
| T-DM1 (Kadcyla) | 0.40 |
| T(LCQ05 + K222R)-AcLysvc0101 | 0.37 |
| T(N297Q + K222R)-AcLysvc0101 | 0.36 |

EC50 = the concentration of an antibody or ADC that gives half-maximal binding.

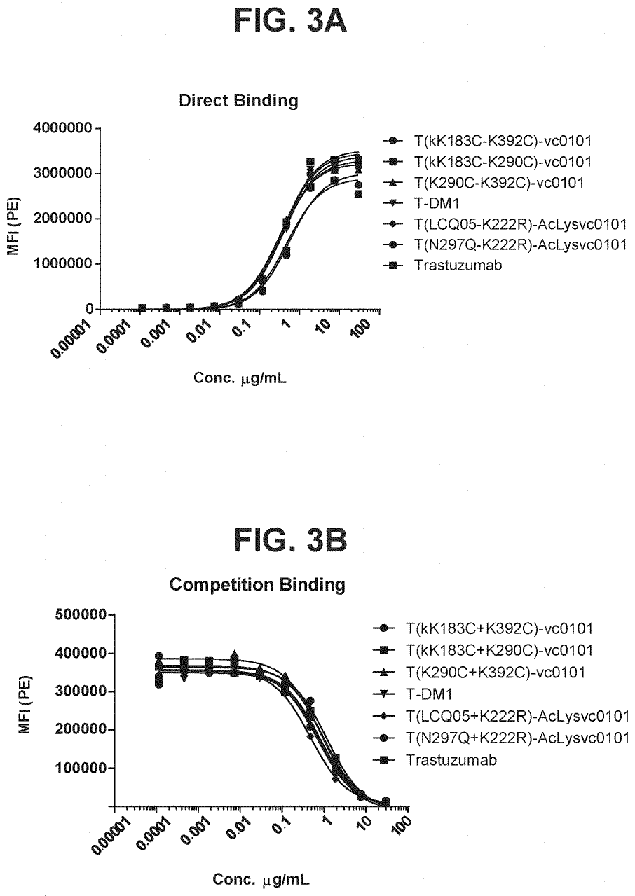

As shown in FIG. 3A and Table 16, ADCs T(LCQ05+K222R)-AcLysvc0101, T(N297Q+K222 R)-AcLysvc0101, T(kK183C+K2900)-vc0101, T(kK183C+K3920)-vc0101, T(K290C+K392C)-vc0101 had similar binding affinities as T-DM1 and trastuzumab by direct binding. This indicates that the modifications to the antibody in the ADCs of the present invention and the addition of the linker-payload did not significantly affect binding.

B. Competitive Binding by FACS

BT474 cells were trypsinized, spun down and re-suspended in fresh media. The cells were then incubated for one hour at 4° C. with serial dilutions of either the ADCs or the unconjugated trastuzumab combined with 1 μg/mL of trastuzumab-PE (custom synthesized 1:1 PE labeled trastuzumab by eBiosciences (San Diego, Calif.)). The cells were then washed twice and then re-suspended in PBS. The mean fluorescence intensity was read using Accuri flow cytometer (BD Biosciences San Jose, Calif.).

As shown in FIG. 3B, ADCs T(LCQ05+K222R)-AcLysvc0101, T(N297Q+K222R)-AcLysvc0101, T(kK183C+K290C)-vc0101, T(kK183C+K392C)-vc0101, T(K290C+K392C)-vc0101 had similar binding affinities as T-DM1 and trastuzumab by competition binding to PE labeled trastuzumab. This indicates that the modifications to the antibody in the ADCs of the present invention and the addition of the linker-payload did not significantly affect binding.

### Example 9: ADC Binding to Human FcRn

It is believed in the art that FcRn interacts with IgG regardless of subtype in a pH dependent manner and protects the antibody from degradation by preventing it from entering the lysosomal compartment where it is degraded. Therefore, a consideration for selecting positions for introduction of reactive cysteines into the wild type IgG1-Fc region was to avoid altering the FcRn binding properties and half-life of the antibody comprising the engineered cysteine.

BIAcore® analysis was performed to determine the steady-state affinity (KD) for the trastuzumab derived monoclonal antibodies and their respective ADCs for binding to human FcRn. BIAcore® technology utilizes changes in the refractive index at the surface layer of a sensor upon binding of the trastuzumab derived monoclonal antibodies or their respective ADCs to human FcRn protein immobilized on the layer. Binding was detected by surface plasmon resonance (SPR) of laser light refracting from the surface. Human FcRn was specifically biotinylated through an engineered Avi-tag using the BirA reagent (Catalog #: BIRA500, Avidity, LLC, Aurora, Colo.) and immobilized onto a streptavidin (SA) sensor chip to enable uniform orientation of the FcRn protein on the sensor. Next, various concentrations of the trastuzumab derived monoclonal antibodies or their respective ADCs or in 20 mM MES (2-(N-morpholino)ethanesulfonic acid pH 6. 0, with 150 mM NaCl, 3 mM EDTA (ethylenediaminetetraacetic acid), 0.5% Surfactant P20 (MES-EP) were injected over the chip surface. The surface was regenerated using HBS-EP+0.05% Surfactant P20 (GE Healthcare, Piscataway, N.J.), pH 7.4, between injection cycles. The steady-state binding affinities were determined for the trastuzumab derived monoclonal antibodies or their respective ADCs, and these were compared with the wild type trastuzumab antibody (comprising no cysteine mutations in the IgG1 Fc region, no TGase engineered tag or site-specific conjugation of a payload).

These data demonstrated that incorporation of engineered cysteine residues into the IgG-Fc region at the indicated positions of the invention did not alter affinity to FcRn (Table 17).

TABLE 17
Steady-State Affinities of Site-Specific
Conjugates Binding Human FcRn

| | K_{D} [nM] | K_{D} [nM] | K_{D} [nM] |
|---|---|---|---|
| | Experi- | Experi- | Experi- |
| | ment 1 | ment 2 | ment 3 |
| Trastuzumab WT | 1050.0 | 705.8 | 859.2 |
| T-DM1 | ND | 500.8 | ND |
| T(K290C + K334C) | 987.0 | ND | ND |
| T(K290C + K334C)-vc0101 | 1218.0 | ND | ND |
| T(K334C + K392C) | 834.1 | ND | ND |
| T(K334C + K392C)-vc0101 | 1404.0 | ND | ND |
| T(κK183C + K290C) | 1173.0 | ND | ND |
| T(κK183C + K290C)-vc0101 | 473.8 | ND | ND |
| T(κK183C + K392C) | 1009.0 | ND | ND |
| T(κK183C + K392C)-vc0101 | 672.5 | ND | ND |
| T(κK183C)-vc0101 | 961.5 | ND | ND |
| T(LCQ05) | 900.9 | ND | ND |
| T(LCQ05)-vc0101 | 1050.0 | ND | ND |
| T(K392C) | ND | 468.3 | ND |
| T(K392C)-vc0101 | ND | 518.8 | ND |
| T(N297Q)-vc0101 | ND | 647.9 | ND |
| T(κK183C + K334C)-vc0101 | ND | 416.5 | ND |
| T(κK183C + K443C) | ND | 542.8 | ND |
| T(κK183C + K443C)-vc0101 | ND | 287.5 | ND |
| T(K290C) | ND | ND | 650.3 |
| T(K290C)-vc0101 | ND | ND | 874.6 |
| T(K290C + K392C)-vc0101 | ND | ND | 554.7 |
| T(K334C) | ND | ND | 631.6 |
| T(K334C)-vc0101 | ND | ND | 791.2 |
| T(K392C + K443C) | ND | ND | 601.7 |
| T(K392C + K443C)-vc0101 | ND | ND | 197.9 |

ND = Not Determined

### Example 10: ADC Binding to Fcγ Receptors

Binding of the ADCs using site-specific conjugation to human Fc-γ receptors was evaluated in order to understand if conjugation to a payload alters binding which can impact antibody related functionality properties such as antibody-dependent cell-mediated cytotoxicity (ADCC). FcγIIIa (CD16) is expressed on NK cells and macrophages, and co-engagement of this receptor with the target expressing cells via antibody binding induces ADCC. BIAcore® analysis was used to examine binding of the trastuzumab derived monoclonal antibodies and their respective ADCs to Fc-γ receptors IIa (CD32a), IIb(CD32b), IIIa (CD16) and FcγRI (CD64).

For this surface plasmon resonance (SPR) assay, recombinant human epidermal growth factor receptor 2 (Her2/neu) extra-cellular domain protein (Sino Biological Inc. , Beijing, P. R. China) was immobilized on a CM5 chip (GE Healthcare, Piscataway, N.J.) and ˜300-400 response units (RU) of either a trastuzumab derived monoclonal antibody or its respective ADC was captured. The T-DM1 was included in this evaluation as a positive control since it has been shown to retain binding properties post-conjugation to Fcγ receptors comparable to the unconjugated trastuzumab antibody. Next, various concentrations of the Fcγ receptors FcγIIa (CD32a), FcγIIb(CD32b), FcγIIIa (CD16a) and FcγRI (CD64) were injected over the surface and binding was determined.

FcγRs IIa, IIb and IIIa exhibited rapid on/off rates and therefore the sensorgrams were fit to steady state model to obtain K_{D} values. FcγRI exhibited slower on/off rates so data was fit to a kinetic model to obtain K_{D} values.

Conjugation of payload at the engineered cysteine positions 290 and 334 showed a moderate loss in FcγR affinity, specifically to CD16a, CD32a and CD64 compared to their unconjugated counterpart antibodies and T-DM1 (Table 18). However, simultaneous conjugation at sites 290, 334 and 392 resulted in a substantial loss of affinity to CD16a, CD32a and CD32b, but not CD64 as observed with the T(K290C+K334C)-vc0101 and T(K334C+K392C)-vc0101 (Table 18). Interestingly, T(κK183C+K290C)-vc0101 exhibited comparable binding to all FcγR evaluated in this study despite harboring drug payload on the K290C position (Table 18). As expected the transglutaminase mediated conjugated T(N297Q+K222R)-AcLysvc0101 did not bind to any of the Fcγ receptors evaluated since location of the acyl donor glutamine-containing tag removes N-linked glycosylation. Contrary, T(LCQ05+K222R)-AcLysvc0101 retained full binding to the Fcγ receptors as the glutamine-containing tag is engineered within the human Kappa light chain constant region.

Taken together, these results suggested that location of the conjugated payload can impact binding of the ADC to FcγR and may impact the antibody functionality of the conjugate.

TABLE 18
Binding Affinity of Site-Specific Conjugates for Fcγ
Receptors binding to the CD16a, CD32a, CD32b and CD64

| | K_{D} [M] | | | |
|---|---|---|---|---|
| | FcγRIIIa | FcγRIIa | FcγRIIb | FcγRI |
| | (CD16a) | (CD32a) | (CD32b) | (CD64) |
| | [μM] | [μM] | [μM] | [pM] |
| Trastuzumab WT mAb | 0.36 | 0.74 | 4.08 | 23 |
| T-DM1 ADC | 0.30 | 0.53 | 2.97 | 27 |
| T(K290C)-vc0101 | 1.20 | 1.70 | 3.74 | 185 |
| T(K334C)-vc0101 | 0.81 | 1.42 | 4.74 | ND |
| T(K290C + K334C)- | 5.14 | 6.30 | 6.38 | 110 |
| vc0101 | | | | |
| T(K334C + K392C)- | 2.38 | 4.18 | 11.30 | 43 |
| vc0101 | | | | |
| T(K392C)-vc0101 | 0.45 | 0.73 | 4.33 | ND |
| T(κK183C + K290C)- | 0.47 | 0.70 | 3.63 | 37 |
| 0101 | | | | |
| T(LCQ05 + K222R)- | 0.43 | 0.62 | 3.41 | 32 |
| AcLysvc0101 | | | | |
| T(N297Q − K222R)- | NB | NB | NB | NB |
| AcLysvc0101 | | | | |

ND = Not Determined,
NB = No Binding

### Example 11: ADCC Activities

In ADCC assays, Her2-expressing cell lines BT474 and SKBR3 were used as target cells while NK-92 cells (an interleukin-2 dependent natural killer cell line derived from peripheral blood mononuclear cells from a 50 year old Caucasian male by Conkwest) or human peripheral blood mononucleocytes (PBMC) isolated from the freshly drawn blood from a healthy donor (#179) were used as effector cells.

Target cells (BT474 or SKBR3) of 1×10⁴ cells/100 μl/well were placed in 96-well plate and cultured overnight in RPM11640 media at 37° C./5% CO₂. The next day, the media was removed and replaced with 60 μl assay buffer (RPM11640 media containing 10 mM HEPES), 20 μl of 1 μg/ml antibody or ADC, followed by addition of 20 μl 1×10⁵ (for SKBR3) or 5×10⁵ (for BT474) PBMC suspension or 2.5×10⁵ NK92 cells for both cell lines to each well to achieve effector to target ratio of 50:1 for BT474 or of 25:1 for SKBR3 for PBMC, 10:1 for NK92. All samples were run in triplicate.

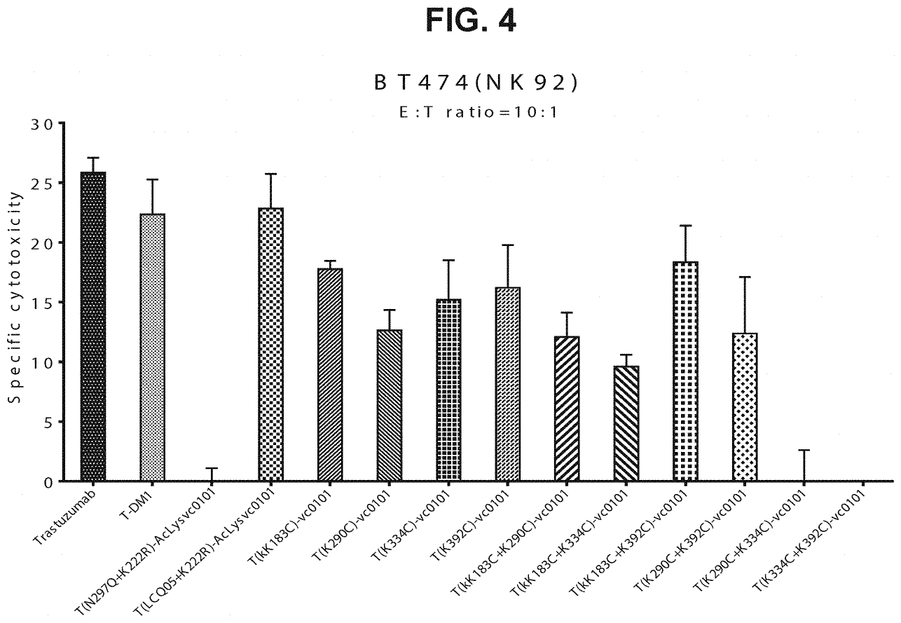

Assay plates were incubated at 37° C./5% CO₂ for 6 hours and then equilibrated to room temperature. LDH release from cell lysis was measured using CytoTox-One™ reagent at an excitation wavelength of 560 nm and an emission wavelength of 590 nm. As a positive control, 8 μL of Triton was added to generate a maximum LDH release in control wells. The specific cytotoxicity shown in FIG. 4 was calculated using the following formula:

$$\% Specific Cytotoxicity = \frac{Experimental - effector spontaneous - target spontaneous}{Target maximum - Target spontaneous} \times 100$$

“Experimental” corresponds to the signal measured in one of the condition described above.
“Effector spontaneous” corresponds to the signal measured in the presence of PBMC alone.
“Target spontaneous” corresponds to the signal measured in the presence of target cells alone.
“Target Maximum” corresponds to the signal measured in the presence of detergent-lysed target cells alone.

FIG. 4 shows the ADCC activities tested for trastuzumab, T-DM1 and vc0101 ADC conjugates. The data conform the reported ADCC activities of Trastuzumab and T-DM1. Since the mutation of N297Q is at the glycosylation site, T(N297Q+K222R)-AcLysvc0101 was not expected to have ADCC activities which was also confirmed in the assays. For single mutant (K183C, K290C, K3340, K392C including LCQ05) ADCs, ADCC activities were maintained. Surprisingly, for double mutant (K183C+K290C, K183C+K392C, K183C+K334C K290C+K392C, K290C+K334C, K334C+K392C) ADCs, ADCC activities were maintained in all except two double mutant ADCs associated with K334C site (K290C+K334C and K334C+K392C).

### Example 12: In vitro Cytotoxicity Assays

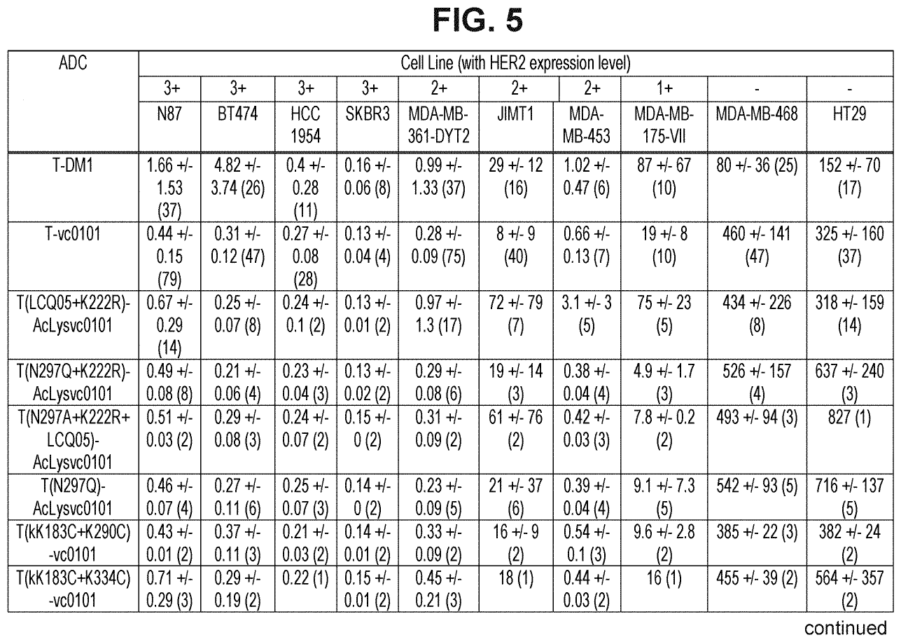

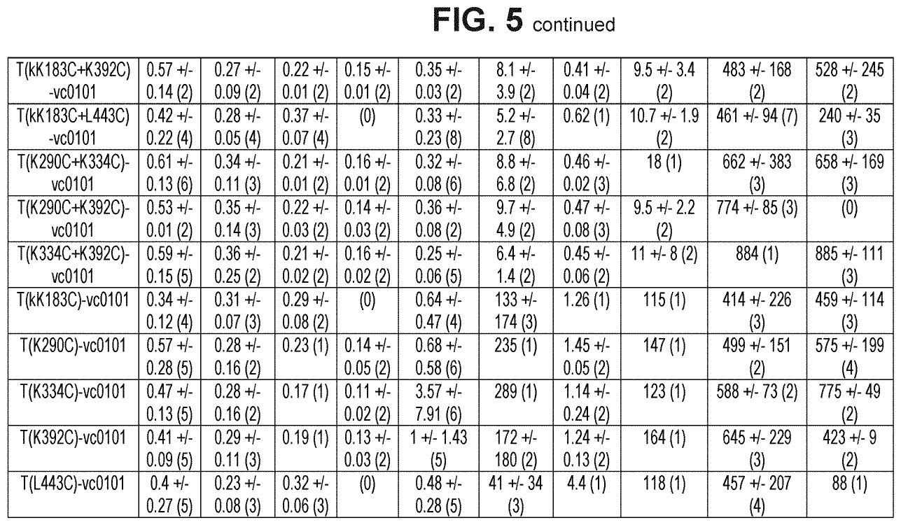

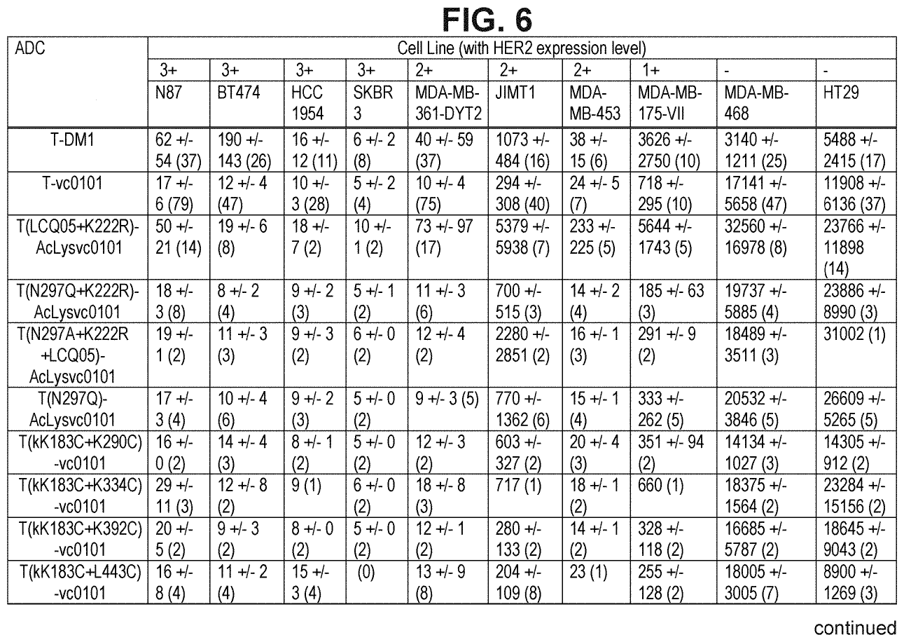

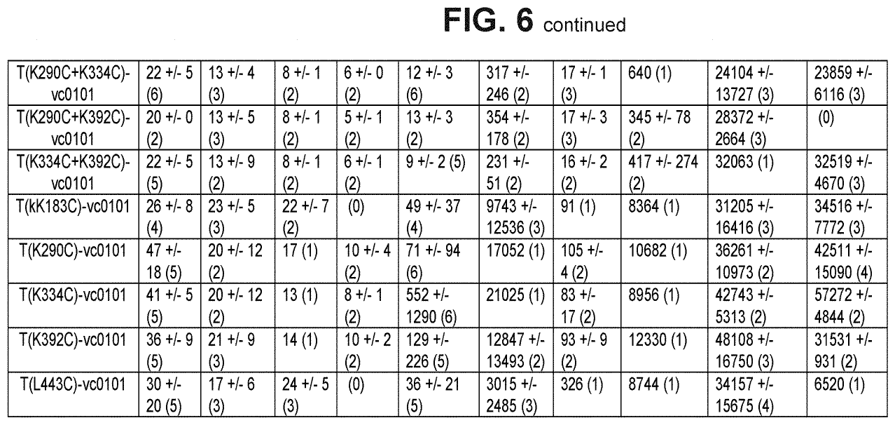

Antibody-drug conjugates were prepared as indicated in Example 3. Cells were seeded in 96-well plates at low density, then treated the following day with ADCs and unconjugated payloads at 3-fold serial dilutions at 10 concentrations in duplicate. Cells were incubated for 4 days in a humidified 37° C./5% CO₂ incubator. The plates were harvested by incubating with CellTiter® 96 AQueous One MTS Solution (Promega, Madison, Wis.) for 1.5 hours and absorbance measured on a Victor plate reader (Perkin-Elmer, Waltham, Mass.) at wavelength 490 nm. IC₅₀ values were calculated using a four-parameter logistic model with XLfit (IDBS, Bridgewater, N.J.) and reported as nM payload concentration in FIG. 5 and ng/ml antibody concentration in FIG. 6. The IC₅₀ are shown +/−the standard deviation with the number of independent determinations in parenthesis.

The ADCs containing vc-0101 or AcLysv-0101 linker-payloads were highly potent against Her2-positive cell models and selective against Her2-negative cells, compared with the benchmark ADC, T-DM1 (Kadcyla).

ADCs synthesized with site-specific conjugation to trastuzumab showed high level potency and selectivity against Her2 cell models. Notably, several trastuzumab-vc0101 ADCs are more potent than T-DM1 in moderate or low Her2-expressing cell models. For example, the in vitro cytotoxicity IC₅₀ for T(kK183C+K290C)-vc0101 in MDA-MB-175-VII cells (with 1+Her2 expression) is 351 ng/ml, compared with 3626 ng/ml for T-DM1 (˜10-fold lower). For cells with 2++ level Her2 expression such as MDA-MB-361-DYT2 and MDA-MB-453 cells, the IC₅₀ for T(kK183C+K290C)-vc0101 is 12-20 ng/ml, compared with 38-40 ng/ml for T-DM1.

### Example 13: Xenograft Models

Trastuzumab derived ADCs of the invention tested in an N87 gastric cancer xenograft model, 37622 lung cancer xenograft model, and a number of breast cancer xenograft models (i.e., HCC 1954, JIMT-1, MDA-MB-361(DYT2) and 144580 (PDX) models). For each model described below the first dose was given on Day 1. The tumors were measured at least once a week and their volume was calculated with the formula: tumor volume (mm³)=0.5×(tumor width²)(tumor length). The mean tumor volumes(±S. E. M.) for each treatment group were calculated having a maximum of 8-10 animals and a minimum of 6-8 animals to be included.

A. N87 Gastric Xenografts

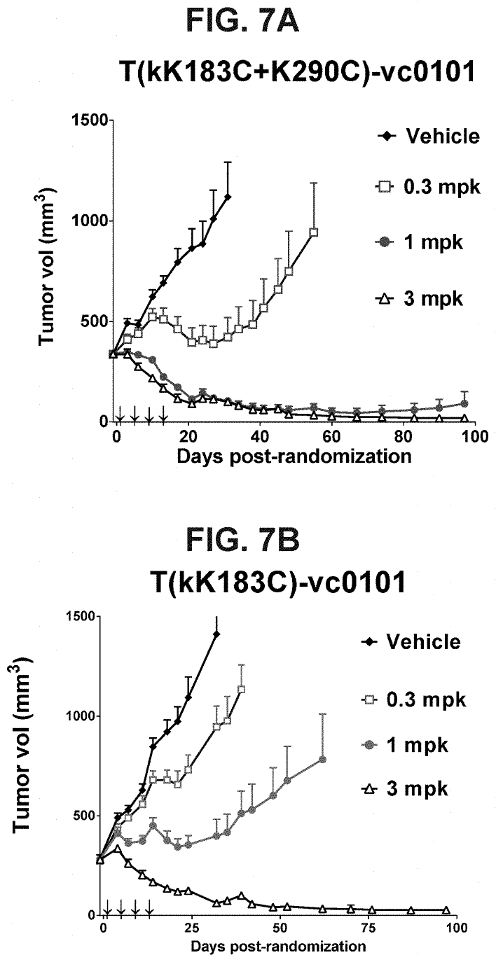

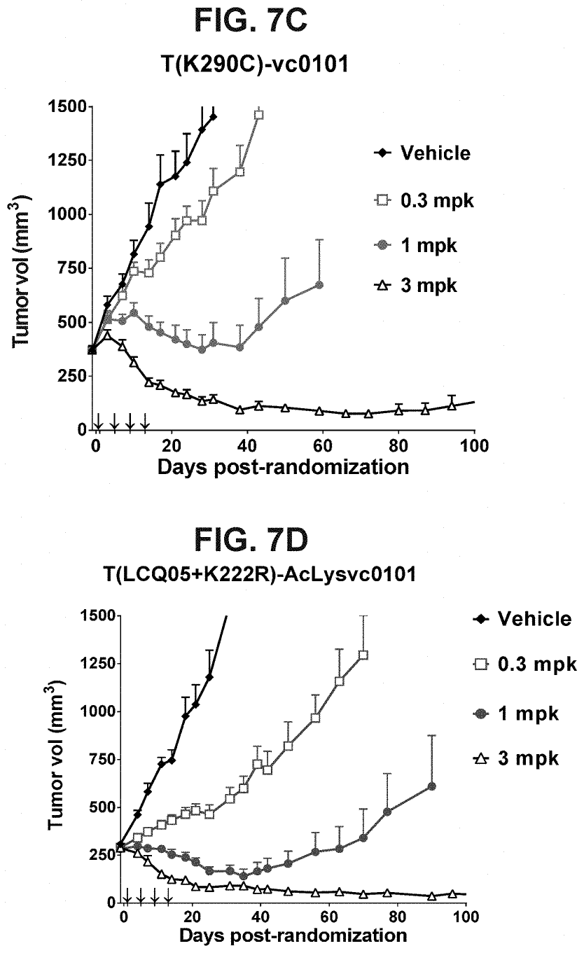

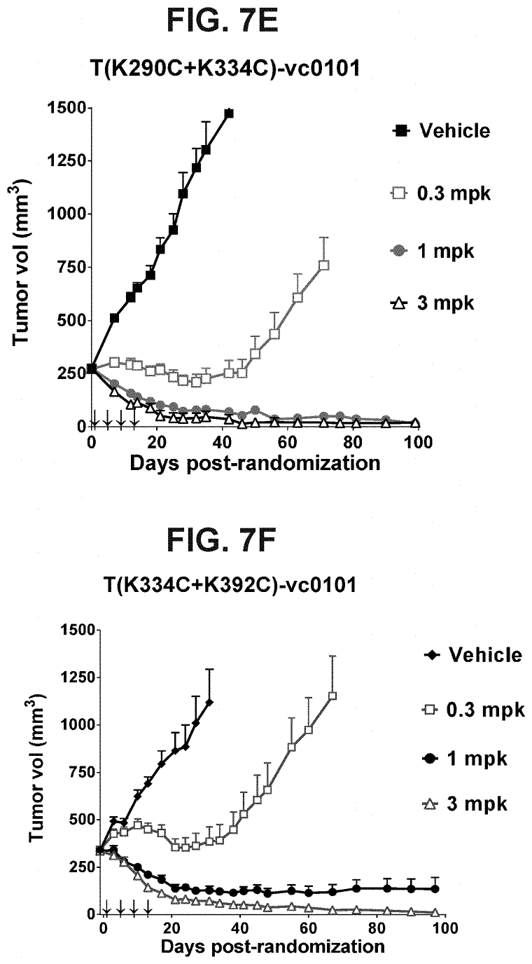

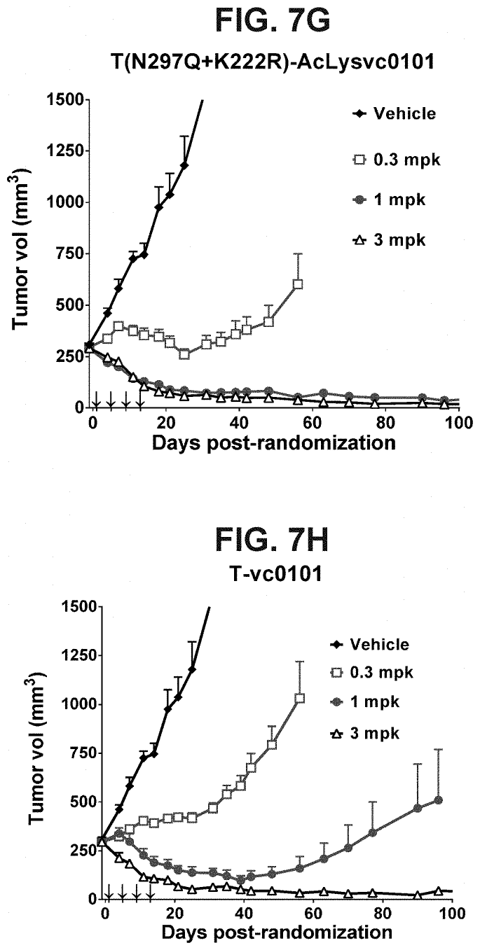

The effects of Trastuzumab derived ADCs were examined in immunodeficient mice on the in vivo growth of human tumor xenografts that were established from the N87 cell line (ATCC CRL-5822) which has high level HER2 expression. To generate xenografts, nude (Nu/Nu, Charles River Lab, Wilmington, Mass.) female mice were implanted subcutaneously with 7.5×10⁶ N87 cells in 50% Matrigel (BD Biosciences). When the tumors reached a volume of 250 to 450 mm³, the tumors were staged to ensure uniformity of the tumor mass among various treatment groups. The N87 gastric model was dosed 4 times intravenously 4 days apart (Q4d×4) with PBS vehicle, Trastuzumab ADCs (at 0.3, 1 and 3 mg/kg) or T-DM1 (1, 3 and 10 mg/kg) (FIG. 7).

The data demonstrates that Trastuzumab derived ADCs inhibited growth of N87 gastric xenografts in a dose-dependent manner (FIGS. 7A-7H).

As illustrated in FIG. 71, T-DM1 had delayed tumor growth at 1 and 3 mg/kg and had complete regression of tumors at 10 mg/kg. However, T(kK183C+K290C)-vc0101 provided complete regression at 1 and 3 mg/kg and partial regression at 0.3 mg/kg (FIG. 7A). The data shows that T(kK183C+K290C)-vc0101 is significantly more potent (˜10 times) than T-DM1 in this model.

Similar in vivo efficacy from ADCs with DAR4 (FIGS. 6E, 6F and 6G) were obtained compared to 183+290 (FIG. 7A). In addition, single mutants were evaluated that are DAR2 ADCs (FIGS. 7B, 7C and 7D). In general, these ADCs are less efficacious compared to DAR4 ADCs but more efficacious than T-DM1. Among DAR2 ADCs, it appears LCQ05 is the most potent ADC based on the in vivo efficacy data.

B. HCC1954 Breast Xenografts

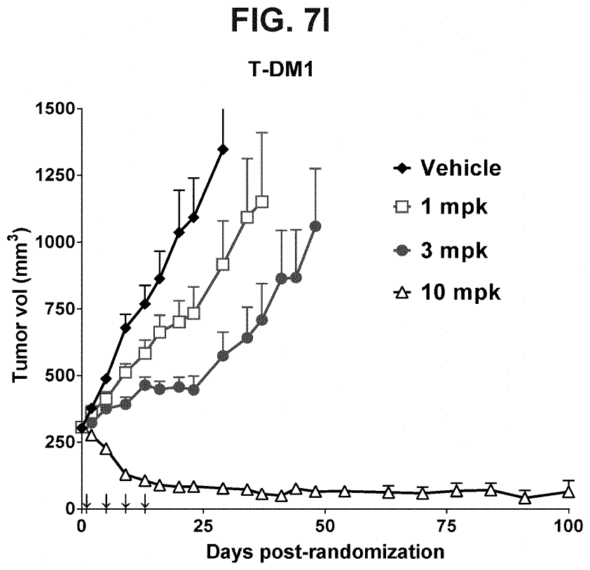

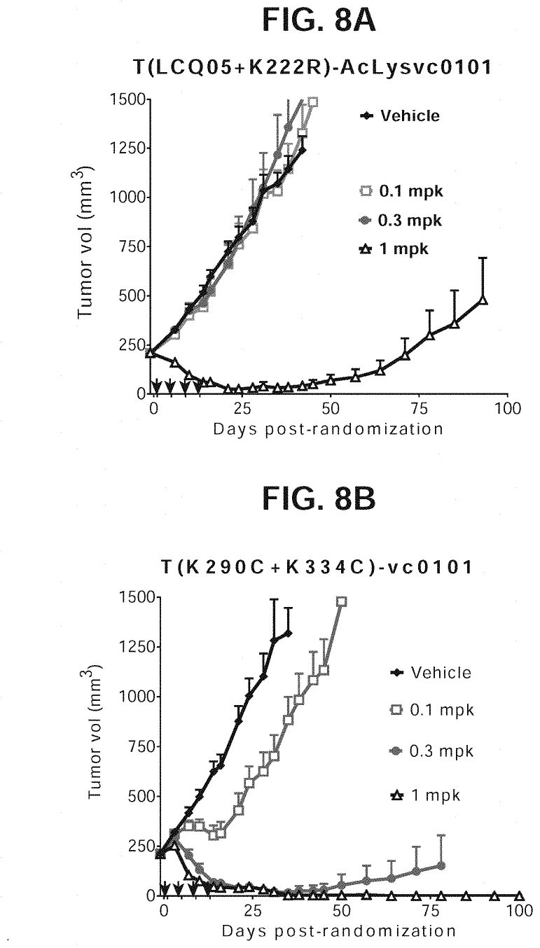

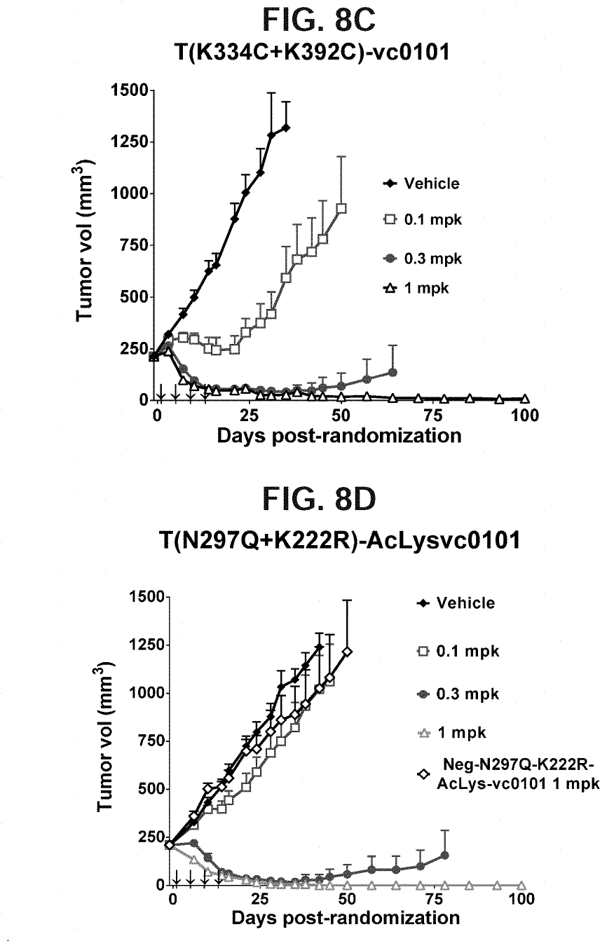

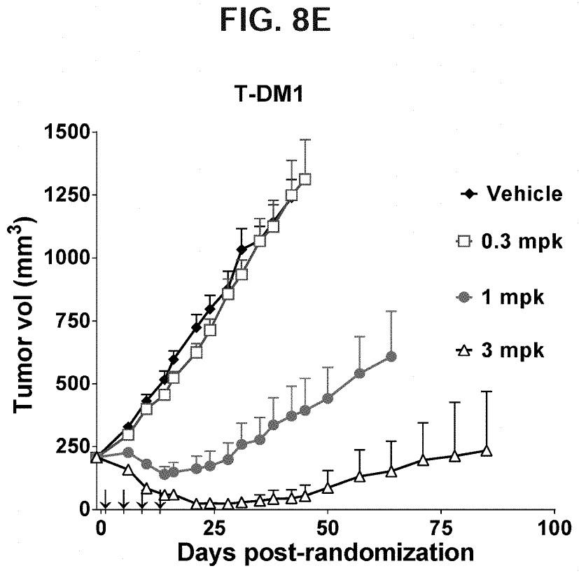

HCC1954 (ATCC #CRL-2338) is a high HER2 expression breast cancer cell line. To generate xenografts, SHO female mice (Charles River, Wilmington, Mass.) were implanted subcutaneously with 5×10⁶ HCC1954 cells in 50% Matrigel (BD Biosciences). When the tumors reached a volume of 200 to 250 mm³, the tumors were staged to ensure uniformity of the tumor mass among various treatment groups. The HCC1954 breast model was dosed intravenously Q4d×4 with PBS vehicle, Trastuzumab derived ADCs and negative control ADC (FIGS. 8A-8E).

The data demonstrates that Trastuzumab ADCs inhibited growth of HCC1954 breast xenografts in a dose-dependent manner. Comparing the 1 mg/kg dose, vc0101 conjugates were more efficacious than T-DM1. Comparing the 0.3 mg/kg dose, DAR4 loaded ADCs (FIGS. 8B, 8C and 8D) are more efficacious than a DAR2 loaded ADC (FIG. 8A). Further, the negative control ADC at 1 mg/kg had very minimal impact on tumor growth compared to vehicle control (FIG. 8D). However, T(N297Q+K222R)-AcLysvc0101 completely regressed the tumors indicating the target specificity.

C. JIMT-1 Breast Xenografts

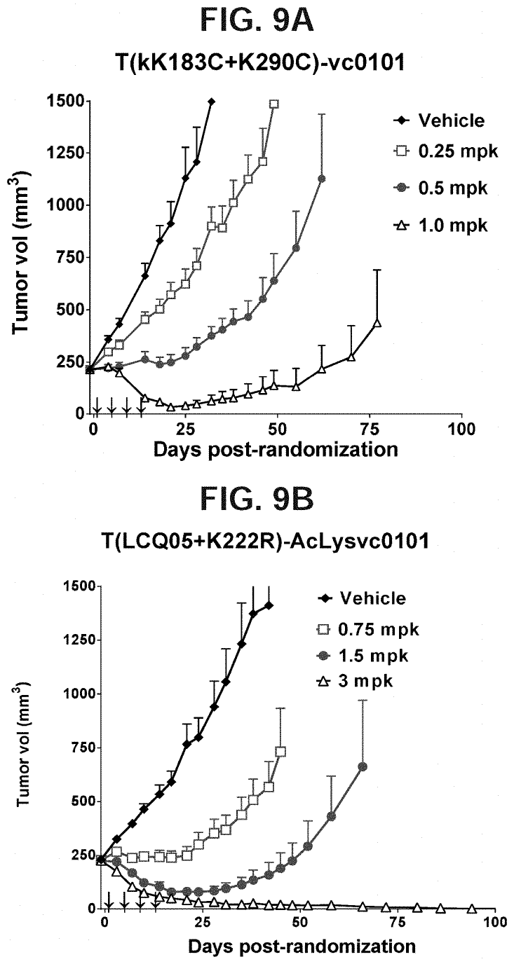

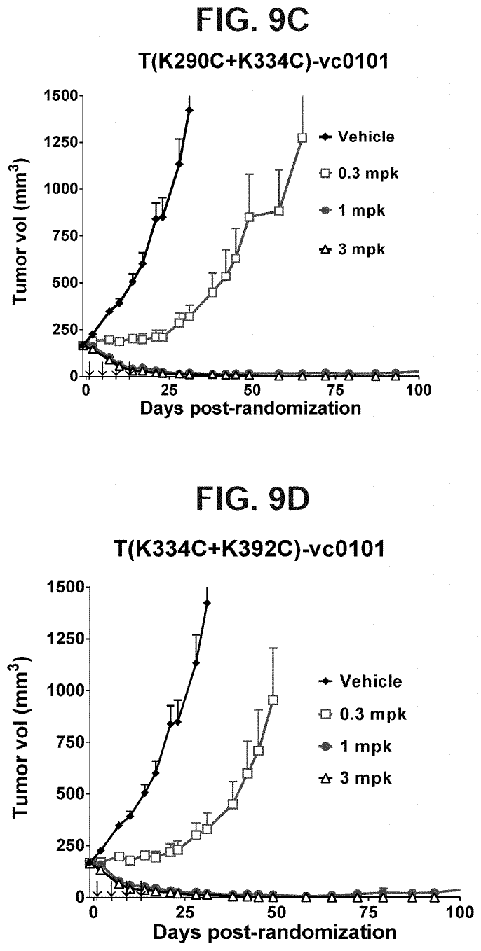

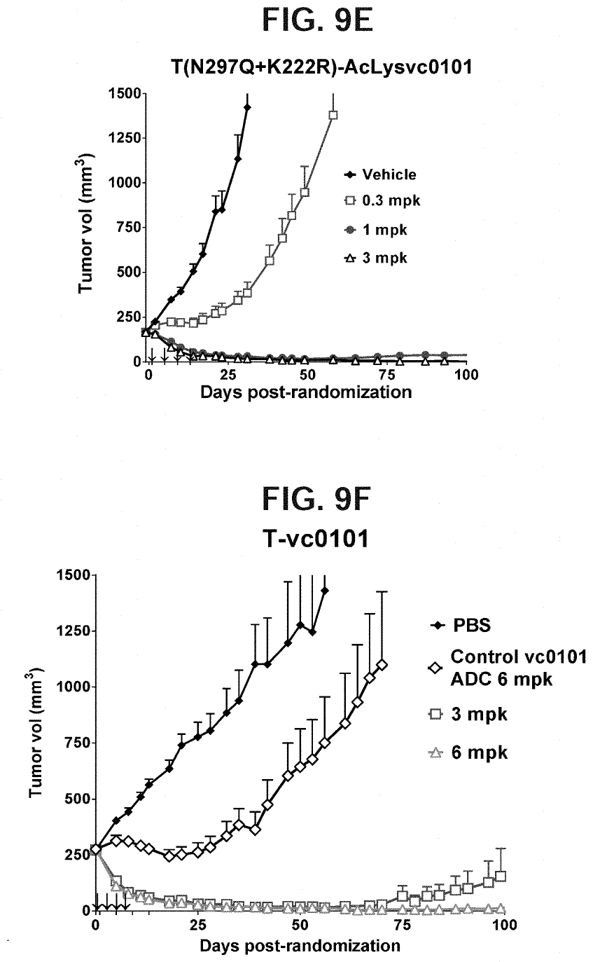

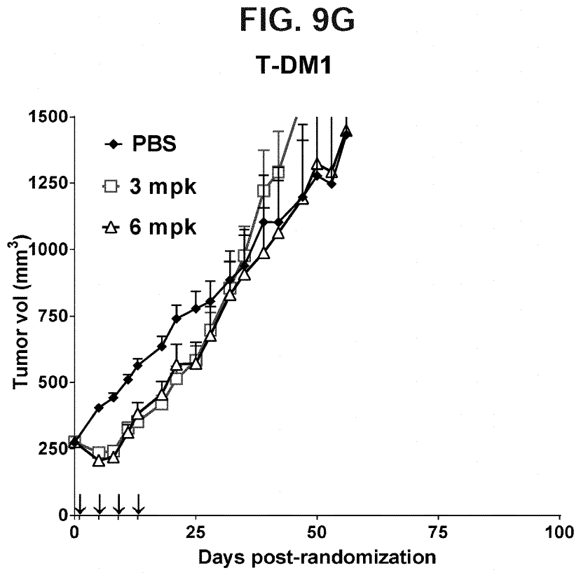

JIMT-1 is a breast cancer cell line expressing moderate/low Her2 and is inherently resistant to trastuzumab. To generate xenografts, nude (Nu/Nu) female mice were implanted subcutaneously with 5×10⁶ JIMT-1 cells (DSMZ #ACC-589) in 50% Matrigel (BD Biosciences). When the tumors reached a volume of 200 to 250 mm³, the tumors were staged to ensure uniformity of the tumor mass among various treatment groups. The JIMT-1 breast model was dosed intravenously Q4d×4 with PBS vehicle, T-DM1 (FIG. 9G), trastuzumab derived ADCs using site specific conjugation (FIGS. 9A-9E), trastuzumab derived ADC using conventional conjugation (FIG. 9F) and negative control huNeg-8.8 ADC.

The data demonstrates that all the tested vc0101 conjugates cause tumor reduction in a dose-dependent manner. These ADCs can cause tumor regression at 1 mg/kg. However, T-DM1 is inactive in this moderate/low Her2 expressing model even at 6 mg/kg.

D. MDA-MB-361(DYT2) Breast Xenografts

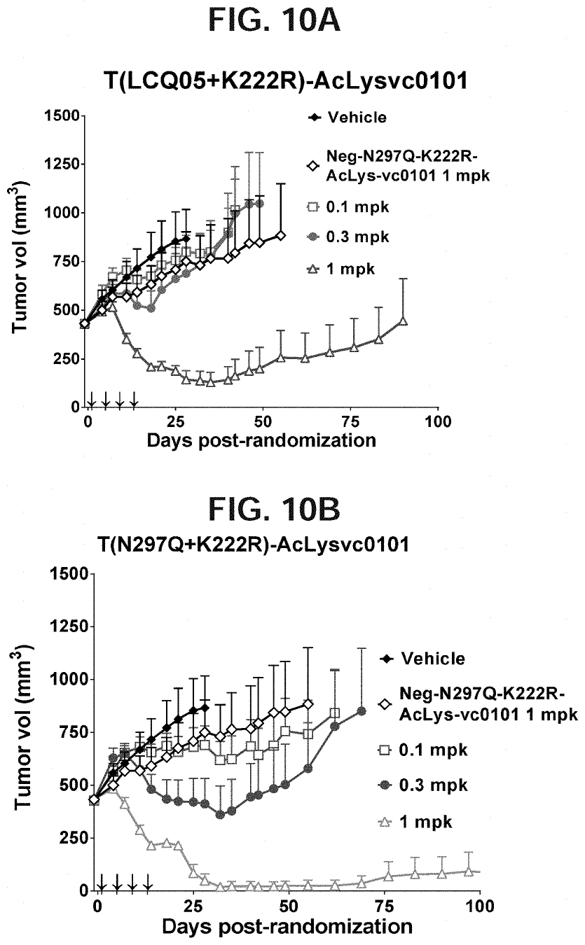

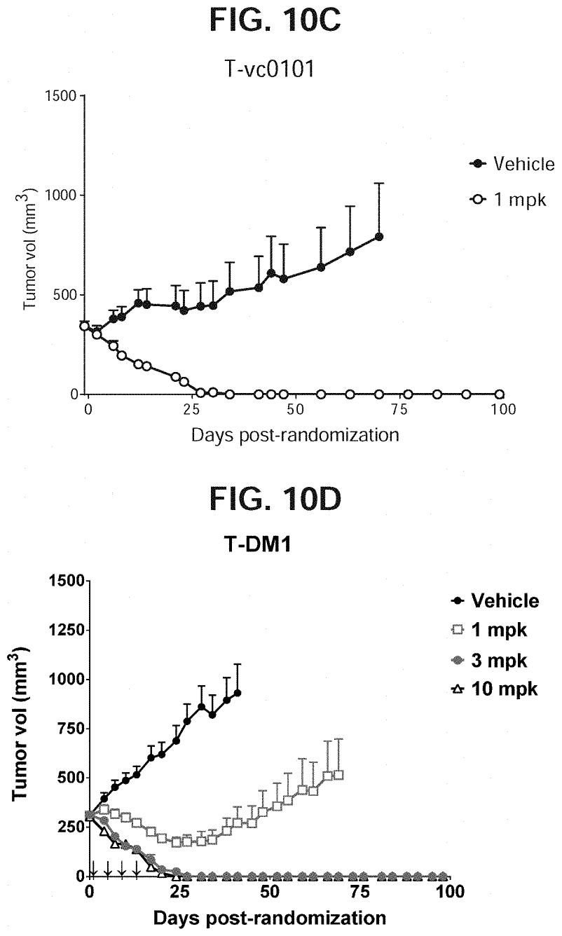

MDA-MB-361(DYT2) is a breast cancer cell line expressing moderate/low Her2. To generate xenografts, nude (Nu/Nu) female mice were irradiated at 100 cGy/min for 4 minutes and three days later implanted subcutaneously with 1.0×10⁷ MDA-MB-361(DYT2) cells (ATCC #HTB-27) in 50% Matrigel (BD Biosciences). When the tumors reached a volume of 300 to 400 mm³, the tumors were staged to ensure uniformity of the tumor mass among various treatment groups. The DYT2 breast model was dosed intravenously Q4d×4 with PBS vehicle, trastuzumab derived ADCs using site specific and conventional conjugation, T-DM1 and negative control ADC (FIGS. 10A-10D).

The data demonstrates that trastuzumab ADCs inhibited growth of DYT2 breast xenografts in a dose-dependent manner. Although DYT2 is moderate/low Her2 expression cell lines, it is more sensitive to micro-tubule inhibitors than other Her2 low/moderate expressing cell lines.

E. 144580 Patient-Derived Breast Cancer Xenografts

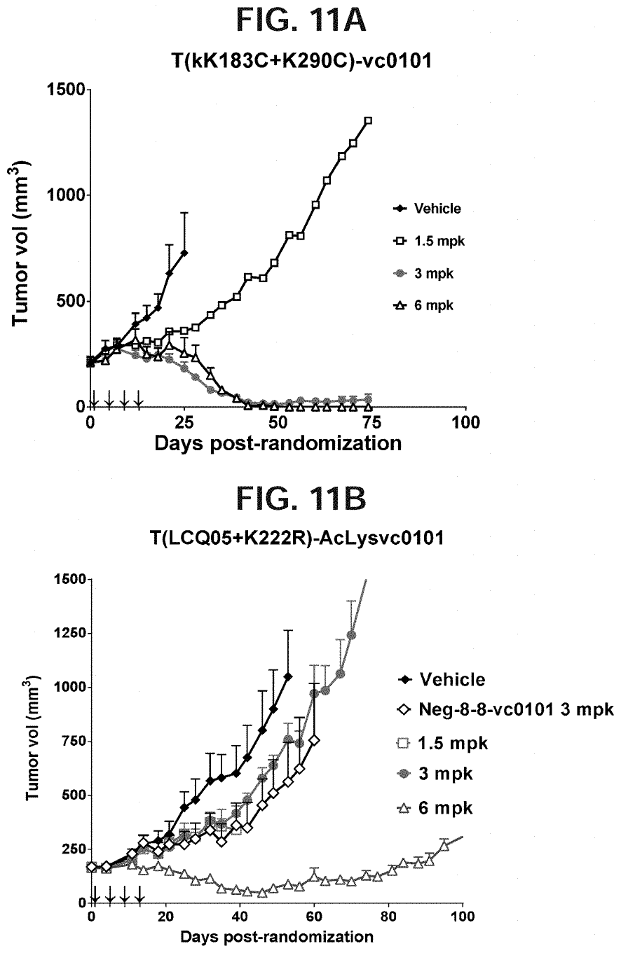

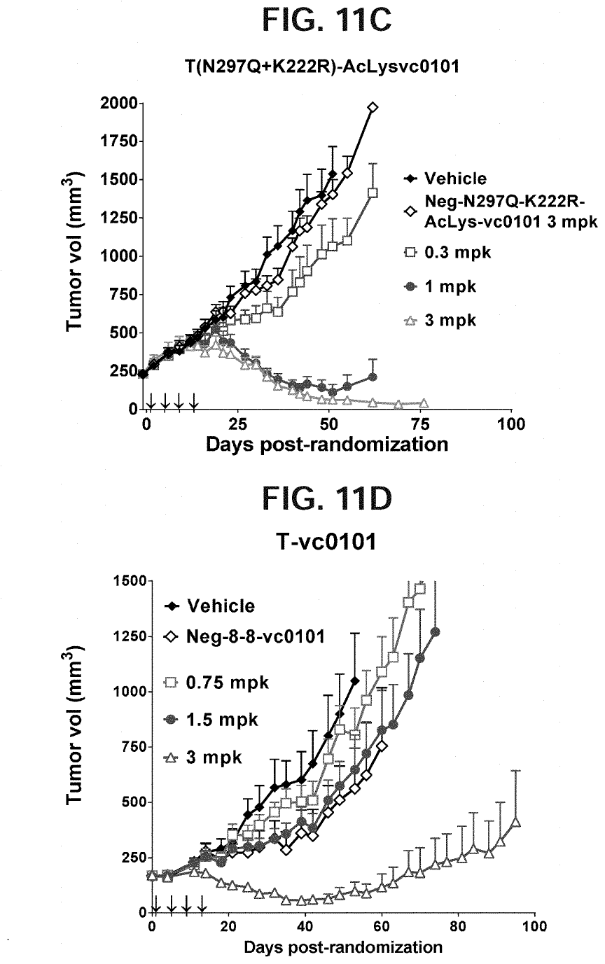

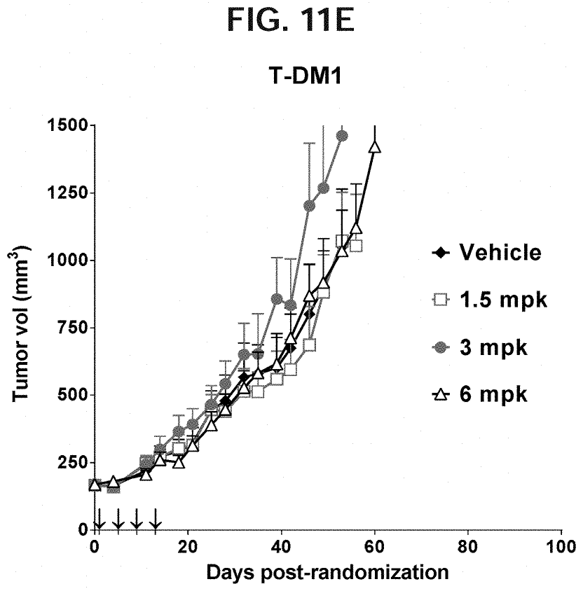

The effects of Trastuzumab derived ADCs were examined in immunodeficient mice on the in vivo growth of human tumor xenografts that were established from fragments of freshly resected 144580 breast tumors obtained in accordance with appropriate consent procedures. The tumor characterization of 144580 when fresh biopsy was taken was as a triple negative (ER−, PR—, and HER2−) breast cancer tumor. The 144580 breast patient-derived xenografts were subcutaneously passaged in vivo as fragments from animal to animal in nude (Nu/Nu) female mice. When the tumors reached a volume of 150 to 300 mm³, they were staged to ensure uniformity of the tumor size among various treatment groups. The 144580 breast model was dosed intravenously four times every four days (Q4d×4) with PBS vehicle, trastuzumab ADCs using site specific conjugation, trastuzumab derived ADC using conventional conjugation and negative control ADC (FIGS. 11A-11E).

In this HER2− (by clinical definition) PDX model, T-DM1 was ineffective at all doses tested (1,5, 3 and 6 mg/kg) (FIG. 10E). For DAR4 vc0101 ADCs (FIGS. 11A, 11C and 11D), 3 mg/kg is able to cause tumor regression (even at 1 mg/kg in FIG. 11C). The DAR2 vc0101 ADC (FIG. 11B) is less efficacious than DAR4 ADCs at 3 mg/kg. However, the DAR 2 vc0101 ADC is efficacious at 6 mg/kg unlike T-DM1.

F. 37622 Patient-Derived Non-Small Cell Lung Cancer Xenograft

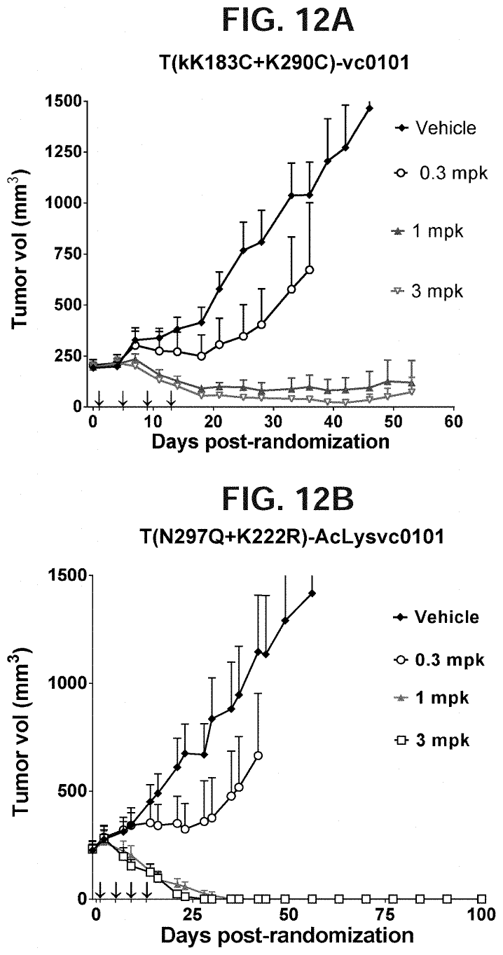

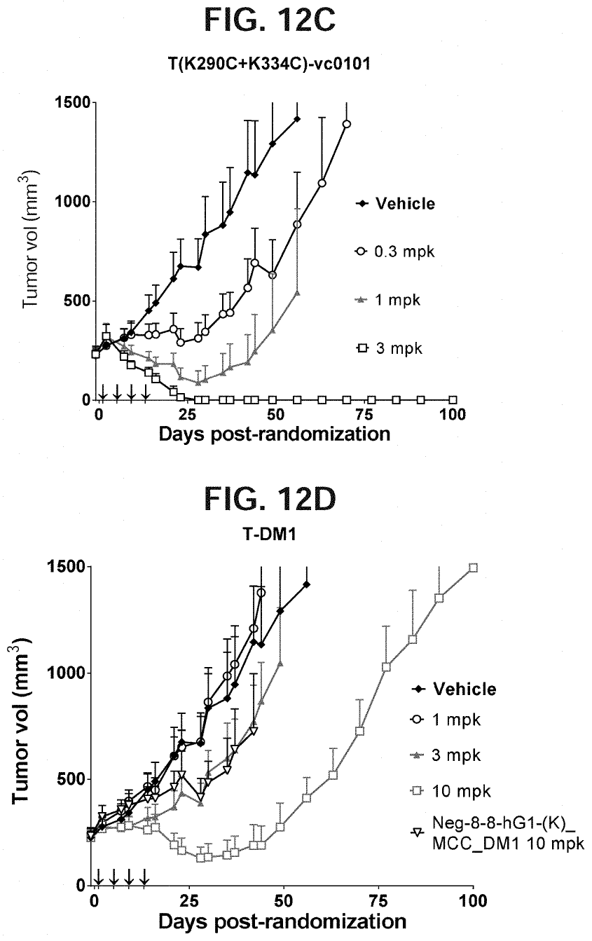

Several ADCs were tested in patient-derived Non-Small Cell Lung Cancer xenograft model of 37622 obtained in accordance with appropriate consent procedures. The 37622 patient-derived xenografts were subcutaneously passaged in vivo as fragments from animal to animal in nude (Nu/Nu) female mice. When the tumors reached a volume of 150 to 300 mm³, they were staged to ensure uniformity of the tumor size among various treatment groups. The 37622 PDX model was dosed intravenously four times every four days (Q4d×4) with PBS vehicle, trastuzumab derived ADCs using site specific conjugation, T-DM1 and negative control ADC (FIGS. 12A-12D).

Expression of Her2 was profiled by modified Hercept test and was classified as 2+ with more heterogeneity than seen in cell lines. The ADCs conjugated with vc0101 as a linker-payload (FIGS. 12A-12C) were efficacious at 1 and 3 mg/kg causing tumor regression. However, T-DM1 only provided some therapeutic benefit at 10 mg/kg (FIG. 12D). It appears vc0101 ADCs are 10-times more potent than T-DM1 by comparing results at 10 mg/kg from T-DM1 to 1 mg/kg from vc0101 ADCs. It is possible that the bystander effect is important for efficacy for a heterogeneic tumor.

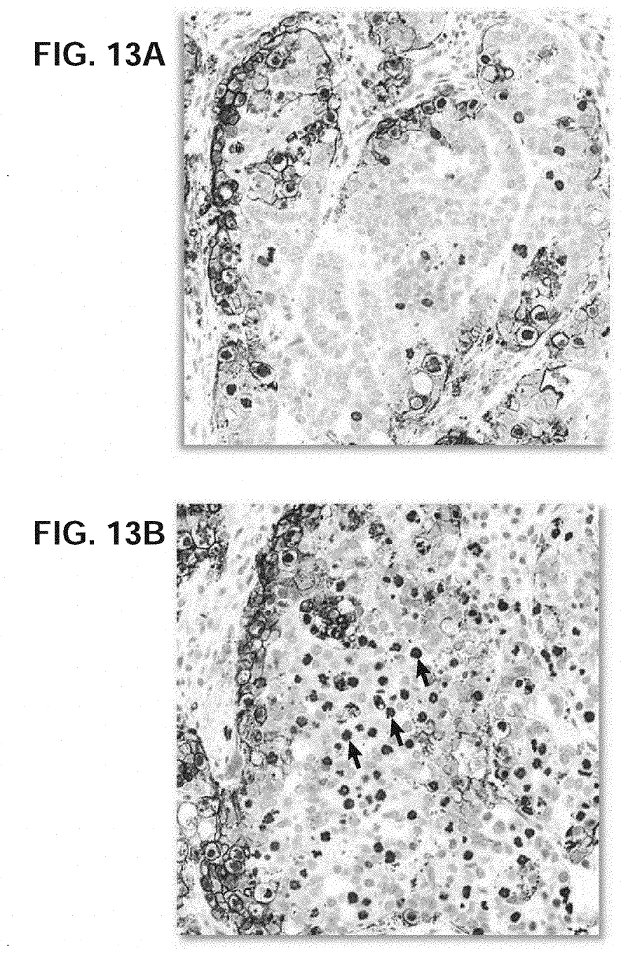

The released metabolite of the T-DM1 ADC has been shown to be the lysine-capped mcc-DM1 linker payload (i.e., Lys-mcc-DM1) which is a membrane impermeable compound (Kovtun et al., 2006, Cancer Res 66:3214-21; Xie et al., 2004, J Pharmacol Exp Ther 310:844). However, the released metabolite from the T-vc0101 ADC is auristatin 0101, a compound with more membrane permeability than Lys-mcc-DM1. The ability of a released ADC payload to kill neighboring cells is known as the bystander effect. Due to a release of a membrane permeable payload, T-vc0101 is able to elicit a strong bystander effect whereas T-DM1 is not. FIG. 13 shows immunohistocytochemistry from N87 cell line xenograft tumors which received a single dose of either T-DM1 at 6 mg/kg (FIG. XA) or T-vc0101 at 3 mg/kg (FIG. XB) and then harvested and processed in formalin fixation 96 hours later. Tumor sections were stained for human IgG to detect ADC bound to tumor cells and phosphhistone H3 (pHH3) to detect mitotic cells as readout of the proposed mechanism of action for the payloads of both ADCs.

ADC is detected in the periphery of the tumors in both cases. In T-DM1 treated tumors (FIG. 13A), the majority of pHH3 positive tumor cells are located near the ADC. However, in T-vc0101 treated tumors (FIG. 13B), the majority of pHH3 positive tumor cells extend beyond the location of the ADC (black arrows highlight a few examples) and are in the tumor interior. This suggests that an ADC with a cleavable linker and a membrane permeable payload can elicit a strong bystander effect in vivo.

### Example 14: In vitro T-DM1 Resistance Models

A. Generation of T-DM1 Resistant Cells in vitro

N87 cells were passaged into two separate flasks and each flask was treated identically with respect to the resistance-generation protocol to enable biological duplicates. Cells were exposed to five cycles of T-DM1 conjugate at approximately IC₈₀ concentrations (10 nM payload concentration) for 3 days, followed by approximately 4 to 11 days recovery without treatment. After the five cycles at 10 nM of the T-DM1 conjugate, the cells were exposed to six additional cycles of 100 nM T-DM1 in a similar fashion. The procedure was intended to simulate the chronic, multi-cycle (on/off) dosing at maximally tolerated doses typically used for cytotoxic therapeutics in the clinic, followed by a recovery period. Parental cells derived from N87 are referred to as N87, and cells chronically exposed to T-DM1 are referred to as N87-TM. Moderate- to high-level drug resistance developed within 4 months for N87-TM cells. Drug selection pressure was removed after ˜3-4 months of cycle treatments when the level of resistance no longer increased after continued drug exposure. Responses and phenotypes remained stable in the cultured cell lines for approximately 3-6 months thereafter. Thereafter, a reduction in the magnitude of the resistance phenotype as measured by cytotoxicity assays was occasionally observed, in which case early passage cryo-preserved T-DM1 resistant cells were thawed for additional studies. All reported characterizations were conducted after removal of T-DM1 selection pressure for at least 2-8 weeks to ensure stabilization of the cells. Data were collected from various thawed cryopreserved populations derived from a single selection, over approximately 1-2 years after model development to ensure consistency in the results.

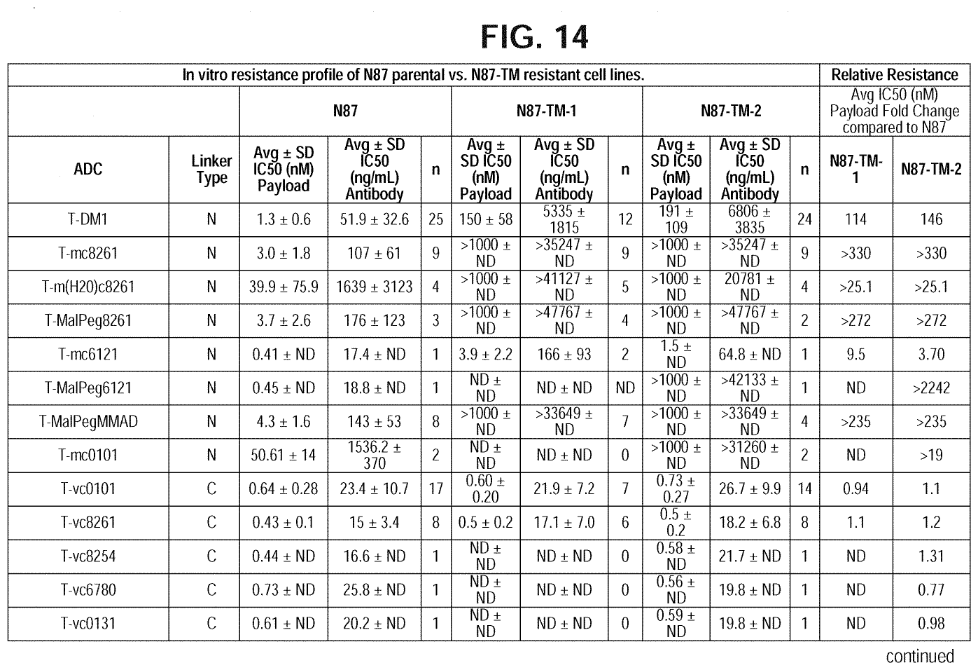

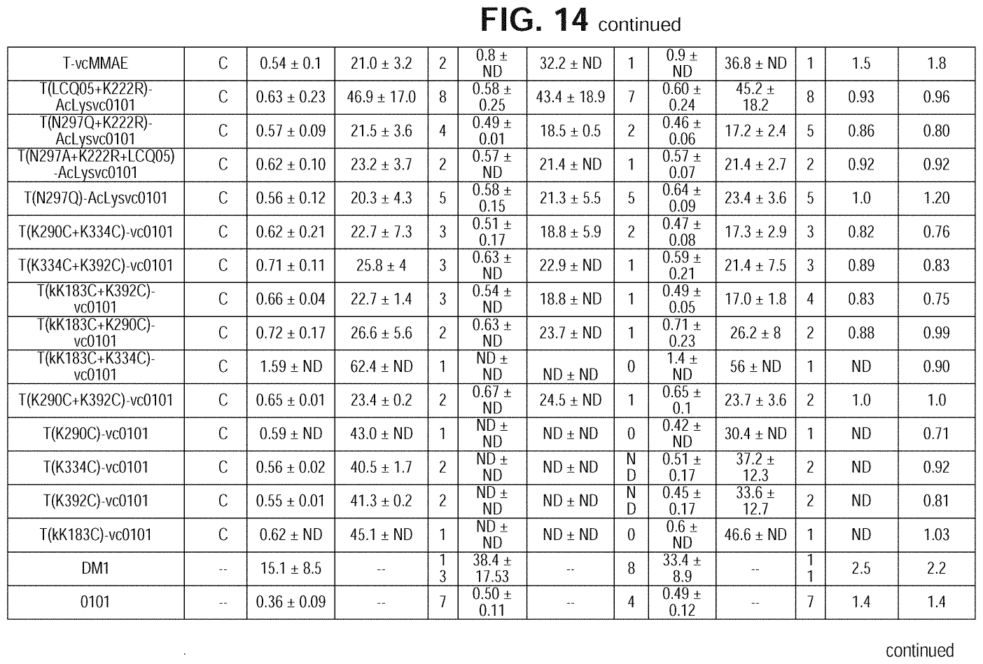

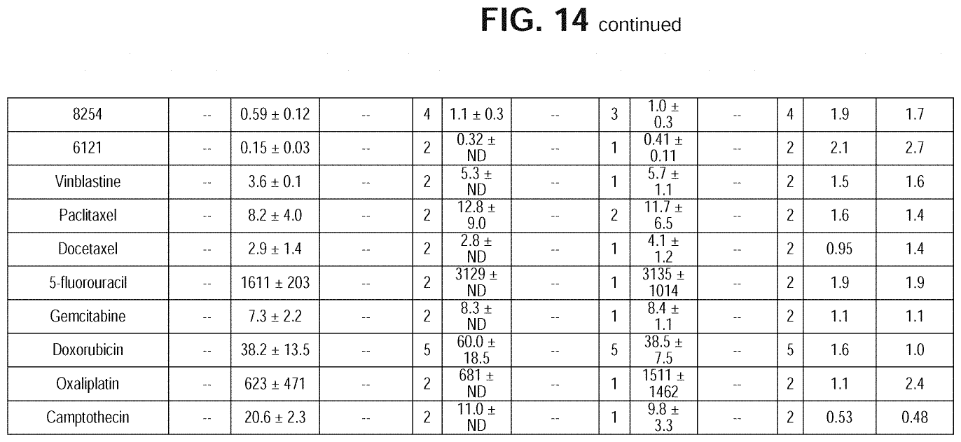

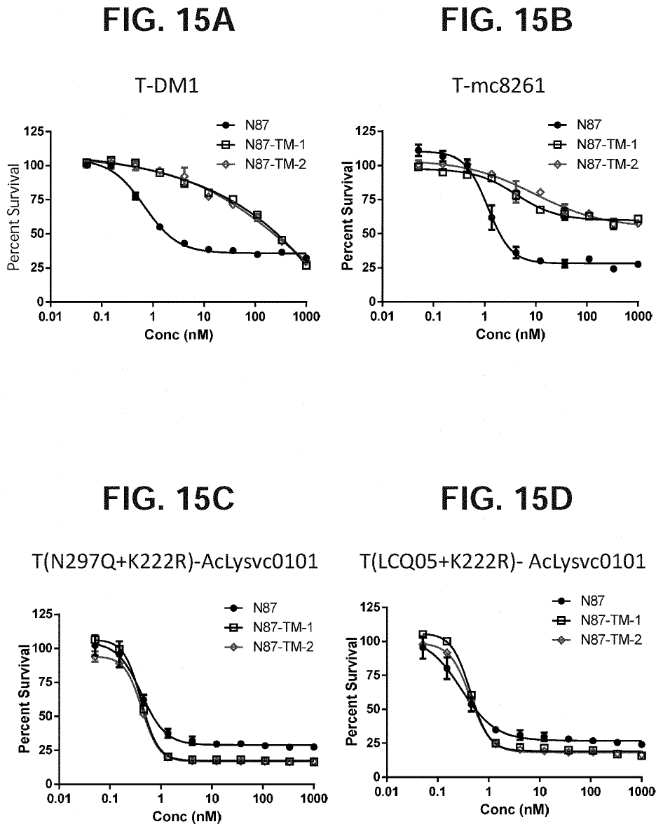

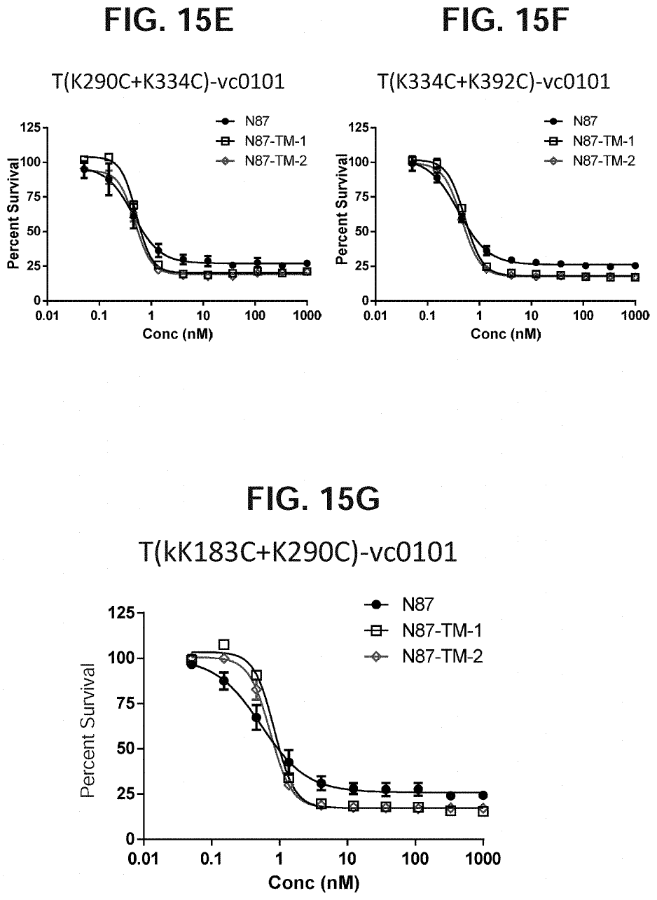

The gastric cancer cell line N87 was selected for resistance to trastuzumab-maytansinoid antibody-drug conjugate (T-DM1) by treatment cycles at doses that were approximately the IC₈₀ (˜10 nM payload concentration) for the respective cell line. Parental N87 cells were inherently sensitive to the conjugate (IC₅₀=1.7 nM payload concentration; 62 ng/ml antibody concentration) (FIG. 14). Two populations of parental N87 cells were exposed to the treatment cycles and, after only approximately four months exposure cycling at 100 nM T-DM1, these two populations (henceforth named N87-TM-1 and N87-TM-2) became refractory to the ADC by 114- and 146-fold, respectively, compared with parental cells (FIG. 14 and FIG. 15A). Interestingly, minimal cross-resistance (˜2.2-2.5×) to the corresponding unconjugated maytansinoid free drug, DM1, was observed (FIG. 14).

B. Cytotoxicity Studies

ADCs were prepared as indicated in Example 3. Unconjugated maytansine analog (DM1) and auristatin analogs were prepared by Pfizer Worldwide Medicinal Chemistry (Groton, Conn.). Other standard-of-care chemotherapeutics were purchased from Sigma (St. Louis, Mo.). Cells were seeded in 96-well plates at low density, then treated the following day with ADCs and unconjugated payloads at 3-fold serial dilutions at 10 concentrations in duplicate. Cells were incubated for 4 days in a humidified 37° C./5% CO₂ incubator. The plates were harvested by incubating with CellTiter® 96 AQueous One MTS Solution (Promega, Madison, Wis.) for 1.5 hours and absorbance measured on a Victor plate reader (Perkin-Elmer, Waltham, Mass.) at wavelength 490 nm. IC₅₀ values were calculated using a four-parameter logistic model with XLfit (IDBS, Bridgewater, N.J.).

The cross-resistance profile to other trastuzumab derived ADCs was determined. Significant cross-resistance to many trastuzumab derived ADCs composed of non-cleavable linkers and delivering payloads with anti-tubulin mechanisms of action was observed (FIG. 14). For example, in N87-TM vs. N87-parental cells, >330- and >272-fold reduced potency was observed to T-mc8261 (FIG. 14 and FIG. 15B) and T-MalPeg8261 (FIG. 14), which represent an auristatin-based payload linked to trastuzumab via non-cleavable maleimidocaproyl or Mal-PEG linkers, respectively. Over 235-fold resistance was observed in N87-TM cells against T-mcMalPegMMAD, another trastuzumab ADC with a different non-cleavable linker delivering monomethyl dolastatin (MMAD) (FIG. 14).

Remarkably, it was observed that the N87-TM cell line retained sensitivity to payloads when delivered via a cleavable linker, even though these drugs functionally inhibit similar targets (i.e., microtubule depolymerization). Examples of ADCs which overcome resistance include, but are not limited to, T(N297Q+K222R)-AcLysvc0101 (FIG. 14 and FIG. 15C), T(LCQ05+K222R)-AcLysvc0101 (FIG. 14 and FIG. 15D), T(K290C+K334C)-vc0101 (FIG. 10 and FIG. 11E), T(K334C+K392C)-vc0101 (FIG. 14 and FIG. 15F) and T(kK183C+K290C)-vc0101 (FIG. 14 and FIG. 15G). These represent trastuzumab-based ADCs delivering the auristatin analog 0101, but where the payloads are released intracellularly by proteolytic cleavage of the vc linker.

In order to determine whether these ADC-resistant cancer cells were broadly resistant to other therapies, the N87-TM cell models were treated with a panel of standard-of-care chemotherapeutics with various mechanisms of action. In general, small molecule inhibitors of microtubule and DNA function remained effective against the N87-TM resistant cell lines (FIG. 14). While these cells were made resistant against an ADC delivering an analog of the microtubule depolymerizing agent, maytansine, minimal or no cross-resistance was observed to several tubulin depolymerizing or polymerizing agents. Similarly, both cell lines retained sensitivity to agents which interfere with DNA function, including topoisomerase inhibitors, anti-metabolites, and alklyating/cross-linking agents. In general, the N87-TM cells were not refractory to a broad range of cytotoxics, ruling out generic growth or cell cycle defects which might mimic drug resistance.

Both N87-TM populations also retained sensitivity to the corresponding unconjugated drugs (i.e., DM1 and 0101; FIG. 14). Hence, N87-TM cells made refractory to a trastuzumab-maytansinoid conjugate displayed cross-resistance to other microtubule-based ADCs when delivered via non-cleavable linkers, but remained sensitive to unconjugated microtubule inhibitors and other chemotherapeutics.

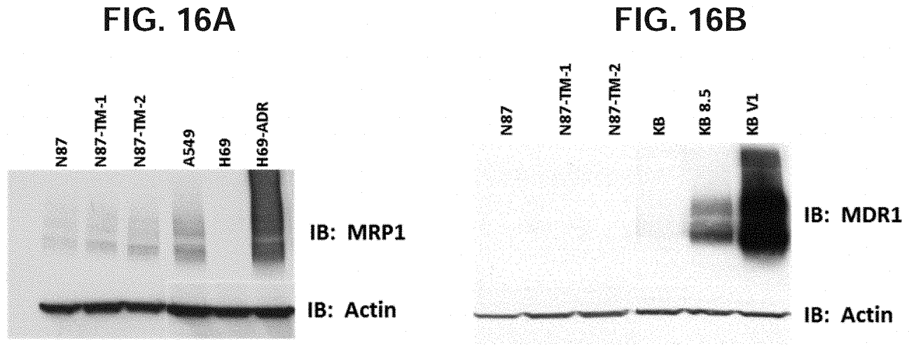

To determine the molecular mechanism of resistance to T-DM1 in the N87-TM cells protein expression levels of MDR1 and MRP1 drug efflux pumps were determined. This was because small molecule tubulin inhibitors are known substrates of the MDR1 and MRP1 drug efflux pumps (Thomas and Coley, 2003, Cancer Control 10(2):159-165). The protein expression levels of these two proteins from total cell lysates of the parental N87 and N87-TM resistant cells was determined (FIG. 16). Immunoblot analysis showed that the N87-TM resistant cells do not significantly overexpress the MRP1 (FIG. 16A) or MDR1 (FIG. 16B) proteins. Taken together, these data combined with the lack of cross-resistance to known substrates of drug efflux pumps (e. g. paclitaxel, doxorubicin) in the N87-TM cells suggests that drug efflux pump overexpression is not the molecular mechanism of T-DM1 resistance in N87-TM cells.

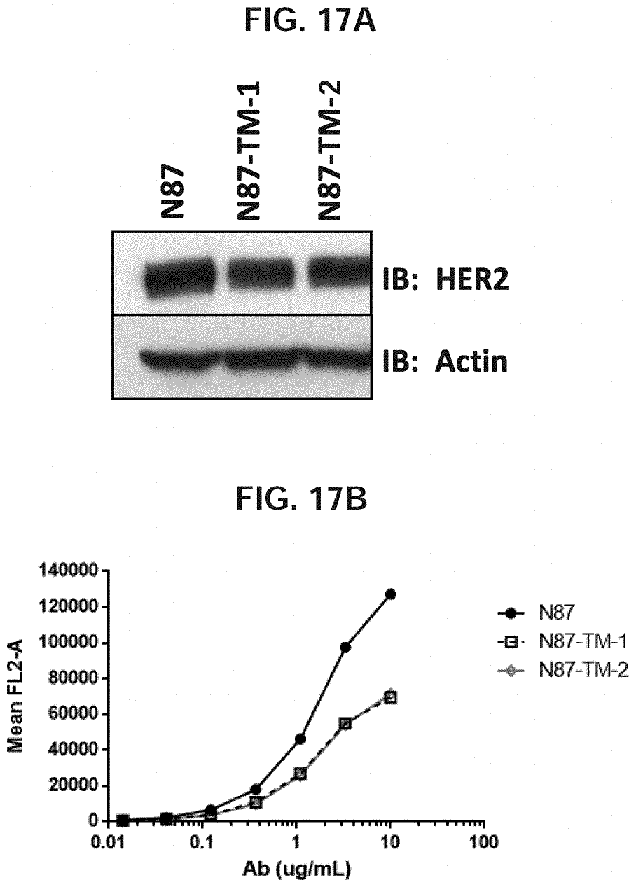

Since the mechanism of action for ADCs requires binding to a specific antigen, antigen depletion or reduced antibody binding may account for T-DM1 resistance in N87-TM cells. To determine if the antigen for T-DM1 had been significantly depleted in N87-TM cells, HER2 protein expression levels from total cell lysates of the parental N87 and N87-TM resistant cells were compared (FIG. 17A). Immunoblot analysis showed that the N87-TM cells did not have a markedly reduced amount of HER2 protein expression compared with the parental N87 cells.

The amount of antibody binding to cell surface HER2 antigens of the N87-TM cells was determined. In a cell surface binding study using fluorescence activated cell sorting, the N87-TM cells did have ˜50% decrease in trastuzumab binding to cell surface antigens (FIG. 17B). Since N87 cells are high expressers of HER2 protein among cancer cell lines (Fujimoto-Ouchi et al., 2007, Cancer Chemother Pharmacol 59(6):795-805), a ˜50% reduction in HER2 antibody binding in these cells probably does not represent the driving mechanism of resistance to T-DM1 in N87-TM cells. Evidence supporting this interpretation is that the N87-TM resistant cells remain sensitive to other HER2 binding trastuzumab derived ADCs with different linkers and payloads (FIG. 14).

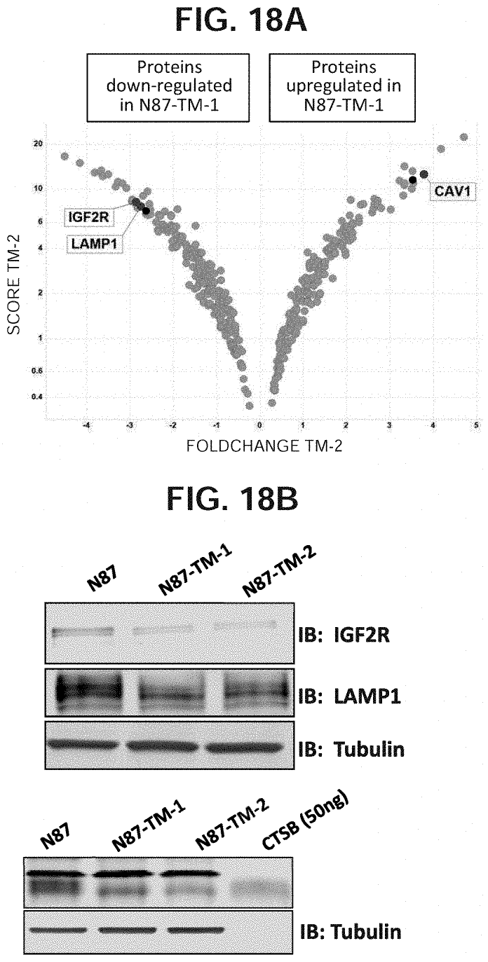

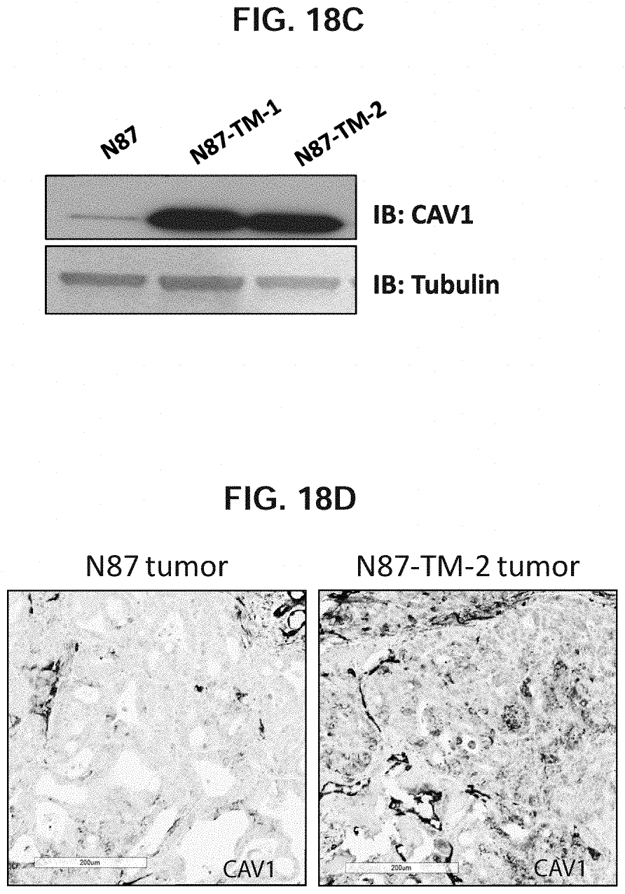

In order to determine potential mechanisms of T-DM1 resistance in an unbiased approach, the parental N87 and N87-TM resistant cell models were profiled via a proteomic approach in order to globally identify changes in membrane protein expression levels that may be responsible for T-DM1 resistance. Significant expression level changes in 523 proteins between both cell line models was observed (FIG. 18A). To validate a selection of these predicted protein changes, immunoblots on N87 and N87-TM whole cell lysates were performed for proteins predicted to be under-expressed (IGF2R, LAMP1, CTSB) (FIG. 18B) and over-expressed (CAV1) (FIG. 18C) in the N87-TM cells relative to the N87 cells. In vivo tumors were generated by subcutaneous implantation of the N87 and N87-TM-2 cells into NSG mice to assess if protein changes observed in vivo mimic those seen in vitro. N87-TM-2 tumors retained over-expression of the CAV1 protein compared with the N87 tumors (FIG. 18D). While CAV1 staining in the mouse stroma in both models is expected, epithelial CAV1 staining was only seen in the N87-TM-2 model.

C. In vivo Efficacy Studies

In order to determine if the resistance observed in cell culture was recapitulated in vivo, parental N87 cells and N87-TM-2 cells were expanded and injected into the flanks of Female NOD scid gamma (NSG) immunodeficient mice (NOD. Cg-Prkdcscid II2rgtm1WjI/SzJ) obtained from The Jackson Laboratory (Bar Harbor, Me.). Mice were injected subcutaneously in the right flank with suspensions of either N87 or N87-TM cells (7.5×10⁶ cells per injection, with 50% Matrigel). Mice were randomized into study groups when tumors reached ˜0.3 g (˜250 mm³). T-DM1 conjugate or vehicle, were administered intravenously in saline on day 0 and repeated for a total of four doses, four days apart (Q4D×4). Tumors were measured weekly and mass calculated as volume=(width×width×length)/2. Time-to-event analysis (tumor doubling) was conducted and significance evaluated by Log-rank (Mantel-Cox) test. No weight loss was observed in mice in all treatment groups in these studies.

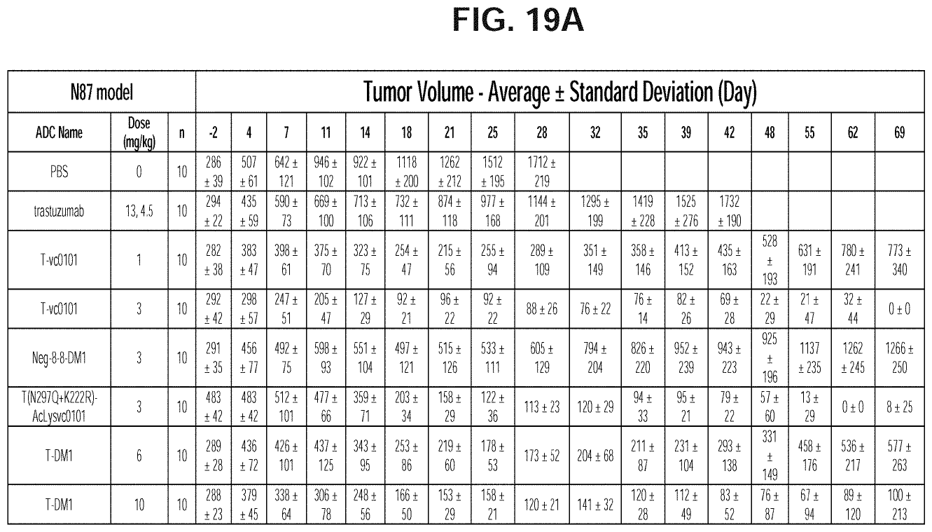

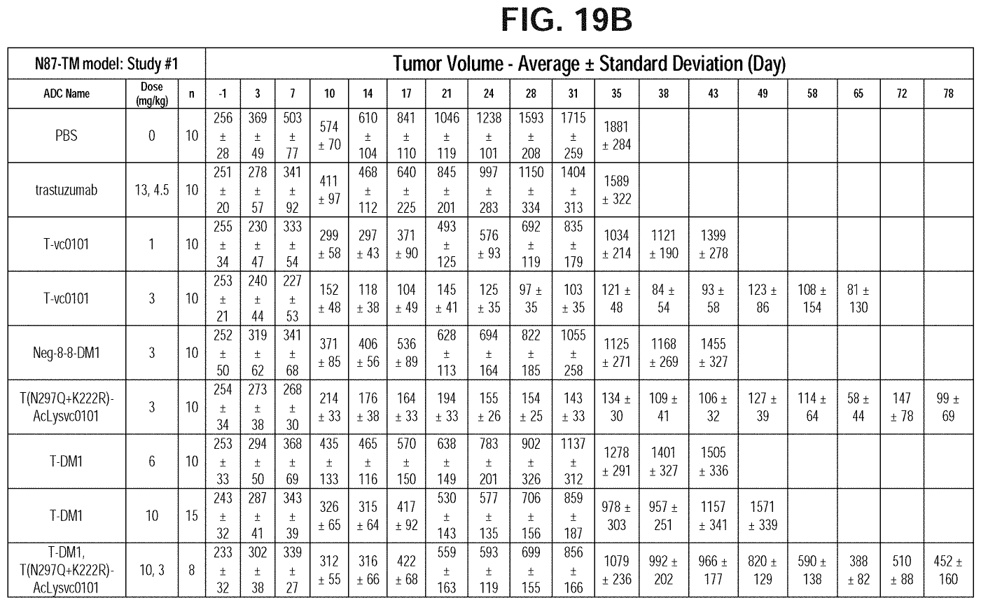

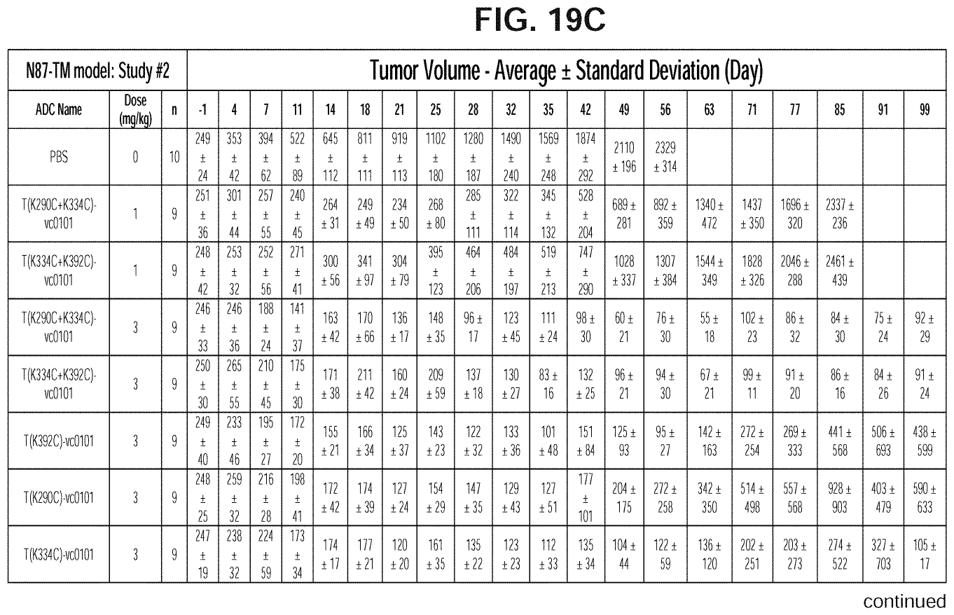

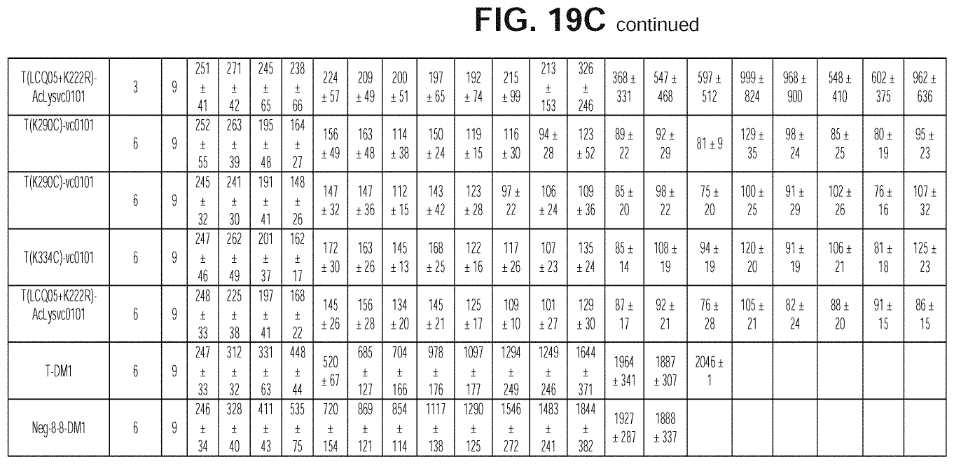

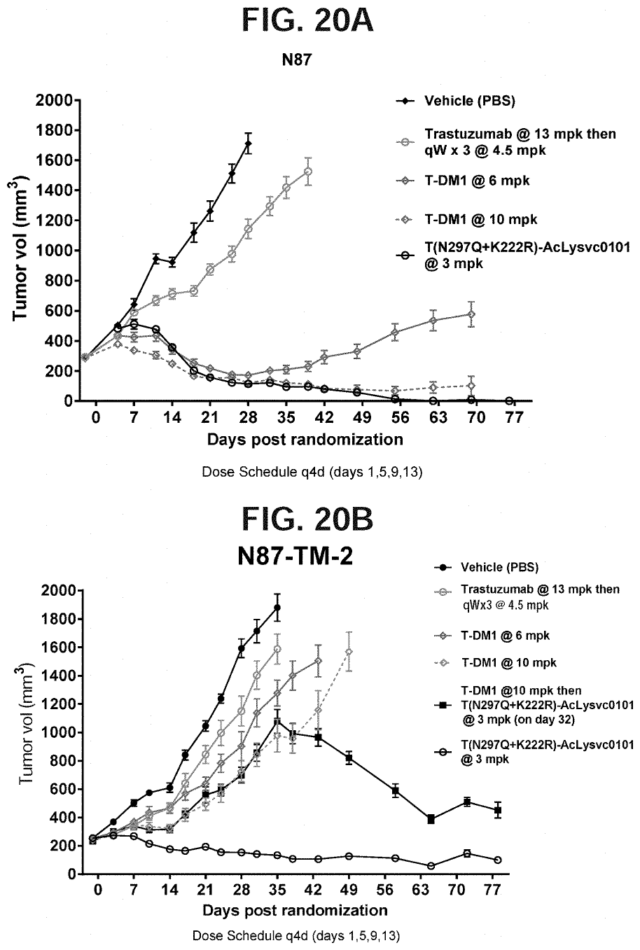

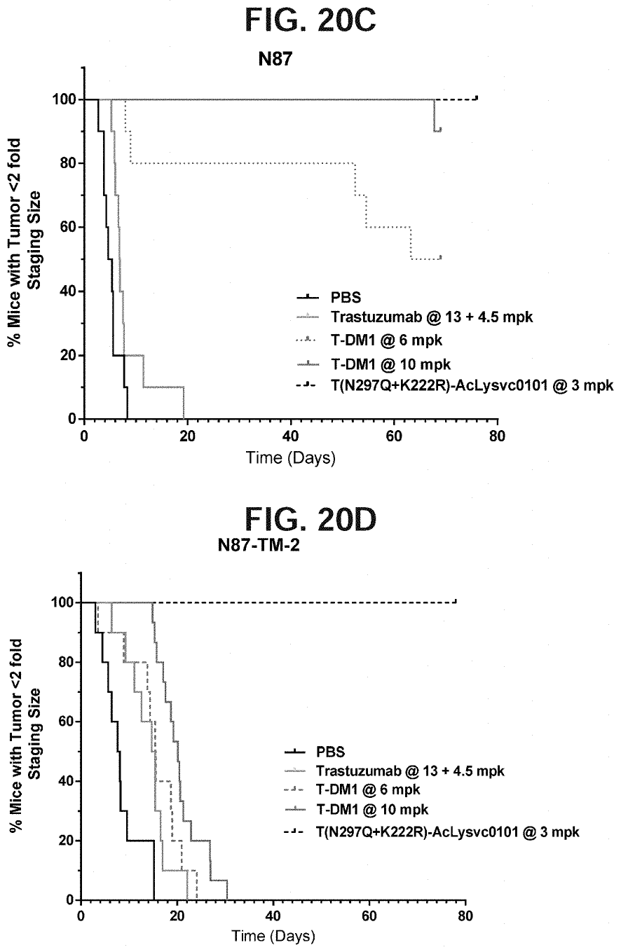

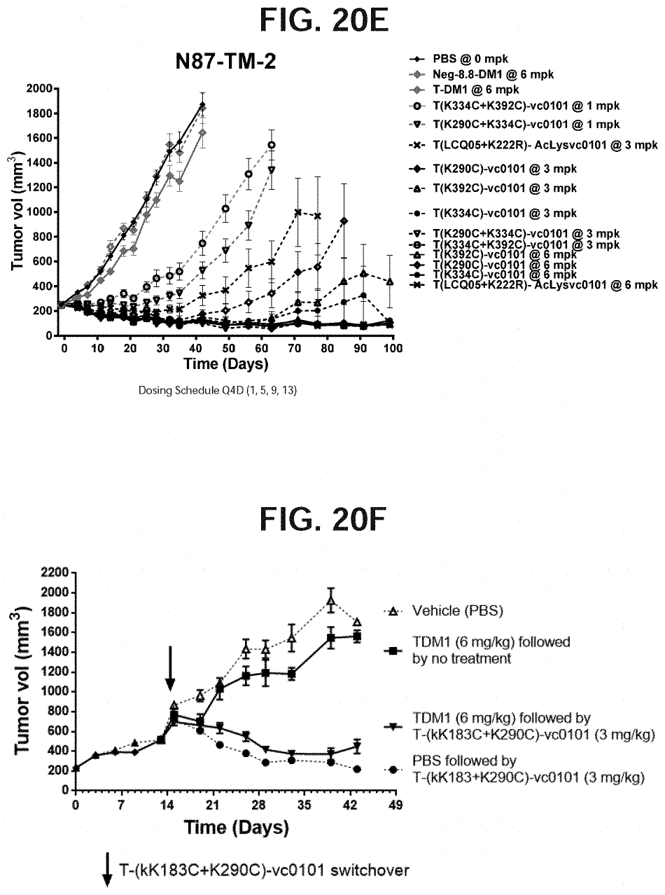

Mice were treated with the following agents: (1) vehicle control PBS, (2) trastuzumab antibody at 13 mg/kg, followed by 4. 5 mg/kg; (3) T-DM1 at 6 mg/kg; (4) T-DM1 at 10 mg/kg; (5) T-DM1 at 10 mg/kg, then T(N297Q+K222R)-AcLysvc0101 at 3 mg/kg; (6) T(N297Q+K222R)-AcLysvc0101 at 3 mg/kg. Tumor sizes were monitored and results are indicated in FIG. 20. The N87 (FIG. 19 and FIG. 20A) and N87-TM-2 (FIG. 19 and FIG. 20B) tumors showed an ADC efficacy profile similar to that seen in the in vitro cytotoxicity assays (FIGS. 19 and 20B), wherein the N87-TM drug resistant cells were refractory to T-DM1 but still responded to trastuzumab derived ADCs with cleavable linkers. In fact, tumors that were refractory to T-DM1 and grew to about 1 gram were switched to therapy with T(N297Q+K222R)-AcLysvc0101 and effectively regressed (FIG. 20B). In a time-to-event analysis of this study, T-DM1 at 6 and 10 mg/kg prevented tumor doubling in >50% of mice for at least 60 days in the N87 model, but T-DM1 failed to do so in the N87-TM-2 model (FIGS. 20C and 20D). T(N297Q+K222R)-AcLysvc0101 dosed at 3 mg/kg prevented any tumor doubling of both N87 and N87-TM tumors in the mice for the duration of the study (˜80 days) (FIGS. 20C and 20D).

In another study, all cleavable linked ADCs that overcame T-DM1 resistance in vitro remained effective in this N87-TM2 tumor model that was non-responsive to T-DM1 (FIG. 19 and FIG. 20E).

It was then assessed whether T(kK183+K290C)-vc0101 ADC could inhibit the growth of tumors which were refractory to TDM1. N87-TM tumors treated with either vehicle or T-DM1 grew through these treatments, however tumors switched to T(kK183C+K290C)-vc0101 therapy at day 14 immediately regressed (FIG. 20F).

### Example 15: In vivo T-DM1 Resistant Models

A. Generation of T-DM1 Resistant Cells In Vivo

All animal studies were approved by the Pfizer Pearl River Institutional Animal Care and Use Committee according to established guidelines. To generate xenografts, nude (Nu/Nu) female mice were implanted subcutaneously with 7.5×10⁶ N87 cells in 50% Matrigel (BD Biosciences). The animals were randomized when average tumor volume reach ˜300 mm³ into two groups: 1) vehicle control (n=10) and 2) T-DM1 treated (n=20). T-DM1 ADC (6.5 mg/kg) or vehicle (PBS) were administered intravenously in saline on day 0 and then the animals were dosed weekly with 6.5 mg/kg for up to 30 weeks. Tumors were measured twice per week or weekly and mass calculated as volume=(width×width×length)/2. No weight loss was observed in mice in all treatment groups in these studies.

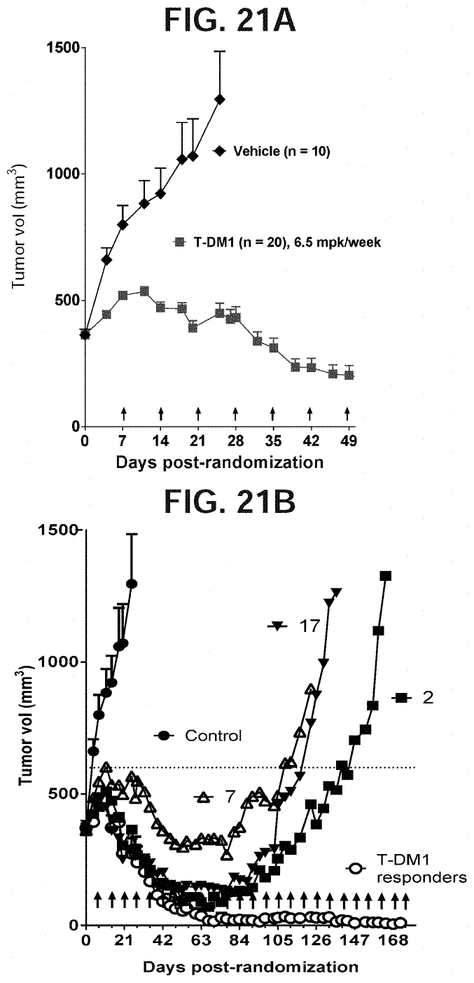

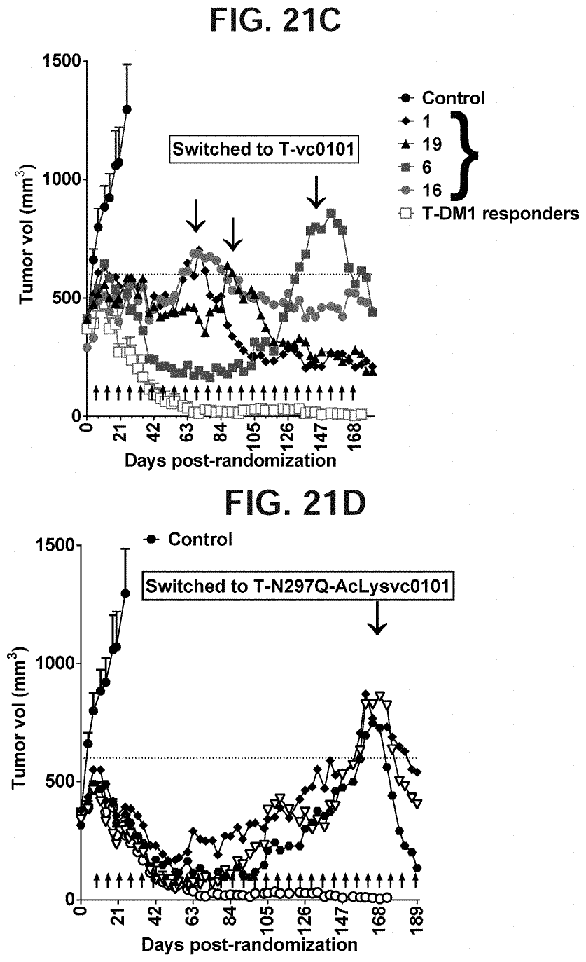

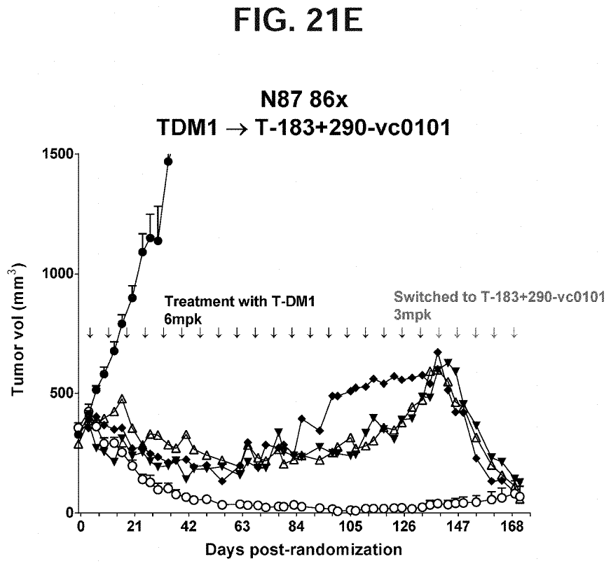

Animals were considered refractory or relapsed under T-DM1 treatment when the individual tumor volume reached ˜600 mm³ (doubled original size of tumor at randomization). Compared to control group, most tumors initially responded to T-DM1 treatment as shown in FIG. 21A. More specifically, 17 out of 20 mice responded to initial T-DM1 treatment but significant number of tumors (13 out of 20) relapsed under T-DM1 treatment. Over time the implanted N87 tumor cells became resistant to T-DM1 (FIG. 21B). Three tumors that did not initially responded to T-DM1 treatment were harvested for Her2 expression determination by IHC indicating no HER2 expression changes. The remaining 10 relapsed tumors are described below.

Four tumors which initially responded to T-DM1 treatment and then relapsed were switched to T-vc0101 treatment weekly at 2.6 mg/kg on day 77 (mice 1 and 16), 91 (mouse 19), 140 (mouse 6). As shown in FIG. 19C, T-DM1 resistant tumors generated in vivo responded to T-vc0101 indicating acquired T-DM1 resistant tumors are sensitive to vc0101 ADC treatment.

Another three tumors initially responded to T-DM1 treatment and then relapsed were switched to T(N297Q+K222R)-AcLysvc0101 treatment weekly at 2.6 mg/kg on day 110 (mice 4, 13, and 18). As shown in FIG. 21D, T-DM1 resistant tumors generated in vivo also responded to T(N297Q+K222R)-AcLysvc0101. A follow-on experiment was performed to evaluate T(kK183C+K290C)-vc0101, similar results were obtained indicating that T-DM1 resistant tumors generated in vivo were sensitive to T(kK183C+K290C)-vc0101 treatment as shown in FIG. 21E.

In summary, all T-DM1 refractory tumors having follow-on treatment were sensitive to the vc0101 ADC treatment (7 of 7) indicating that in vivo resistant T-DM1 tumors can be treated with cleavable vc0101 conjugates.

Additional three tumors (mouse 7, 17 and 2 as shown in FIG. 21B) initially responded to T-DM1 and then relapsed were excised for in vitro characterization. After 2-5 months of culturing the excised tumors in vitro these cells were evaluated for resistance to T-DM1 and characterized in vitro (see Sections B and C of this Example below).

B. Cytotoxicity Studies

Cells relapsed from T-DM1 treatment and cultured in vitro (as described in Section A of this Example) were seeded in 96-well plates and dosed the following day with 4-fold serial dilutions of the ADCs or unconjugated payloads. Cells were incubated for 96 hours in a humidified 37° C./5% CO₂ incubator. CellTiter Glo Solution (Promega, Madison, Wis.) was added to the plates and absorbance measured on a Victor plate reader (Perkin-Elmer, Waltham, Mass.) at wavelength 490 nm. IC₅₀ values were calculated using a four-parameter logistic model with XLfit (IDBS, Bridgewater, N.J.).

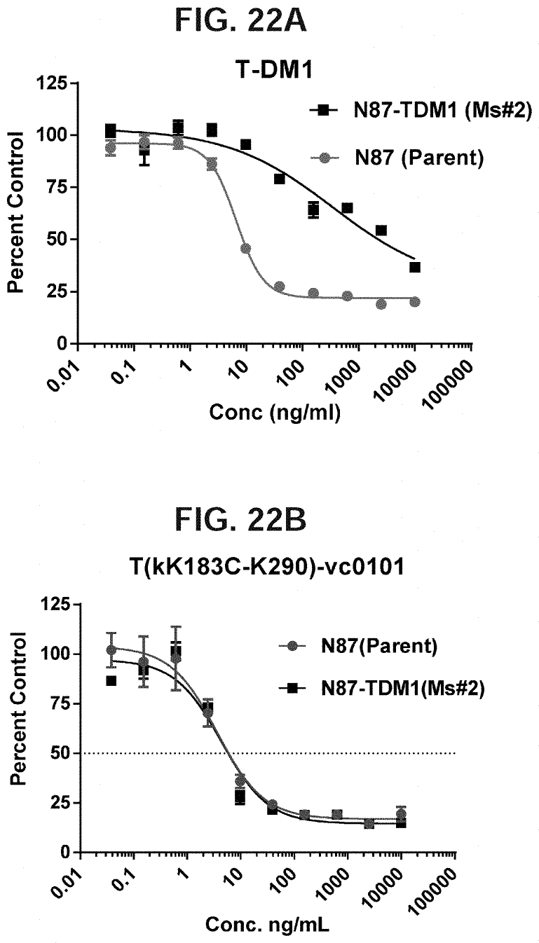

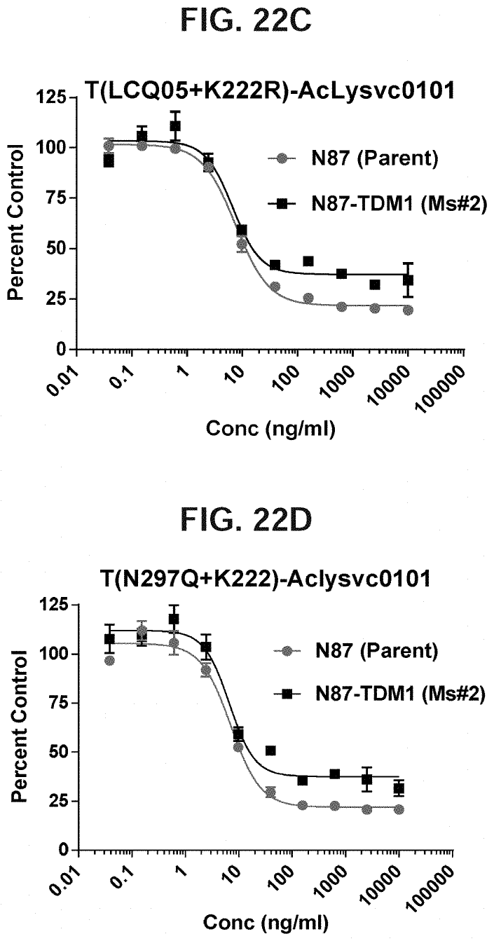

Cytotoxicity screening results are summarized in Tables 19 and 20. The cells were resistant to T-DM1 (FIG. 22A) when compared to the parental but sensitive to cleavable vc0101 conjugates T-vc0101 (data not shown), T(kK183C+K290C)-vc0101 (FIG. 22B), T(LCQ05+K222R)-AcLysvc0101 (FIG. 22C) and T(N297Q+K222R)-AcLysvc0101 (FIG. 22D) (Table 19). The T-DM1 resistant cells were surprisingly sensitive to the parent payload DM1 as well as the 0101 payload (Table 20).

TABLE 19
Resistant Cell Sensitivity to ADCs

| | | N87-T- | N87-T- | N87-T- | |
|---|---|---|---|---|---|
| | | DM1 | DM1 | DM1 | Fold |
| | N87 | Mouse | Mouse | Mouse | Resis- |
| ADC | parental | #7 | #17 | #2 | tance |
| T-DM1 | 16 | 1388 | 944 | 3700 | ~60-230 |
| T(kK183C + K290C)- | 5 | — | — | 5 | 1 |
| vc0101 | | | | | |
| T(LCQ05 + K222R)- | 25 | 9 | 10 | 18 | ~1 |
| AcLysvc0101 | | | | | |
| T(N297Q + K222R)- | 9 | 7 | 13 | 16 | ~1 |
| AcLysvc0101 | | | | | |
| T(K334C + K392C)- | 6 | — | 11 | 4 | ~1 |
| vc0101 | | | | | |
| T(K290C + K334C)- | 6 | — | 16 | 4 | ~1-2 |
| vc0101 | | | | | |

IC₅₀ values are shown for each of the cell lines

TABLE 20
Resistant Cell Line Sensitivity to Free Payload

| Cell Line | DM1-Sme | Aur-0101 | Doxorubicin |
|---|---|---|---|
| N87 | 10 | 0.5 | 48 |
| N87-T-DM1_Ms2 | 23 | 0.40 | 46 |
| N87-T-DM1_Ms7 | 20 | 0.60 | 79 |
| N87-T-DM1_Ms17 | 27 | 0.28 | 34 |

C. Her2 Expression by FACS and Western Blot

Her2 expression was characterized on cells relapsed from T-DM1 treatment and cultured in vitro (as described in Section A of this Example). For FACS analysis, cells were trypsinized, spun down and resuspended in fresh media. The cells were then incubated for one hour at 4° C. with 5 μg/mL of Trastuzumab-PE (custom synthesized 1:1 PE labeled Trastuzumab by eBiosciences (San Diego, Calif.)). The cells were then washed twice and then resuspended in PBS. The mean fluorescence intensity was read using Accuri flow cytometer (BD Biosciences San Jose, Calif.).

For western blot analysis, the cells were lysed using RIPA lysis buffer (with protease inhibitors and phosphatase inhibitor) on ice for 15 minutes then vortexed and spun down at maximum speed in a microcentrifuge at 4° C. The supernatant was collected and 4× sample buffer and reducing agent were added to the samples normalizing for total protein in each sample. The samples were run on a 4-12% Bis tris gel and transferred on to nitrocellulose membrane. The membranes were blocked for an hour and incubated with HER2 antibody (Cell Signalling, 1:1000) over night at 4° C. The membranes were then washed 3 times in 1× TBST and incubated with an anti-mouse HRP antibody (Cell Signalling, 1:5000) for 1 hour washed 3 times and probed.

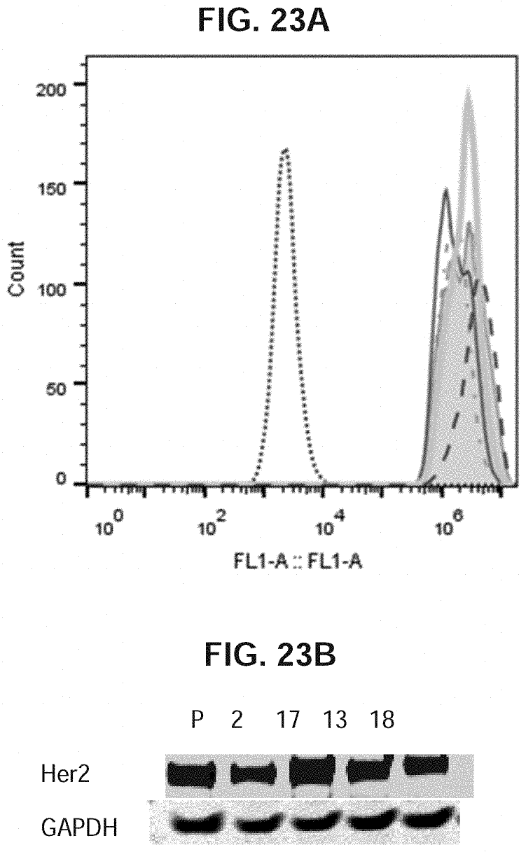

The HER2 expression levels of the T-DM1 relapsed tumors were similar to the control tumors (without T-DM1 treatment) as evaluated by FACS (FIG. 23A) and western blot (FIG. 23B)

D. T-DM1 Resistance is Not Due to Expression of Drug Efflux Pumps

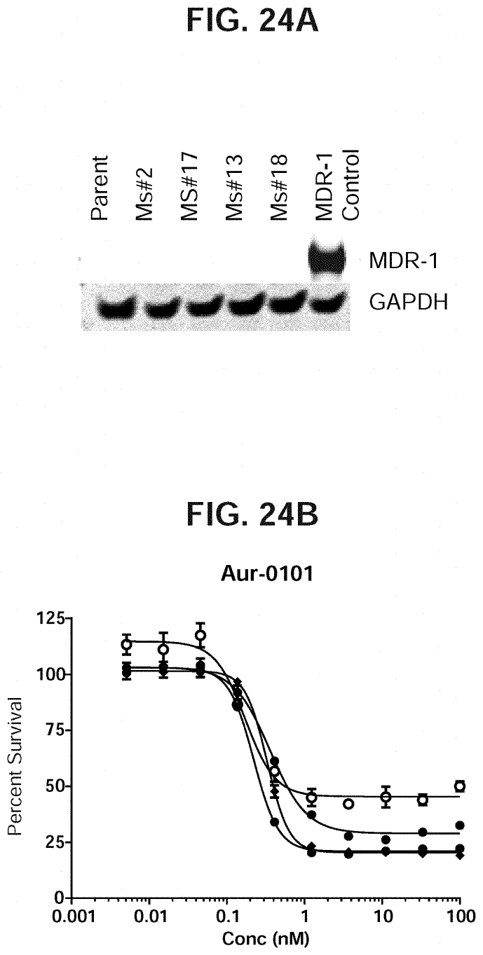

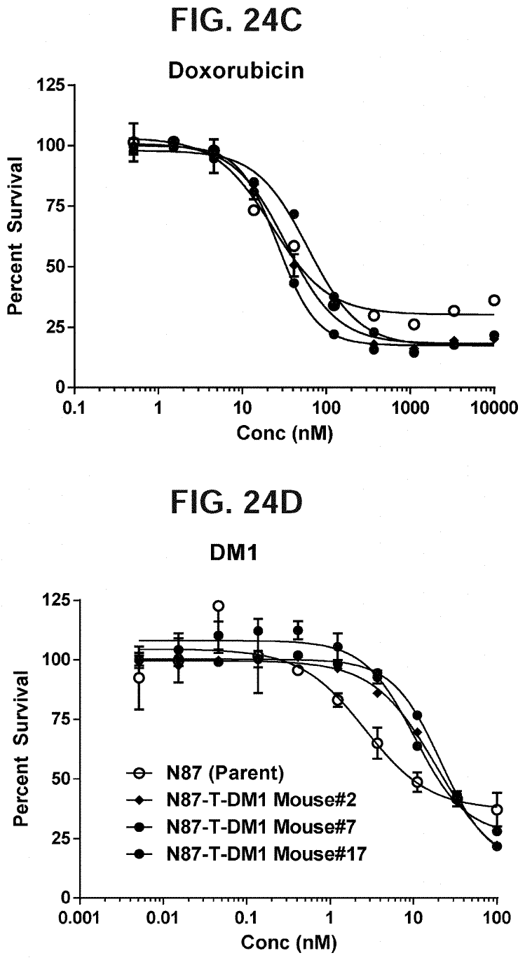

The cell lines do not express MDR1 by western blot (FIG. 24A) and cells are not resistant to MDR-1 substrate free drug 0101 (FIG. 24B). No resistance to doxorubicin (FIG. 24C) was observed indicating that resistant mechanism is not through MRP1. However, the cells are still resistant to free DM1 (FIG. 24D).

### Example 16: Pharmacokinetics (PK)

Exposure of conventional or site specific vc0101 antibody drug conjugates were determined after an IV bolus dose administration of either 5 or 6 mg/kg to cynomolgus monkeys. Concentrations of total antibody (total Ab; measurement of both conjugated mAb and unconjugated mAb) and ADC (mAb that is conjugated to at least one drug molecule) was measured using ligand binding assays (LBA). The ADC in was made using vc0101 in all cases except for T(LCQ05) were AcLysvc0101 was used. Conventional conjugation (not site specific conjugation) was used to make the ADC from trastuzumab.

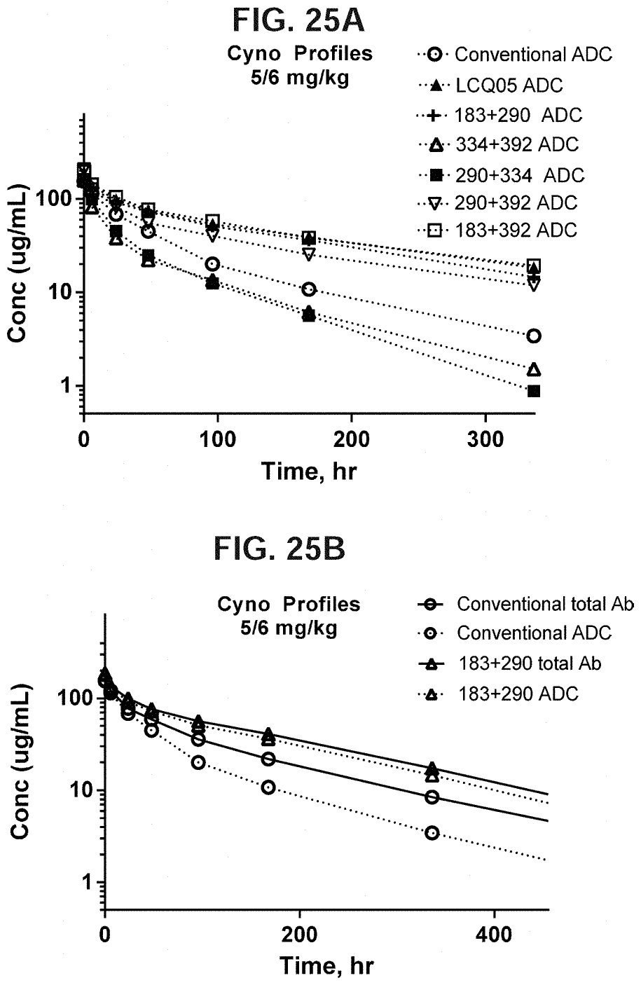

Concentration vs time profiles and pharmacokinetics/toxicokinetics of both total Ab and trastuzumab ADC (T-vc0101) (5 mg/kg) or T(kK183C+K290C) site specific ADC (6 mg/kg) after dose administration to cynomolgus monkeys (FIG. 25A and Table 21). Exposure of the T(kK183C+K290C) site specific ADC has both increased exposure and stability when compared to the conventional conjugate.

Concentration vs time profiles and pharmacokinetics/toxicokinetics of the ADC analyte of trastuzumab (T-vc0101) (5 mg/kg) or T(kK183C+K290C), T(LCQ05), T(K334C+K392C), T(K290C+K334C), T(K290C+K392C) and T(kK183C+K392C) site specific ADC (6 mg/kg) after dose administration to cynomolgus monkeys (FIG. 25B and Table 21). Exposure several site specific ADC (T(LCQ05), T(kK183C+K290C), T(K290C+K392C) and T(kK183C+K392C)) are higher compared to that of the trastuzumab ADC using conventional conjugation. However, exposure of two other site specific ADC (T(K290C+K334C) and T(K334C+K392C)) do not have higher exposure than the trastuzumab ADC indicating that not all site specific ADCs will have pharmacokinetic properties better than the trastuzumab ADC made using conventional conjugation.

TABLE 21
Pharmacokinetics

| | | | | AUC |
|---|---|---|---|---|
| | Dose | | Cmax | (0-336 h) |
| mAb/ADC | (mg/kg) | Analyte | (μg/mL) | (μg · h/mL) |
| trastuzumab | 5 | Total Ab | 157 | 11100 |
| | | ADC | 154 | 7660 |
| T(K290C + K334C) | 6 | Total Ab | 165 | 5770 |
| | | ADC | 163 | 5060 |
| T(K334C + K392C) | 6 | Total Ab | 159 | 5320 |
| | | ADC | 157 | 4770 |
| T(LCQ05) | 5 | Total Ab | 165 ± 19 | 16400 ± 1020 |
| | | ADC | 164 ± 22 | 16300 ± 989 |
| T(kK183C + K290C) | 6 | Total Ab | 187 | 16800 |
| | | ADC | 181 | 15300 |
| T(K183C + K392C) | 6 | Total Ab | 195 | 18500 |
| | | ADC | 196 | 16900 |
| T(K290C + K392C) | 6 | Total Ab | 205 | 13300 |
| | | ADC | 208 | 12300 |

### Example 17: Relative Retention Values by Hydrophobic Interaction Chromatography vs. Exposure (AUC) in Rats

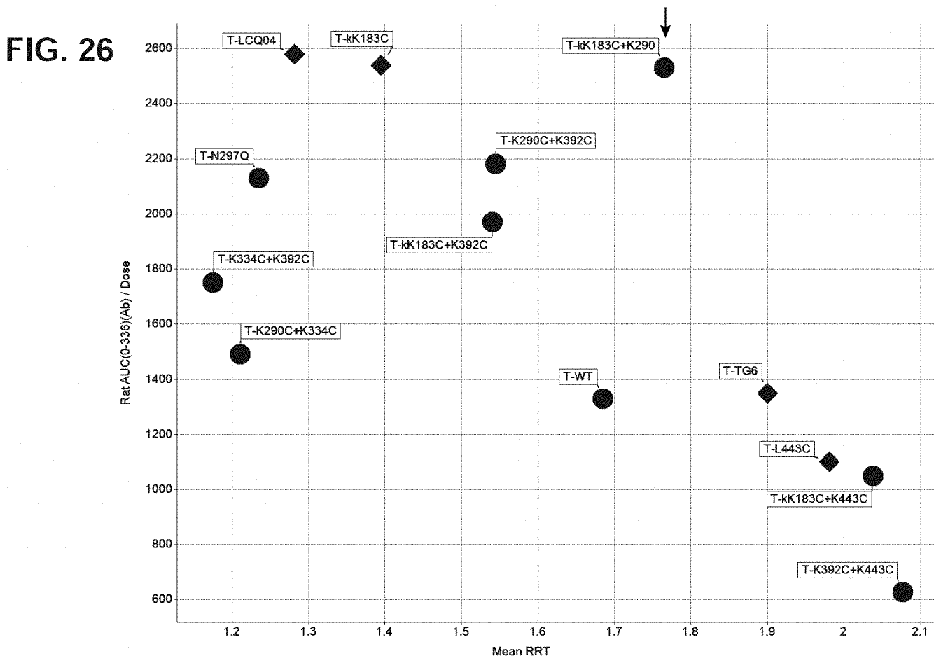

Hydrophobicity is a physical property of a protein that can be assessed by hydrophobicity interaction chromatography (HIC), and the retention times of protein samples differ based on their relative hydrophobicity. ADCs can be compared with their respective antibody by calculating a relative retention time (RRT), which is the ratio of the HIC retention time of the ADC divided by the HIC retention time of the respective antibody. Highly hydrophobic ADCs have higher RRT, and it is possible that these ADCs may also have more pharmacokinetic liability, specifically lower area-under-the-curve (AUC, or exposure). When the HIC values of ADCs with various site mutations were compared with their measured AUC in rats, the distribution in FIG. 26 was observed.

ADCs with RRT 9 showed lower AUC values, while ADCs with lower RRT tended to have higher AUC, although the relationship was not direct. The ADC T(kK183C+K290C)-vc0101 was observed to have a relatively higher RRT (mean value of 1.77) and therefore was expected to have a relatively lower AUC. Surprisingly, the observed AUC was relatively high, hence it was not obvious to predict the exposure of this ADC from the hydrophobicity data.

### Example 18: Toxicity Studies

In two independent exploratory toxicity studies, a total of ten male and female cynomolgus monkeys were divided into 5 dosage groups (1/gender/dosage) and dosed IV once every 3 weeks (study days 1, 22 and 43). On study day 46 (3 days after the 3^{rd} dose administration) animals were euthanized and protocol specified blood and tissue samples were collected. Clinical observations, clinical pathology, macroscopic and microscopic pathology evaluations were conducted in life and post necropsy. For anatomic pathology evaluation, severity of histopathology findings was recorded on a subjective, relative, study specific basis.

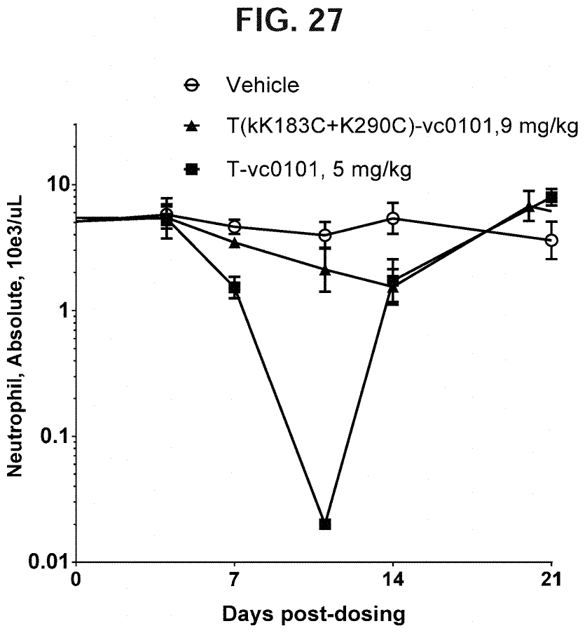

In cynomolgus monkey exploratory toxicity studies at 3 and 5 mg/kg, T-vc0101 caused transient but marked (390/μl) to severe (40/μl to non-detectable) neutropenia at Day 11 post the first dose. In contrast at 9 mg/kg, all cynomolgus monkeys dosed with T(kK183C+K290C)-vc0101 had none to minimal neutropenia with neutrophil counts well above 500/μl at any time-points tested (FIG. 27). In fact, T(kK183C+K290C)-vc0101 dosed animals showed average neutrophil counts (>1000 μL) at day 11 and 14 as compared to vehicle controls.

Microscopically in the bone marrow at 3 and 5 mg/kg, the cynomolgus monkey dosed with T-vc0101 had compound-related increased M/E ratio. Increased myeloid/erythroid (M/E) ratio consisted of decreased erythroid precursors combined with an increase of primarily mature granulocytes. In contrast, at 6 and 9 mg/kg, only the male dosed with T(kK183C+K290C)-vc0101 at 6 mg/kg/dose had minimal to mild increased cellularity of mature granulocytes (data not shown).

Therefore, the hematologic and microscopic data clearly indicated that the ADC conjugate based on site-specific-mutation technology, T(kK183C+K290C)-vc010 clearly improved the T-vc010 induced bone marrow toxicity and neutropenia.

### Example 19: ADC Crystal Structure

The crystal structures were obtained for T(K290C+K334C)-vc0101, T(K290C+K392C)-vc0101 and T(K334C+K392C)-vc0101. These particular ADCs were chosen for crystallography since conjugation to the K290C+K334C and K334C+K392C double cysteine-variants, but not the K290C+K392C, abolished ADCC activity.

The conjugated Fc regions were prepared for crystallography using papain cleavage of the ADCs. Crystals of the same morphology were obtained for the three conjugated IgG1-Fc regions using the same conditions: 100 mM NaCitrate pH 5.0+100 mM MgCl₂+15% PEG 4K.

Wild type human IgG1-Fc structures deposited in the PDB are relatively similar showing that the CH2-CH2 domains contact each other through Asn297-linked glycans (carbohydrate or glycan antennas) and that the CH3-CH3 domains form a stable interface that is relatively constant between structures. Fc structures exist in either a “closed” or “open” confirmation and the deglycosylated Fc structure adopts the “open” structure conformation thus demonstrating that the glycan antennas hold the CH2 regions together. Additionally, a published structure of an unconjugated Phe241Ala-IgG1 Fc mutant (Yu et al. “Engineering Hydrophobic

Protein-Carbohydrate interactions to fine-tune monoclonal antibodies”. JACS 2013) shows one partially disordered CH2 domain since this mutation leads to destabilization of CH2-glycan interface and CH2-CH2 interface since aromatic Phe residue cannot stabilize the carbohydrate.

The “CH2 domain” of a human IgG Fc region (also referred to as “Cγ2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton et al., 1985, Molec. Immunol. 22: 161-206).

The “CH3 domain” comprises the stretch of residues C-terminal to a CH2 domain in an Fc region (i.e. from about amino acid residue 341 to about amino acid residue 447 of an IgG).

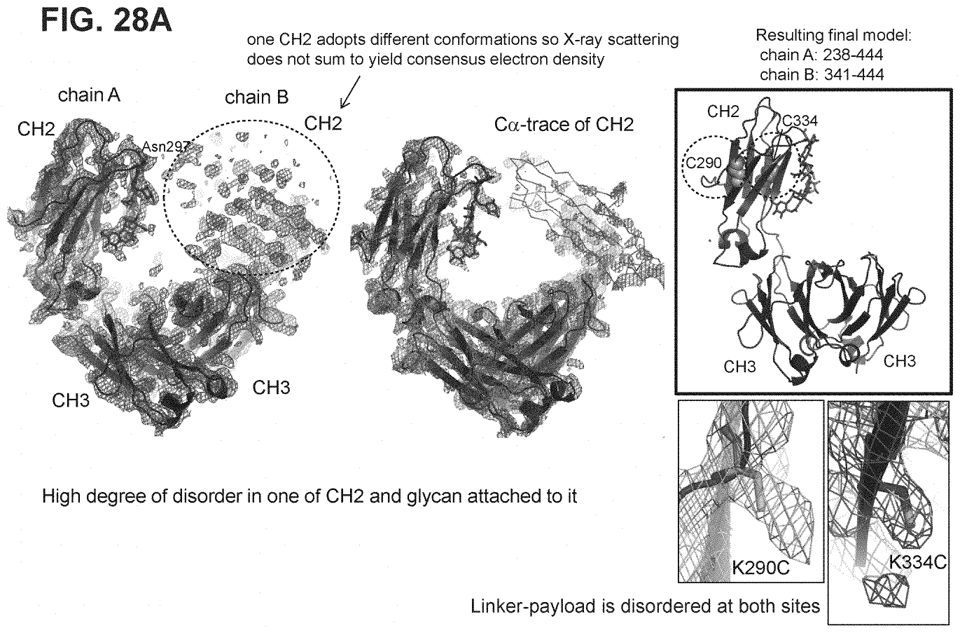

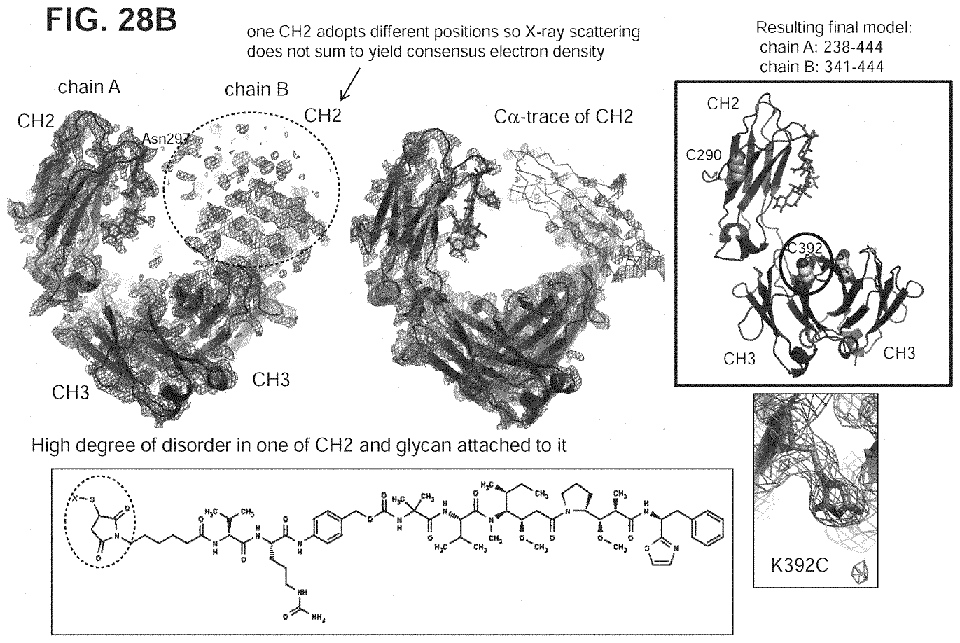

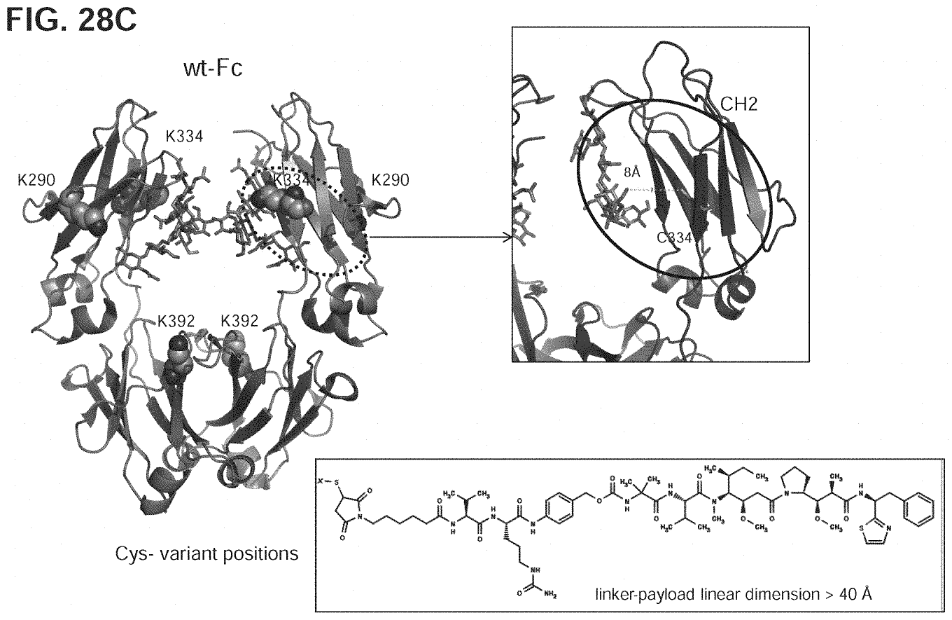

The solved structures for both T(K290C+K334C)-vc0101 and T(K290C+K392C)-vc0101 Fc regions were similar showing that the Fc dimer contained one CH2 and both CH3s that were highly ordered (like wild type Fc). However, they also contain a disordered CH2 with glycan attached (FIG. 28A and FIG. 28B). The higher degree of destabilization of one CH2 domain was attributed to the close proximity of conjugation sites to glycan antennas. Considering 0101 payload geometry, conjugation at any of K290, K334, K392 sites could perturb the overall trajectory of the glycan away from the CH2 surface destabilizing the glycan and the CH2 structure itself and as a result the CH2-CH2 interface (FIG. 28C). A higher degree of heterogeneity is available to these 0101 site-specifically conjugated double cysteine-Fc-variants relative to WT-Fc, Phe241Ala-Fc or deglycosylated-Fc. When engineered cysteine-variant positions were mapped on the structure of WT-Fc in complex with FcYR type IIb, it showed that conjugation at C334 could directly interfere with binding to FcYRIIb (FIG. 28C). This heterogeneity in CH2 positioning caused by mutation or conjugation could result in significant decrease in FcRIIb binding. Therefore these results suggested that either conformation heterogeneity or conjugation of 0101 to certain combinations of engineered cysteines within the IgG1-Fc could affect ADCC activity for the double cysteine variants containing the K334C site, or perhaps both.

### Example 20: Use of Site Specific Conjugation in Other Antibodies

The sites and modifications used to make the site specific HER2 ADCs of the invention can be used on antibodies directed to other antigens and still effect an improved efficacy over conventionally conjugated ADCs.

Antibody A (directed to a tumor-associated antigen) was made into an ADC using conventional conjugation as well as site specific conjugation. In both cases the linker used was vc and the drug used was auristatin 0101. For site specific conjugation, sites K183 on the antibody light chain and K290 in the CH2 region of the antibody heavy chain (using the EU index of Kabat) were altered to cysteine (C) to allow for conjugation to the linker/payload.

Methods used to make the site specific ADC similar to those used to make T(kK183C+K290C)-vc0101 (supra). The conjugation efficiency was 61% and the conjugated antibody had an average DAR of 4, with similar HIC-RRT profile as T(kK183C+K290C)-vc0101.

Thermal stability of the site specific conjugate (SSC) was assessed and the lowest melting point of the SSC was 65° C., indicating sufficient stability. Binding the SSC to its target tumor antigen was also assessed in comparison to unconjugated antibody and no reduction of binding capacity was detected in ELISA based binding assays, indicating good retention of binding affinity after conjugation with the payload linker vc0101.

In vitro cytotoxicity was then assessed in tumor cells lines with elevated expression of the tumor antigen. The SSC had comparable cytotoxic potency with the conventional ADC in the cytotoxicity assay using multiple tumor lines. In vivo efficacy studies were further performed in xenograft tumor models inoculated with the tumor cells over-expressing the tumor antigen. In one model, at the dosage level of 3 mg/kg, the SSC led to complete tumor regression after 4 doses administered twice per week. Tumor regression was maintained until around 60 days after the last dose when tumor regrowth was observed. In contrast, while conventional ADC dosed at the same schedule also led to tumor regression after 4 doses, tumor regrowth was observed about 30 days after the last dose, much earlier than that in the SSC. Similar findings of better maintenance of tumor regression efficacy were observed in efficacy studies in the other tumor model. These data indicate that the site specific conjugate of antibody A based on kK183C+K290C had better maintained exposure in dosed animals than the conventionally prepared ADC, suggesting improved stability of the SSC results in better pharmacokinetic parameters.

### Example 21. Different Conjugation Sites Results in Different ADC Properties

A. General Procedure for the Synthesis of Cys-Mutant ADCs:

The following two LP were used:

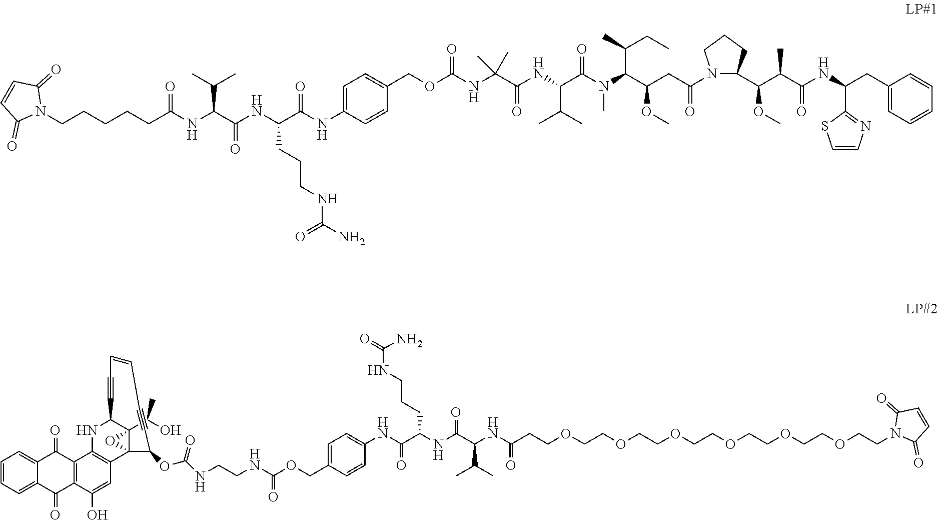 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)C(C)(C)NC(=O)OCc1ccc(NC(=O)[C@H](CCCNC(N)=O)NC(=O)[C@@H](NC(=O)CCCCCN2C(=O)C=CC2=O)C(C)C)cc1)C(C)C ; CC(C)[C@H](NC(=O)CCOCCOCCOCCOCCOCCOCCN1C(=O)C=CC1=O)C(=O)N[C@@H](CCCNC(N)=O)C(=O)Nc1ccc(COC(=O)NCCNC(=O)O[C@@H]2C#C/C=C\C#C[C@@H]3Nc4c(cc(O)c5c4C(=O)c4ccccc4C5=O)[C@]24O[C@@]34[C@@H](C)O)cc1]

A solution of trastuzumab containing incorporating an engineered cyststeine residue (as shown in the table below) was prepared in 50 mM phosphate buffer, pH 7.4. PBS, EDTA (0.5 M stock), and TCEP (0.5 M stock) were added such that the final protein concentration was 10 mg/mL, the final EDTA concentration was 20 mM, and the final TCEP concentration was approximately 6.6 mM (100 molar eq.). The reaction was allowed to stand at rt for 48 h then buffer exchanged into PBS using GE PD-10 Sephadex G25 columns per the manufacturer's instructions. The resulting solution was treated with approximately 50 equivalents of dehydroascorbate (50 mM stock in 1:1 EtOH/water). The antibody was allowed to stand at 4° C. overnight and subsequently buffer exchanged into PBS using GE PD-10 Sephadex G25 columns per the manufacturer's instructions. Slight variations of the above procedure were employed on some mutants.

The antibody thus prepared was diluted to ˜2.5 mg/mL in PBS containing 10% DMA (vol/vol) and treated with LP#1 (10 molar eq.) as a 10 mM stock solution in DMA. After 2 h at rt, the mixture was buffer exchanged into PBS (per above) and purified by size-exclusion chromatography on a Superdex200 column. The monomeric fractions were concentrated and filter sterilized to give the final ADC. See Table 22 below for product characterization.

TABLE 22
Summary of ADC properties:

| | | | | | | | | HIC |
|---|---|---|---|---|---|---|---|---|
| | | | | LCMS | LCMS | | | relative |
| | | LCMS | HIC | Observed | Expected | | HIC RT | retention |
| | Trastuzumab | DAR | DAR | Mass | Mass | % | Main Peak | time |
| Example | mutant | (mol/mol) | (mol/mol) | Shift | Shift | monomer | (min) | (RRT) |
| ADC#1 | 114C | 1.9 | 1.74 | 1342 | 1341 | 94% | 7.15 | 1.40 |
| ADC#2 | kappa-183C | 2 | 2 | 1341 | 1341 | 99% | 7.05 | 1.38 |
| ADC#3 | 290C | 2.1 | 2.1 | 1341 | 1341 | 99% | 7.85 | 1.53 |
| ADC#4 | 334C | 2.1 | 2.1 | 1341 | 1341 | 99% | 5.90 | 1.15 |
| ADC#5 | 347C | 1.9 | NA | 1341 | 1341 | 99% | 8.41 | 1.64 |
| ADC#6 | 375C | 2 | NA | 1340 | 1341 | 99% | 6.23 | 1.22 |
| ADC#7 | 380C | 2 | 1.9 | 1341 | 1341 | 99% | 7.93 | 1.55 |
| ADC#8 | 388C | 1.9 | NA | 1340 | 1341 | 97% | 8.75 | 1.71 |
| ADC#9 | 392C | 2.1 | 2.1 | 1341 | 1341 | 98% | 6.60 | 1.29 |
| ADC#10 | 421C | 1.9 | NA | 1342 | 1341 | 93% | 8.20 | 1.60 |
| ADC#11 | 443C | 2 | 2 | 1344 | 1341 | 90% | 9.10 | 1.78 |
| ADC#12 | kappa183C + 334C | 3.7 | NA | 1341 | 1341 | 95% | 7.00 | 1.37 |
| ADC#13 | kappa183C + 392C | 4 | 4 | 1342 | 1341 | 97% | 7.70 | 1.50 |
| ADC#14 | 290C + 334C | 4 | 4 | 1342 | 1341 | 97% | 6.03 | 1.18 |
| ADC#15 | 334C + 392C | 4 | 4 | 1343 | 1341 | 97% | 5.91 | 1.15 |
| ADC#16 | 392C + 443C | 3.2 | NA | 1340 | 1341.68 | 97% | 10.85 | 2.12 |
| Trastuzumab | NA | | | | | | 5.12 | 1.00 |

B. General Analytical Methods for Conjugation Examples:

LCMS: Column=Waters BEH300-C4, 2.1×100 mm (P/N=186004496); Instrument=Acquity UPLC with an SQD2 mass spec detector; Flow rate=0.7 mL/min; Temperature=80° C.; Buffer A=water+0.1% formic acid; Buffer B=acetonitrile+0.1% formic acid. The gradient ran from 3% B to 95% B over 2 minutes, holds at 95% B for 0.75 min, and then re-equilibrates at 3% B. The sample was reduced with TCEP or DTT immediately prior to injection. The eluate was monitored by LCMS (400-2000 daltons) and the protein peak was deconvoluted using MaxEnt1. DAR was reported as a weight average loading.

SEC: Column: Superdex200 (5/150 GL); Mobile phase: Phosphate buffered saline containing 2% acetonitrile, pH 7.4; Flow rate=0.25 mL/min; Temperature=ambient; Instrument: Agilent 1100 HPLC.

HIC: Column: TSKGel Butyl NPR, 4.6 mm×3.5 cm (P/N=S0557-835); Buffer A=1.5 M ammonium sulfate containing 10 mM phosphate, pH 7; Buffer B=10 mM phosphate, pH 7+20% isopropyl alcohol; Flow rate=0.8 mL/min; Temperature=ambient; Gradient=0% B to 100% B over 12 minutes, hold at 100% B for 2 minutes, then re-equilibrate at 100% A; Instrument: Agilent 1100 HPLC.

C. Determination of Hydrophobicity of Site Specific vc0101 Conjugates

ADC#1-ADC#16 were evaluated by hydrophobic interaction chromatography (method above) in order to determine the relative hydrophobicity of the various conjugates. ADC hydrophobicity has been reported to correlate with total antibody exposure.

Conjugates to sites 334, 375, and 392 exhibited to smallest shift in retention time as compared to the unmodified antibody while conjugates to sites 421, 443, and 347 showed the largest shift in retention time. The relative hydrophobicity of each ADC was calculated by dividing the retention time of the ADC by the retention time of the unmodified antibody, thus resulting in a “relative retention time” or “RRT”. An RRT of -1 indicates that the ADC has approximately the same hydrophobicity as the unmodified antibody. The RRT for each ADC is shown in Table 22.

D. ADC Plasma Stability of Site Specific vc0101 Conjugates

ADC samples (˜1.5 mg/mL) were diluted into mouse, rat or human plasma to yield a final solution of 50 μg/mL ADC in plasma. Samples were incubated at 37° C. under 5% CO₂, and aliquots were taken at three time points (0, 24 h, and 72 h). Each time point of ADC samples from the plasma incubation (25 μL) was deglycosylated with IgG0 at 37° C. for 1 h. Following the deglycosylation, a capture antibody (biotinylated goat anti-human IgG1 Fcγ fragment specific at 1 mg/mL for mouse and rat plasma, or biotinylated anti-trastuzumab antibody at 1 mg/mL for human plasma) was added and the mixture was heated at 37° C. for 1 h followed by gentle shaking at room temperature for a second hour. Dynabead MyOne Streptavidin T1 magnetic beads were added to the samples and incubated at room temperature for 1 h with gentle shaking. The sample plate was then washed with 200 μL PBS+0.05% Tween-20, 200 μL PBS and HPLC grade water. The bound ADC was eluted with 55 μL of 2% of formic acid (FA) (v/v). 50 μL aliquot of each sample were transferred into a new plate followed by an additional 5 μL of 200 mM TCEP.

The intact protein analysis was carried out with Xevo G2 Q-TOF mass spectrometer coupled with nanoAcquity UPLC (Waters) using BEH300 C4, 1.7 μm, 0.3×100 mm iKey column. The mobile phase A (MPA) consisted of 0.1% FA in water (v/v) and the mobile phase B (MPB) consisted of 0.1% FA in acetonitrile (v/v). The chromatographic separation was achieved at a flow rate of 0.3 μL/min using a linear gradient of MPB from 5% to 90% over 7 min. The LC column temperature was set at 85° C. Data acquisition was conducted with MassLynx software version 4.1. The mass acquisition range was from 700 Da to 2400 Da. Data analysis including deconvolution was performed using Biopharmalynx version 1.33.

Loading and succinimide ring opening (a +18 dalton peak) was monitored over time. The loading data is reported as % DAR loss compared to 0 h DAR. The ring-opening data is reported as the % of ring-opened species as compared to total species present at 72 h. Several site mutants resulted in very stable ADCs (334C, 421C, and 443C) while some sites lost significant amounts of linker-payload (380C and 114C). The rate of ring-opening varied considerably between the sites. Several sites such as 392C, 183C, and 334C resulted in very little ring opening while other sites such as 421C, 388C, and 347C resulted in rapid and spontaneous ring

Sites that result in rapid and spontaneous ring opening may be useful for the generation of conjugates that have reduced hydrophobicity and/or increased PK exposure. This finding runs counter to the prevailing understanding that ring stability correlates with plasma stability. In some aspects therefore, conjugation at one or more of sites 421C, 388C, and 347C can be particularly advantageous when using a linker-payload with a high hydrophobicity. In some aspects, high hydrophobicity is a relative retention time (RRT) value (as measured by HIC) of 1.5 or more. In some aspects, high hydrophobicity is a RRT value of 1.7 or more. In some aspects, high hydrophobicity is a RRT value of 1.8 or more. In some aspects, high hydrophobicity is a RRT value of 1.9 or more. In some aspects, high hydrophobicity is a RRT value of 2.0 or more.

TABLE 23
Plasma stability of various ADCs

| | Conjugation | % DAR | % Succinamide |
|---|---|---|---|
| ADC# | Site | Loss @ 72-h | hydrolysis @ 72-h |
| ADC#4 | C334 | 0% | 18% |
| ADC#10 | C421 | 0% | 100 |
| ADC#11 | C443 | 0% | 40% |
| ADC#8 | C388 | −1.3% | 100% |
| ADC#9 | C392 | 3.0% | 0% |
| ADC#3 | C290 | 9.5% | 21% |
| ADC#5 | C347 | 10% | 66% |
| ADC#2 | kC183 | 11% | 29% |
| ADC#6 | C375 | 12% | 46% |
| ADC#1 | C114 | 20% | 33% |
| ADC#7 | C380 | 49% | 29% |

E. Glutathione Stability of Site Specific vc0101 Conjugates

The ADC samples were diluted into aqueous glutathione to yield a final GSH concentration of 0.5 mM and final protein concentration of ˜0.1 mg/mL in a phosphate buffer, pH 7.4. The samples were then incubated at 37° C. and aliquots were removed at three time points to determine the DAR (T-0, T-3 day, T-6 day). The aliquot from each time point was treated with TCEP and analyzed by LC-MS per the method described in example #21.A.

Loading and succinimide ring opening (a +18 dalton peak) was monitored over time. The loading data is reported as % DAR loss compared to 0 h DAR. (Table 24) The ring-opening data is reported as the % of ring-opened species as compared to total species present at 72 h. Several site mutants resulted in very stable ADCs (334C, 421C, and 443C) while some sites lost significant amounts of linker-payload (380C and 114C). The rate of ring-opening varied considerably between the sites. Several sites such as 392C, 183C, and 334C resulted in very little ring opening while other sites such as 421C, 388C, and 347C resulted in considerable ring-opening. The results of this assay correlates quite well with the plasma stability results (Example 21.D) suggesting that thiol-mediated deconjugation is the major pathway of payload loss in plasma. Combined, these results suggest that particular sites such as 334, 443, 290, and 392 may be especially useful for the conjugation of payload-linkers that are readily lost through a thiol-mediated deconjugation. Such payload-linkers include those that utilize the common maleimide-caproyl (mc) and maleimide-caproyl-ValCit (vc) linkages (e.g. vc-101, vc-MMAE, mc-MMAF etc).

TABLE 24
Glutathione stability of various vc0101 site specific conjugates

| | Conjugation | % DAR | % Succinamide |
|---|---|---|---|
| ADC# | Site | Loss @ 72-h | hydrolysis @ 72-h |
| ADC#1 | A114C | 12% | 41% |
| ADC#2 | kK183C | 7% | 17% |
| ADC#4 | K334C | 4% | 26% |
| ADC#5 | Q347C | 10% | 71% |
| ADC#6 | S375C | 18% | 47% |
| ADC#7 | E380C | 79% | 50% |
| ADC#8 | E388C | 19% | 100% |
| ADC#9 | K392C | 0% | 17% |
| ADC#10 | N421C | 0% | 80% |
| ADC#11 | L443C | 12% | 41% |
| ADC#3 | K290C | 17% | 33% |

F. Pharmacokinetic Evaluation of Select Site Specific vc0101 Conjugates in Mice

Non-tumor bearing athymic female nu/nu (nude) mice (6-8 weeks of age) were obtained from Charles River Laboratories. All procedures using mice were approved by the Institutional Animal Care and Use Committee according to established guidelines. Mice (n=3 or 4) were administered a single intravenous dose of an ADC at 3 mg/kg based on the antibody component. Blood samples were collected from each mouse via the tail vein at 0.083, 6, 24, 48, 96, 168 and 336 hours post-dose. The total antibody (T_{ab}) and ADC concentrations were determined by a LBA where a sheep anti-human IgG antibody was used for capture, a goat anti-human IgG antibody was used for detection of T_{ab} or an anti-payload antibody was used for detection of ADC. Plasma concentration data for each animal was analyzed using Watson LIMS version 7.4 (Thermo). Exposure varied based on site. The ADCs made from the 290C and 443C mutants exhibited the lowest exposure, while ADCs made from the kappa-183C and 392C sites exhibited the highest exposure. For many applications, sites with a high exposure may be preferred, as this will lead to increased duration of therapeutic agent. However, for certain applications, it may be preferable to use a conjugate with a lower exposure (such as 290C and 443C. In particular, applications where a lower exposure (i.e. lower PK) is desirable may include, but are not limited to, use in the brain, the CNS, and the eye. Indications include cancer, especially of the brain, CNS and/or eye.

TABLE 25
PK exposure of various site-specific vc0101 ADCs

| | | tAb AUC | ADC AUC |
|---|---|---|---|
| ADC# | Site | (0-last) (mg*h/mL) | (0-last) (mg*h/mL) |
| ADC#2 | Kappa- | 7150 | 5980 |
| | 183C | | |
| ADC#3 | 290C | 4240 | 3480 |
| ADC#4 | 334C | 5130 | 4500 |
| ADC#5 | 347C | 5080 | 4070 |
| ADC#8 | 388C | 6100 | 3680 |
| ADC#9 | 392C | 6400 | 6010 |
| ADC#11 | 443C | 4430 | 4500 |

G. Cathepsin Cleavage of Site Specific vc0101 Conjugates

Cathepsin B was activated using 6 mM dithiothreitol (DTT) in 150 mM sodium acetate, pH 5.2 for 15 min at 37° C. 50 ng of the activated cathepsin-B was then mixed with 20 uL of 1 mg/mL of ADC at a final concentration of 2 mM DTT, 50 mM sodium acetate, pH 5.2. Reactions were quenched using 10 uM E-64 cysteine protease inhibitor in 250 mM borate buffer, pH 8.5 following incubation at 37° C. for 20 min, 1 h, 2h and 4 h. After the assay, the samples were reduced using TCEP and analyzed by LC/MS using the conditions described in Example 21.A. The data showed that the rate of linker cleavage depends heavily on the site of conjugation. Particular sites are cleaved very quickly, such as 443C, 388C, and 290C while other sites are cleaved very slowly, such as 334C, 375C, and 392C. In some aspects, it may be advantageous to conjugate to sites that lend themselves to slow cleavage. In other aspects, quick cleavage is preferred. For example, it may be preferable to release the payload quickly to reduce time spent in the endosome. In further aspects rapid payload cleavage can be advantageously permit penetration of the payload where the conjugated molecule may not be able to do so, such as certain solid tumors. In further aspects, rapid cleavage can permit the payload to be delivered to adjacent cells that do not express the antibody's antigen, thus permitting treatment of a heterogenous tumor, for example.

TABLE 26
Linker cleavage kinetics of various site-specific vc0101 ADCs

| | | % Linker | % Linker | % Linker | % Linker |
|---|---|---|---|---|---|
| | | cleavage | cleavage | cleavage | cleavage |
| ADC# | Mutant | @ 20 min | @ 1 h | @ 2 h | @ 4 h |
| ADC#1 | 114C | 29% | 71% | 100% | 100% |
| ADC#2 | Kappa-183C | 31% | 95% | 100% | 100% |
| ADC#3 | 290C | 54% | 100% | 100% | 100% |
| ADC#4 | 334C | 0% | 0% | 0% | 13% |
| ADC#5 | 347C | 42% | 89% | 100% | 100% |
| ADC#6 | 375C | 0% | 0% | 0% | 5% |
| ADC#7 | 380C | 15% | 48% | 83% | 92% |
| ADC#8 | 388C | 82% | 100% | 100% | 100% |
| ADC#9 | 392C | 0% | 0% | 0% | 0% |
| ADC#10 | 421C | 31% | 61% | 73% | 100% |
| ADC#11 | 443C | 100% | 100% | 100% | 100% |

H. Thermal Stability of Site Specific vc0101 Conjugates

The ADC was diluted to 0.2 mg/mL in PBS (pH 7.4) containing 10 mM EDTA. The ADCs were placed in a sealed vial and heated to 45° C. An aliquot (10 μL) was removed at 1-week increments to evaluate the level of high molecular weight species (HMWS) and low molecular weight species (LMWS) that formed over time by size exclusion chromatography (SEC). The SEC conditions are outlined in Example 21.A. Under these conditions, the monomer eluted at approximately 3.6 minutes. Any protein material eluting to the left of the monomer peak was counted as HMWS and any protein material eluting to the right of the monomer peak was counted as LMWS. Results are shown in Table 27 below. Select ADCs showed excellent thermal stability, such as kappa-183C, 375C, and 334C, while other ADCs showed significant decomposition, such as 443C and 392C+443C.

TABLE 27
Thermal stability of various site-specific vc0101 ADCs

| | Site | Day 1 | Day 1 | Day 1 | Day 21 | Day 21 | Day 21 |
|---|---|---|---|---|---|---|---|
| ADC# | mutant | (HMWS) | (LMWS) | (Monomer) | (HMWS) | (LMWS) | (Monomer) |
| ADC#1 | 114C | 3.31% | 3.00% | 93.60% | 1.70% | 5.30% | 93.80% |
| ADC#2 | Kappa- | 0.40% | 0.60% | 99.00% | 0.40% | 1.30% | 98.30% |
| | 183C | | | | | | |
| ADC#3 | 290C | 0.90% | 0.30% | 98.70% | 2.00% | 2.80% | 95.20% |
| ADC#4 | 334C | 0.80% | 0.40% | 98.80% | 1.10% | 2.60% | 96.30% |
| ADC#5 | 347C | 1.10% | 0.40% | 98.50% | 1.20% | 1.50% | 97.30% |
| ADC#6 | 375C | 0.70% | 0.60% | 98.70% | 0.80% | 2.10% | 97.20% |
| ADC#7 | 380C | 0.90% | 0.30% | 98.80% | 1.60% | 1.70% | 96.60% |
| ADC#8 | 388C | 1.90% | 0.70% | 97.40% | 1.20% | 2.10% | 96.70% |
| ADC#9 | 392C | 1.20% | 0.50% | 98.30% | 1.40% | 2.40% | 96.10% |
| ADC#10 | 421C | 2.60% | 4.30% | 93.00% | 2.60% | 6.10% | 91.30% |
| ADC#11 | 443C | 5.20% | 4.60% | 90.10% | 5.80% | 6.30% | 87.40% |
| ADC#12 | 183C + | 4.60% | 0.50% | 94.90% | 5.70% | 1.90% | 92.40% |
| | 334C | | | | | | |
| ADC#13 | 183C + | 2.10% | 0.70% | 97.10% | 2.10% | 1.60% | 96.30% |
| | 392C | | | | | | |
| ADC#14 | 290C + | 2.80% | 0.60% | 96.60% | 4.30% | 1.90% | 93.70% |
| | 334C | | | | | | |
| ADC#15 | 334C + | 1.90% | 0.70% | 97.40% | 2.70% | 2.40% | 94.90% |
| | 392C | | | | | | |
| ADC#16 | 392C + | 2.80% | 0.60% | 96.60% | 8.80% | 2.90% | 88.30% |
| | 443C | | | | | | |

I. Efficacy of Various vc0101 Site-Mutants

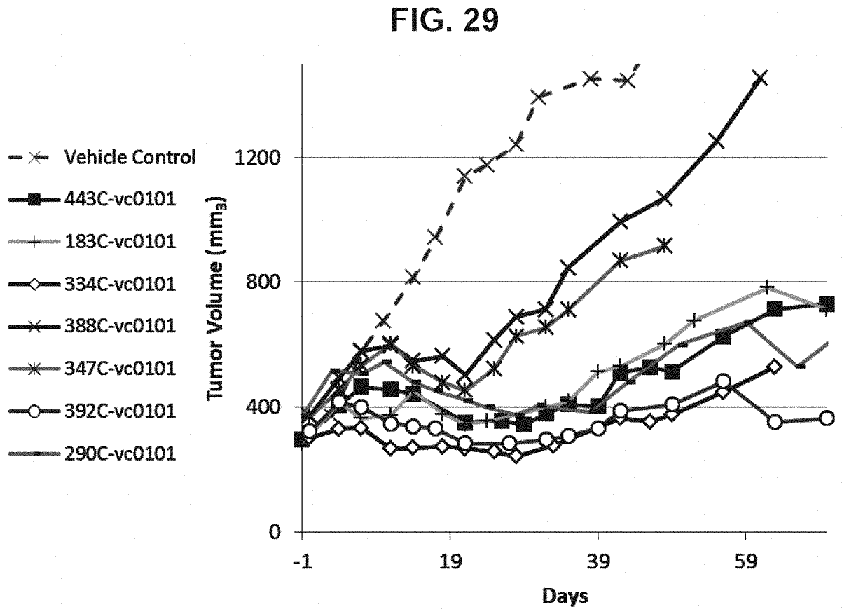

In vivo efficacy studies of antibody-drug conjugates were performed in a target-expressing xenograft model using the N87 cell line. Approximately 7.5 million tumor cells in 50% matrigel were implanted subcutaneously into 6-8 weeks old nude mice until the tumor sizes reach between 250 and 350 mm³. The drug was dosed through bolus tail vein injection. Animals were injected with 10, 3, or 1 mg/kg of antibody drug conjugate a total of four times, once every 4 days (on days 1, 5, 9, and 13). All experimental animals are monitored for body weight changes weekly. Tumor volume is measured twice a week for the first 50 days and once weekly thereafter by a Caliper device and calculated with the following formula: Tumor volume =(length×width²)/2. Animals are humanely sacrificed before their tumor volumes reach 2500 mm³. The tumor size is generally observed to decrease after the first week of treatment. Animals were monitored continuously for tumor re-growth after the treatment has discontinued (up to 100 days post-treatment). Data from the 3mpk dosing group is shown in FIG. 29. ADCs generated from the 388C and 347C mutants exhibited slightly lower potency than ADCs from the 334C, kappa-183C, 392C, and 443C mutants.

J. Conjugation of an Uncialamycin Payload to Various Mutants

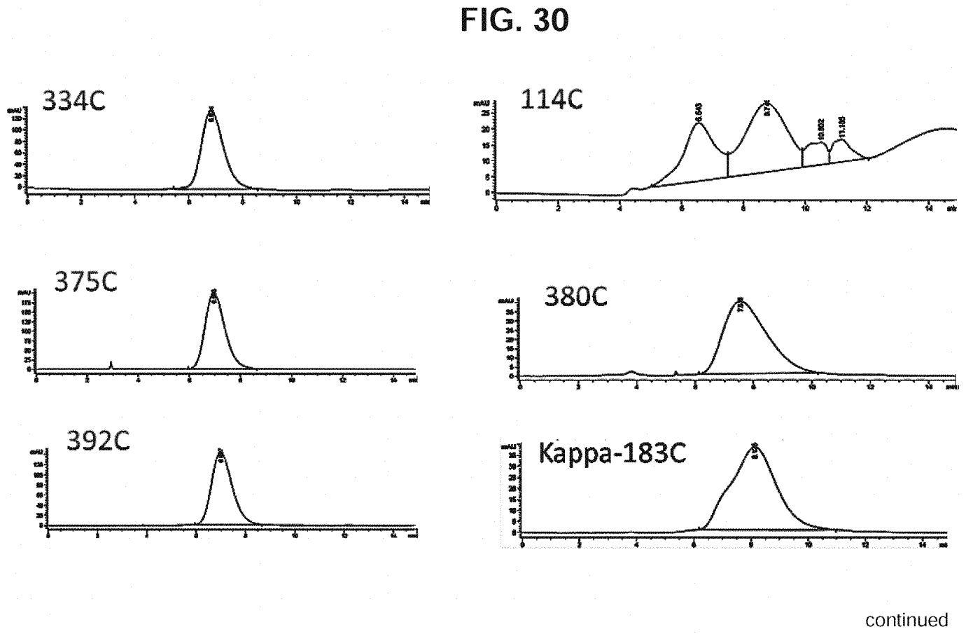

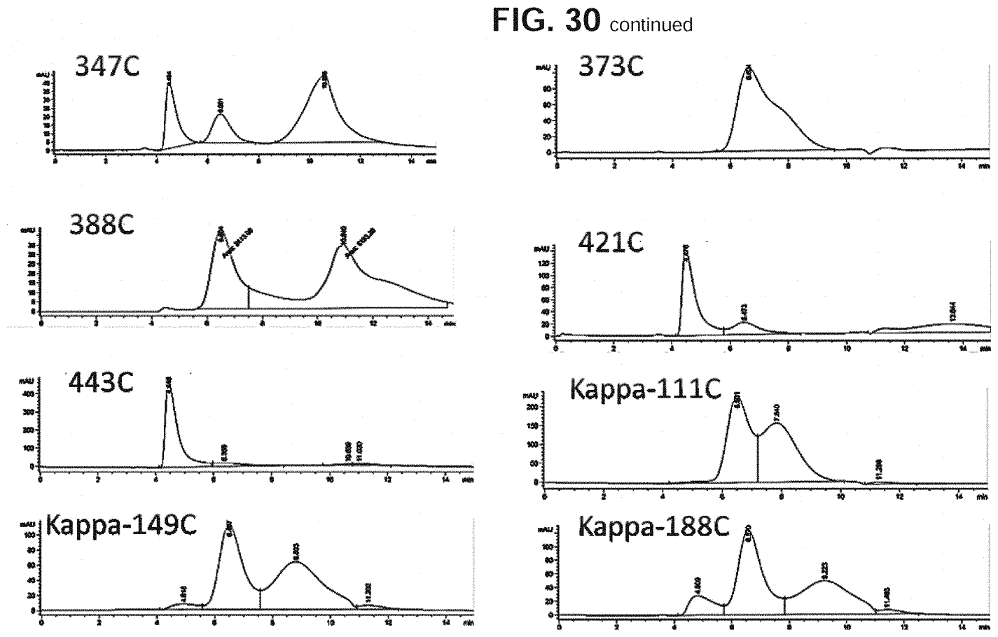

A panel of trastuzumab cysteine mutants were prepared for conjugation as described in Example #1. The resulting mutants (5 mg/mL) were treated with LP#2 (6 molar eq.) in PBS containing 10% DMA. After 2 h at rt the reactions were evaluated by LCMS to determine loading and by SEC to determine proper folding and lack of aggregation. The results are summarized in Table 28 and the raw SEC results are shown in FIG. 30.

As can be seen, various site mutants result in well behaved monomeric ADCs (334C, 375C, and 392C). Other site mutants fail to load (e. g. 246C, k149C, k111C), aggregate (e. g. 443C, 421C, 347C), or result in late-eluting ADCs that are perhaps partially unfolded (e. g. 380C, 388C, 290C, and k183C). Taken together, these results suggest that particular payloads may require optimization in order to identify sites that result in biophysically stable and properly folded ADCs.

TABLE 28
Tabulated results for the conjugation
of various trastuzumab mutants to LP#2

| | LCMS | % Agg | % Monomer | % Right shifted |
|---|---|---|---|---|
| Mutant name | DAR | (rt < 6 min) | (rt = 6-7 min) | (rt > 7 min) |
| PT-A114C | 2.0 | 4 | 42 | 53 |
| PT-K246C | 0.8 | 1 | 99 | 0 |
| PT-K290C | 2.0 | 1 | 38 | 60 |
| PT-K334C | 1.5 | 2.6 | 97 | 0 |
| PT-Q347C | 1.4 | 21 | 15 | 63 |
| PT-Y373C | 1.2 | 0 | 63 | 36 |
| PT-S375C | 1.8 | 0.6 | 99 | 0 |
| PT-E380C | 1.8 | 0 | 0 | 100 |
| PT-E388C | 1.3 | 0 | 32 | 68 |
| PT-K392C | 2 | 2.5 | 97 | 2.5 |
| PT-N421C | 1.4 | 77 | 22 | 0 |
| PT-L443C | 1.8 | 87 | 8 | 0 |
| PT-kA111C | 0.7 | 0 | 48 | 50 |
| PT-kK149C | 0.9 | 3 | 44 | 50 |
| PT-kK183C | 1.9 | 0 | 12 | 87 |
| PT-kK188C | 1.4 | 11 | 47 | 39 |

K. Summary

As demonstrated by the Examples, site of conjugation can impact LP deconjugation, LP metabolism, tAb exposure, rate of linker cleavage, ADC aggregation, ADC hydrophobicity, and in vivo PK profile.

Depending on the specific applications of the ADC molecules, a number of candidate conjugation sites can be used to solve specific problems. For example, if less hydrophobicity is desired, sites 334, 375, 392, or a combination thereof may be preferred, as they exhibited smallest shift in retention time as compared to the unmodified antibody. In another example, sites that result in rapid and spontaneous ring opening (e. g. , 421C, 388C, 347C, or a combination thereof) may be useful for the generation of conjugates that have reduced hydrophobicity and/or increased PK exposure. Sites such as 334, 443, 290, 392, or a combination thereof may be especially useful for the conjugation of payload-linkers that are readily lost through a thiol-mediated deconjugation.

### Example 22: Site-Specific Tubulysin ADCs

A. General Procedure for the Synthesis of Cys-Mutant ADCs:

The following two LP were used. Detailed synthesis schemes of tubulysin-based LPs are described in detail in U.S. Provisional Application 62/289,485, filed Feb. 1, 2016, and is herein incorporated by reference in its entirety.

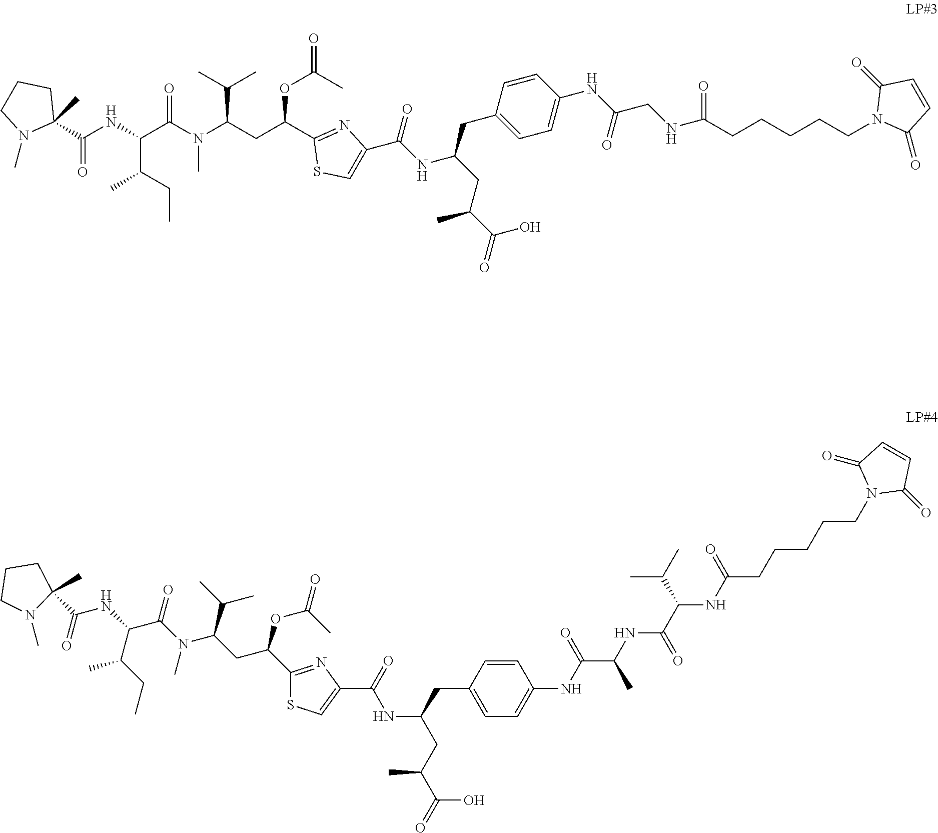 [SMILES: CC[C@H](C)[C@H](NC(=O)[C@@]1(C)CCCN1C)C(=O)N(C)[C@H](C[C@@H](OC(C)=O)c1nc(C(=O)N[C@@H](Cc2ccc(NC(=O)CNC(=O)CCCCCN3C(=O)C=CC3=O)cc2)C[C@H](C)C(=O)O)cs1)C(C)C ; CC[C@H](C)[C@H](NC(=O)[C@@]1(C)CCCN1C)C(=O)N(C)[C@H](C[C@@H](OC(C)=O)c1nc(C(=O)N[C@@H](Cc2ccc(NC(=O)[C@H](C)NC(=O)[C@@H](NC(=O)CCCCCN3C(=O)C=CC3=O)C(C)C)cc2)C[C@H](C)C(=O)O)cs1)C(C)C]

Method A: Conjugation of commercial HERCEPTIN antibody with linker payload via internal disulfides. A solution of trastuzumab antibody (˜15 mg/mL) was prepared in 50 mM phosphate buffered saline (pH 7.0) containing 50 mM EDTA. Tris(2-carboxyethyl)phosphine hydrochloride (TCEP) was added (˜2.0 molar equivalents) as a 5 mM solution in distilled water. The resulting solution was heated to 37° C. for 1 h. Upon cooling, the reaction was treated with appropriate volumes PBS and dimethyl acetamide (DMA) to bring the resulting solution to ˜5 mg/mL in PBS containing ˜10% DMA (vol/vol). The appropriate linker payload was added as a 10 mM stock in DMA (˜7 eq) and the reaction was allowed to stand or was gently agitated at room temperature. After 70 mins, the reaction was buffer exchanged into PBS using GE PD-10 Sephadex G25 columns per the manufacturer's instructions. The resulting material was concentrated slightly (by ultrafiltration) and purified by size-exclusion chromatography on a Superdex200 column. The monomeric fractions were concentrated and filter sterilized to give the final ADC.

Method B: Site-specific conjugation of linker-payloads to a trastuzumab antibody containing engineered cysteine residues. A solution of trastuzumab containing incorporating an engineered cyst residue, such as cite 118, 334, and 392 (using the EU index of Kabat, see WO2013093809) was prepared in 50 mM phosphate buffer, pH 7.4. PBS, EDTA (0.5 M stock), and TCEP (0.5 M stock) were added such that the final protein concentration was ˜10 mg/mL, the final EDTA concentration was ˜20 mM, and the final TCEP concentration was approximately ˜6.6 mM (100 molar eq.). The reaction was allowed to stand at rt for 2-48 h and then buffer exchanged into PBS using GE PD-10 Sephadex G25 columns per the manufacturer's instructions. Alternative methods such as diafiltration or dialysis are also useful in particular circumstances. The resulting solution was treated with approximately 50 equivalents of dehydroascorbate (50 mM stock in 1:1 EtOH/water). The antibody was allowed to stand at 4° C. overnight and subsequently buffer exchanged into PBS using GE PD-10 Sephadex G25 columns per the manufacturer's instructions. Again, alternative methods such as diafiltration or dialysis are also useful in particular circumstances.

The antibody thus prepared was diluted to ˜2.5 mg/mL in PBS containing 10% DMA (vol/vol) and treated with the appropriate linker-payload (10 molar eq.) as a 10 mM stock solution in DMA. After 2 h at rt, the mixture was buffer exchanged into PBS (per above) and purified by size-exclusion chromatography on a Superdex 200 column. The monomeric fractions were concentrated and filter sterilized to give the final ADC.

TABLE 29
Structure of Tubulysin ADCs and Payload Linkers used to prepare them

| | | LP | | Gen- |
|---|---|---|---|---|
| | | used | Anti- | eral |
| | | for | body | meth- |
| ADC# | Structure | synthesis | used | od |
| ADC#T1 | 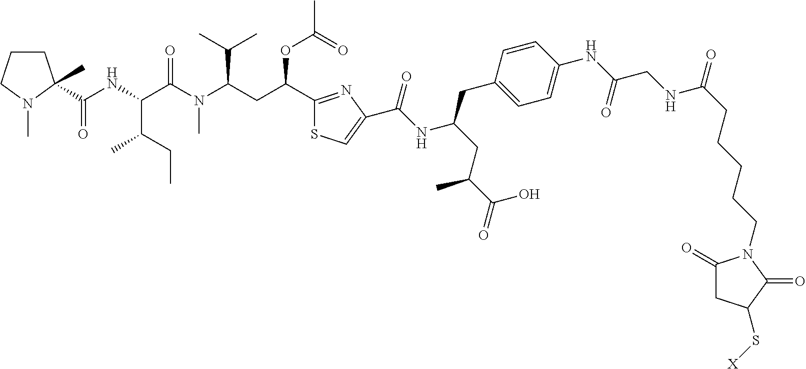 | LP#3 | Tras | A |
| ADC#T2 | 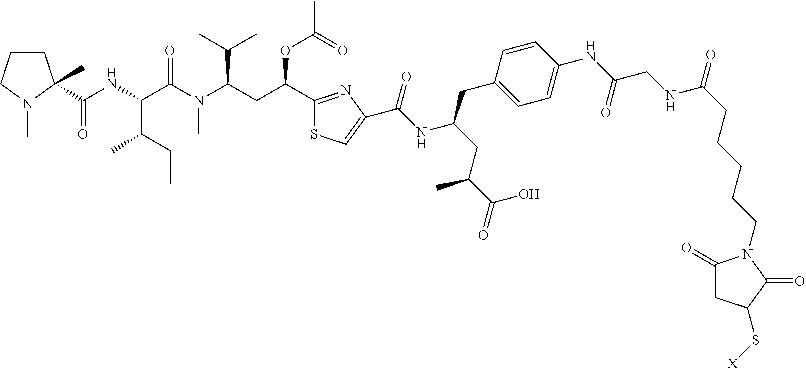 | LP#3 | Tras- C392 | B |
| ADC#T3 | 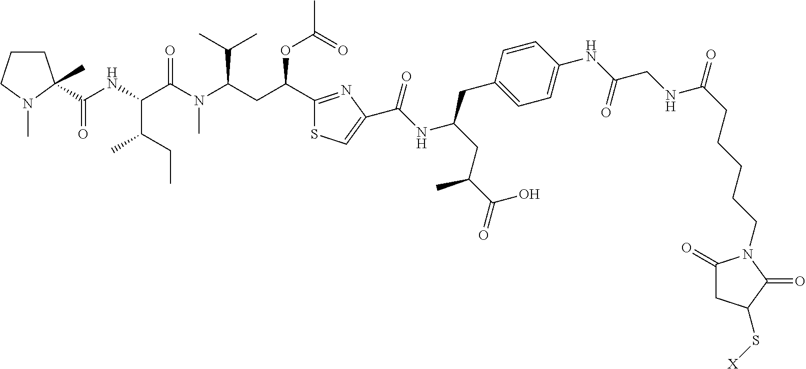 | LP#3 | Tras- C334 | B |
| ADC#T4 | 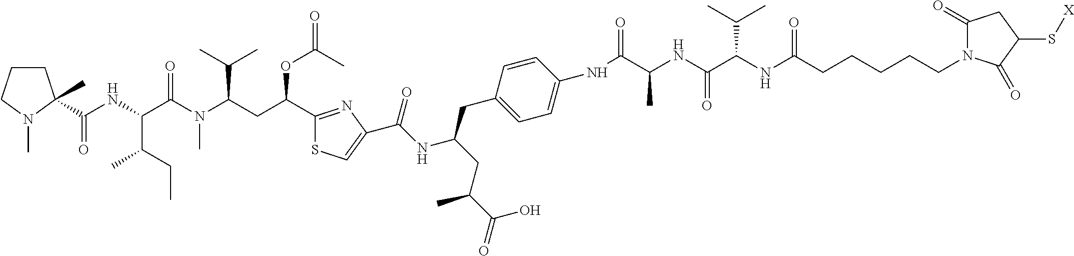 | LP#4 | Tras- C114 | B |
| ADC#T5 | 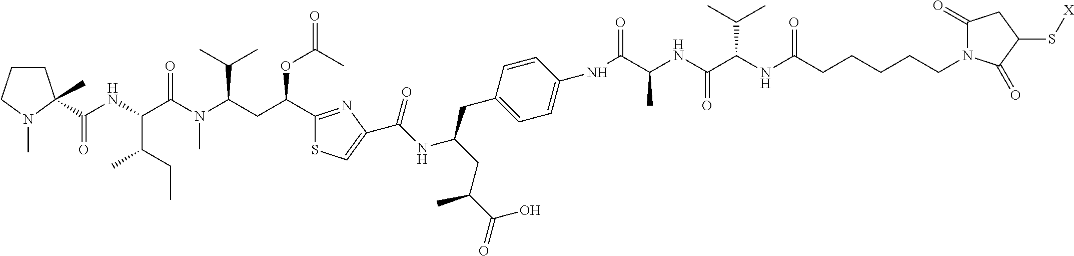 | LP#4 | Tras- C334 | B |
| ADC#T6 | 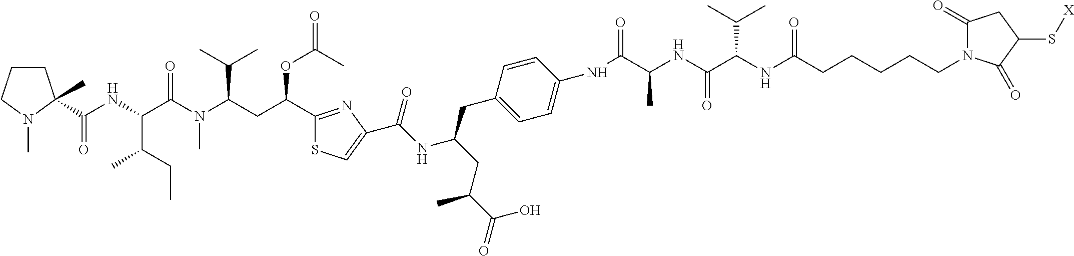 | LP#4 | Tras- C392 | B |

TABLE 30
Analytical Characterization of ADCs

| | | | | Drug per | Drug per |
|---|---|---|---|---|---|
| | | | Observed Δ | Antibody | Antibody |
| | | | mass for | ratio | ratio |
| | | HPLC-HIC | the Heavy | (DAR) | (DAR) |
| | Isolated | retention | Chain (HC) | (LC/MS | (HIC |
| ADC# | yield | time | portion | Method) | Method) |
| ADC#T1 | 79% | 5.43 | 994 | 4.4 | NA |
| ADC#T2 | 75% | 5.54 | 993 | 2.0 | 2.0 |
| ADC#T3 | 52% | 5.19 | 993 | 2.0 | 2.0 |
| ADC#T4 | 34% | NA | 1108 | 2.0 | NA |
| ADC#T5 | 66% | 5.25 | 1102 | 2.0 | 2.0 |
| ADC#T6 | 68% | 5.54 | 1106 | 2.0 | 2.0 |

B. In Vitro Cell Assay Procedure

Target expressing (BT474 (breast cancer), N87 (gastric cancer), HCC1954 (breast cancer), MDA-MB-361-DYT2 (breast cancer)) or non-expressing (HT-29) cells were seeded in 96-well cell culture plates for 24 hours before treatment. Cells were treated with 3-fold serially diluted antibody-drug conjugates or free compounds (no antibody conjugated to the drug) in duplicate at 10 concentrations. Cell viability was determined by CellTiter 96® AQᵤₑₒᵤₛ One Solution Cell Proliferation MTS Assay (Promega) 96 hours after treatment. Relative cell viability was determined as percentage of untreated control. IC₅₀ values were calculated using a four parameter logistic model #203 with XLfit v4.2. Results are shown in Tables 31

TABLE 31
In vitro Cytotoxicity data for selected ADCs

| | N87 | | MDA-MB-361-DYT2 | | HT29 | |
|---|---|---|---|---|---|---|
| | | IC₅₀ of | | IC₅₀ of | | IC₅₀ of |
| | IC₅₀ | Antibody | IC₅₀ | Antibody | IC₅₀ | Antibody |
| ADC# | (nM) | (ng/mL | (nM) | (ng/mL | (nM) | (ng/mL |
| ADC#T1 | 0.353 | 14.403 | 1.59 | 57.5 | 400.7 | 15835.752 |
| ADC#T2 | 0.568 | 43.106 | >1000.000 | >74977.416 | >1000.000 | >74977.416 |
| ADC#T3 | 0.454 | 33.943 | >1000.000 | >72740.113 | 809.98 | 59870.304 |
| ADC#T4 | 0.684 | 55.316 | 0.139 | 9.588 | >1000.000 | >74031.891 |
| ADC#T5 | 0.442 | 33.35 | 0.204 | 15.238 | >1000.000 | >72655.218 |
| ADC#T6 | 0.309 | 23.138 | 0.104 | 7.804 | >1000.000 | >74977.817 |

C. In Vitro Plasma Stability Assay of ADCs

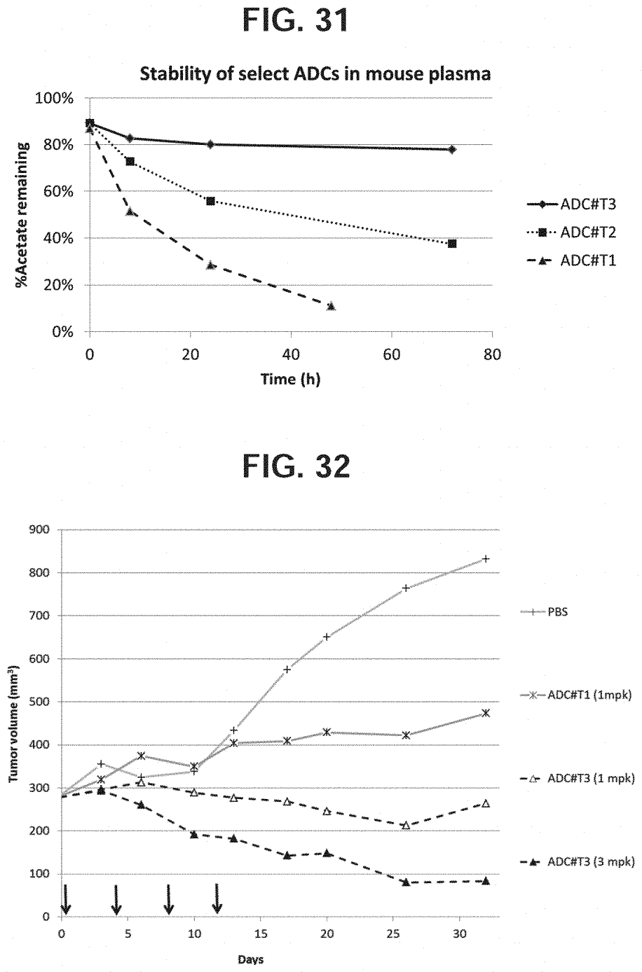

ADC samples (˜1.5 mg/mL) were diluted into mouse plasma (Lampire Biological Laboratories) to yield a final solution of 10% ADC, 90% plasma. Three time points were analyzed to determine their DAR (T-0, T-24hr and T-48 hr). Each time point underwent an immunoprecipitation process to enrich the ADC. Briefly, each aliquot was diluted 1:1 in 20% MPER (Thermo Fisher Scientific) and equal amounts of biotinylated mouse anti-human Fc and goat anti-human kappa antibodies (SouthernBiotech) were added. The samples were incubated for two hours at 4° C. followed by the addition of stretpadvidin Dynabeads (Thermo Fisher Scientific). Samples were processed on a KingFisher instrument with four washing steps consisting of 10% MPER, 0.05% TWEEN 20, and twice with PBS. The ADC was eluted off the beads with 0.15% formic acid. Samples were pH adjusted to 7.8 with 2 M Tris pH 8.5 and their N-linked glycans were removed with PNGaseF (New England Biolabs). The samples were reduced with TCEP and analyzed by LC-MS for % acetate hydrolysed ADC by height of mass shift of 993 (Parent) vs 951 (Deacetylated). The results are shown in FIG. 31. The results illustrate that attachment of tubulysin LP#3 to preferred sites, such as K3340 and K3920, can result in improve ADC plasma stability. This, in turn, is likely to result in improved in vivo exposure and improved efficacy. It is believed that the improved stability imparted by these sites will translate to other payload classes that suffer from metabolism liabilities.

D. In Vivo Stability of ADCs

Blood samples were obtained at 72 h after the final dose of ADC from select tumor bearing mice from N87 xenograft study. Samples were taken from the 3mpk dosing group. The ADC samples thus obtained were deglycosylated by treating with PNGase (New England Biolab) at 37° C. for 1 hour. Following the incubation, a capture antibody (biotinylated goat anti-human Fc at 1.0 mg/mL, Jackson ImmunoResearch) was added and the mixture was heated at 37° C. for one hour followed by gentle shaking at room temperature for a second hour. Dynabead MyOne Streptavidin T1 beads (Invitrogen) were added to the samples and incubated at room temperature for at least 30 minutes while gently shaking. The sample plate was then washed with 200 μL PBS+0.05% Tween 20, 200 μL PBS, and HPLC grade water. The bound ADC was eluted with 55 μL 2% of formic acid (v/v). Fifty microliters of each sample were transferred into a new plate followed by an additional 5 μL of 200 mM TCEP.

The intact protein analysis was carried out with Xevo G2 QTof mass spectrometer coupled with Nano Acquity (waters) and BEH300 C₄, 1.7 μm, 0.3×100 mm column (Waters), using Masslynx v4.1 as acquisition software. The column temperature was set at 85° C. Mobile phase A consisted of 0.1% TFA (TFA) in water. Mobile phase B consisted of 0.1% TFA in acetonitrile: 1-propanol (1:1, v/v). The chromatographic separation was achieved at a flow rate of 18 μL/min using a linear gradient of mobile phase B from 5 to 90% over 7 minutes. Data analysis including deconvolution was performed using Biopharmalynx v1.33 (Waters). The results are shown in Table 32. The results indicate that the tubulysin conjugate at the 334 site (ADC#T3) has improved in vivo stability as compared to the hinge (conventional) conjugate ADC#T1.

TABLE 32
In vivo Stability of ADCs (as measured by PAR)

| | DAR at 0 h | DAR at 72 h | % DAR |
|---|---|---|---|
| Example | post-dose | post-dose | remaining at 72 h |
| ADC#T1 | 3.8 | 0.6 | 16% |
| ADC#T3 | 1.8 | 1.7 | 95% |

E. In Vivo N87 Tumor Xenograft Model:

In vivo efficacy studies of antibody-drug conjugates were performed with target expressing xenograft models using the N87 cell lines. For efficacy study, 7.5 million tumor cells in 50% matrigel are implanted subcutaneously into 6-8 weeks old nude mice until the tumor sizes reach between 250 and 350 mm. Dosing is done through bolus tail vein injection. Depending on the tumor response to treatment, animals are injected with 1-10 mg/kg of antibody drug conjugates treated four times every four days. All experimental animals are monitored for body weight changes weekly. Tumor volume is measured twice a week for the first 50 days and once weekly thereafter by a Caliper device and calculated with the following formula: Tumor volume 5=(length×width)/2. Animals are humanely sacrificed before their tumor volumes reach 2500 mm. The tumor size is observed to decrease after the first week of treatment. Animals may be monitored continuously for tumor re-growth after the treatment has discontinued. Results of the testing of ADC #T1 and #T3 in the N87 mouse xenograft in vivo screening model is shown in FIG. 32. The results illustrate that attachment of LP#3 to preferred sites, such as K334C, may result in improved in vivo efficacy. The improved efficacy is likely the result of improved ADC stability of ADC#T3 (the 334C conjugate) as compared to ADC#T1 (the conventional hinge conjugate). Note that the efficacy of ADC#T3 is significantly greater than ADC#T1 in spite of the fact that ADC#T3 is one half of the DAR of ADC#T1.

### Example 23: Site-Specific ADCs using Anti-EDB Antibody

In this example, efficacy and PK profile of ADCs based on anti-EDB antibodies were investigated. Sequences of exemplary anti-EDB antibody, L19, are shown in Table 33. The data provided in this example also include L19 mutants where certain mutations (which did not affect the binding affinity of L19) were introduced. The CDR sequences of these mutants are identical to that of L19. For example, EDB-(H16-K222R) is a L19 mutant used for transglutaminase-based ADC conjugation. Additional details of these ADCs, and ADCs targeting EDB in general, are described in detail in U.S. Provisional Application 62/409,081, filed Oct. 17, 2016, and is herein incorporated by reference in its entirety.

TABLE 33
Sequences of anti-EDB antibodies

| SEQ | | |
|---|---|---|
| ID | | |
| NO. | Description | Sequence |
| 65 | EDB-L19 VH | EVQLLESGGGLVQPGGSLRLSCAASGFTFSSFSMSWVRQAPGKGLEWVSSISG |
| | Protein | SSGTTYYADSVKGRFTISRDNSKNTLYLQMNSLRAEDTAVYYCAKPFPYFDYW |
| | | GQGTLVTVSS |
| 66 | EDB L19 VH CDR1 | SFSMS |
| | Kabat | |
| 67 | EDB-L19 VH CDR1 | GFTFSSF |
| | Chothia | |
| 68 | EDB-L19 VH CDR2 | SISGSSGTTYYADSVKG |
| | Kabat | |
| 69 | EDB-L19 VH CDR2 | SGSSGT |
| | Chothia | |
| 70 | EDB-L19 VH CDR3 | PFPYFDY |
| | Kabat/Chothia | |
| 71 | EDB-L19 HC | EVQLLESGGGLVQPGGSLRLSCAASGFTFSSFSMSWVRQAPGKGLEWVSSISG |
| | Human IgG1 | SSGTTYYADSVKGRFTISRDNSKNTLYLQMNSLRAEDTAVYYCAKPFPYFDYW |
| | Protein | GQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWLNGKEY |
| | | KCKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNVFSC |
| | | SVMHEALHNHYTQKSLSLSPGK |
| 72 | EDB-L19 VL | EIVLTQSPGTLSLSPGERATLSCRASQSVSSSFLAWYQQKPGQAPRLLIYYAS |
| | Protein | SRATGIPDRFSGSGSGTDFTLTISRLEPEDFAVYYCQQTGRIPPTFGQGTKVE |
| | | IK |
| 73 | EDB-L19 VL CDR1 | RASQSVSSSFLA |
| | Kabat/Chothia | |
| 74 | EDB-L19 VL CDR2 | YASSRAT |
| | Kabat/Chothia | |
| 75 | EDB-L19 VL CDR3 | QQTGRIPPT |
| | Kabat/Chothia | |
| 76 | EDB-L19 LC | EIVLTQSPGTLSLSPGERATLSCRASQSVSSSFLAWYQQKPGQAPRLLIYYAS |
| | Human Kappa | SRATGIPDRFSGSGSGTDFTLTISRLEPEDFAVYYCQQTGRIPPTFGQGTKVE |
| | Protein | IKRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFNR |
| | | GEC |
| 77 | EDB-(K290C) HC | EVQLLESGGGLVQPGGSLRLSCAASGFTFSSFSMSWVRQAPGKGLEWVSSISG |
| | Protein | SSGTTYYADSVKGRFTISRDNSKNTLYLQMNSLRAEDTAVYYCAKPFPYFDYW |
| | | GQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDK |
| | | KVEPKSCDKTHTCPPCPAPELLGGPSVFLFPPKPKDTLMISRTPEVTCVVVDV |
| | | SHEDPEVKFNWYVDGVEVHNAKTCPREEQYNSTYRVVSVLTVLHQDWLNGKEY |
| | | KCKVSNKALPAPIEKTISKAKGQPREPQVYTLPPSRDELTKNQVSLTCLVKGF |
| | | YPSDIAVEWESNGQPENNYKTTPPVLDSDGSFFLYSKLTVDKSRWQQGNVFSC |
| | | SVMHEALHNHYTQKSLSLSPG |
| 78 | EDB-(κK183C) LC | EIVLTQSPGTLSLSPGERATLSCRASQSVSSSFLAWYQQKPGQAPRLLIYYAS |
| | Protein | SRATGIPDRFSGSGSGTDFTLTISRLEPEDFAVYYCQQTGRIPPTFGQGTKVE |
| | | IKRTVAAPSVFIFPPSDEQLKSGTASVVCLLNNFYPREAKVQWKVDNALQSGN |
| | | SQESVTEQDSKDSTYSLSSTLTLSCADYEKHKVYACEVTHQGLSSPVTKSFNR |
| | | GEC |

23.1. In Vitro Binding of EDB ADCs

To assess the relative binding of anti-EDB antibodies and ADCs to EDB, MaxiSorp 96-well plates were coated with 0.5 or 1 μg/ml of human 7-EDB-89 in PBS and incubated overnight at 4° C. with gentle shaking. Plates were then emptied, washed with 200 μl PBS and blocked with 100 μl of Blocking Buffer (ThermoScientific) for 3 hours at room temperature. Blocking buffer was removed, wells were washed with PBS and incubated with 100 μl of anti-EDB antibodies or ADCs which were serially diluted (4-fold) in ELISA Assay Buffer (EAB; 0.5% BSA/0.02% Tween-20/PBS). The first column of the plate was left empty and the last column of the plate was filled with EAB as blank controls. The plate was incubated at room temperature for 3 hours. Reagents were removed and plate washed with 200 μl of 0.03% Tween-20 in PBS (PBST). Anti-human IgG-Fc-HRP (Thermo/Pierce) diluted 1:5000 in EAB was added as 100 μl to the wells and incubated for 15 minutes at room temperature. The plate was washed with 200 μl of PBST, then 100 μl of BioFX TMB (Fisher) was added and the color allowed to develop for 4 minutes at room temperature. The reaction was stopped with 100 μl of 0.2N sulfuric acid and absorbance at 450 nm was read on a Victor plate reader (Perkin Elmer, Waltham, Mass.).

Table 34 provides the relative binding of anti-EDB antibodies and ADCs to human 7-EDB-89 protein fragment bound to a 96-well plate in ELISA format. All antibodies and ADCs targeting EDB bound to the target protein with similar affinity in the range of 19 pM to 58 pM. In contrast, non-EDB targeting antibodies and ADCs have high EC₅₀ values >10,000 pM.

TABLE 34
Anti-EDB antibody and ADC binding to human EDB.

| ADC or | | Avg EC₅₀ | |
|---|---|---|---|
| Antibody ID | ADC or Antibody Name | (pM) | SD |
| Ab1 | EDB-L19 | 27.0 | — |
| ADC1 | EDB-L19-vc-0101 | 37.8 | 12.8 |
| ADC11 | Neg-vc-0101 | >10,000 | — |
| Ab2 | EDB-(κK183C-K290C) | 30.2 | 1.6 |
| ADC2 | EDB-(κK183C-K290C)-vc-0101 | 58.4 | 17.0 |
| ADC12 | Neg-(κK183C-K290C)-vc-0101 | >10,000 | ND |
| Ab3 | EDB-mut1 | 15.0 | — |
| ADC3 | EDB-mut1-vc-0101 | 37.1 | 14.6 |
| Ab4 | EDB-mut1(κK183C-K290C) | 44.8 | 8.7 |
| ADC4 | EDB-mut1(κK183C-K290C)-vc-0101 | 56.7 | 13.5 |
| ADC5 | EDB-L19-diS-DM1 | 21.3 | — |
| ADC6 | EDB-L19-diS-C₂OCO-1569 | 30.7 | — |
| ADC7 | EDB-L19-VC-9411 | 37.5 | — |
| ADC15 | Neg-vc-9411 | >10,000 | — |
| ADC8 | EDB-L19-diS-4574 | 31.9 | — |
| Ab5 | EDB-(H16-K222R) | 19.3 | — |
| ADC9 | EDB-(H16-K222R)-AcLys-vc-8314 | 39.4 | 2.5 |
| ADC17 | Neg-(H16-K222R)-AcLys-vc-8314 | >10,000 | — |

Mean EC₅₀ ± standard deviation and number (n) of determinations.
ND = not determined.

23.2: In Vitro Cytotoxicity of Anti-EDB ADCs

Cell Culture. WI38-VA13 are SV40-transformed human lung fibroblasts obtained from ATCC and maintained in MEM Eagles media (Cell-Gro), supplemented with 10% FBS, 1% MEM non-essential amino acids, 1% sodium pyruvate, 100 units/ml penicillin-streptomycin, and 2 mM GlutaMax. HT29 are derived from human colorectal carcinoma (ATCC) and maintained in DMEM media supplemented with 10% FBS and 1% glutamine.

EDB+ FN transcript detection. For gene expression and transcript analysis of EDB+ FN, adherent proliferating WI38-VA13 and HT29 cells were dissociated from cell-culture flasks with TrypLE Express (Gibco). The RNeasy Mini Kit (Qiagen) was used to purify total RNA from the collected cell pellets. The residual DNA was removed by RNase-Free DNase Set (Qiagen) during RNA purification. High Capacity RNA-to-cDNA Kit (Applied Biosystems) was used for reverse transcription of total RNA to cDNA. The cDNA was analyzed by quantitative real-time PCR using TaqMan Universal Master Mix II, with UNG (Applied Biosystems). EDB+ FN1 signal was detected by TaqMan primer Hs01565271_m1 and normalized with the average of both signals from ACTB (TaqMan primer Hs99999903_m1) and GAPDH (TaqMan primer Hs99999905_m1). All primers were from ThermoFisher Scientific. Data from a representative experiment is shown.

EDB+ FN protein detection by western blotting. For detection of EDB+FN by western blotting, adherent proliferating WI38-VA13 and HT29 cells were harvested by cell scraping. Cell lysates were prepared in Cell Lysis Buffer (Cell Signaling Technology) with protease inhibitors and phosphatase inhibitors. Tumor lysate was prepared in either RIPA Lysis Buffer or 2× Cell Lysis Buffer (Cell Signaling Technology) with protease inhibitors and phosphatase inhibitors. Protein lysates were analyzed by SDS-PAGE and followed by western blotting. Proteins were transferred to nitrocellulose membrane and then blocked with 5% milk/TBS, followed by incubation with EDB-L19 antibody and anti-GAPDH antibody (Cell Signaling Technology) overnight at 4° C. After washing, the anti-EDB blot was incubated with ECL HRP-linked anti-human IgG secondary antibody (GE Healthcare) for 1 hour at room temperature. After washing, the EDB+ FN signal was developed by Pierce ECL 2 Western Blotting Substrate (Thermo Scientific) and detected by X-ray films. The anti-GAPDH blot was incubated with Alexa Fluor 680 conjugated anti-rabbit IgG secondary antibody (Invitrogen) in blocking buffer for 1 hour at room temperature. After washing, the GAPDH signal was detected by LI-COR Odyssey Imaging System. Densitometric analysis of EDB western blots was conducted using the Bio-Rad GS-800 Calibrated Imaging Densitometer and quantified using Quantity One version 4.6.9 software. Data from a representative experiment is shown.

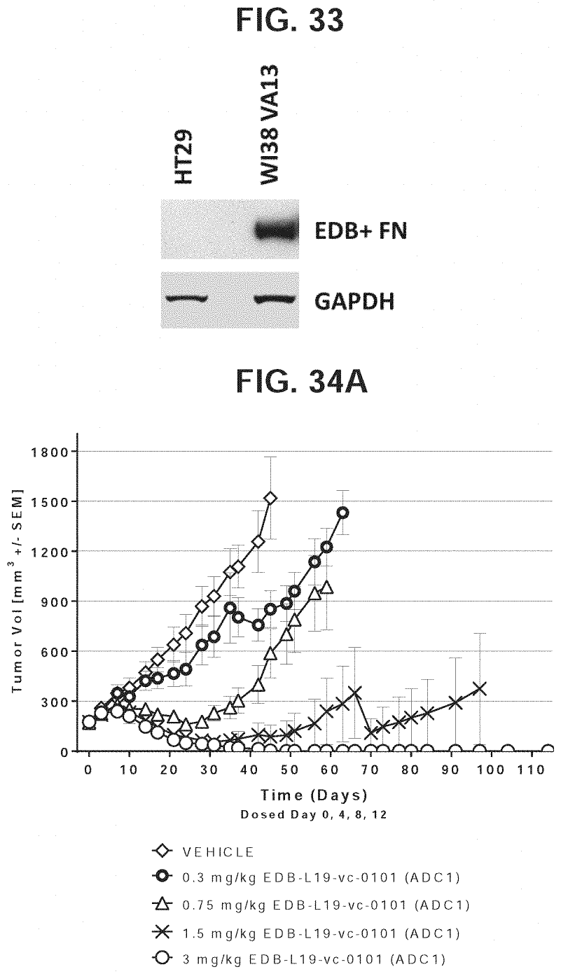

FIG. 33 shows EDB+ FN1 expression by western blot in WI38-VA13 and HT29 cells. EDB+ FN is expressed in the WI38-VA13 cell line but not in HT29 colon carcinoma cell line when grown in vitro.

EDB+ FN Protein detection by flow cytometry. EDB-L19 antibody was used to measure the expression of EDB+ FN on the cell surface of WI38-VA13 or HT29 cells by flow cytometry. Cells were dissociated by non-enzymatic cell dissociation buffer (Gibco) and incubated with cold flow buffer (FB, 3% BSA/PBS+Ca+Mg) on ice for blocking. Cells were then incubated with primary antibodies on ice in FB. After the incubation, cells were washed with cold PBS−Ca−Mg and then incubated with viability stain (Biosciences) to discriminate live and dead cells, according to the manufacture's procedure. The signals were analyzed on a BD Fortessa flow cytometer and data were analyzed using BD FACS DIVA software. Data from a representative experiment is shown.

Table 35 summarizes the results from western blot, qRT-PCR and flow cytometry. The data demonstrates that WI38-VA13 is EDB+ FN positive and HT29 is EDB+ FN negative.

TABLE 35
Characterization of EDB+ FN
expression in WI38 VA13 and HT29 cells

| | | Western | Flow cytometric binding |
|---|---|---|---|
| Cell | qRT-PCR | (normalized | (MFI-GeoMean |
| Line | (2^{(-ddC(t)}) | density (OD/mm2)) | (EDB unstained)) |
| WI38-VA13 | 0.224247 | 475.397 | 4480 |
| HT29 | 0.000049 | 0.093 | 2 |

In vitro cytotoxicity assays. Proliferating WI38-VA13 or HT29 cells were harvested from culture flasks with non-enzymatic cell dissociation buffer and cultured overnight in 96-well plates (Corning) at 1000 cells/well in a humidified chamber (37° C., 5% CO₂). The next day, cells were treated with anti-EDB ADCs or isotype control non-EDB-binding ADCs by adding 50 μl of 3× stocks in duplicate at 10 concentrations. In some experiments, cells were plated at 1500 cells/well and treated the same day. Cells were then incubated with anti-EDB ADCs or isotype control non-EDB-binding ADCs for four days. On harvest day, 50 μl of Cell Titer Glo (Promega) was added to the cells and incubated 0.5 hours at room temperature. Luminescence was measured on a Victor plate reader (Perkin Elmer, Waltham, Mass.). Relative cell viability was determined as a percentage of untreated control wells. IC₅₀ values were calculated using four-parameter logistic model #203 with XLfit v4.2 (IDBS).

Table 36 shows the IC₅₀ (ng/ml of antibody) of the anti-EDB ADC treatments in cytotoxicity assays performed on WI38-VA13 (EDB+ FN positive tumor cell line) and HT29 colon carcinoma cells (EDB+ FN negative tumor cell line). The anti-EDB ADCs induced cell death in the EDB+ FN expressing cell line. The IC₅₀ values were similar for all anti-EDB ADCs having vc-0101 linker-payload, in the range of approximately 184 ng/ml to 216 ng/ml (EDB-L19-vc-0101, EDB-(KK183C-K2900)-vc-0101, EDB-mut1-vc-0101, EDB-mut1(KK1830 -K2900)-vc-0101). The negative control vc-0101 ADCs were substantially less potent, with IC₅₀ values approximately 70- to 200-fold higher than anti-EDB-vc-0101 ADCs. All vc-0101 ADCs had 46- to 83-fold higher IC₅₀ values in the EDB+ FN negative tumor cell line, HT29. Therefore, anti-EDB ADCs were dependent on EDB+ FN expression for their in vitro cytotoxicity.

Other auristatin-based anti-EDB ADCs with “vc” protease-cleavable linkers, EDB-L19-vc-9411and EDB-L19-vc-1569, also showed potent cytotoxicity in WA38-VA13 cells with high selectivity of about 50- to 180-fold compared with the corresponding negative control ADCs and selectivity of about 25- to 140-fold compared with the non-expressing cell line. The EDB-L19-diS-DM1 ADC had similar potency as the vc-0101 ADCs, however much lower selectivity compared with the negative control ADC (about 3-fold) and with HT29 cells (about 0.9-fold).

TABLE 36
In vitro cytotoxicity of anti-EDB ADCs and control non-EDB-binding ADCs.

| | | WI38-VA13 | | | HT29 | | |
|---|---|---|---|---|---|---|---|
| ADC | | Avg | | | Avg | | |
| ID# | ADC Name | IC₅₀ | SD | n | IC₅₀ | SD | n |
| ADC1 | EDB-L19-vc-0101 | 184 | 143 | 23 | 15,346 | 4448 | 5 |
| ADC11 | Neg-vc-0101 | 19,585 | 6762 | 16 | 10,731 | 8193 | 24 |
| ADC2 | EDB-(κK183C-K290C)-vc-0101 | 198 | 176 | 6 | 9,276 | 83 | 2 |
| ADC12 | Neg-(κK183C-K290C)-vc-0101 | >40,000 | ND | 4 | 21,913 | 2635 | 2 |
| ADC3 | EDB-mut1-vc-0101 | 184 | 138 | 7 | 10,577 | 2065 | 2 |
| ADC4 | EDB-mut1(κK183C-K290C)-vc-0101 | 216 | 94 | 6 | 15,584 | 58 | 3 |
| ADC5 | EDB-L19-diS-DM1 | 268 | 150 | 8 | 237 | 180 | 2 |
| ADC13 | Neg-diS-DM1 | 879 | 82 | 5 | ND | ND | ND |
| ADC6 | EDB-L19-diS-C2OCO-1569 | 21 | 8 | 6 | 5 | 3 | 2 |
| ADC14 | Neg-diS-C2OCO-1569 | 36 | 6 | 3 | ND | ND | ND |
| ADC7 | EDB-L19-vc-9411 | 46 | 22 | 3 | 1,153 | — | 1 |
| ADC15 | Neg-vc-9411 | 2,514 | 260 | 3 | 1,243 | — | 1 |
| ADC8 | EDB-L19-diS-4574 | 487 | 406 | 4 | 429 | 228 | 2 |
| ADC16 | Neg-diS-4574 | 1,279 | — | 1 | ND | ND | ND |
| ADC9 | EDB-(H16-K222R)-AcLys-vc-CPI | 34 | 30 | 5 | 3,449 | — | 1 |
| ADC17 | Neg-AcLys-vc-CPI | 2,656 | 876 | 3 | 15,110 | 15,408 | 2 |
| ADC10 | EDB-L19-vc-1569 | 40 | 11 | 2 | 5,702 | — | 1 |
| ADC18 | Neg-vc-1569 | 7283 | — | 1 | ND | ND | ND |

Mean IC₅₀ ± standard deviation and number (n) of determinations.
ND = not determined.

### 23.3: In Vivo Efficacy of Site-Specific EDB ADCs

Anti-EDB ADCs were evaluated in cell line xenograft (CLX), patient derived xenograft (PDX) and syngeneic tumor models. Expression of EDB+ FN was detected using an immunohistochemical (IHC) assay as previously described herein.

To generate CLX models, 8×10⁶ to 10×10⁶ cells of H-1975, HT29, or Ramos tumor lines were implanted into female athymic nude mice subcutaneously. Ramos and H-1975 cells for inoculation were suspended in 50% and 100% Matrigel (BD Biosciences), respectively. For the Ramos model, the animals received whole body irradiation (4 Gy) before cell inoculation to facilitate the establishment of tumors. When the average tumor volume reached approximately 160 to 320 mm³, the animals were randomized into treatment groups, with 8-10 mice in each group. ADCs or vehicle (PBS) were administered intravenously on day 0 and then the animals were dosed once every 4 days for 4 to 8 doses. Tumors were measured once or twice weekly and tumor volume was calculated as volume (mm³)=(width×width×length)/2. The body weight of animals was monitored for 4 to 9 weeks and no animal weight loss was observed in any treatment groups.

To generate PDX models, tumors were collected from donor animals and tumor fragments approximately 3×3 mm were implanted subcutaneously into the flank of female athymic nude mice (for PDX-NSX-11122 model) or NOD SCID mice (for PDX-PAX-13565 and PDX-PAX-12534 models) by using a 10 gage trocar. When average tumor volume reached approximately 160 to 260 mm³ the mice were randomized into treatment groups, with 7-10 mice in each group. ADCs or vehicle (PBS) dosing regime and administration route as well as tumor measurement procedures are the same as described above for CLX models. The body weight of animals was monitored for 5 to 14 weeks and no animal weight loss was observed in any treatment groups. Tumor growth inhibition is plotted as an average of tumor size±SEM.

Expression of EDB+ FN. As shown in Table 37, expression of EDB+ FN in the H-1975, HT29 and Ramos CLX models, PDX-NSX-11122, PDX-PAX-13565 and PDX-PAX-12534 PDX models and EMT-6 syngeneic syngeneic tumor models was measured by binding of EDB-L19 antibody and subsequent detection in IHC assay. The CLX HT-29 was a moderate expressing CLX however was negative when examined in vitro due to the predominance of protein expression in the CLX being derived from the tumor stroma.

TABLE 37
Expression of EDB+ FN

| Efficacy | | EDB + FN Overall |
|---|---|---|
| Model | Tumor Type | Expression |
| PDX-NSX-11122 | NSCLC PDX | High |
| EMT-6 | Syngeneic mouse mammary | High |
| | carcinoma (breast) | |
| PDX-PAX-13565 | Pancreatic adenocarcinoma PDX | Moderate/High |
| H-1975 | NSCLC CLX | Moderate/High |
| HT29 | Colorectal cancer CLX | Moderate |
| Ramos | Burkitt's lymphoma CLX | Moderate |
| PDX-PAX-12534 | Pancreatic adenocarcinoma PDX | Low/Moderate |

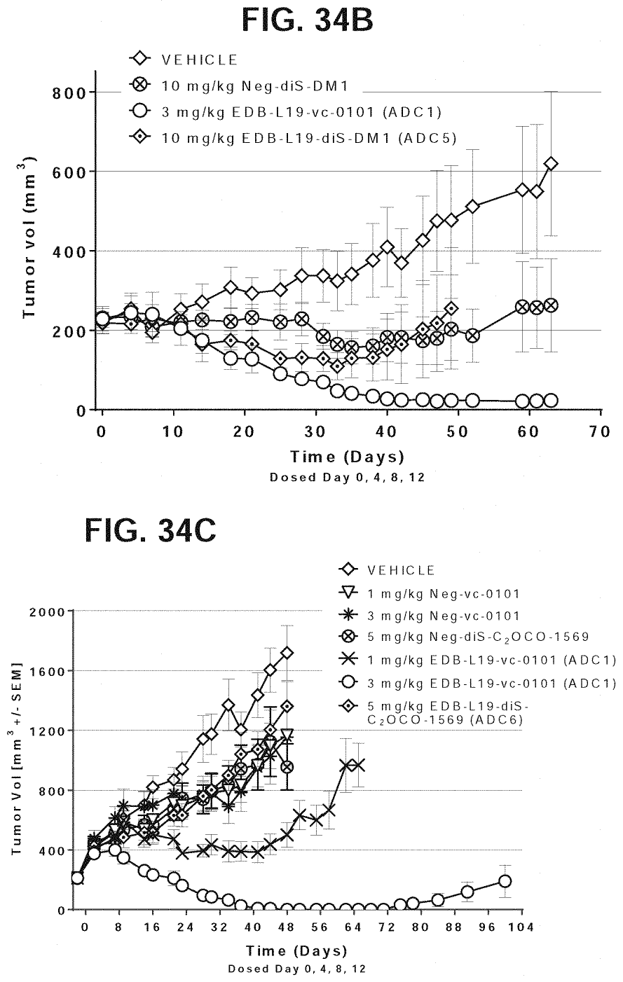

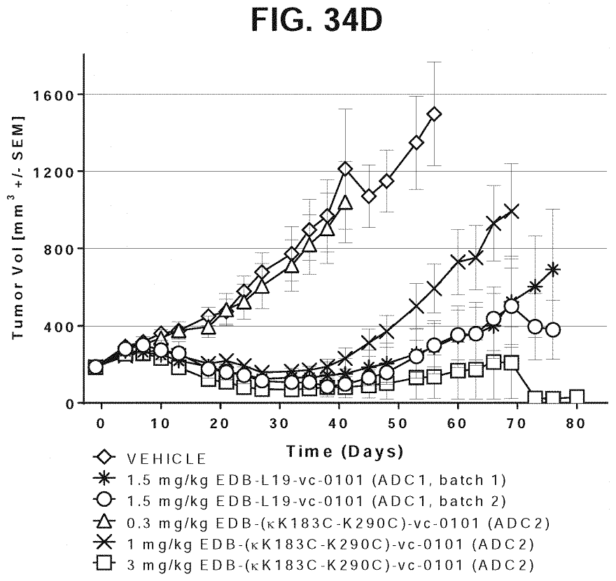

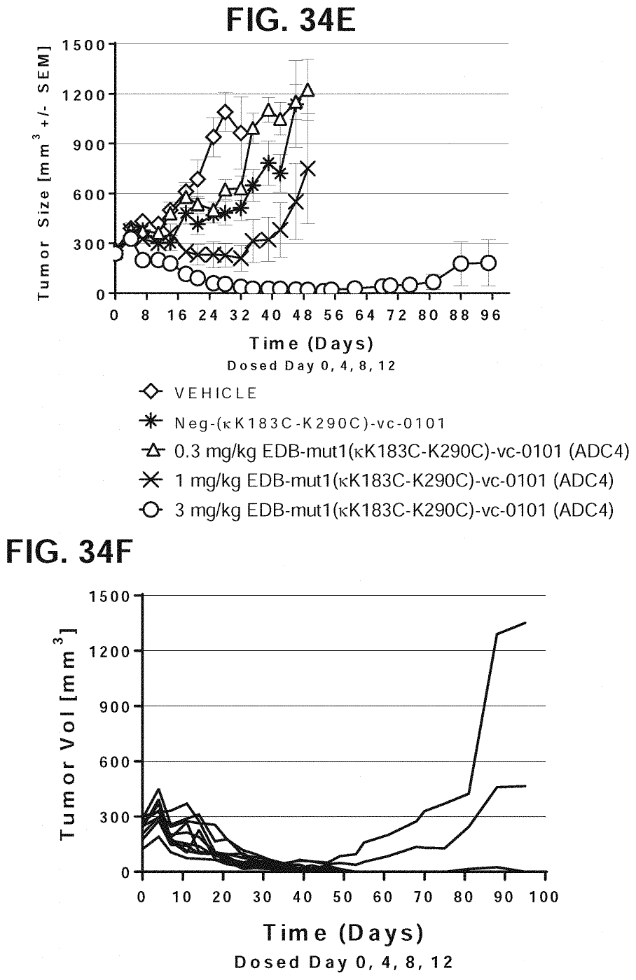

PDX-NSX-11122 NSCLC PDX. The effects of various ADCs were evaluated in PDX-NSX-11122, a NSCLC PDX model of human cancer that expresses high levels of EDB+ FN. FIG. 34A shows the anti-tumor activity for EDB-L19-vc-0101 (ADC1) at 0.3, 0.75, 1.5 and 3 mg/kg. The data demonstrates that EDB-L19-vc-0101 (ADC1) showed tumor regression in a dose dependent manner at 3 mg/kg and 1.5 mg/kg.

Anti-tumor efficacy of vc-linked ADCs was compared to disulfide-linked ADCs. FIGS. 34B and 34C show the anti-tumor activity of EDB-L19-vc-0101 (ADC1) at 3 mg/kg as compared to 10 mg/kg of disulfide linked EDB-L19-diS-DM1 (ADC5), and EDB-L19-vc-0101 (ADC1) at 1 and 3 mg/kg as compared to 5 mg/kg of disulfide linked EDB-L19-diS-C₂OCO-1569 (ADC6), respectively. As shown in FIGS. 34B and 34C, EDB-L19-vc-0101 (ADC1) demonstrated greater efficacy as compared to isotype negative control ADCs and ADCs that were generated using a disulfide linker, EDB-L19-diS-DM1 and EDB-L19-dis-C₂OCO-1569. Further, animals bearing tumors that were treated with EDB-L19-vc-0101 (ADC1) had delayed tumor growth at 1 mg/kg and complete regressions at 3 mg/kg. The data demonstrates that EDB-L19-vc-0101 (ADC1) inhibits growth of PDX-NSX-11122 NSCLC xenografts in a dose-dependent manner.

The activity of site-specific and conventionally conjugated ADCs was evaluated. FIG. 34D shows the anti-tumor efficacy of the site-specific conjugated EDB-(KK183C+K290C)-vc-0101 (ADC2) compared to the conventionally conjugated EDB-L19-vc-0101 (ADC1) at the doses of 0.3, 1 and 3 mg/kg and 1.5 mg/kg, respectively. The dose-level based efficacy was comparable and the EDB-(KK183C+K290C)-vc-0101 (ADC2) led to tumor regression in a dose dependent manner.

The activity of vc-0101 anti-EDB ADCs having various mutations was assessed. FIG. 34E shows the anti-tumor efficacy of site-specific conjugated EDB-mut1(κK183C-K2900)-vc-0101 (ADC4) at the doses of 0.3, 1 and 3 mg/kg. EDB-mut1(κK183C-K290C)-vc-0101 (ADC4) induced tumor regression at 1 and 3 mg/kg. FIG. 34F shows the tumor growth inhibition curves for the 10 individual tumor bearing mice in the EDB-mut1(κK183C-K290C)-vc-0101 (ADC4) group dosed at 3 mg/kg of FIG. 34E. The tumor regressions in the 3 mg/kg group were complete and durable in 8 of 10 mice (80%) at the end of the study (95 days).

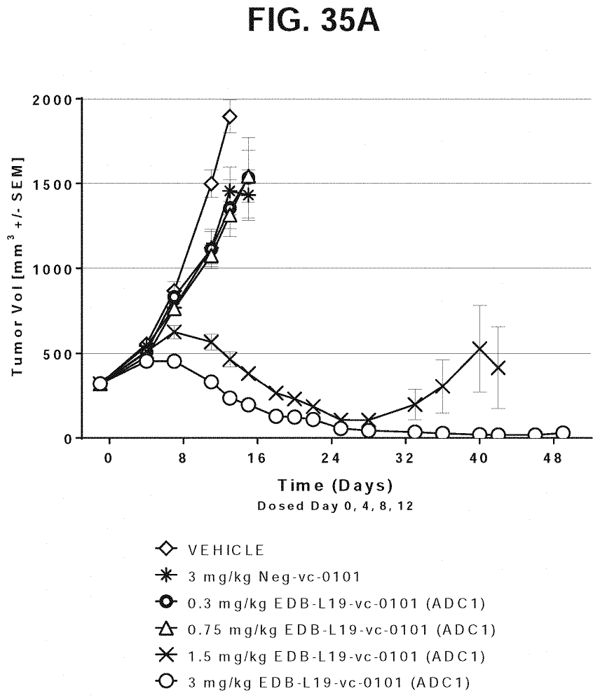

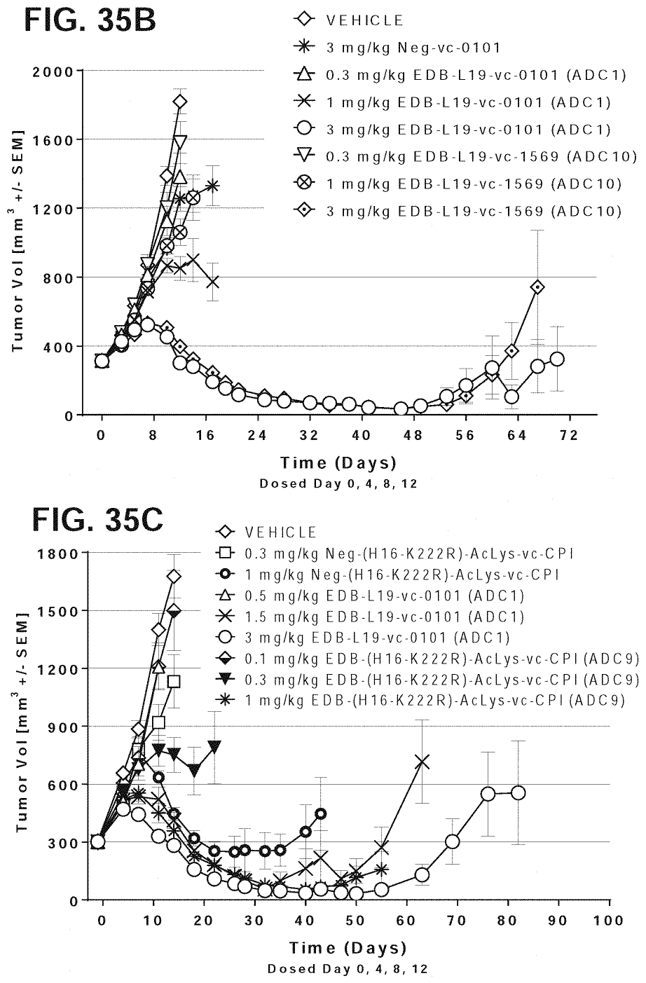

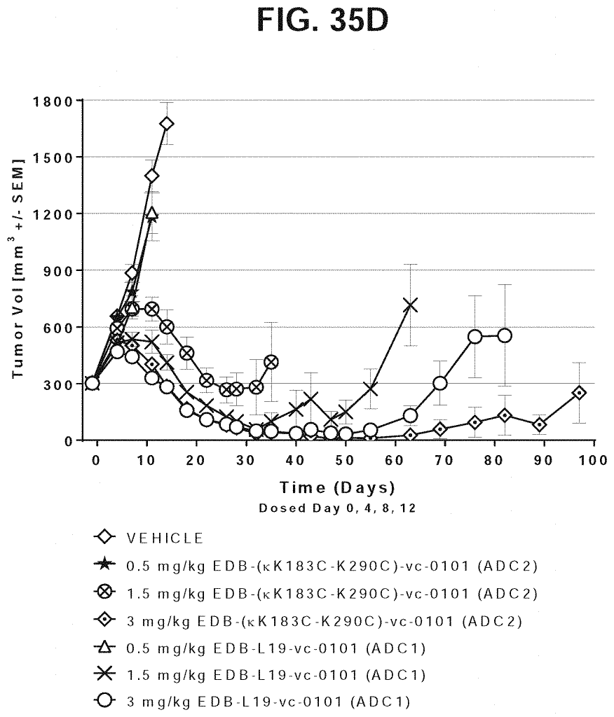

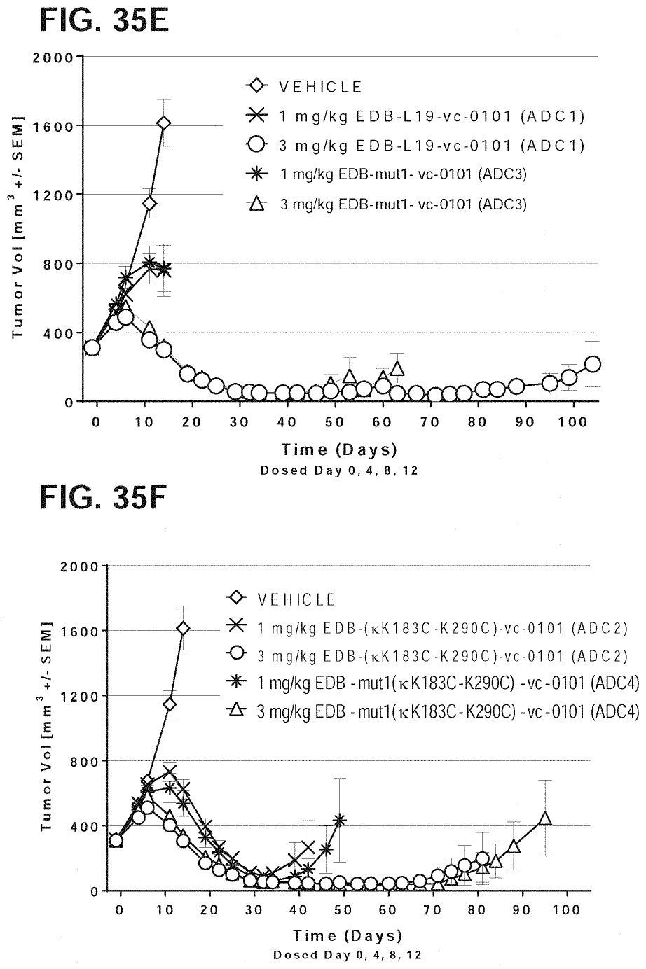

H-1975 NSCLC CLX. The effects of various vc-linked auristatin and CPI ADCs were evaluated in H-1975, a moderate to high EDB+ FN expressing NSCLC CLX model of human cancer. FIG. 35A shows EDB-L19-vc-0101 (ADC1) assessed for anti-tumor activity at 0.3, 0.75, 1.5 and 3 mg/mg. The data demonstrates that EDB-L19-vc-0101 (ADC1) showed tumor regression in a dose dependent manner at 3 mg/kg, and at as low as 1.5 mg/kg. FIG. 35B shows EDB-L19-vc-0101 (ADC1) and EDB-L19-vc-1569 (ADC10) were evaluated for anti-tumor activity at 0.3, 1 and 3 mg/kg. The data demonstrates that EDB-L19-vc-0101 (ADC1) and EDB-L19-vc-1569 (ADC10) showed tumor regression in a dose dependent manner.

The anti-tumor activity of vc-linked auristatin ADCs were compared to CPI ADCs. As shown in FIG. 35C, EDB-L19-vc-0101 (ADC1) and EDB-(H16-K222R)-AcLys-vc-CPI (ADC9) were assessed at 0.5, 1.5 and 3 mg/kg and 0.1, 0.3 and 1 mg/kg, respectively. EDB-L19-vc-0101 (ADC1) and EDB-(H16-K222R)-AcLys-vc-CPI (ADC9) a both showed tumor regression at the highest doses evaluated.

The activity of site-specific and conventionally conjugated anti-EDB ADCs was evaluated. FIG. 35D shows the anti-tumor efficacy of the site-specific conjugated EDB-(KK183C+K290C)-vc-0101 (ADC2) compared to conventionally conjugated EDB-L19-vc-0101 (ADC1) at the doses of 0.5, 1.5 and 3 mg/kg. The dose-level based efficacy was comparable and the EDB-(KK183C+K290C)-vc-0101 (ADC2) led to tumor regression in a dose dependent manner.

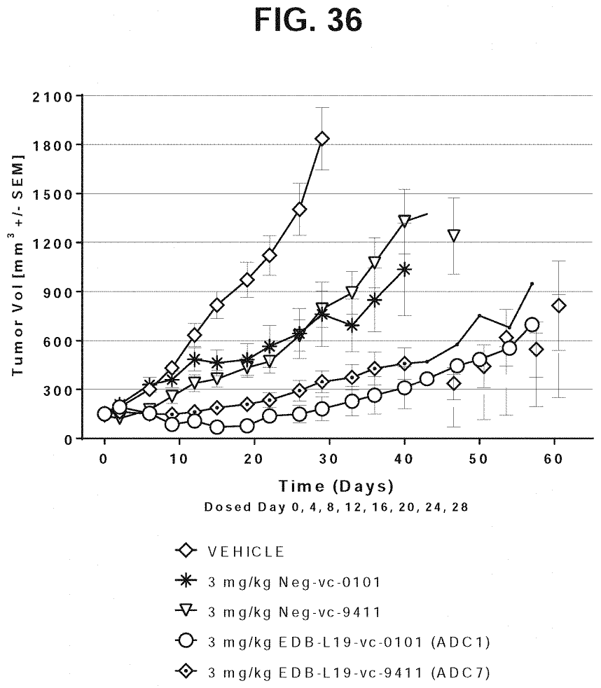

The activity of vc-0101 anti-EDB ADCs having various mutations was assessed. FIG. 35E shows the anti-tumor efficacy of EDB-L19-vc-0101 (ADC1) and EDB-mutl-vc-0101 (ADC3) at 1 and 3 mg/kg. FIG. 35F shows the anti-tumor efficacy of site-specific EDB-(KK183C+K290C)-vc-0101 (ADC2) and EDB-mut1(κK183C-K290C)-vc-0101 (ADC4) at 1 and 3 mg/kg. The 4 ADCs demonstrated similar efficacy in the H-1975 model irrespective of whether they contained the κK183C-K290C mutations. In addition, all ADCs tested resulted in robust anti-tumor efficacy including tumor regressions at 3 mg/kg. These data demonstrate that the introduction of the κK183C-K290C mutations did not negatively impact the efficacy of the ADCs. HT29 Colon CLX. The effects of various vc-linked auristatin ADCs were evaluated in HT29, a moderate EDB+ FN expressing colon CLX model of human cancer. As shown in FIG. 36, EDB-L19-vc-0101 (ADC1) and EDB-L19-vc-9411 (ADC7) were tested for anti-tumor activity at 3 mg/kg. Both EDB-L19-vc-0101 (ADC1) and EDB-L19-vc-9411 (ADC7) showed tumor regression at the 3 mg/kg dose over time.

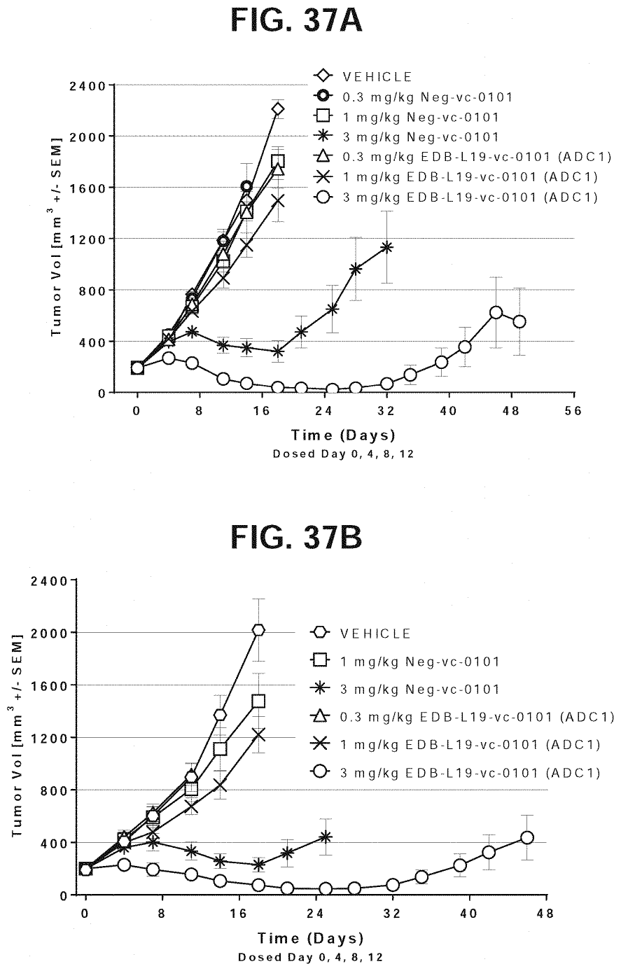

PDX-PAX-13565 and PDX-PAX-12534 Pancreatic PDXs. The anti-tumor efficacy of EDB-L19-vc-0101 (ADC1) was evaluated in human pancreatic PDX models. As shown in FIG. 37A, EDB-L19-vc-0101 (ADC1) was assessed at 0.3, 1 and 3 mg/kg in PDX-PAX-13565, a moderate to high EDB+FN expressing pancreatic PDX. As shown in FIG. 37B, EDB-L19-vc-0101 (ADC1) was assessed at 0.3, 1 and 3 mg/kg in PDX-PAX-12534, a low to moderate EDB+FN expressing pancreatic PDX. EDB-L19-vc-0101 (ADC1) demonstrated tumor regression in a dose dependent manner in both pancreatic PDX models evaluated.

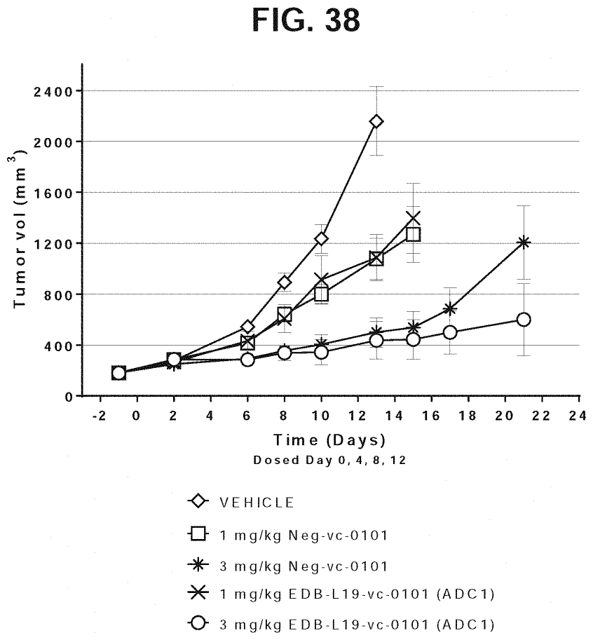

Ramos Lymphoma CLX. The anti-tumor efficacy of EDB-L19-vc-0101 (ADC1) was evaluated in Ramos, a moderate EDB+ FN expressing lymphoma CLX model. EDB-L19-vc-0101 (ADC1) was assessed for anti-tumor activity at 1 and 3 mg/kg. As shown in FIG. 38, EDB-L19-vc-0101 (ADC1) showed tumor regression at the 3 mg/kg dose in a dose dependent manner.

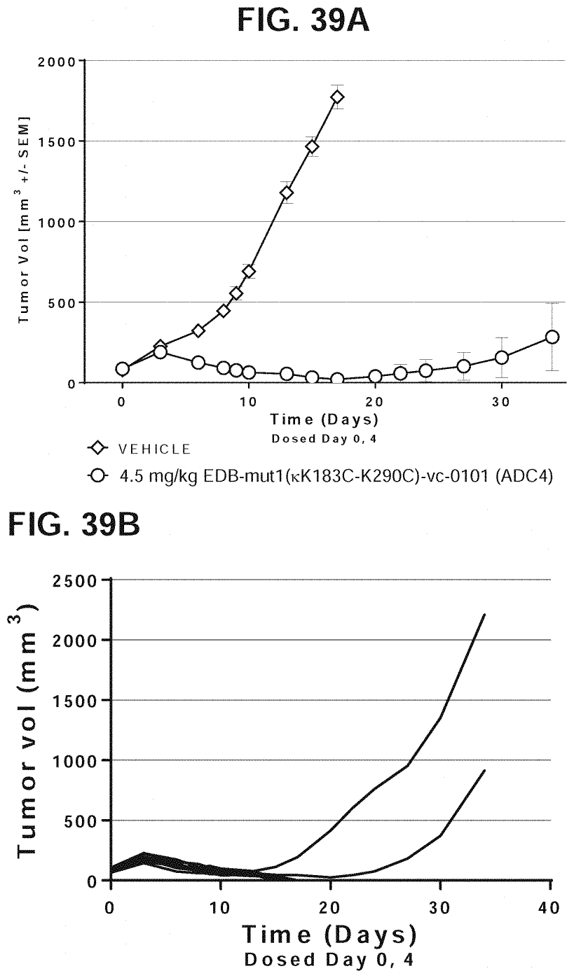

EMT-6 Breast Syngeneic Model. The anti-tumor efficacy of EDB-mutl(KK183C-K290C)-vc-0101 (ADC4) was evaluated in EMT-6, a mouse syngeneic breast carcinoma model in an immuncompetent background. As shown in FIG. 39A, EDB-mut1(κK183C-K290C)-vc-0101 (ADC4) demonstrated tumor growth inhibition at 4.5 mg/kg. The tumor growth inhibition was plotted as an average of tumor size in eleven tumor bearing animals±SEM. FIG. 39B shows the tumor growth inhibition curves for the 11 individual tumor bearing mice in the EDB-mut1(κK183C-K290C)-vc-0101 (ADC4) group dosed at 4.5 mg/kg. The tumor regressions in the 4.5 mg/kg group were complete and durable in 9 of 11 mice (82%) at the end of the study (34 days).

### 23.4: Pharmacokinetics (PK) of Site Specific EDB ADCs

Exposure of conventionally conjugated EDB-L19-vc-0101 (ADC1) and site-specific conjugated EDB-mut1(κK183C-K2900)-vc-0101 (ADC4) conjugated antibody drug conjugates were determined after an intravenous (IV) bolus dose administration of either 5 or 6 mg/kg in cynomolgus monkeys, respectively. Concentrations of total antibody (total Ab; measurement of both conjugated mAb and unconjugated mAb), ADC (mAb that is conjugated to at least one drug molecule) were measured using ligand binding assays (LBA) and concentrations of the released payload 0101 were measured using mass spectrometery. Quantitation of total Ab and ADC concentrations was achieved by ligand binding assay (LBA) using the Gyrolab® workstation with fluorescence detection. The Biotinylated capture protein used was a sheep anti-hIgG and the detection antibody was Alexa Fluor 647 goat anti-hIgG for total antibody or Alexa Fluor 647 anti-0101 mAb for ADC (data was processed by the Watson v 7.4 LIMS system). In vivo samples were prepared for unconjugated payload analysis using protein precipitation and injected onto an AB Sciex AP15500 (QTRAP) mass spectrometer using positive Turbo IonSpray electrospray ionization (ESI) and multiple reaction monitoring (MRM) mode. The transitions of 743.6→188.0 and 751.6→188.0 were used for the analyte and deuterated internal standard, respectively. Data acquisition and processing were carried out with Analyst software version 1.5.2 (Applied Biosystems/MDS Sciex, Canada).

The pharmacokinetics of total Ab, ADC and released payload from EDB-L19-vc-0101 ADC (at 5 mg/kg) and EDB-mut1(κK183C-K290C)-vc-0101 ADC (6 mg/kg) dosed cynomolgus monkeys are shown in Table 38. Exposure of the site-specific conjugated EDB-mut1(κK183C-K290C)-vc-0101 ADC showed both increased exposure (˜2.3× increase as measured by dose normalized AUC) and increased conjugation stability when compared to the conventional conjugate. Conjugation stability was assessed by both the higher ADC/Ab ratio (84% versus 75%) and by the lower released payload exposure (dose normalized AUC; 0.0058 versus 0.0082 μg*h/m L) for the site-specific conjugated EDB-mut2(κK183C-K290C)-vc-0101 ADC compared to the conventional EDB-L19-vc-0101 ADC, respectively. NA=not applicable.

TABLE 38
Summary of pharmacokinetics in non-human primates.

| | Dose | | Cₘₐₓ | AUC₀₋₅₀₄ | Terminal | AUC/ | ADC/Ab |
|---|---|---|---|---|---|---|---|
| ADC | (mg/kg) | Analyte | (μg/mL) | (μg*hr/mL) | T_{1/2} (day) | Dose | (%) |
| EDB-L19- | 5 | Ab | 114 ± 27 | 6907 ± 1997 | 5.1 ± 2.2 | 1381 ± 399 | — |
| vc-0101 | | ADC | 110 ± 31 | 5190 ± 1453 | 4.6 ± 1.0 | 1038 ± 291 | 75 ± 2 |
| (ADC1) | | Payload | 0.00053 ± 0.00025 | 0.0411 ± 0.0160 | NA | 0.0082 ± 0.0032 | — |
| EDB- | 6 | Ab | 164 ± 36 | 17600 ± 3045 | 6.4 ± 1.3 | 2933 ± 507 | — |
| mut1(κK183C- | | ADC | 156 ± 30 | 14567 ± 2122 | 5.9 ± 1.1 | 2428 ± 354 | 84 ± 3 |
| K290C)-vc-0101 | | Payload | 0.00024 ± 0.00021 | 0.0349 ± 0.0030 | NA | 0.0058 ± 0.0005 | — |
| (ADC4) | | | | | | | |

### 23.5: Toxicity Studies for Site Specific EDB ADCs

The nonclinical safety profile conventional EDB-L19-vc-0101 (ADC1) and site-specific conjugated EDB-mut1(κK183C-K2900)-vc-0101 (ADC4) was characterized in exploratory repeat-dose (Q3W×3) studies in Wistar-Han rats and cynomolgus monkeys. The rat and cynomolgus monkey were considered pharmacologically relevant nonclinical species for toxicity evaluation due to 100% protein sequence homology with human EDB+ FN, as well as similar binding affinity of the antibodies EDB-L19 (Ab1) and EDB-mut1(κK183C-K290C) (Ab4) to rat, human and monkey by Biacore assay, as demonstrated in Example 2.

EDB-L19-vc-0101 (ADC1) was evaluated in Wistar Han rats and cynomolgus monkeys up to 10 and 5 mg/kg/dose, respectively, and EDB-mut1(κK183C-K290C)-vc-0101 (ADC4) was evaluated in cynomolgus monkeys up to 12 mg/kg/dose. Rats or monkeys were dosed intravenously once every 3 weeks (on Days 1, 22 and 43) and were euthanized on Day 46 (3 days after the 3^{rd} dose). Animals were evaluated for clinical signs, changes in body weight, food consumption, clinical pathology parameters, organ weights, and macroscopic and microscopic observations. No mortality or significant changes in clinical condition of animals were noted in these studies.

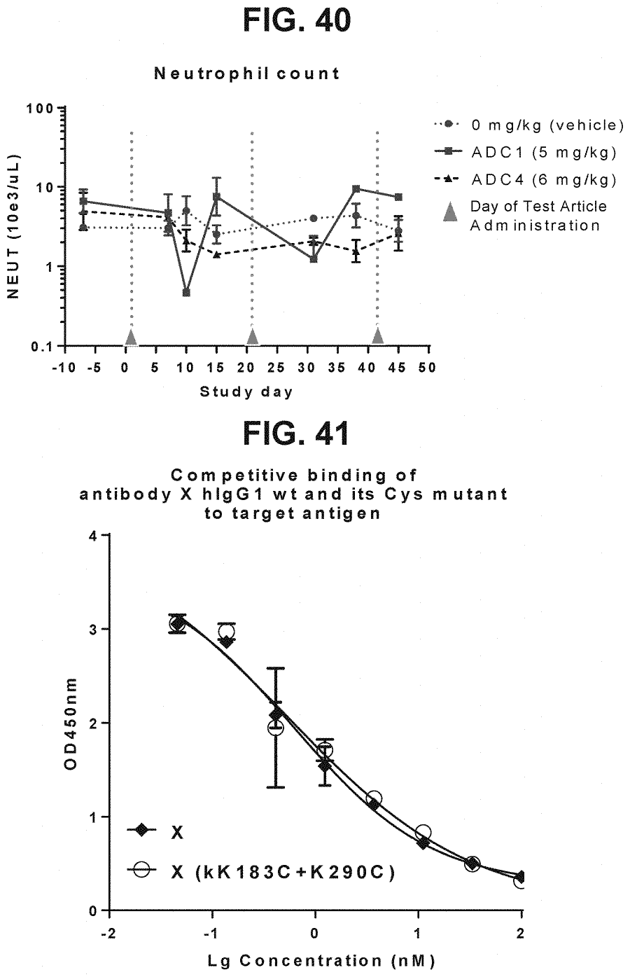

There was no indication of target-dependent toxicity in EDB+ FN expressing tissues/organs in rats and monkeys. In both species, the major toxicity was reversible myelosuppression with associated hematological changes. In monkeys, marked transient neutropenia was seen with conventionally conjugated EDB-L19-vc-0101 (ADC1) at 5 mg/kg/dose while only minimal effects on neutrophil counts were seen with site-specific conjugated EDB-mut1(κK183C-K290C)-vc-0101 (ADC4) at 6 mg/kg/dose, as shown in Table 39 and FIG. 40. Points represent mean and error bars represent ±1 standard deviation (SD) from the mean.

The data demonstrates significant alleviation of myelosuppression by site-specific conjugation. The toxicity profile of EDB-L19-vc-0101 (ADC1) and EDB-mut1(κK183C-K290C)-vc-0101 (ADC4) was consistent with target-independent effects of these conjugates and the highest non-severely toxic doses (HNSTD) for EDB-L19-vc-0101 (ADC1) and EDB-mut1(κK183C-K2900)-vc-0101 (ADC4) were determined to be 5 mg/kg/dose and 12 mg/kg/dose, respectively.

TABLE 39
Absolute neutrophil counts in cynomolgus
monkeys over the study duration.

| | | | | | EDB-mut1(κK183C- | |
|---|---|---|---|---|---|---|
| | | | EDB-L19-vc-0101 | | K290C)-vc-0101 | |
| | 0 mg/kg (vehicle) | | (5 mg/kg) | | (6 mg/kg) | |
| | Animal | Animal | Animal | Animal | Animal | Animal |
| Day | # 1 | # 2 | # 1 | # 2 | # 1 | # 2 |
| −7 | 3.26 | 2.9 | 8.48 | 4.67 | 2.41 | 7.42 |
| 7 | 3.54 | 2.52 | 7.08 | 2.29 | 3.96 | 4.3 |
| 10 | 3.16 | 6.83 | 0.11 | 0.82 | 2.66 | 1.56 |
| 15 | 3.06 | 1.98 | 11.41 | 3.65 | 1.37 | 1.44 |
| 31 | 3.87 | 4.17 | 0.39 | 2.07 | 1.91 | 2.22 |
| 38 | 3.09 | 5.63 | 16.17 | 2.73 | 1.97 | 1.13 |
| 45 | 3.53 | 2.07 | 13.02 | 1.83 | 1.4 | 3.78 |

### Example 24: Site-Specific ADCs with Antibodies Targeting Tumor Antigen

### 24.1. Generation of Site-Specific ADC Conjugate

24.1.1 Generation of Double cyc Mutant (kK183C+K290C).

To confirm that double cys mutant, kK183C+K290C, mediated site-specific drug conjugation confers significant benefits as compared to conventional antibody-drug conjugates, another antibody against a tumor-associated antigen, Antibody X (X hereinafter) was investigated. Antibody X was humanized from its mouse parental antibody. To prepare for site specific antibody drug conjugates with auristatin 0101, we generated X-hIgG1/kappa with kK183C mutation in the kappa light chain and K290C mutation in the hIgG1 heavy chain constant regions. The protein preparations for both antibody X and its double cys mutant version, X(kK183C+K290C), were generated and their relative binding activity with its target antigen was assessed in a competition ELISA. In this assay, both antibody X and cys mutant X(kK183C+K290C) were tested for their ability to compete against their common parental antibody for binding to target antigen immobilized on the ELISA plate. As shown in FIG. 41, antibody X and X(kK183C+K290C) had equivalent competitive binding activity to target antigen, indicating the cys mutations in the constant region of heavy and light chains did not affect antibody's binding activity to target antigen.

Methods.

Competition ELISA. 96 well plates (hi-bound CoStar plates) were coated with target antigen-Fc fusion protein. 1 to 3 serially diluted antibody X and cys mutant X(kK183C+K290C) solutions in blocking buffer (1% bovine serum albumin in PBST), in the presence of a constant concentration of biotinylated parental antibody were applied to the plate. After incubation for 2 hours, the plates were washed and HRP-conjugated streptavidin (Southern Biotech) diluted 1:5000 in blocking buffer was applied. Incubation with streptavidin was allowed for 40 minutes before the plates were developed with TMB solution for 10 minutes. The developing reaction was then stopped by adding 0.18 M H2SO4 and absorbance at 450 nm measured. Data plotting and analyses were performed with Microsoft Excel and Graphpad-Prism software.

24.1.2 Generation of X-vc0101 and X(kK183C+K290C)-vc0101 Conjugates

24.1.2.1 Generation of Conventional ADC (X-vc0101)

The conventional ADC was prepared via partial reduction of antibody X with tris (2-carboxyethyl) phosphine (TCEP) followed by reaction of reduced cysteine residues with maleimide functionalized linker-payload vc0101. In particular, the antibody was partially reduced via the addition of 2.2 molar excess of TCEP in 100 mM HEPES buffer, pH 7.0 and 1 mM diethylenetriaminepentaacetic acid (DTPA) for 2 hours at 37° C. The vc0101 was then added to the reaction mixture at a linker-payload/antibody ratio of 7:1 and reacted for additional 1 hour at 25° C. in the presence of 15% v/v of dimethylacetamide (DMA). N-ethylmaleimide (NEM) was added to cap the unreacted thiols, followed by addition of L-cysteine to quench any unreacted linker-payload. The reaction mixture was dialyzed overnight at 4° C. in PBS, pH 7.4 and purified by size exclusion chromatography (SEC; AKTA avant, Superdex 200 resin). The purified ADC was buffer exchanged into 20 mM histidine, 85 mg/mL sucrose, pH5.8 and stored at −70° C. The ADC was characterized via analytical SEC for purity; HIC and LC-ESI MS to calculate the Drug-Antibody Ratio (DAR). The protein concentration was determined via UV spectrophotometer.

24.1.2.2 Generation of Site-Specific ADC X (kK183C+K290C)-vc0101

The engineered double cys mutant, X(kK183C+K290C), was completely reduced with a 12-fold molar excess of TCEP in 100 mM HEPES buffer, pH 7.0 and 1 mM DTPA for 6 hours at 37° C. followed by desalting to remove excess TCEP. The reduced antibody was incubated in 2 mM dehydro ascorbic acid (DHA) for 16 hours at 4° C. to reform the inter-chain disulfide bonds. After desalting, the maleimide functionalized linker-payload vc0101 was added at a linker-payload/antibody molar ratio of 10:1 and reacted for an additional 2 hours at 25° C. in the presence of 15% v/v of dimethylacetamide (DMA). The reaction mixture was desalted and purified via hydrophobic interaction chromatography (HIC, AKTA avant, Butyl HP resin). The purified ADC was buffer exchanged into 20 mM histidine, 85 mg/mL sucrose, pH5.8 and stored at −70° C. The ADC was characterized via SEC for purity; HIC, reverse phase UPLC and LC-ESI MS to calculate the DAR. The protein concentration was determined via UV spectrophotometer.

24.1.2.3 ADC Drug Distribution

Compounds were prepared for HIC analysis by diluting samples to approximately 1 mg/ml with PBS. The samples were analyzed by auto-injection of 15 μl onto an Agilent 1200 HPLC with a TSK-GEL Butyl NPR column (4.6×3.5 mm, 2.5 μm pore size; Tosoh Biosciences part #14947). The system includes an auto-sampler with a thermostat, a column heater and a UV detector. The gradient method was used as follows: Mobile phase A: 1.5 M ammonium sulfate, 50 mM potassium phosphate dibasic (pH7); Mobile phase B: 20% isopropyl alcohol, 50 mM potassium phosphate dibasic (pH 7); T=0 min. 100% A; T=12 min. 0% A.

Drug distribution profiles are shown in Table 40. While both ADCs features similar average DAR, ADC using site-specific conjugation (X(κK183C+K290C)-vc0101) showed primarily one peak (94% is 4 DAR) and ADCs using conventional conjugation (X-vc0101) showed a mixture of differentially loaded conjugates (51% is 4 DAR). This homogeneous drug distribution profile is a major advantage of site-specific ADC over conventional ADC.

TABLE 40
Drug Distribution of ADCs (analyzed by HIC)

| | Avg. | 0 | 2 | 3 | 4 | 6 | 8 |
|---|---|---|---|---|---|---|---|
| ADC | DAR | DAR | DAR | DAR | DAR | DAR | DAR |
| X-vc0101 | 3.8 | 2% | 26% | 0% | 51% | 19% | 2% |
| X (κK183C + | 3.9 | 0% | 0% | 6.3% | 93.7% | 0% | 0% |
| K290C)-vc0101 | | | | | | | |

24.2. Evaluation of J145 ADCs as a Single Agent in Calu-6, Human Non-Small Cell Lung Caner Cell Line, Xenograft Model

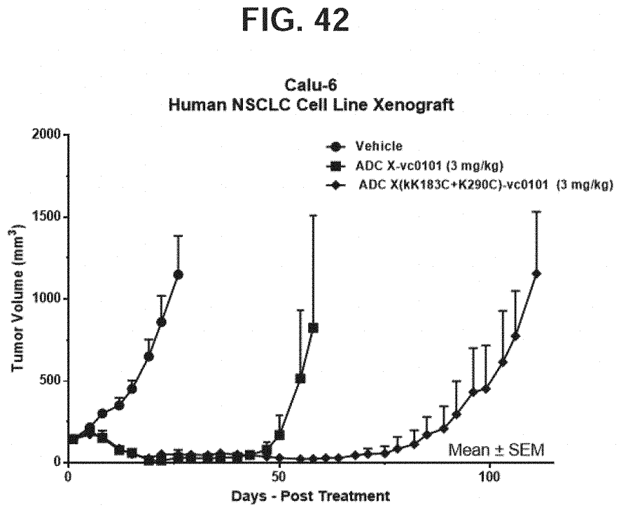

Dosing at 3 mg/kg in tumor-bearing animals of ADC X-vc0101 (conventional conjugate) and ADC X(kK183C+K290C)-vc0101 resulted in tumor regression in both groups after the last dose of drug candidates by day 15, with an average tumor volume of 60 mm³ and 53 mm³ respectively By study day 26, when the vehicle group was euthanized, both the treatment groups showed consistent tumor regressions. From day 47 to 58, two out of five animals in conventional conjugate group escaped the treatment effects and stated growing rapidly, however X(kK183C+K2900)-vc0101 consistently showed tumor regressions. The average tumor volumes for conventional conjugate and ADC X(kK183C+K290C)-vc0101 on day 58 were 825 mm³ and 23 mm³ respectively. (Table 41 and FIG. 42)

By study day 61, the conventional ADC group lost an animal due to sacrifice based on tumor volume that exceeded 3520 mm³. The X(kK183C+K290C)-vc0101 group showed consistent tumor regressions up to day 82 and later on showed re-growth of the tumors, and the largest mass present at the end of the study was 1881 mm3 on study day 111. (Table 41 and FIG. 42)

The ADC X(kK183C+K290C)-vc0101 showed a consistent anti-tumor effect on Calu-6 human NSCLC CDX model in a Q4DX4 dosing regimen at 3 mg/kg up to day 82 of the study. ADC X-vc0101, at initial time points of the study showed comparable tumor cell kill, however over the course of the study tumors escaped the treatment effects by day 47 and rapidly increased in size.

In conclusion, ADC X(kK183C+K290C)-vc0101 has better anti-tumor activity on Calu-6, human NSCLC CDX model with more surviving animals up to day 111.

TABLE 41
Tumor volumes of individual mice treated with ADCs or vehicle control.
(Individual tumor volumes represented as mm³ from n = 5 animals
per each of the treatment groups through day 111; Ave: average)

| | | | | | | | ADC | | | | | | ADC | | | | | |
|---|---|---|---|---|---|---|---|---|---|---|---|---|---|---|---|---|---|---|
| | | | | | | | X(kK183C + K290C)-vc0101 | | | | | | X-vc0101 | | | | | |
| | Vehicle | | | | | | (3 mg/kg) | | | | | | (3 mg/kg) | | | | | |
| Days | 1 | 2 | 3 | 4 | 5 | Ave | 1 | 2 | 3 | 4 | 5 | Ave | 1 | 2 | 3 | 4 | 5 | Ave |
| 1 | 149 | 127 | 192 | 135 | 112 | 143 | 115 | 140 | 184 | 130 | 150 | 143 | 133 | 172 | 156 | 113 | 142.2 | 143 |
| 5 | 211 | 167 | 309 | 218 | 167 | 214 | 121 | 173 | 240 | 171 | 155 | 172 | 178 | 288 | 163 | 166 | 161.2 | 191 |
| 8 | 334 | 250 | 350 | 331 | 236 | 300 | 121 | 167 | 300 | 107 | 100 | 159 | 140 | 219 | 125 | 143 | 124.9 | 150 |
| 12 | 448 | 248 | 458 | 353 | 240 | 349 | 61 | 96 | 150 | 60 | 64 | 86 | 72 | 111 | 65 | 74 | 63.8 | 77 |
| 15 | 416 | 270 | 591 | 511 | 460 | 450 | 57 | 50 | 70 | 49 | 38 | 53 | 70 | 74 | 49 | 52 | 55.2 | 60 |
| 19 | 519 | 307 | 770 | 838 | 815 | 650 | 25 | 23 | 30 | 32 | 19 | 26 | 1 | 29 | 7 | 23 | 9.8 | 14 |
| 22 | 657 | 347 | 1238 | 1018 | 1040 | 860 | 34 | 137 | 25 | 26 | 35 | 51 | 0 | 20 | 17 | 15 | 15.6 | 13 |
| 26 | 1069 | 489 | 1895 | 1379 | 912 | 1149 | 27 | 161 | 25 | 0 | 28 | 48 | 27 | 34 | 19 | 31 | 33.5 | 29 |
| 29 | | | | | | | 30 | 149 | 21 | 0 | 37 | 47 | 18 | 27 | 36 | 15 | 43.2 | 28 |
| 33 | | | | | | | 20 | 135 | 52 | 0 | 22 | 46 | 13 | 25 | 41 | 29 | 25.9 | 27 |
| 36 | | | | | | | 22 | 98 | 70 | 58 | 35 | 57 | 10 | 14 | 33 | 48 | 32.5 | 28 |
| 40 | | | | | | | 30 | 108 | 30 | 48 | 26 | 48 | 25 | 33 | 22 | 66 | 25.3 | 34 |
| 43 | | | | | | | | 100 | 29 | 52 | 0 | 45 | 56 | 75 | 22 | 62 | 17.5 | 47 |
| 47 | | | | | | | | 84 | 23 | 35 | 0 | 36 | 40 | 269 | 30 | 40 | 12.2 | 78 |
| 50 | | | | | | | | 89 | 15 | 12 | 0 | 29 | 28 | 648 | 57 | 90 | 13.3 | 167 |
| 55 | | | | | | | | 43 | 27 | 18 | 0 | 22 | 11 | 2164 | 27 | 368 | 0 | 514 |
| 58 | | | | | | | | 40 | 32 | 20 | 0 | 23 | 5 | 3520 | 0 | 601 | 0 | 825 |
| 61 | | | | | | | | 57 | 40 | 13 | 0 | 27 | | | | | | |
| 64 | | | | | | | | 56 | 42 | 13 | 0 | 28 | | | | | | |
| 68 | | | | | | | | 55 | 95 | 15 | 13 | 44 | | | | | | |
| 71 | | | | | | | | 47 | 145 | 0 | 16 | 52 | | | | | | |
| 75 | | | | | | | | 17 | 187 | 0 | 20 | 56 | | | | | | |
| 78 | | | | | | | | | 230 | 0 | 23 | 84 | | | | | | |
| 82 | | | | | | | | | 282 | 23 | 32 | 112 | | | | | | |
| 85 | | | | | | | | | 384 | 34 | 93 | 170 | | | | | | |
| 89 | | | | | | | | | 478 | 43 | 101 | 207 | | | | | | |
| 92 | | | | | | | | | 702 | 53 | 128 | 294 | | | | | | |
| 96 | | | | | | | | | 970 | 148 | 175 | 431 | | | | | | |
| 99 | | | | | | | | | 979 | 146 | 228 | 451 | | | | | | |
| 103 | | | | | | | | | 1229 | 203 | 414 | 616 | | | | | | |
| 106 | | | | | | | | | 1286 | 340 | 698 | 775 | | | | | | |
| 111 | | | | | | | | | 1881 | 612 | 972 | 1155 | | | | | | |

Methods.

Tumor xenografts were initiated in cohorts of seven female, athymic nude mice, 5-8 weeks of age, by subcutaneous injection of 5×10⁶ Calu-6 (ATCC, Cat #HTB-56) human lung tumor cells in a volume of 0.1 ml per mouse suspended in 50% Matrigel (BD Biosciences, Cat #356234) made with Eagle's Minimum Essential Medium (ATCC, Cat #30-2003) culture medium containing 10% fetal bovine serum (HyClone #SH30088.03H1) into the right flank. Dosing of test articles was initiated when mean tumor size reached 100-150 mm³, where tumor volume was calculated as: (mm³)=(a×b²/2) where ‘b’ is the smallest diameter and ‘a’ is the largest diameter. Tumor volume and body weight data were collected twice weekly. Blood samples (10 μl each) were collected from two mice in each group, 30 hours after the first dose and 30 hours after the last (fourth) dose. Blood was diluted into 190 μL of HBS-EP buffer and immediately stored at −80° C. Tumor masses were collected by necropsy in the same animals from which blood was collected, 30 hours after the last (fourth) dose, and snap frozen.

The ADC X(kK183C+K2900)-vc0101 was administered intravenously to tumor-bearing animals at doses of 6 mg/kg, 3 mg/kg, 1 mg/kg, and 0.3 mg/kg in a Q4D×4 dosing schedule (four doses administered every four days).

The ADC X-vc0101 (conventional conjugate) was administered intravenously to tumor-bearing animals at a dose of 3 mg/kg in a Q4D×4 dosing schedule (four doses administered every four days).

24.3. Pharmacokinetic Studies.

X-vc0101 and X(kK183C+K290C)-vc0101 showed comparable PK/TK profiles at the same dose level of 6 mg/kg, including Cmax, exposure (AUC) and half life (t_{1/2}) (Table 42). Similar exposure levels (i.e. AUC) of total Ab and ADC were observed for both X-vc0101 and X(kK183C+K2900)-vc0101 at 6 mg/kg and for X(kK183C+K2900)-vc0101 at additional higher doses of 9 and 12 mg/kg, when exposure reached much higher levels. This indicates that the ADCs dosed in animals remained largely intact throughout the course of the experiment for both compounds and that both compounds had similar stability in vivo.

TABLE 42
Mean Pharmacokinetics Parameters for X-vc0101 and X(kK183C +
K290C)-vc0101 Total Antibody and ADC in Monkey after IV Administration

| | | Total Antibody | | | | Antibody-Drug Conjugate (ADC) | | | |
|---|---|---|---|---|---|---|---|---|---|
| | Dose | Cₘₐₓ | Tₘₐₓ | AUC | t_{1/2} | Cₘₐₓ | Tₘₐₓ | AUC | t_{1/2} |
| Compound | (mg/kg) | (μg/mL) | (Hrs) | (μg*Hrs/mL) | (Hrs) | (μg/mL) | (Hrs) | (μg*Hrs/mL) | (Hrs) |
| X-vc0101 | 6 | 158 | 0.25 | 13100 | 77.9 | 160 | 0.25 | 10430 | 68.8 |
| X(kK183C + | 6 | 157 | 0.25 | 17700 | 84.4 | 160 | 0.25 | 14500 | 87.7 |
| K290C)-vc0101 | 9 | 233 | 0.25 | 32700 | 101 | 227 | 0.25 | 25300 | 72.3 |
| | 12 | 363 | 0.25 | 55600 | 173 | 371 | 0.25 | 44400 | 140 |

Cₘₐₓ = Maximum drug concentration;
Tₘₐₓ = time to Cₘₐₓ,
AUC = area under the concentration-time curve;
t_{1/2} = half-life, AUC was calculated from 0-504 hours

Methods

Exposure of conventional (X-vc0101) or site specific (X(kK183C+K290C)-vc0101) antibody drug conjugates (ADC) was determined after an IV bolus administration of either 6 mg/kg of X-vc0101, or 6, 9 and 12 mg/kg of X(kK183C+K290C)-vc0101) to cynomolgus monkeys. The plasma samples were collected at pre-dose, 0.25, 6, 24, 72, 168, 336 and 504 hours following the IV administration of each dose. Concentrations of total antibody (total Ab; measurement of both conjugated mAb and unconjugated mAb) and ADC (mAb that is conjugated to at least one drug molecule) was determined using ligand binding assays (LBA).The pharmacokinetics (PK)/toxicokinetics (TK) parameters at each dose were calculated from the concentration vs. time profiles of the total Ab and ADC for both X-vc0101 and X(kK183C+K2900)-vc0101 (Table 42).

### 24.4. Toxicity Studies

In two independent exploratory toxicity studies, male and female cynomolgus monkeys were dosed IV once every 3 weeks (study days 1, 22 and 43). On study day 46 (3 days after the 3^{rd} dose administration) animals were euthanized and protocol specified blood and tissue samples were collected. Clinical observations, clinical pathology, macroscopic and microscopic pathology evaluations were conducted in-life and post necropsy. For anatomic pathology evaluation, severity of histopathology findings was recorded on a subjective, relative, study specific basis.

In one of these studies, cynomolgus monkeys (2/sex/group) were administered vehicle or X-hIgG1-vc0101 at 6 mg/kg/dose. In the other study, monkeys (1/sex/group) were administered vehicle or X(kK183C+K290C)-vc0101 at 6, 9, and 12 mg/kg/dose. One male and one female administered X-hIgG1-vc0101 at 6 mg/kg/dose were electively euthanized on study day 11 because of clinical signs and clinical pathology data suggesting severe febrile neutropenia. In contrast, at the same dosage level when similar exposure were observed (see previous section) all cynomolgus monkeys dosed with X(kK183C+K290C)-vc0101 survived until scheduled necropsy on study day 46. Microscopically in the bone marrow at 6 mg/kg, all of the cynomolgus monkeys (4 in total) administered X-hIgG1-vc0101 had compound-related minimal to moderate decreased cellularity of all cell types (myleloid and erythroid), whereas there were no microscopic findings in the bone marrow in cynomolgus monkeys administered X(kK183C+K290C)-vc0101(2 in total). At higher dosages of 9 and 12 mg/kg when much higher exposure levels were observed, only minimal to mild increase in myeloid/erythroid (M/E) ratio resulting from increased numbers of primarily mature neutrophils and decreased numbers of cells of the erythroid lineage, was a finding in the bone marrow in cynomolgus monkeys administered X(kK183C+K290C)-vc0101at 9 mg/kg/dose (2 in total), but not in the monkeys administered X(kK183C+K290C)-vc0101 at 12 mg/kg/dose (2 in total).

TABLE 43
Mortality and Microscopic Findings in the Bone Marrow

| | X-hIgG1-vc0101 | | | | X(kK183C + K290C)-vc0101 | | | | | | | |
|---|---|---|---|---|---|---|---|---|---|---|---|---|
| | Males | | Females | | Males | | | | Females | | | |
| Dose (mg/kg/dose) | 0 | 6 | 0 | 6 | 0 | 6 | 9 | 12 | 0 | 6 | 9 | 12 |
| Mortality* | — | 1 | — | 1 | — | — | — | — | — | — | — | — |
| Microscopic Findings | | | | | | | | | | | | |
| Bone marrow (number of animals examined) | 2 | 2 | 2 | 2 | 1 | 1 | 1 | 1 | 1 | 1 | 1 | 1 |
| Decreased celluarity: all types | | | | | | | | | | | | |
| (multifocal or diffuse) | | | | | | | | | | | | |
| Minimal (Grade 1) | — | — | — | 1 | — | — | — | — | — | — | — | — |
| Mild (Grade 2) | — | 1 | — | — | — | — | — | — | — | — | — | — |
| Moderate (Grade 3) | — | 1 | — | 1 | — | — | — | — | — | — | — | — |
| Increased myeloid/erythroid ratio | | | | | | | | | | | | |
| Minimal (Grade 1) | — | — | — | — | — | — | — | — | — | — | 1 | — |
| Mild (Grade 2) | — | — | — | — | — | — | 1 | — | — | — | — | — |

*Animals were electively euthanized on Day 11 because of clinical signs and clinical pathology data suggesting febrile neutropenia
—: No test article-related finding present.

Therefore, the mortality and microscopic data demonstrated that the ADC conjugate based on site-specific-mutation technology, X(kK183C+K2900)-vc0101, clearly improved the X-hIgG1-vc0101-induced bone marrow toxicity and neutropenia.

### Example 25: Site-Specific ADCs with Antibody 1.1

### 25.1. Preparation of Antibody 1.1 for Site-Specific Conjugation

The method of preparing 1.1 antibody for site-specific conjugation through reactive cysteine residues was generally performed as described in PCT Publication WO2013/093809. One residue on the Kappa light chain constant region (K183 using the Kabat numbering scheme) and one on the IgG1 heavy chain constant region (K290 using the EU index of Kabat) were altered to a cysteine (C) residue by site-directed mutagenesis.

### 25.2. Production of Stably Transfected Cells Expressing Her2-PT Engineered Cysteine Variant Antibodies

To produce 1.1-κK183C-K2900 for conjugation studies, CHO cells were transfected with DNA encoding 1.1-κK183C-K2900 and stable high production pools were isolated using standard procedures well-known in the art. A three-column process, i.e. Protein-A affinity capture followed by a TMAE column and then CHA-TI column, was used to isolate 1.1-κK183C-K2900 from the concentrated CHO pool starting material. Using these purification processes, the 1.1-κK183C-K2900 preparation contained 98.6% peak-of-interest (POI) as determined by analytical size-exclusion chromatography (Table 44). The results shown in Table 44 demonstrate that acceptable levels of high molecular mass species (HMMS) was detected following elution of 1.1-κK183C+K290C from the Protein A resin and that this undesirable HMMS species could be removed using TMAE and CHA-TI chromatography. The data also demonstrated that the Protein A binding site in the human IgG1 constant region was not altered by the presence of the engineered cysteine residue at position 290 (EU index numbering).

TABLE 44
Production Summary of 1.1-κK183C-K290 Antibody

| | POI | HMMS | LMMS | Recovery |
|---|---|---|---|---|
| Purification Step | % aSEC | % aSEC | % aSEC | % |
| Protein A | 95.65 | 4.35 | 0 | NA |
| TMAE | 95.87 | 4.13 | 0 | 92% F |
| CHA-TI | 98.85 | 1.15 | 0 | 62% |
| UF/DF Final Product | 98.595 | 1.405 | 0 | 96% |

### 25.3. Site-Specific Conjugation of Antibody 1.1

Conjugation of the maleimide functionalized linker-payload to 1.1-KK183C-K290C was achieved by complete reduction of the antibody with a 15-fold molar excess of Tris(2-carboxyethyl)phosphine hydrochloride (TCEP) in 100 mM HEPES (4-(2-Hydroxyethyl)-1-piperazineethanesulfonic acid buffer), pH 7.0 and 1 mM diethylenetriaminepentaacetic acid (DTPA) for 6 hours at 37° C. followed by desalting to remove excess TCEP. The reduced 1.1-κK183C-K290C antibody was incubated in 1.5 mM dehydro ascorbic acid (DHA), 100 mM HEPES, pH 7.0 and 1 mM DTPA for 16 hours at 4° C. to reform the inter-chain disulfide bonds. The desired linker-payload was added to the reaction mixture at a linker-payload/antibody molar ratio of 7 and reacted for an additional 1 hour at 25° C. in the presence of 15% v/v of dimethylacetamide (DMA). After the 1 hour incubation period, 6-fold excess L-Cys was added to quench any unreacted linker-payload. The reaction mixture was purified via hydrophobic interaction chromatography (HIC) using Butyl-sepharose HP column (GE Lifesciences). The method utilized 1 M KPO4, 50 mM Tris pH 7.0 for binding and the ADC was eluted with 50 mM Tris, pH 7.0 over 10 CVs. The ADC was further characterized via size-exclusion chromatography (SEC) for purity, hydrophobic interaction chromatography (HIC), and liquid chromatography electrospray ionization tandem mass spectrometry (LC-ESI MS) or reverse phase chromatography (RP) to calculate drug-antibody ratio (loading or DAR). ADCs can be compared with their respective antibody by calculating a relative retention time (RRT), which is the ratio of the HIC retention time of the ADC divided by the HIC retention time of the respective antibody. The protein concentration was determined via UV spectrophotometer. Results shown in Table 45 show that 1.1-κK183C-K290C engineered cysteine antibody was efficiently conjugated to the maleimide functionalized linker-payload vc0101 producing a homogeneous ADC with the predicted and desirable number of payloads (i.e. DAR 3.9).

TABLE 45
Properties of the 1.1-κK183C + K290C-vc0101 site-specific conjugate.

| | DAR | DAR | Free drug | HIC | % | % |
|---|---|---|---|---|---|---|
| ADC | (LCMS) | (RP) | (μg/ mg Ab) | RRT | Purity | Yield |
| 1.1-κK183C-K290C- | 3.9 | 3.9 | 0.01 | 1.62 | 99.2 | 58.9 |
| hG1_mcValCitPABC_Aur0101 | | | | | | |

25.4. Bioanalytical Characterization of 1.1-kK183C-K290C Antibody and 1.1-kK183C-K290C-vc0101 ADC

25.4.1Thermal Stability

Differential Scanning calorimetry (DCS) was used to determine the thermal stability of the 1.1-kK183C-K290C antibody and the corresponding Aur-06380101 site-specific conjugate. For this analysis, samples formulated in 20 mM Histidine pH 5.8, 8.5% sucrose, 0.05 mg/ml EDTA were dispensed into the sample tray of a MicroCal VP-Capillary DSC with Autosampler (GE Healthcare Bio-Sciences, Piscataway, N.J.), equilibrated for 5 minutes at 10° C. and then scanned up to 110° C. at a rate of 100° C. per hour. A filtering period of 16 seconds was selected. Raw data was baseline corrected and the protein concentration was normalized. Origin Software 7.0 (OriginLab Corporation, Northampton, Mass.) was used to fit the data to an MN2-State Model with an appropriate number of transitions.

The 1.1-kK183C-K290C antibody exhibited excellent thermal stability with the first melting transition (Tm1) at 72.78° C. and the resultant 1.1-kK183C-K290C-vc0101 site-specific conjugate (SSC) showed good and comparable stability with both the T-kK183C-K290C-vc0101 and EDB-(kK183C-K94R-K290C)-vc0101 SSCs described herein as determined by the first melting transition (Tm1)>65° C. (Table 46). Taken together these results demonstrated that the engineered cysteine 1.1-(kK183C-K94R-K290C) antibody was thermally stable and that site-specific conjugation of 0101 via a vc linker yielded a conjugate with good thermal stability.

TABLE 46
Thermal Stability of the 1.1-kK183C-K290C Antibody and
Corresponding Site-Specific Auristatin 0101 Conjugate

| Antibody | Tm1 | Tm2 | Tm3 | Apparent Fab Tm |
|---|---|---|---|---|
| or ADC | (° C.) | (° C.) | (° C.) | (° C.) |
| 1.1-kK183C- | 72.78 ± | 83.02 ± | 85.60 ± | 84.9 |
| K290C | 0.09 | 0.64 | 0.21 | |
| 1.1-kK183C- | 65.40 ± | 82.04 ± | 85.09 ± | 84.5 |
| K290C-vc0101 | 0.17 | 0.75 | 0.15 | |

25.4.2 Integrity of 1.1-kK183C-K290C Antibody and Corresponding Site-Specific Auristatin 0101 Conjugate

Non-reducing Caliper Capillary Gel Electrophoresis (Caliper LabChip GXII: Perkin Elmer Waltham, Mass.) analysis was performed to determine the purity and integrity of the 1.1-kK183C-K290C antibody and corresponding vc0101 site-specific conjugate. Results show that the cysteine engineered 1.1-kK183C-K290C antibody exhibited good integrity with % IgG at >96% and the analogous site-specific conjugate preparation containing <8% fragmented ADC. The integrity of the 1.1-kK183C-K290C-vc0101 site-specific conjugate was higher than observed for the EDB-(kK183C-K94R-K290C)-vc0101 which was purified using an alternate method (i.e. size-exclusion chromatography versus hydrophobic interaction chromatography) and was significantly improved relative to the ADC that was prepared using conventional conjugation methodologies (i.e. EDB-L19-vc-0101) as shown in Table 47.These results support that site-specific conjugation via engineered cysteines K290C and K183C in the IgG1 and Kappa constant regions, respectively, yield an ADC with significantly improved integrity compared to that prepared using conventional conjugation methodologies to endogenous cysteines within the antibody constant regions.

TABLE 47
Integrity of the 1.1-kK183C-K290C Antibody
and Site-Specific vc0101 Conjugate

| | | | % Non IgG | |
|---|---|---|---|---|
| | | % Non | % | % |
| Antibody or ADC | % IgG | IgG | LMMS | HMMS |
| 1.1-kK183C-K290C | 96.77 | 3.23 | 2.18 | 1.05 |
| 1.1-kK183C-K290C- | 93.27 | 6.73 | 6.60 | 0.13 |
| vc0101 | | | | |
| EDB-(kK183C-K94R- | 97.8 | 2.2 | ND | ND |
| K290C) | | | | |
| EDB-(kK183C-K94R- | 80.7 | 19.3 | ND | ND |
| K290C)-vc0101 | | | | |
| EDB-L19-VC-0101 | 1.4 | 98.6 | ND | ND |

ND = Not determined

25.5. Pharmacokinetics (PK) of 1.1-kK183C-K290C-vc0101

Exposure of site-specifically conjugated 1.1-κK183C+K2900-vc0101 ADC was determined after an IV bolus dose administration of either 6 or 12 mg/kg to cynomolgus monkeys. Concentrations of total antibody (total Ab; measurement of both conjugated Ab and unconjugated Ab) and ADC (Ab that is conjugated to at least one drug molecule) was measured using ligand binding assays (LBA).

Concentration versus time profiles and pharmacokinetics/toxicokinetics of total Ab and 1.1-κK183C+K290C-vc0101 site-specific ADC are shown in Table 48. Exposure of the 1.1-κK183C+K2900-vc0101 ADC increased in an approximately dose dependent manner. Additionally the exposure of κK183C+K2900-vc0101 ADC was both similar and comparable to the Trastuzumab site-specific conjugate T(kK183C+K290C) described herein that has both increased exposure and stability when compared to the conventionally conjugated ADC (Table 44).

TABLE 48
Pharmacokinetics

| | Dose | | | Cmax | AUC (0-456 h) |
|---|---|---|---|---|---|
| ADC | (mg/kg) | Day | Modality | (μg/mL) | (μg · h/mL) |
| 1.1 | 6 | 1 | Total Ab | 241 | 20300 |
| | | | ADC | 247 | 25500 |
| | 12 | 1 | Total Ab | 324 | 44700 |
| | | | ADC | 337 | 55800 |
| HER2 | 6 | 1 | Total Ab | 187 | 18400 |
| | | | ADC | 181 | 16600 |
| | 12 | 1 | Total Ab | 368 | 45300 |
| | | | ADC | 352 | 39600 |

TABLE 49
Residue numbering chart

| Heavy Chain | | | | Heavy Chain | | | |
|---|---|---|---|---|---|---|---|
| (CH2 domain, except A114) | | | | (CH3 domain) | | | |
| | | Kabat | Position | | | Kabat | Position |
| Resi- | EU | num- | at SEQ | Resi- | EU | num- | at SEQ |
| due | index | bering | ID No: 62 | due | index | bering | ID No. 62 |
| A114 | 118 | 114 | N/A | E345 | 345 | 366 | 115 |
| K246 | 246 | 259 | 16 | Q347 | 347 | 368 | 117 |
| D249 | 249 | 262 | 19 | S354 | 354 | 375 | 124 |
| D265 | 265 | 278 | 35 | R355 | 355 | 376 | 125 |
| S267 | 267 | 280 | 37 | L358 | 358 | 381 | 128 |
| D270 | 270 | 283 | 40 | K360 | 360 | 383 | 130 |
| N276 | 276 | 289 | 46 | Q362 | 362 | 385 | 132 |
| Y278 | 278 | 291 | 48 | K370 | 370 | 393 | 140 |
| E283 | 283 | 300 | 53 | Y373 | 373 | 396 | 143 |
| K290 | 290 | 307 | 60 | S375 | 375 | 398 | 145 |
| R292 | 292 | 309 | 62 | D376 | 376 | 399 | 146 |
| E293 | 293 | 310 | 63 | A378 | 378 | 401 | 148 |
| E294 | 294 | 311 | 64 | E380 | 380 | 405 | 150 |
| Y300 | 300 | 319 | 70 | E382 | 382 | 407 | 152 |
| V302 | 302 | 321 | 72 | Q386 | 386 | 414 | 156 |
| V303 | 303 | 322 | 73 | E388 | 388 | 416 | 158 |
| L314 | 314 | 333 | 84 | N390 | 390 | 418 | 160 |
| N315 | 315 | 334 | 85 | K392 | 392 | 420 | 162 |
| E318 | 318 | 337 | 88 | T393 | 393 | 421 | 163 |
| K320 | 320 | 339 | 90 | D401 | 401 | 430 | 171 |
| I332 | 332 | 351 | 102 | F404 | 404 | 435 | 174 |
| E333 | 333 | 352 | 103 | T411 | 411 | 442 | 181 |
| K334 | 334 | 353 | 104 | D413 | 413 | 444 | 183 |
| I336 | 336 | 355 | 106 | K414 | 414 | 445 | 184 |
| | | | | R416 | 416 | 447 | 186 |
| | | | | Q418 | 418 | 449 | 188 |
| | | | | Q419 | 419 | 450 | 189 |
| | | | | N421 | 421 | 452 | 191 |
| | | | | M428 | 428 | 459 | 198 |
| | | | | A431 | 431 | 462 | 201 |
| | | | | L432 | 432 | 463 | 202 |
| | | | | T437 | 437 | 468 | 207 |
| | | | | Q438 | 438 | 469 | 208 |
| | | | | K439 | 439 | 470 | 209 |
| | | | | L443 | 443 | 474 | 213 |
| | | | | S444 | 444 | 475 | 214 |
| Kappa Light Chain | | | | Lambda Light Chain | | | |
| | | Kabat | Position | | | Kabat | Position |
| Resi- | EU | num- | at SEQ | Resi- | EU | num- | at SEQ |
| due | index | bering | ID No. 63 | due | index | bering | ID No: 64 |
| A111 | 111 | 111 | 4 | K110 | N/A | 110 | 4 |
| K149 | 149 | 149 | 42 | A111 | N/A | 111 | 5 |
| K183 | 183 | 183 | 76 | L125 | N/A | 125 | 19 |
| K188 | 188 | 188 | 81 | K149 | N/A | 149 | 43 |
| K207 | 207 | 207 | 100 | V155 | N/A | 155 | 49 |
| N210 | 210 | 210 | 103 | G158 | N/A | 158 | 52 |
| | | | | T161 | N/A | 161 | 55 |
| | | | | P183 | N/A | 183 | 76 |
| | | | | Q185 | N/A | 185 | 78 |
| | | | | S188 | N/A | 188 | 81 |
| | | | | H189 | N/A | 189 | 82 |
| | | | | S191 | N/A | 191 | 84 |
| | | | | T197 | N/A | 197 | 90 |
| | | | | V205 | N/A | 205 | 96 |
| | | | | E206 | N/A | 206 | 97 |
| | | | | K207 | N/A | 207 | 98 |
| | | | | T208 | N/A | 208 | 99 |
| | | | | A210 | N/A | 210 | 101 |

TABLE 50
Nucleic Acid Sequences

| SEQ | | |
|---|---|---|
| ID | | |
| NO. | Description | Sequence |
| 79 | T(K290C) | GCGTCGACCAAGGGCCCATCGGTCTTCCCCCTGGCACCCTCCTCCAAGAGCACCTC |
| | heavy chain | TGGGGGCACAGCGGCCCTGGGCTGCCTGGTCAAGGACTACTTCCCCGAACCGGTGA |
| | constant | CGGTGTCGTGGAACTCAGGCGCCCTGACCAGCGGCGTGCACACCTTCCCGGCTGTC |
| | region | CTACAGTCCTCAGGACTCTACTCCCTCAGCAGCGTGGTGACCGTGCCCTCCAGCAG |
| | DNA (anti- | CTTGGGCACCCAGACCTACATCTGCAACGTGAATCACAAGCCCAGCAACACCAAGG |
| | HER2) | TGGACAAGAAAGTTGAGCCCAAATCTTGTGACAAAACTCACACATGCCCACCGTGC |
| | | CCAGCACCTGAACTCCTGGGGGGACCGTCAGTCTTCCTCTTCCCCCCAAAACCCAA |
| | | GGACACCCTCATGATCTCCCGGACCCCTGAGGTCACATGCGTGGTGGTGGACGTGA |
| | | GCCACGAAGACCCTGAGGTCAAGTTCAACTGGTACGTGGACGGCGTGGAGGTGCAT |
| | | AATGCCAAGACATGCCCGCGGGAGGAGCAGTACAACAGCACGTACCGTGTGGTCAG |
| | | CGTCCTCACCGTCCTGCACCAGGACTGGCTGAATGGCAAGGAGTACAAGTGCAAGG |
| | | TCTCCAACAAAGCCCTCCCAGCCCCCATCGAGAAAACCATCTCCAAAGCCAAAGGG |
| | | CAGCCCCGAGAACCACAGGTGTACACCCTGCCCCCATCCCGGGAGGAGATGACCAA |
| | | GAACCAGGTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCCTCCCGTG |
| | | CTGGACTCCGACGGCTCCTTCTTCCTCTATAGCAAGCTCACCGTGGACAAGAGCAG |
| | | GTGGCAGCAGGGGAACGTCTTCTCATGCTCCGTGATGCATGAGGCTCTGCACAACC |
| | | ACTACACGCAGAAGAGCCTCTCCCTGTCCCCGGGT |
| 80 | T(kK183C) | CGGACCGTGGCCGCTCCCTCCGTGTTCATCTTCCCACCCTCCGACGAGCAGCTGAA |
| | light chain | GTCCGGCACCGCCTCCGTCGTGTGCCTGCTGAACAACTTCTACCCCCGCGAGGCCA |
| | constant | AGGTGCAGTGGAAGGTGGACAACGCCCTGCAGTCCGGCAACTCCCAGGAATCCGTC |
| | region | ACCGAGCAGGACTCCAAGGACAGCACCTACTCCCTGTCCTCCACCCTGACCCTGTC |
| | DNA (anti- | CTGCGCCGACTACGAGAAGCACAAGGTGTACGCCTGCGAAGTGACCCACCAGGGCC |
| | HER2) | TGTCCAGCCCCGTGACCAAGTCCTTCAACCGGGGCGAGTGC |
| 81 | EDB-K290C | GAGGTGCAGCTGTTGGAGTCTGGGGGAGGCTTGGTACAGCCTGGGGGGTCCCTGAG |
| | Heavy Chain | ACTCTCCTGTGCAGCCTCTGGATTCACCTTTAGCAGTTTTTCGATGAGCTGGGTCC |
| | nucleotide | GCCAGGCTCCAGGGAAGGGGCTGGAGTGGGTCTCATCTATTAGTGGTAGTTCGGGT |
| | sequence | ACCACATACTACGCAGACTCCGTGAAGGGCCGGTTCACCATCTCCAGAGACAATTC |
| | | CAAGAACACGCTGTATCTGCAAATGAACAGCCTGAGAGCCGAAGACACGGCCGTAT |
| | | ATTACTGTGCGAAACCGTTTCCGTATTTTGACTACTGGGGCCAGGGAACCCTGGTC |
| | | ACCGTCTCGAGTGCGTCGACCAAGGGCCCATCGGTCTTCCCCCTGGCACCCTCCTC |
| | | CAAGAGCACCTCTGGGGGCACAGCGGCCCTGGGCTGCCTGGTCAAGGACTACTTCC |
| | | CCGAACCGGTGACGGTGTCGTGGAACTCAGGCGCCCTGACCAGCGGCGTGCACACC |
| | | TTCCCGGCTGTCCTACAGTCCTCAGGACTCTACTCCCTCAGCAGCGTGGTGACCGT |
| | | GCCCTCCAGCAGCTTGGGCACCCAGACCTACATCTGCAACGTGAATCACAAGCCCA |
| | | GCAACACCAAGGTGGACAAGAAAGTTGAGCCCAAATCTTGTGACAAAACTCACACA |
| | | TGCCCACCGTGCCCAGCACCTGAACTCCTGGGGGGACCGTCAGTCTTCCTCTTCCC |
| | | CCCAAAACCCAAGGACACCCTCATGATCTCCCGGACCCCTGAGGTCACATGCGTGG |
| | | TGGTGGACGTGAGCCACGAAGACCCTGAGGTCAAGTTCAACTGGTACGTGGACGGC |
| | | GTGGAGGTGCATAATGCCAAGACATGCCCGCGGGAGGAGCAGTACAACAGCACGTA |
| | | CCGTGTGGTCAGCGTCCTCACCGTCCTGCACCAGGACTGGCTGAATGGCAAGGAGT |
| | | ACAAGTGCAAGGTCTCCAACAAAGCCCTCCCAGCCCCCATCGAGAAAACCATCTCC |
| | | AAAGCCAAAGGGCAGCCCCGAGAACCACAGGTGTACACCCTGCCCCCATCCCGGGA |
| | | TGAGCTGACCAAGAACCAGGTCAGCCTGACCTGCCTGGTCAAAGGCTTCTATCCCA |
| | | GCGACATCGCCGTGGAGTGGGAGAGCAATGGGCAGCCGGAGAACAACTACAAGACC |
| | | ACGCCTCCCGTGCTGGACTCCGACGGCTCCTTCTTCCTCTACAGCAAGCTCACCGT |
| | | GGACAAGAGCAGGTGGCAGCAGGGGAACGTCTTCTCATGCTCCGTGATGCATGAGG |
| | | CTCTGCACAACCACTACACGCAGAAGAGCCTCTCCCTGTCTCCGGGT |
| 82 | EDB-K290C | GCGTCGACCAAGGGCCCATCGGTCTTCCCCCTGGCACCCTCCTCCAAGAGCACCTC |
| | Heavy Chain | TGGGGGCACAGCGGCCCTGGGCTGCCTGGTCAAGGACTACTTCCCCGAACCGGTGA |
| | constant | CGGTGTCGTGGAACTCAGGCGCCCTGACCAGCGGCGTGCACACCTTCCCGGCTGTC |
| | region | CTACAGTCCTCAGGACTCTACTCCCTCAGCAGCGTGGTGACCGTGCCCTCCAGCAG |
| | nucleotide | CTTGGGCACCCAGACCTACATCTGCAACGTGAATCACAAGCCCAGCAACACCAAGG |
| | sequence | TGGACAAGAAAGTTGAGCCCAAATCTTGTGACAAAACTCACACATGCCCACCGTGC |
| | | CCAGCACCTGAACTCCTGGGGGGACCGTCAGTCTTCCTCTTCCCCCCAAAACCCAA |
| | | GGACACCCTCATGATCTCCCGGACCCCTGAGGTCACATGCGTGGTGGTGGACGTGA |
| | | GCCACGAAGACCCTGAGGTCAAGTTCAACTGGTACGTGGACGGCGTGGAGGTGCAT |
| | | AATGCCAAGACATGCCCGCGGGAGGAGCAGTACAACAGCACGTACCGTGTGGTCAG |
| | | CGTCCTCACCGTCCTGCACCAGGACTGGCTGAATGGCAAGGAGTACAAGTGCAAGG |
| | | TCTCCAACAAAGCCCTCCCAGCCCCCATCGAGAAAACCATCTCCAAAGCCAAAGGG |
| | | CAGCCCCGAGAACCACAGGTGTACACCCTGCCCCCATCCCGGGATGAGCTGACCAA |
| | | GAACCAGGTCAGCCTGACCTGCCTGGTCAAAGGCTTCTATCCCAGCGACATCGCCG |
| | | TGGAGTGGGAGAGCAATGGGCAGCCGGAGAACAACTACAAGACCACGCCTCCCGTG |
| | | CTGGACTCCGACGGCTCCTTCTTCCTCTACAGCAAGCTCACCGTGGACAAGAGCAG |
| | | GTGGCAGCAGGGGAACGTCTTCTCATGCTCCGTGATGCATGAGGCTCTGCACAACC |
| | | ACTACACGCAGAAGAGCCTCTCCCTGTCTCCGGGT |
| 83 | EDB-kK183C- | GAAATTGTGTTAACGCAGTCTCCAGGCACCCTGTCTTTGTCTCCAGGGGAAAGAGC |
| | huKappa Light | CACCCTCTCCTGCAGGGCCAGTCAGAGTGTTAGCAGCAGCTTTTTAGCCTGGTACC |
| | Chain | AGCAGAAACCTGGCCAGGCTCCCAGGCTCCTCATCTATTATGCATCCAGCAGGGCC |
| | nucleotide | ACTGGCATCCCAGACAGGTTCAGTGGCAGTGGGTCTGGGACAGACTTCACTCTCAC |
| | sequence | CATCAGCAGACTGGAGCCTGAAGATTTTGCAGTGTATTACTGTCAGCAGACGGGTC |
| | | GTATTCCGCCGACGTTCGGCCAAGGGACCAAGGTGGAAATCAAACGAACTGTGGCT |
| | | GCACCATCTGTCTTCATCTTCCCGCCATCTGATGAGCAGTTGAAATCTGGAACTGC |
| | | CTCTGTTGTGTGCCTGCTGAATAACTTCTATCCCAGAGAGGCCAAAGTACAGTGGA |
| | | AGGTGGATAACGCCCTCCAATCGGGTAACTCCCAGGAGAGTGTCACAGAGCAGGAC |
| | | AGCAAGGACAGCACCTACAGCCTCAGCAGCACCCTGACGCTGAGCTGCGCAGACTA |
| | | CGAGAAACACAAAGTCTACGCCTGCGAAGTCACCCATCAGGGCCTGAGCTCGCCCG |
| | | TCACAAAGAGCTTCAACAGGGGAGAGTGT |
| 84 | EDB-kK183C- | CGAACTGTGGCTGCACCATCTGTCTTCATCTTCCCGCCATCTGATGAGCAGTTGAA |
| | huKappa Light | ATCTGGAACTGCCTCTGTTGTGTGCCTGCTGAATAACTTCTATCCCAGAGAGGCCA |
| | Chain | AAGTACAGTGGAAGGTGGATAACGCCCTCCAATCGGGTAACTCCCAGGAGAGTGTC |
| | constant | ACAGAGCAGGACAGCAAGGACAGCACCTACAGCCTCAGCAGCACCCTGACGCTGAG |
| | region | CTGCGCAGACTACGAGAAACACAAAGTCTACGCCTGCGAAGTCACCCATCAGGGCC |
| | nucleotide | TGAGCTCGCCCGTCACAAAGAGCTTCAACAGGGGAGAGTGT |
| | sequence | |

The various features and embodiments of the present invention, referred to in individual sections above apply, as appropriate, to other sections, mutatis mutandis. Consequently features specified in one section may be combined with features specified in other sections, as appropriate. All references cited herein, including patents, patent applications, papers, text books, and cited sequence Accession numbers, and the references cited therein are hereby incorporated by reference in their entirety. In the event that one or more of the incorporated literature and similar materials differs from or contradicts this application, including but not limited to defined terms, term usage, described techniques, or the like, this application controls.

What is claimed is:

1. A polypeptide comprising an antibody heavy chain constant domain comprising an engineered cysteine residue at position 290, according to the numbering of the EU index of Kabat.

2. The polypeptide of claim 1, wherein said constant domain comprises an IgG heavy chain CH₂ domain.

3. A polypeptide comprising an antibody heavy chain constant domain comprising an engineered cysteine residue at a position corresponding to residue 60 of SEQ ID NO:61, when said constant domain is aligned with SEQ ID NO:61.

4. The polypeptide of claim 3, wherein said engineered cysteine residue is located at position 290 of an IgG CH₂ domain, according to the numbering of the EU index of Kabat.

5. The polypeptide of claim 1, wherein said constant domain further comprises an engineered cysteine residue at a position selected from the group consisting of: 118, 246, 249, 265, S267, 270, 276, 278, 283, 292, 293, 294, 300, 302, 303, 314, 315, 318, 320, 332, 333, 334, 336, 345, 347, 354, 355, 358, 360, 362, 370, 373, 375, 376, 378, 380, 382, 386, 388, 390, 392, 393, 401, 404, 411, 413, 414, 416, 418, 419, 421, 428, 431, 432, 437, 438, 439, 443, 444, and any combination thereof, according to the numbering of the EU index of Kabat.

6. The polypeptide of claim 1, wherein said constant domain further comprises an engineered cysteine residue at a position selected from the group consisting of: 118, 334, 347, 373, 375, 380, 388, 392, 421, 443, and any combination thereof, according to the numbering of the EU index of Kabat.

7. The polypeptide of claim 1, wherein said constant domain further comprises an engineered cysteine residue at position 334, according to the numbering of the EU index of Kabat.

8. An antibody or antigen binding fragment thereof comprising the polypeptide of claim 1.

9. An antibody or antigen binding fragment thereof comprising:
(a) the polypeptide of claim 1, and
(b) an antibody light chain constant region comprising (i) an engineered cysteine residue at position 183, according to the numbering of Kabat; or (ii) an engineered cysteine residue at a position corresponding to residue 76 of SEQ ID NO:63, when said light chain constant domain is aligned with SEQ ID NO:63.

10. The antibody or antigen binding fragment thereof of claim 9, wherein said light chain constant region comprises a kappa light chain constant domain (CLK).

11. The antibody or antigen binding fragment thereof of claim 9, wherein said light chain constant region comprises a lambda light chain constant domain (CLX).

12. A compound comprising the antibody or antigen binding fragment thereof of claim 8, wherein the antibody is conjugated to a therapeutic agent via said engineered cysteine site.

13. The compound of claim 12, wherein the therapeutic agent is selected from the group consisting of: a cytotoxic agent, a cytostatic agent, a chemotherapeutic agent, a toxin, a radionuclide, a DNA, an RNA, an siRNA, a microRNA, a peptide nucleic acid, a non-natural amino acid, a peptide, an enzyme, a fluorescent tag, biotin, and any combination thereof.

14. The compound of claim 12, wherein the therapeutic agent is conjugated to the polypeptide or the antibody or antigen binding fragment thereof via a linker.

15. The compound of claim 14, wherein the linker is cleavable.

16. The compound of claim 14, where the linker comprises vc, mc, MalPeg6, m(H20)c, m(H20)cvc, or a combination thereof.

17. The compound of claim 14, wherein the linker comprises vc.

18. The compound of claim 12, wherein the therapeutic agent is an auristatin or tubulysin.

19. The compound of claim 12, wherein the therapeutic agent is an auristatin.

20. The compound claim 19, wherein the auristatin is selected from the group consisting of 0101, 8261, 6121, 8254, 6780, 0131, MMAD, MMAE, and MMAF.

21. A pharmaceutical composition comprising the compound of claim 12 and a pharmaceutically acceptable carrier.

22. A method of treating cancer, an autoimmune disease, an inflammatory disease, or an infectious disease, comprising administering to a subject in need thereof a therapeutically effective amount of the compound of claim 12.


SEQUENCE LISTING (84 sequences)

SEQ ID NO: 1 - protein, 120 aa, Artificial Sequence [sequence omitted]

SEQ ID NO: 2 - protein, 5 aa, Artificial Sequence
  Synthetic Construct
DTYIH

SEQ ID NO: 3 - protein, 17 aa, Artificial Sequence
  Synthetic Construct
RIYPTNGYTRYADSVKG

SEQ ID NO: 4 - protein, 11 aa, Artificial Sequence
  Synthetic Construct
WGGDGFYAMDY

SEQ ID NO: 5 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 107 aa, Artificial Sequence [sequence omitted]

SEQ ID NO: 8 - protein, 11 aa, Artificial Sequence
  Synthetic Construct
RASQDVNTAVA

SEQ ID NO: 9 - protein, 7 aa, Artificial Sequence
  Synthetic Construct
SASFLYS

SEQ ID NO: 10 - protein, 9 aa, Artificial Sequence
  Synthetic Construct
QQHYTTPPT

SEQ ID NO: 11 - protein, 107 aa, Artificial Sequence [sequence omitted]

SEQ ID NO: 12 - protein, 214 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPCPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPCPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSLSPG

SEQ ID NO: 26 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSLSPG

SEQ ID NO: 27 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPG

SEQ ID NO: 28 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPG

SEQ ID NO: 29 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSLSPG

SEQ ID NO: 32 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSLSPG

SEQ ID NO: 33 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 450 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSLSPG

SEQ ID NO: 38 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSLSPG

SEQ ID NO: 39 - protein, 329 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSCSPG

SEQ ID NO: 40 - protein, 449 aa, Artificial Sequence
  Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLDSDGSFFLYSKLTVDKSRWQQGNVFSCSVMHEALHNHYTQKSLSCSPG

SEQ ID NO: 41 - protein, 107 aa, Artificial Sequence
  Synthetic Construct
RTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 42 - protein, 213 aa, Artificial Sequence
  Synthetic Construct
DIQMTQSPSSLSASVGDRVTITCRASQDVNTAVAWYQQKPGKAPKLLIYSASFLYSGVPSRFSGSRSGTFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 43 - protein, 115 aa, Artificial Sequence
  Synthetic Construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGLLQGPP

SEQ ID NO: 44 - protein, 222 aa, Artificial Sequence
  Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGLLQGPP

SEQ ID NO: 45 - protein, 8 aa, Artificial Sequence
  Synthetic Construct
GGLLQGPP

SEQ ID NO: 46 - protein, 7 aa, Artificial Sequence
  Synthetic Construct
GGLLQGG

SEQ ID NO: 47 - protein, 5 aa, Artificial Sequence
  Synthetic Construct
LLQGA

SEQ ID NO: 48 - protein, 7 aa, Artificial Sequence
  Synthetic Construct
GGLLQGA

SEQ ID NO: 49 - protein, 7 aa, Artificial Sequence
  Synthetic Construct
LLQGPGK

SEQ ID NO: 50 - protein, 6 aa, Artificial Sequence
  Synthetic Construct
LLQGPG

SEQ ID NO: 51 - protein, 6 aa, Artificial Sequence
  Synthetic Construct
LLQGPA

SEQ ID NO: 52 - protein, 5 aa, Artificial Sequence
  Synthetic Construct
LLQGP

SEQ ID NO: 53 - protein, 4 aa, Artificial Sequence
  Synthetic Construct
LLQP

SEQ ID NO: 54 - protein, 6 aa, Artificial Sequence
  Synthetic Construct
LLQPGK

SEQ ID NO: 55 - protein, 8 aa, Artificial Sequence
  Synthetic Construct
LLQGAPGK

SEQ ID NO: 56 - protein, 7 aa, Artificial Sequence
  Synthetic Construct
LLQGAPG

SEQ ID NO: 57 - protein, 6 aa, Artificial Sequence
  Synthetic Construct
LLQGAP

SEQ ID NO: 58 - protein, 8 aa, Artificial Sequence
  Synthetic Construct
  MISC_FEATURE (4)..(4): Xaa can be Gly or Pro
  MISC_FEATURE (5)..(5): Xaa can be Ala, Gly, Pro, or absent
  MISC_FEATURE (6)..(6): Xaa can be Ala, Gly, Lys, Pro, or absent
  MISC_FEATURE (7)..(7): Xaa can be Gly, Lys or absent
  MISC_FEATURE (8)..(8): Xaa can be Lys or absent
LLQXXXXX

SEQ ID NO: 59 - protein, 8 aa, Artificial Sequence
  Synthetic Construct
  MISC_FEATURE (4)..(4): Xaa can be any naturally occurring amino acid
  MISC_FEATURE (5)..(5): Xaa can be any naturally occurring amino acids or absent
  MISC_FEATURE (6)..(6): Xaa can be any naturally occurring amino acids or absent
  MISC_FEATURE (7)..(7): Xaa can be any naturally occurring amino acids or absent
  MISC_FEATURE (8)..(8): Xaa can be any naturally occurring amino acids or absent
LLQXXXXX

SEQ ID NO: 60 - protein, 8 aa, Artificial Sequence
  Synthetic Construct
GGLLQGPP

SEQ ID NO: 61 - protein, 110 aa, Artificial Sequence
  Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 62 - protein, 217 aa, Artificial Sequence
  Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 107 aa, Artificial Sequence [sequence omitted]

SEQ ID NO: 64 - protein, 106 aa, Artificial Sequence
  Synthetic Construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 65 - protein, 116 aa, Artificial Sequence [sequence omitted]

SEQ ID NO: 66 - protein, 5 aa, Artificial Sequence
  Synthetic Construct
SFSMS

SEQ ID NO: 67 - protein, 7 aa, Artificial Sequence
  Synthetic Construct
GFTFSSF

SEQ ID NO: 68 - protein, 17 aa, Artificial Sequence
  Synthetic Construct
SISGSSGTTYYADSVKG

SEQ ID NO: 69 - protein, 6 aa, Artificial Sequence
  Synthetic Construct
SGSSGT

SEQ ID NO: 70 - protein, 7 aa, Artificial Sequence
  Synthetic Construct
PFPYFDY

SEQ ID NO: 71 - protein, 446 aa, Artificial Sequence
  Synthetic Construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 108 aa, Artificial Sequence [sequence omitted]

SEQ ID NO: 73 - protein, 12 aa, Artificial Sequence
  Synthetic Construct
RASQSVSSSFLA

SEQ ID NO: 74 - protein, 7 aa, Artificial Sequence
  Synthetic Construct
YASSRAT

SEQ ID NO: 75 - protein, 9 aa, Artificial Sequence
  Synthetic Construct
QQTGRIPPT

SEQ ID NO: 76 - protein, 215 aa, Artificial Sequence
  Synthetic Construct
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 445 aa, Artificial Sequence
  Synthetic Construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 215 aa, Artificial Sequence
  Synthetic Construct
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 79 - DNA, 987 bp, Artificial Sequence
  Synthetic Construct
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacatgcccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 80 - DNA, 321 bp, Artificial Sequence
  Synthetic Construct
cggaccgtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtcctgcgccgactacgagaagcacaaggtgtacgcctgcgaagtgacccaccagggcctgtccagccccgtgaccaagtccttcaaccggggcgagtgc

SEQ ID NO: 81 - DNA, 1335 bp, Artificial Sequence
  Synthetic Construct
gaggtgcagctgttggagtctgggggaggcttggtacagcctggggggtccctgagactctcctgtgcagcctctggattcacctttagcagtttttcgatgagctgggtccgccaggctccagggaaggggctggagtgggtctcatctattagtggtagttcgggtaccacatactacgcagactccgtgaagggccggttcaccatctccagagacaattccaagaacacgctgtatctgcaaatgaacagcctgagagccgaagacacggccgtatattactgtgcgaaaccgtttccgtattttgactactggggccagggaaccctggtcaccgtctcgagtgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacatgcccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 82 - DNA, 987 bp, Artificial Sequence
  Synthetic Construct
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacatgcccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggt

SEQ ID NO: 83 - DNA, 645 bp, Artificial Sequence
  Synthetic Construct
gaaattgtgttaacgcagtctccaggcaccctgtctttgtctccaggggaaagagccaccctctcctgcagggccagtcagagtgttagcagcagctttttagcctggtaccagcagaaacctggccaggctcccaggctcctcatctattatgcatccagcagggccactggcatcccagacaggttcagtggcagtgggtctgggacagacttcactctcaccatcagcagactggagcctgaagattttgcagtgtattactgtcagcagacgggtcgtattccgccgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagctgcgcagactacgagaaacacaaagtctacgcctgcgaagtcacccatcagggcctgagctcgcccgtcacaaagagcttcaacaggggagagtgt

SEQ ID NO: 84 - DNA, 321 bp, Artificial Sequence
  Synthetic Construct
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagctgcgcagactacgagaaacacaaagtctacgcctgcgaagtcacccatcagggcctgagctcgcccgtcacaaagagcttcaacaggggagagtgt